(19) United States
(12) Patent Application Publication
(10) Pub. No.: US 2011/0230419 A1
(43) Pub. Date: Sep. 22, 2011

(54) IAP BINDING COMPOUNDS

(75) Inventors: Mikkel Dybro Lundorf, Roskilde (DK); Kim Birkebaek Jenson, Redovre (DK); Sanne Schrøder Glad, Ballerup (DK); Alek Haahr Gouliaev, Sjaelland (DK); Anette Holtmann, Ballerup (DK); Michael Anders Godskesen, Vedbaek (DK)

(73) Assignee: Nuevolution A/S, Copenhagen (DK)

(21) Appl. No.: 12/999,267

(22) PCT Filed: Jun. 16, 2009
(86) PCT No.: PCT/DK09/50129

§ 371 Date: May 20, 2011

Related U.S. Application Data
(60) Provisional application No. 61/061,789, filed on Jun. 16, 2008.

(30) Foreign Application Priority Data
Jun. 16, 2008 (DK) PA 2008 00836

(51) Int. Cl. A61K 38/07; C07D 405/14; C07D 403/06; C07D 401/14; C07D 471/10; C07K 5/103; C07K 5/083; C07D 495/04; A61K 31/4025; A61K 31/4439; A61K 31/454; A61K 31/439; A61K 38/06; A61P 35/00

(52) U.S. Cl. 514/19.3; 548/524; 546/279.1; 546/208; 546/16; 530/330; 530/331; 546/187; 546/256; 548/304.1; 514/422; 514/343; 514/326; 514/278; 514/21.9

(57) ABSTRACT

The present invention relates to compounds of formula (I), or pharmaceutically acceptable salts, solvates or prodrugs thereof, that bind to Inhibitor of Apoptosis Proteins (IAPs). The compounds of the invention may be used as diagnostic and therapeutic agents in the treatment of proliferative diseases, such as cancer, for promoting apoptosis in proliferating cells, and for sensitizing cells to inducers of apoptosis. The present invention furthermore provides a polymeric compound of formulas (VI) or (VII), comprising either at least two monomeric units of compounds of formula (I), or at least one monomeric unit of a compound of formula (I) and an entity E. The present invention further relates to pharmaceutical compositions comprising said compounds of formulas (I), (VI), and (VII) and the use of said compounds in medicine.

### FIELD OF INVENTION

The present invention relates to compounds that bind to Inhibitor of Apoptosis Proteins (IAPs). The present invention further relates to pharmaceutical compositions comprising said compounds, the use of said compounds in medicine, preferably use of said compounds in methods for treating proliferative diseases, such as cancer.

### BACKGROUND OF INVENTION

Programmed cell death (apoptosis) is a key mechanism for the development and maintenance of a multicellular organism. The organism only remains healthy if there is an equilibrium between new formation and elimination of cells. The consequence of this equilibrium being out of control is pathological manifestations such as cancer, hepatitis, Parkinson's disease, stroke, cardiac infarction etc. Apoptosis plays a critical role in regulating cell number and in eliminating stressed or damaged cells from normal tissues. Indeed, the network of apoptotic signalling mechanisms inherent in most cell types provides a major barrier to the development and progression of human cancer. Since most commonly used radiation and chemo-therapies rely on activation of apoptotic pathways to kill cancer cells, tumor cells which are capable of evading programmed cell death often become resistant to treatment.

Apoptosis is executed primarily by activated caspases, a family of cysteine proteases with aspartate specificity in their substrates. Caspases are produced in cells as catalytically inactive zymogens and must be proteolytically processed to become active proteases during apoptosis. In normal surviving cells that have not received an apoptotic stimulus, most caspases remain inactive. Even if some caspases are aberrantly activated, their proteolytic activity can be fully inhibited by a family of evolutionarily conserved proteins called IAPs (inhibitors of apoptosis proteins). IAP proteins are central negative regulators of apoptosis and potently suppress apoptosis induced by a large variety of apoptotic stimuli, including chemotherapeutic agents, radiation, and immunotherapy in cancer cells. ((Deveraux & Reed, Genes Dev. 13: 239-252, 1999)(Salvesen et al., Nat. Rev. Mol. Cell. Biol. 3: 401 (2002)).

IAPs contain so-called BIR (baculoviral IAP repeat) domains. Several distinct mammalian IAPs including XIAP, survivin, and Livin/ML-IAP (Kasof &amp; Gomes, J. Biol. Chem. 276: 3238-3246, 2001; Vucic et al. Curr. Biol. 10: 1359-1366, 2000; Ashhab et al. FEBS Lett. 495: 56-60, 2001), have been identified, and they all exhibit anti-apoptotic activity in cell culture (Deveraux & Reed, 1999, supra). As IAPs are expressed in most cancer cells, they may directly contribute to tumor progression and subsequent resistance to drug treatment.

X-linked IAP (XIAP) is the most potent inhibitor in suppressing apoptosis among all of the IAP members (Holcik et al., Apoptosis 6: 253 (2001); LaCasse et al., Oncogene 17: 3247 (1998); Takahashi et al., J. Biol. Chem. 273: 7787 (1998); Deveraux et al., Nature 388: 300 (1997); Sun et al., Nature 401: 818 (1999); Deveraux et al., EMBO J. 18: 5242 (1999); Asselin et al., Cancer Res. 61: 1862 (2001)). XIAP plays a key role in the negative regulation of apoptosis in both the death receptor-mediated and the mitochondria-mediated pathways. XIAP functions as a potent endogenous apoptosis inhibitor by directly binding and potently inhibiting three members of the caspase family of enzymes, caspase-3, -7, and -9 (Takahashi et al., J. Biol. Chem. 273: 7787 (1998); Deveraux et al., Nature 388: 300 (1997); Sun et al., Nature 401: 818 (1999); Deveraux et al., EMBO J. 18: 5242 (1999); Asselin et al., Cancer Res. 61: 1862 (2001); Riedl et al., Cell 104: 791 (2001); Chai et al., Cell 104: 769 (2001); Huang et al., Cell 104: 781 (2001)). XIAP contains three baculovirus inhibitor of apoptosis repeat (BIR) domains as well as a C-terminal RING finger. The third BIR domain (BIR3) selectively targets caspase-9, the initiator caspase in the mitochondrial pathway, whereas the linker region between BIR1 and BIR2 inhibits both caspase-3 and caspase-7 (Salvesen et al., Nat. Rev. Mol. Cell. Biol. 3: 401 (2002)). While binding to XIAP prevents the activation of all three caspases, it is apparent that the interaction with caspase-9 is the most critical for its inhibition of apoptosis (Ekert et al., J. Cell Biol. 152: 483 (2001); Srinivasula et al., Nature 410: 112 (2001)). Because XIAP blocks apoptosis at the down-stream effector phase, a point where multiple signaling pathways converge, strategies targeting XIAP may prove to be especially effective to overcome resistance of cancer cells to apoptosis (Fulda et al., Nature Med. 8: 808 (2002); Arnt et al., J. Biol. C2em, 277: 44236 (2002)).

Smac/DIABLO (second mitochondria-derived activator of caspases) (Budiliardjo et al., Annu. Rev. Cell Dev. Biol. 15: 269 (1999); Du et al., Cell 102: 33 (2000); Verhagen et al. Cell 102: 43-53, 2000 is a potent endogenous inhibitor of IAPs. Smac interacts with all IAPs that have been examined to date, including XIAP, c-IAPI, c-IAP2, and survivin (Du et al., 2000, supra; Verhagen et al., 2000, supra). Thus, Smac appears to be a master regulator of apoptosis in mammals.

Similar to mammals, flies contain two IAPs, DIAP1 and DIAP2, that bind and inactivate several Drosophila caspases (Hay, Cell Death Differ. 7: 1045-1056, 2000). DIAP1 contains two BIR domains; the second BIR domain (BIR2) is necessary and sufficient to block cell death in many contexts. In Drosophila cells, the anti-death function of DIAP1 is removed by three pro-apoptotic proteins, Hid, Grim, and Reaper, which physically interact with the BIR2 domain of DIAP 1 and remove its inhibitory effect on caspases. Thus Hid, Grim, and Reaper represent the functional homologs of the mammalian protein Smac. However, except for their N-terminal 10 residues, Hid, Grim, and Reaper share no sequence homology with one another, and there is no apparent homology between the three Drosophila proteins and Smac.

WO 2004/007529 describes IAP binding compounds mimeticing the N-terminal tetrapeptide of IAP-binding proteins such as Smac, the compounds are either oligopeptides or peptidomimetics, and are described to be useful as therapeutic and diagnostic agents in the treatment of cell proliferative disorders. Specifically disclosed are peptidomimetic compounds having an oxazole in the peptide chain.

WO 2005/097791 describes oligopeptidic compounds that inhibit the binding of Smac to IAP. The compounds are described to be useful in the treatment of proliferative diseases including cancer.

WO 2005/069894 and WO 2006/010119 describe conformationally constrained oligopeptidic mimetics of Smac, useful for inhibiting IAP proteins and increasing the sensitivity of cells to inducers of apoptosis.

The use of peptides for in vivo administration as diagnostic or therapeutic agents is associated with certain disadvantages. These include short half-life due to proteolytic degradation in the body, low absorption through intestinal walls and potential immunogenic reactions, as well as expense involved in peptide synthesis. For these reasons, many current efforts in drug development focus on non-peptidic mimetics that mimic the structure and biological activity of bioactive peptides, but possess improved pharmacologic properties and are easier or less expensive to synthesize.

In connection with the Smac tetrapeptides and homologs described above, then, it would be a significant advance in the art to develop partial peptide or non-peptide mimetics of those molecules. It is therefore an object of the present invention to provide novel compounds that possess the IAP-binding and apoptosis-promoting bioactivity of the Smac peptides, while also having the improved properties associated with non-peptide mimetics, for use as diagnostic and therapeutic agents in the treatment of cancer. Furthermore, it is an object of the present invention to provide compounds having an improved IAP-binding (i.e. increased affinity and/or efficacy) compared to previously disclosed IAP-binding ligands, together with compounds having an improved IAP-binding and an improved stability profile, such as e.g. an improved proteolytic stability.

### SUMMARY OF INVENTION

The present invention provides compounds of formula (I)

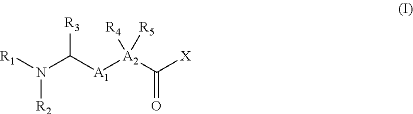

or a pharmaceutically acceptable salt, solvate or prodrug thereof,
wherein

### X is

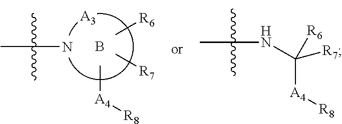

A₁ is selected from the group consisting of a single bond, —C(O)—, —NHC(O)—, —C(O)NH—, —SO₂—, —S(O)—, —C(S)— and —CHZ₁—;
Z₁ is selected from the group consisting of H, C₁-C₆ alkyl, C₂-C₆ alkenyl, C₂-C₆ alkynyl, —(CH₂)ₘ—C₃-C₁₀ cycloalkyl, —(CH₂)ₘ-aryl, —(CH₂)ₘ-heterocyclyl, and —(CH₂)ₘ-heteroaryl, —CH₂—F, —(CH₂)ₘ—O—C₁-C₆ alkyl, —(CH₂)ₘ—O—C₃-C₆ cycloalkyl, —(CH₂)ₘ—O-aryl, —(CH₂)ₘ—O-heterocyclyl, —(CH₂)ₘ—O-heteroaryl, —(CH₂)ₘ—NHC₃-C₆ alkyl, —(CH₂)ₘ—NHC₃-C₆ cycloalkyl, —(CH₂)ₘ—NH-aryl, —(CH₂)ₘ—NH-heterocyclyl and —(CH₂)ₘ—NH-heteroaryl.
A₂ is selected from the group consisting of cycloalkyl, aryl, heterocyclyl, heteroaryl, and —NHC(R⁴R⁵)—, wherein R⁴ and R⁵ independently are attached to cycloalkyl, aryl, heterocyclyl, or heteroaryl via any chemically feasible positions of the ring systems;
A₃ is a ring atom or moiety selected from the group consisting of C, S, O, N, —C(O)—, —NHC(O)—, and —C(O)NH—; when A3 is C it may optionally form a heterocyclic ring together with R⁴;
A₄ is a linker which is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂C(O)—, —CH₂CH₂NH—, —CH₂CH₂O—, —CH₂CH₂S—, —CH₂CH₂SO₂—, —CH₂C(O)CH₂—, —CH₂NHCH₂—, —CH₂OCH₂—, —CH₂SCH₂—, —CH₂SO₂CH₂—, —C(O)CH₂CH₂—, —NHCH₂CH₂—, —OCH₂CH₂—, —SCH₂CH₂—, —SO₂CH₂CH₂—, —CH₂C(O)NH—, —CH₂SO₂NH—, —CH₂NHC(O)—, —CH₂NHSO₂—, —C(O)NHCH₂—, —SO₂NHCH₂—, —NHC(O)CH₂—, —NHSO₂CH₂—, and —NHC(O)NH—;
B is selected from the group consisting of heterocyclic and heteroaromatic ring systems;
R¹ is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted;
R² is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-cycloalkyl, —(CH₂)₁₋₆-aryl, —(CH₂)₁₋₆-heterocyclyl, and —(C₂)₁₋₆-heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, wherein the heterocyclic ring optionally is substituted;
R³ is selected from the group consisting of H, hydroxy, halogen, C₁-C₆ alkyl, C₁-C₆ alkoxy, C₂-C₆ alkenyl, C₂-C₆ alkynyl, and C₃-C₁₀ cycloalkyl, wherein alkyl, alkenyl and alkynyl optionally are substituted;
R⁴ and R⁵ are each independently selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein any alkyl, alkenyl, alkynyl, cycloalkyl, aryl, heterocyclyl, and heteroaryl optionally are substituted;
Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, —N(R⁹)—SO₂—C₁-C₆ alkyl, —N(R⁹)—SO₂—(CH₂)_{q}—C₃-C₇ cycloalkyl, —N(R⁹)—SO₂—(CH₂)_{q}-aryl, —N(R⁹)—SO₂—(CH₂)_{q}-heterocyclyl, —N(R⁹)—SO₂—(CH₂)_{q}-heteroaryl, —SO₂—N(R¹⁰)(R¹¹), —N(R⁹)—C(O)—C₁-C₆ alkyl, —N(R⁹)—C(O)—(CH₂)_{q}—C₃-C₇ cycloalkyl, —N(R⁹)—C(O)—(CH₂)_{q}-aryl, —N(R⁹)—C(O)—(CH₂)_{q}-heterocyclyl, —N(R⁹)—C(O)—(CH₂)_{q}-heteroaryl, —C(O)—N(R¹⁰)(R¹¹), —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —CO(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)_{q}-heterocyclyl, and —O—C(O)—(CH₂)_{q}-heteroaryl, wherein any alkyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, wherein any heterocyclic ring optionally is substituted;
R⁶ and R⁷ are each independently selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein any alkyl, cycloalkyl, aryl, heterocyclyl, and heteroaryl optionally are substituted;
Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —NH(R⁹), —N(R⁹)—SO₂—C₁-C₆ alkyl, —N(R⁹)—SO₂—(CH₂)ᵣ—C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂)ᵣ—C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—N(R¹⁰)(R¹¹), —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)ᵣ-heteroaryl, —CO(O)—C₁-C₁₀ alkyl, —O—C(O)—(CH₂)ᵣ—C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl, wherein any alkyl, cycloalkyl, aryl, heterocyclyl, and heteroaryl optionally are substituted;
R⁸ is selected from the group consisting of C₃-C₁₀ cycloalkyl, aryl, heterocyclyl, heteroaryl, aryl-C₁-C₆ alkyl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted;
R⁹ is selected from the group consisting of H, C₁-C₆ alkyl, trifluoromethyl, trifluoroethyl, C₁-C₆ alkoxy, halogen-C₁-C₆ alkyl, —(CH₂)₀₋₂-aryl, —(CH₂)₀₋₂-heterocyclyl, and —(CH₂)₀₋₂-heteroaryl;
R¹⁰ and R¹¹ are each independently selected from the group consisting of H, C₁-C₄ alkyl, C₃-C₇ cycloalkyl, aryl, —(CH₂)₁₋₆—C₃-C₇ cycloalkyl, —(CH₂)₁₋₆-aryl, wherein alkyl, cycloalkyl, and aryl optionally are substituted, or R¹⁰ together with R¹¹ may form a heterocyclyl ring together with the nitrogen to which they are attached;
m is 0 or an integer from 1 to 5;
n is 0 or an integer from 1 to 6;
p is 0 or an integer from 1 to 6;
q is 0 or an integer from 1 to 6;
r is 0 or an integer from 1 to 6;
with the proviso that when A2 is —NHC(R⁴R⁵)—, then X is not

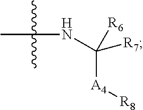

with the proviso that when A₁ is a single bond, A₂ is an oxazol ring, B is a pyrrolidinyl, R¹ and R² is H, R³ is selected from H or methyl, R⁴ and R⁵ is selected from H or methyl, and R⁸ is phenyl, 4-hydroxy-1-phenyl, or 3-indolyl, then at least one of R⁶ and R⁷ is different from H;
with the proviso that when A₁ is a —C(O)—, A₂ is —NHC(R⁴R⁵)—, B is pyrrolidinyl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, A4 is a single bond, B is pyrrolidinyl, R¹ is H, R² is methyl, R³ is methyl, one of R⁴ and R⁵ is cyclohexyl, and one of R⁶ and R⁷ is H, then the other of R⁶ and R⁷ is not benzyloxy; with the proviso that when A₁ is a —C(O)—, A₂ is —NHC(R⁴R⁵)—, B is octahydro-1H-pyrrolo[2,3-c]pyridin-1-yl, 7-oxooctahydro-1H-pyrrolo[2,3-c]pyridin-1-yl, octahydropyrrolo[2,3-c]azepin-1(2H)-yl, 8-oxooctahydropyrrolo[2,3-c]azepin-1(2H)-yl hexahydropyrrolo[3,4-b]pyrrol-1(2H)-yl, or 6-oxohexahydropyrrolo[3,4-b]pyrrol-1(2H)-yl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, B is 7-oxooctahydro-1H-pyrrolo[2,3-c]pyridinyl, A₄ is —CH₂CH₂—, R¹ is H, R² is methyl, R³ is methyl, one of R⁴ and R⁵ is isopropyl, R⁸ is phenyl, and one of R⁶ and R⁷ is H, then the other of R⁶ and R⁷ is not benzyloxy;
with the proviso that when A₁ is a —C(O)—, A₂ is —NHC(R⁴R⁵)—, A₄ contains a —NHC(O)-fragment or is —CH₂—O—, B is pyrrolidinyl, R¹ and R² is H, R³ is methyl, ethyl, propyl or isopropyl, and R⁴ forms a heterocyclic ring with A3, then at least one of R⁶ and R⁷ is not H; and
with the proviso that when A₁ is a —C(O)—, A₂ is —NHC(R⁴R⁵)—, A₄ contains a —NHC(O)-fragment, B is pyrrolidinyl, R³ is methyl, ethyl, propyl or isopropyl, and R⁴ forms a heterocyclic ring with A3, then at least one of R⁶ and R⁷ is not H.

The present invention provides furthermore a polymeric compound of formula (VI)

Y-(L)ₘ-[Y-(L)ₘ]ₙ-Y (VI)

or a pharmaceutically acceptable salt, solvate or prodrug thereof,
wherein
Y is a monomeric unit of formula (I), wherein the first and the second or further monomeric units are the same or different and independently are selected from the compounds as defined in any of claims 1-144;
L is the same or different and is a covalent linker, linking any part of one monomeric unit of formula (I), to any part of a second or further monomeric unit of formula (I);
m is an integer of 1 to 4; and
n is an integer of 0 to 5.

The present invention provides furthermore a compound of formula (VII)

Z-Lₘ-E (VII)

or a pharmaceutically acceptable salt, solvate or prodrug thereof,
wherein
Z is a compound of formula (I) as defined in any of claims 1-144 or a polymeric compound of formula (VI) as defined in any of claims 145-147;
L is a linker linking any part of Z to any part of E;
E is an entity selected from the group consisting of an affinity tag, such as e.g. a hexahistidine tag or biotin, a dye, such as e.g. fluorescein, an oligonucleotide, a protein, such as e.g. an antibody or biotin-binding protein, and a solid support; and m is an integer of 1 to 4.

The present inventors have found that compounds of formula (I), including subformulas II, IIa, IIb, III, IIIa, IIIb, IV, V, which are described herein below, and compounds of formula (VI) and (VII) bind to Inhibitor of Apoptosis Proteins (IAPs), and therefore are useful for treatment of proliferative diseases, for promoting apoptosis in proliferating cells, and for sensitizing cells to inducers of apoptosis. Furthermore the present invention relates to use of said compounds for the preparation of a medicament, preferably a medicament for the treatment of proliferative diseases, and more preferably for the treatment of cancer.

The present invention further provides pharmaceutical composition comprising compounds of formula (I), (VI) and (VII), and optionally one or more pharmaceutically acceptable excipients, diluents or carriers. Preferably for the treatment of proliferative diseases, and more preferably for the treatment of cancer.

Furthermore, the present invention provides a method of treating a proliferative disease in a subject; said method comprises administering to said subject a therapeutically effective amount of a compound according to the invention to a subject in need of such treatment.

### DETAILED DESCRIPTION OF THE INVENTION

A first aspect of the present invention relates to compounds of formula (I)

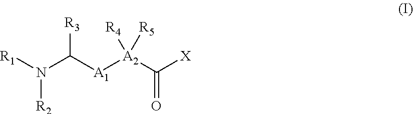

or a pharmaceutically acceptable salt, solvate or prodrug thereof,
wherein

### X is

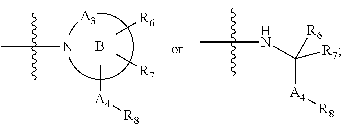

A₁ is selected from the group consisting of a single bond, —C(O)—, —NHC(O)—, —C(O)NH—, —SO₂—, —S(O)—, —C(S)— and —CHZ₁—;
Z₁ is selected from the group consisting of H—C₁-C₆ alkyl, C₂-C₆ alkenyl, C₂-C₆ alkynyl, —(CH₂)ₘ—C₃-C₁₀ cycloalkyl, —(CH₂)ₘ-aryl, —(CH₂)ₘ-heterocyclyl, and —(CH₂)ₘ-heteroaryl, —CH₂—F, —(CH₂)ₘ—O—C₁-C₆ alkyl, —(CH₂)ₘ—O—C₃-C₆ cycloalkyl, —(CH₂)ₘ—O-aryl, —(CH₂)ₘ—O-heterocyclyl, —(CH₂)ₘ—O-heteroaryl, —(CH₂)ₘ—NHC₁-C₆ alkyl, —(CH₂)ₘ—NHC₃-C₆ cycloalkyl, —(CH₂)ₘ—NH-aryl, —(CH₂)ₘ—NH-heterocyclyl and —(CH₂)ₘ—NH-heteroaryl.
A₂ is selected from the group consisting of cycloalkyl, aryl, heterocyclyl, heteroaryl, and —NHC(R⁴R⁵)—, wherein R⁴ and R⁵ independently are attached to cycloalkyl, aryl, heterocyclyl, or heteroaryl via any chemically feasible positions of the ring systems;
A₃ is a ring atom or moiety selected from the group consisting of C, S, O, N, —C(O)—, —NHC(O)—, and —C(O)NH—; when A3 is C it may optionally form a heterocyclic ring together with R⁴;
A₄ is a linker which is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂C(O)—, —CH₂CH₂NH—, —CH₂CH₂O—, —CH₂CH₂S—, —CH₂CH₂SO₂—, —CH₂C(O)CH₂—, —CH₂NHCH₂—, —CH₂OCH₂—, —CH₂SCH₂—, —CH₂SO₂CH₂—, —C(O)CH₂CH₂—, —NHCH₂CH₂—, —OCH₂CH₂—, —SCH₂CH₂—, —SO₂CH₂CH₂—, —CH₂C(O)NH—, —CH₂SO₂NH—, —CH₂NHC(O)—, —CH₂NHSO₂—, —C(O)NHCH₂—, —SO₂NHCH₂—, —NHC(O)CH₂—, —NHSO₂CH₂—, and —NHC(O)NH—;
B is selected from the group consisting of heterocyclic and heteroaromatic ring systems;
R¹ is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-aryl, —(CH₂)₁₋₆-heterocyclyl, and —(C₂)₁₋₆-heteroaryl, wherein any alkyl, alkenyl, alkynyl, cycloalkyl, aryl, heterocyclyl, and heteroaryl optionally are substituted;
R² is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-cycloalkyl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, wherein the heterocyclic ring optionally is substituted;
R³ is selected from the group consisting of H, hydroxy, halogen, C₁-C₆ alkyl, C₁-C₆ alkoxy, C₂-C₆ alkenyl, C₂-C₆ alkynyl, and C₃-C₁₀ cycloalkyl, wherein alkyl, alkenyl and alkynyl optionally are substituted;
R⁴ and R⁵ are each independently selected from the group consisting of H, C₁-C₆ alkyl, 1-C₆ alkoxy, C₂-C₆ alkenyl, C₂-C₆ alkynyl, C₃-C₁₀, cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein any alkyl, alkenyl, alkynyl, cycloalkyl, aryl, heterocyclyl, and heteroaryl optionally are substituted;
Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, —N(R⁹)—SO₂—C₁-C₆ alkyl, —N(R⁹)—SO₂—(CH₂)_{q}—C₃-C₇ cycloalkyl, —N(R⁹)—SO₂—(CH₂)_{q}-aryl, —N(R⁹)—SO₂—(CH₂)_{q}-heterocyclyl, —N(R⁹)—SO₂—(CH₂)_{q}-heteroaryl, —SO₂—N(R¹⁰)(R¹¹), —N(R⁹)—C(O)—C₁-C₆ alkyl, —N(R⁹)—C(O)—(CH₂)_{q}—C₃-C₇ cycloalkyl, —N(R⁹)—C(O)—(CH₂)_{q}-aryl, —N(R⁹)—C(O)—(CH₂)_{q}-heterocyclyl, —N(R⁹)—C(O)—(CH₂)_{q}-heteroaryl, —C(O)—N(R¹⁰)(R¹¹), —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —CO(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)_{q}-heterocyclyl, and —O—C(O)—(CH₂)_{q}-heteroaryl, wherein any alkyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, wherein any heterocyclic ring optionally is substituted;
R⁶ and R⁷ are each independently selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein any alkyl, cycloalkyl, aryl, heterocyclyl, and heteroaryl optionally are substituted;
Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —NH(R⁹), —N(R⁹)—SO₂—C₁-C₆ alkyl, —N(R⁹)—SO₂—(CH₂)ᵣ—C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂), —C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—) N(R¹⁰)(R¹¹), —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)ᵣ-heteroaryl, —OC(O)—C₁-C₁₀ alkyl, —O—C(O)—(CH₂), —C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl, wherein any alkyl, cycloalkyl, aryl, heterocyclyl, and heteroaryl optionally are substituted;
R⁸ is selected from the group consisting of C₃-C₁₀ cycloalkyl, aryl, heterocyclyl, heteroaryl, aryl-C₁-C₆ alkyl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted;
R⁹ is selected from the group consisting of H, C₁-C₆ alkyl, trifluoromethyl, trifluoroethyl, C₁-C₆ alkoxy, halogen-C₁-C₆ alkyl, —(CH₂)₀₋₂-aryl, —(CH₂)₀₋₂-heterocyclyl, and —(CH₂)₀₋₂-heteroaryl;
R¹⁰ and R¹¹ are each independently selected from the group consisting of H, C₁-C₄ alkyl, C₃-C₇ cycloalkyl, aryl, —(C₂)₁₋₆—C₃-C₇ cycloalkyl, —(C₂)₁₋₆-aryl, wherein alkyl, cycloalkyl, and aryl optionally are substituted, or R¹⁰ together with R¹¹ may form a heterocyclyl ring together with the nitrogen to which they are attached;
m is 0 or an integer from 1 to 5;
n is 0 or an integer from 1 to 6;
p is 0 or an integer from 1 to 6;
q is 0 or an integer from 1 to 6;
r is 0 or an integer from 1 to 6;
with the proviso that when A₂ is —NHC(R⁴R⁵)—, then X is not

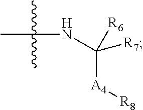

with the proviso that when A₁ is a single bond, A₂ is an oxazol ring, B is a pyrrolidinyl, R¹ and R² is H, R³ is selected from H or methyl, R⁴ and R⁵ is selected from H or methyl, and R⁸ is phenyl, 4-hydroxy-1-phenyl, or 3-indolyl, then at least one of R⁶ and R⁷ is different from H;
with the proviso that when A₁ is a —C(O)—, A2 is —NHC(R⁴R⁵)—, B is pyrrolidinyl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A2 is —NHC(R⁴R⁵)—, A₄ is a single bond, B is pyrrolidinyl, R¹ is H, R² is methyl, R³ is methyl, one of R⁴ and R⁵ is cyclohexyl, and one of R⁶ and R⁷ is H, then the other of R⁶ and R⁷ is not benzyloxy;
with the proviso that when A₁ is a —C(O)—, A₂ is —NHC(R⁴R⁵)—, B is octahydro-1H-pyrrolo[2,3-c]pyridin-1-yl, 7-oxooctahydro-1H-pyrrolo[2,3-c]pyridin-1-yl, octahydropyrrolo[2,3-c]azepin-1(2H)-yl, 8-oxooctahydropyrrolo[2,3-c]azepin-1(2H)-yl hexahydropyrrolo[3,4-b]pyrrol-1(2H)-yl, or 6-oxohexahydropyrrolo[3,4-b]pyrrol-1(2H)-yl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, B is 7-oxooctahydro-1H-pyrrolo[2,3-c]pyridinyl, A₄ is —CH₂CH₂—, R¹ is H, R² is methyl, R³ is methyl, one of R⁴ and R⁵ is isopropyl, R⁸ is phenyl, and one of R⁶ and R⁷ is H, then the other of R⁶ and R⁷ is not benzyloxy;
with the proviso that when A₁ is a —C(O)—, A₂ is —NHC(R⁴R⁵)—, A₄ contains a —NHC(O)-fragment or is —CH₂—O—, B is pyrrolidinyl, R¹ and R² is H, R³ is methyl, ethyl, propyl or isopropyl, and R⁴ forms a heterocyclic ring with A3, then at least one of R⁶ and R⁷ is not H; and
with the proviso that when A₁ is a —C(O)—, A₂ is —NHC(R⁴R⁵)—, A₄ contains a —NHC(O)-fragment, B is pyrrolidinyl, R³ is methyl, ethyl, propyl or isopropyl, and R⁴ forms a heterocyclic ring with A3, then at least one of R⁶ and R⁷ is not H.

The term “alkyl”, as used herein, unless otherwise indicated, includes saturated monovalent hydrocarbon radicals having straight or branched moieties. Examples of alkyl moieties include, but are not limited to, methyl, ethyl, propyl, isopropyl, n-butyl, iso-butyl, sec-butyl, tert-butyl, and neopentyl. Alkyl is preferably C₁-C₆ alkyl, i.e. groups containing from 1 to 6 carbon atoms, and for some embodiments of the present invention, more preferably C₁-C₄ alkyl.

The term “alkenyl”, as used herein, unless otherwise indicated, includes alkyl moieties having at least one carbon-carbon double bond wherein alkyl is as defined above. Examples of alkenyl include, but are not limited to, ethenyl, propenyl, 1-butenyl, and 2-butenyl. Alkenyl is preferably C₂-C₆ alkyl, i.e. groups containing from 2 to 6 carbon atoms, and for some embodiments of the present invention, more preferably C₁-C₄ alkenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl, 2-propynyl, 1-butynyl, and 2-butynyl.

The term “alkoxy”, as used herein, means an —O-alkyl group wherein “alkyl” is as defined above. Alkoxy furthermore refers to polyethers such as —O—(CH₂)₁₋₆—O—CH₃ Examples include, but are not limited to methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy.

The term “cycloalkyl”, as used herein, unless otherwise indicated, includes non-aromatic saturated cyclic alkyl moieties wherein alkyl is as defined above. Cycloalkyl furthermore includes saturated carbocyclic groups consisting of two or more rings, such as spiro ring systems, fused ring systems and bridged ring systems, wherein said rings share one or two carbon atoms. Cycloalkyl also include groups that are substituted with one or more oxo moieties. Examples of cycloalkyl include, but are not limited to, cyclopropyl, cyclobutyl, cyclopentyl, cyclohexyl, cycloheptyl, cyclooclyl, cyclononyl, bicyclo-[3.1.0]-hexyl, norbornyl, spiro[4.5]decyl, spiro[4.4]nonyl, spiro[4.3]octyl, and spiro[4.2]heptyl. Examples of cycloalkyl with oxo moieties are oxocyclopentyl, and oxocyclobutyl. Cycloalkyl is preferably C₃-C₁₀ cycloalkyl, i.e. cycloalkyl groups containing from 3 to 10 carbon atoms, and more preferably C₃-C₇ cycloalkyl.

The term “aryl”, as used herein, unless otherwise indicated, includes six- and ten-membered carbocyclic aromatic radicals derived from an aromatic hydrocarbon by removal of a hydrogen atom. Aryl furthermore includes bicyclic ring systems. Examples of aryl include, but are not limited to phenyl, naphthyl, indenyl, and fluorenyl. Preferred “aryl” is phenyl, naphthyl or indanyl, unless otherwise stated.

The terms “heterocyclic” and “heterocyclyl”, as used herein, refer to non-aromatic cyclic groups containing one or more heteroatoms selected from O, S and N. Preferably from one to four heteroatoms, more preferably from one to three heteroatoms. Furthermore, heterocyclic and heterocyclyl includes two-ringed cyclic groups, such as spiro ring systems, fused ring systems and bridged ring systems, wherein said rings share one or two atoms, and wherein at least one of the rings contains a heteroatom selected from O, S, and N. Heterocyclic and heterocyclyl groups also include groups that are substituted with one or more oxo moieties. Examples of heterocyclyl include, but are not limited to aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholinyl, thiomorpholinyl, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 2,8-diazaspiro[4.5]decanyl, 8-azaspiro[4.5]decanyl, quinolizinyl, quinuclidinyl, 1,4-dioxaspiro[4.5]decyl, 1,4-dioxaspiro[4.4]nonyl, 1,4-dioxaspiro[4.3]octyl, 1,4-dioxaspiro[4.2]heptyl, 2-oxopiperazinyl, and 2-oxopiperidinyl.

Examples of monocyclic heterocyclic group containing nitrogen atom(s) as the sole ring-member heteroatom include, but are not limited to, azetidinyl, 1,2-dihydroazetyl, pyrrolinyl, pyrrolidinyl, piperidyl, and piperazinyl groups.

Examples of monocyclic heterocyclic group containing oxygen atom(s) as sole ring-member heteroatom include, but are not limited to, pyranyl, tetrahydropyranyl, 1,3-dioxolyl, 1,3-dioxanyl, and 1,4-dioxanyl groups.

Examples of monocyclic heterocyclic group containing both nitrogen and oxygen atoms as sole ring-member heteroatoms include, but are not limited to oxazolidinyl, isoxazolidinyl, oxadiazolidinyl, and morpholinyl group.

Examples of monocyclic heterocyclic group containing sulfur atom(s) as sole ring-member heteroatom include, but are not limited to a thienyl group.

Examples of monocyclic heterocyclic group containing both nitrogen and sulfur atoms as sole ring-member heteroatoms include, but are not limited to thiazolidinyl, isothiazolidinyl, thiadiazolidinyl, and thiomorpholinyl group.

Examples of monocyclic heterocyclic group containing both oxygen and sulfur atoms as sole ring-member heteroatoms include, but are not limited to, a thioxanyl group.

The term “Heteroaryl”, as used herein, refers to aromatic groups containing one or more heteroatoms selected from O, S, and N, preferably from one to four heteroatoms, and more preferably from one to three heteroatoms. Heteroaryl furthermore includes multicyclic groups, wherein at least one ring of the group is aromatic, and at least on of the rings contains a heteroatom selected from O, S, and N. Heteroaryl also include ring systems substituted with one or more oxo moieties. Examples of heteroaryl groups include, but are not limited to pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, azaindolyl, imidazolyl, pyrazolyl, pyridyl, pyridazinyl, pyrazinyl, pyrimidinyl, tetrazolyl, pyrazolinyl, and pyrazolidinyl.

Bicyclic heteroaromatic group include, for example, bicyclic heteroaromatic groups comprising a condensed or bridged ring. Examples of bicyclic heteroaromatic groups containing nitrogen atom(s) as sole ring-member heteroatom include, but are not limited to, indolyl, indolinyl, isoindolyl, indolizinyl, benzimidazolyl, benzotriazolyl, indazolyl, quinolyl, tetrahydroquinolinyl, tetrahydroisoquinolinyl, quinolizinyl, isoquinolyl, phthalazinyl, naphthyridinyl, quinoxalinyl, dihydroquinoxalinyl, quinazolinyl, cinnolinyl, and 2,3-dihydrobenzopyrrolyl groups. Examples of bicyclic heteroaromatic groups containing oxygen atom(s) as sole ring-member heteroatom include, but are not limited to, benzofuranyl, isobenzofuranyl, chromenyl, isochromanyl, benzo-1,3-dioxolyl, benzo-1,4-dioxanyl, and 2,3-dihydrobenzofuranyl groups. Examples of bicyclic heteroaromatic groups containing sulfur atom(s) as sole ring-member heteroatom include, but are not limited to, benzothienyl and 2,3-dihydrobenzothienyl groups. Examples of bicyclic heteroaromatic groups containing nitrogen and oxygen atom(s) as sole ring-member heteroatoms include, but are not limited to, benzomorpholinyl and benzomorpholonyl groups. Examples of bicyclic heteroaromatic groups containing nitrogen and sulfur atom(s) as sole ring-member heteroatoms include, but are not limited to, a benzothiazolyl group.

The point of attachment of any cycloalkyl, aryl, heterocyclyl, and heteroaryl group may be via any atom in the ring system including (where appropriate) a heteroatom. Accordingly, in relation to embodiments of compounds of formula I, wherein A2 is ring system, such as a cycloalkyl, aryl, heterocyclyl, and heteroaryl group, A2 is attached to A1, R⁴, R⁵ and the carbonyl group of formula (I), via any chemically feasible positions of the ring systems. Likewise, when any cycloalkyl, aryl, heterocyclyl, and heteroaryl are mentioned herein by there prefix -yl name, without identification of the attachment point by number, such as for example pyridyl, then it refers to any attachment point for the relevant ring system, such as for example pyridin-2-yl, pyridin-3-yl, and pyridin-4-yl (i.e. 2-pyridyl, 3-pyridyl and 4-pyridyl).

The term “4 membered”, “5 membered”, “6 membered” and “7 membered”, as used herein, refers to ring systems having 4, 5, 6 or 7 non-hydrogen ring atoms, respectively. Examples of 4 membered rings include, but are not limited to, cyclobutane, azetidine, oxetane, oxetane, and thietane.

Examples of 5 membered rings include, but are not limited to, cyclopentane, pyrrolidine, tetrahydrofuran, tetrahydrothiophene, and cyclopenta-1,3-diene.

Examples of 6 membered rings include, but are not limited to, cyclohexane, piperidine, tetrahydro-2H-pyran, tetrahydro-2H-thiopyran, piperazine, morpholine, cyclohexene, and pyridine. Examples of 7 membered rings include, but are not limited to, cycloheptane, azepane, oxepane, and thiepane.

When moieties like aryl-C₁-C₆ alkyl, or heteroaryl-C₁-C₆ alkyl, are used herein it refers to e.g. an aryl being attached to the remaining part of formula (I) via a C₁-C₆ alkyl group. Accordingly, phenylethyl is intended to mean a phenyl group being attached via an ethyl group.

The term “optionally substituted”, as used herein, refers to the optional possibility that a hydrogen atom of a moiety, such as e.g., alkyl, alkenyl, alkynyl, cycloalkyl, aryl, heterocyclyl, heterocyclic ring, and heteroaryl moiety, can be substituted with one or more substituents. The term “substituted”, as used herein, refers to that a hydrogen atom is substituted with one or more substituents. Likewise “unsubstituted” is intended to mean that hydrogen is the only substituent at said moieties. Preferably the one or more substituents are 1 to 4 substituents, more preferably 1 to 3 substituents, even more preferably 1 to 2 substituents, and most preferably 1 substituent, unless otherwise stated. For the compounds of formula I, unless otherwise stated, substituents are selected from the group consisting of halogen, hydroxy, —CN, —NO₂, —NH₂, —SH, —C(O)—NH₂, —COOH, trifluoromethyl, trifluoroethyl, C₁-C₆ alkyl, substituted C₁-C₆ alkyl, C₁-C₆ alkoxy, C₂-C₆ alkenyl, C₂-C₆ alkynyl, —C(O)—C₁-C₆ alkyl, —C(O)—C₃-C₁₀ cycloalkyl, —C(O)-aryl, —C(O)-heterocyclyl, —C(O)-heteroaryl, —C(O)—NH—C₁-C₆ alkyl, —C(O)—NH(C₃-C₁₀ cycloalkyl), —C(O)—NH-aryl, —C(O)—NH-heterocyclyl, —C(O)—NH-heteroaryl, —C(O)—N(C₁-C₆ alkyl)(C₁-C₆ alkyl), —C(O)—N(C₁-C₆ alkyl)(C₃-C₁₀ cycloalkyl), —C(O)—N(C₁-C₆ alkyl)(aryl), —C(O)—N(C₁-C₆ alkyl)(heterocyclyl), —C(O)—N(C₁-C₆ alkyl)(heteroaryl), —NH—C(O)—C₁-C₆ alkyl, —NH—C(O)—(C₃-C₁₀ cycloalkyl), —NH—C(O)-aryl, —NH—C(O)-heterocyclyl, —NH—C(O)-heteroaryl, —N(C₁-C₆ alkyl)C(O)—(C₁-C₆ alkyl), —N(C₁-C₆ alkyl)C(O)—(C₃-C₁₀ cycloalkyl), —N(C₁-C₆ alkyl)C(O)-(aryl), —N(C₁-C₆ alkyl)C(O)-(heterocyclyl), —N(C₁-C₆ alkyl)C(O)-(heteroaryl), —N(C₃-C₁₀ cycloalkyl)C(O)—(C₁-C₆ alkyl), —N(C₃-C₁₀ cycloalkyl)C(O)—(C₃-C₁₀ cycloalkyl), —N(C₃-C₁₀ cycloalkyl)C(O)-(aryl), —N(C₃-C₁₀ cycloalkyl)C(O)-(heterocyclyl), —N(C₃-C₁₀ cycloalkyl)C(O)-(heteroaryl), —N(C₁-C₆ alkyl)(C₁-C₆ alkyl), —N(C₁-C₆ alkyl)(C₃-C₁₀ cycloalkyl), —N(C₁-C₆ alkyl)(aryl), —N(C₁-C₆ alkyl)(heterocyclyl), —N(C₁-C₆ alkyl)(heteroaryl), —SO₂—C₁-C₆ alkyl, —SO₂—C₃-C₁₀ cycloalkyl, —SO₂-aryl, —SO₂-heterocyclyl, —SO₂-heteroaryl, —NH—SO₂—C₁-C₆ alkyl, —NH—SO₂—(C₃-C₁₀ cycloalkyl), —NH—SO₂-aryl, —NH—SO₂-heterocyclyl, —NH—SO₂-heteroaryl, —N(C₁-C₆ alkyl)-SO₂—(C₁-C₆ alkyl), —N(C₁-C₆ alkyl)-SO₂—(C₃-C₁₀ cycloalkyl), —N(C₁-C₆ alkyl)-SO₂— (aryl), —N(C₁-C₆ alkyl)-SO₂-(heterocyclyl), —N(C₁-C₆ alkyl)-SO₂— (heteroaryl), —N(C₃-C₁₀ cycloalkyl)-SO₂—(C₁-C₆ alkyl), —N(C₃-C₁₀ cycloalkyl)-SO₂—(C₃-C₁₀ cycloalkyl), —N(C₃-C₁₀ cycloalkyl)-SO₂— (aryl), —N(C₃-C₁₀ cycloalkyl)-SO₂— (heterocyclyl), —N(C₃-C₁₀ cycloalkyl)-SO₂— (heteroaryl), —SO₂—NH—C₁-C₆ alkyl, —SO₂—NH(C₃-C₁₀ cycloalkyl), —SO₂—NH-aryl, —SO₂—NH-heterocyclyl, —SO₂—NH-heteroaryl, —SO₂—N(C₁-C₆ alkyl)(C₁-C₆ alkyl), —SO₂—N(C₁-C₆ alkyl)(C₃-C₁₀ cycloalkyl), —SO₂—N(C₁-C₆ alkyl)(aryl), —SO₂—N(C₁-C₆ alkyl)(heterocyclyl), —SO₂—N(C₁-C₆ alkyl)(heteroaryl), —S(O)—C₁-C₆ alkyl, —O—C(O)—C₁-C₆ alkyl, —C(O)—O—C₁-C₆ alkyl, C₃-C₁₀ cycloalkyl, aryl, heterocyclyl, and heteroaryl.

In a preferred embodiment of the invention the substituents for compounds of formula (I), unless otherwise stated, are selected from the group consisting of fluoro, chloro, hydroxy, —CN, —NO₂, —NH₂, —SH, —C(O)—NH₂, —COOH, trifluoromethyl, methylsulfinyl, methylsulfonyl, —O—C(O)-methyl, —O—C(O)-ethyl, —C(O)—O-methyl, —C(O)—O-ethyl, C₁-C₆ alkyl, substituted C₁-C₆ alkyl, C₁-C₆ alkoxy, C₂-C₆ alkenyl, C₂-C₆ alkynyl, —C(O)—C₁-C₃ alkyl, —C(O)—C₃-C₆ cycloalkyl, —C(O)-aryl, —C(O)-heterocyclyl, —C(O)-heteroaryl, —C(O)—NH—C₁-C₃ alkyl, —C(O)—NH(C₃-C₆ cycloalkyl), —C(O)—NH-aryl, —C(O)—NH-heterocyclyl, —C(O)—NH-heteroaryl, —C(O)—N(C₁-C₃ alkyl)(C₁-C₃ alkyl), —C(O)—N(C₁-C₃ alkyl)(C₃-C₆ cycloalkyl), —C(O)—N(C₁-C₃ alkyl)(aryl), —C(O)—N(C₁-C₃ alkyl)(heterocyclyl), —C(O)—N(C₁-C₃ alkyl)(heteroaryl), —NH—C(O)—C₁-C₃ alkyl, —NH—C(O)—(C₃-C₆ cycloalkyl), —NH—C(O)-aryl, —NH—C(O)-heterocyclyl, —NH—C(O)-heteroaryl, —N(C₁-C₃ alkyl)C(O)—(C₁-C₃ alkyl), —N(C₁-C₃ alkyl)C(O)—(C₃-C₆ cycloalkyl), —N(C₁-C₃ alkyl)C(O)-(aryl), —N(C₁-C₃ alkyl)C(O)-(heterocyclyl), —N(C₁-C₃ alkyl)C(O)-(heteroaryl), —N(C₁-C₃ alkyl)(C₁-C₃ alkyl), —N(C₁-C₃ alkyl)(C₃-C₆ cycloalkyl), —N(C₁-C₃ alkyl)(aryl), —N(C₁-C₃ alkyl)(heterocyclyl), —N(C₁-C₃ alkyl)(heteroaryl), —SO₂—C₁-C₃ alkyl, —SO₂—C₃-C₆ cycloalkyl, —SO₂-aryl, —SO₂-heterocyclyl, —SO₂-heteroaryl, —NH—SO₂—C₁-C₃ alkyl, —NH—SO₂—(C₃-C₆ cycloalkyl), —NH—SO₂-aryl, —NH—SO₂-heterocyclyl, —NH—SO₂-heteroaryl, —N(C₁-C₃ alkyl)-SO₂—(C₁-C₃ alkyl), —N(C₁-C₃ alkyl)-SO₂—(C₃-C₆ cycloalkyl), —N(C₁-C₃ alkyl)-SO₂— (aryl), —N(C₁-C₃ alkyl)-SO₂-(heterocyclyl), —N(C₁-C₃ alkyl)-SO₂— (heteroaryl), —SO₂—NH—C₁-C₃ alkyl, —SO₂—NH(C₃-C₆ cycloalkyl), —SO₂—NH-aryl, —SO₂—NH-heterocyclyl, —SO₂—NH-heteroaryl, —SO₂—N(C₁-C₃ alkyl)(C₁-C₃ alkyl), —SO₂—N(C₁-C₃ alkyl)(C₃-C₆ cycloalkyl), —SO₂—N(C₁-C₃ alkyl)(aryl), —SO₂—N(C₁-C₃ alkyl)(heterocyclyl), —SO₂—N(C₁-C₃ alkyl)(heteroaryl), C₃-C₁₀ cycloalkyl, aryl, heterocyclyl, and heteroaryl; more preferably, the substituents are selected from the group consisting of fluoro, chloro, hydroxy, —CN, —NO₂, —NH₂, —SH, —C(O)—NH₂, —COOH, methylsulfinyl, methylsulfonyl, C₁-C₆ alkyl, C₁-C₆ alkoxy, C₂-C₆ alkenyl, and C₂-C₆ alkynyl; even more preferably, the substituents are selected from the group consisting of fluoro, chloro, hydroxy, —CN, —NO₂, —NH₂, —SH, —C(O)—NH₂, —COOH, methylsulfinyl, methylsulfonyl, methyl, ethyl, propyl, isopropyl, tert-butyl, methoxy, and ethoxy.

The term “Halogen”, as used herein, includes fluoro, chloro, bromo and iodo; preferably fluoro and chloro.

In a preferred embodiment of formula (I) A1 is selected from the group consisting of a single bond, —C(O)—, SO₂, —S(O)—, and —CHZ₁—; more preferably A1 is selected from the group consisting of a single bond, —C(O)— and —CHZ₁—, and even more preferably A1 may be a single bond. Alternatively A1 may be —C(O)—. In this embodiment Z₁ may be as described herein above or preferably be selected from the group consisting of H, C₁-C₄ alkyl, —CH₂—F, —CH₂—C₃-C₆ cycloalkyl, —CH₂-aryl, —CH₂-heterocyclyl, —CH₂-heteroaryl, —CH₂—OC₁-C₆ alkyl, —CH₂—OC₃-C₆ cycloalkyl, —CH₂—O-aryl, —CH₂—O-heterocyclyl, —CH₂—O-heteroaryl, —CH₂—NHC₃-C₆ alkyl, —CH₂—NHC₃-C₆ cycloalkyl, —CH₂—NH-aryl, —CH₂—NH-heterocyclyl, and —CH₂—NH-heteroaryl, wherein any alkyl, cycloalkyl, aryl, heterocyclyl or heteroaryl optionally may be substituted by one or more substituents as defined herein above.

In a preferred embodiment of formula (I) A2 is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems. Any of these cycloalkyl, aryl, heterocyclyl, and heteroaryl may each independently be as defined herein above, and may optionally be substituted by one or more substituents as defined herein above, or more preferably be selected from the group consisting of cyclopropyl, cyclobutyl, cyclopentyl, cyclohexyl, azetidinyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azetidinonyl, azepanylyl, oxazetidinyl, diazetidinyl, oxazepanylyl, diazepanylyl, pyrrolidinonyl, piperidinonyl, azepanylonyl, thioxoazetidinyl, phenyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, 5,6-dihydro-4H-cyclopenta[c]furanyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

In a more preferred embodiment of formula (I) A2 is selected from 5- or 6-membered cycloalkyl, aryl, heterocyclyl, and heteroaryl, and wherein R⁴ and R⁵ independently are attached to cycloalkyl, aryl, heterocyclyl, or heteroaryl via any chemically feasible positions of the ring systems. Any of these 5- or 6-membered cycloalkyl, aryl, heterocyclyl, and heteroaryl may each independently be as defined herein above, and may optionally be substituted by one or more substituents as defined herein above, or more preferably be selected from the group consisting of cyclopentyl, cyclohexyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azepanylyl, oxazepanyl, diazepanyl, pyrrolidinonyl, piperidinonyl, azepanylonyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, 5,6-dihydro-4H-cyclopenta[c]furanyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

In an even more preferred embodiment of formula (I) A2 is selected from 5-membered cycloalkyl, heterocyclyl, and heteroaryl, wherein R⁴ and R⁵ independently are attached to cycloalkyl, aryl, heterocyclyl, or heteroaryl via any chemically feasible positions of the ring systems. Any of these 5-membered cycloalkyl, aryl, heterocyclyl, and heteroaryl may each independently be as defined herein above, and may optionally be substituted by one or more substituents as defined herein above, or more preferably be selected from the group consisting cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrazolyl, triazolyl, and tetrazolyl. In an even more preferred embodiment of formula (I) A2 is selected from the group consisting of cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, pyrrolyl, furanyl, isoxazolyl, thienyl, thiazolyl, isothiazolyl, imidazolyl, oxadiazolyl, thiadiazolyl, oxathiazolyl, pyrazolyl, triazolyl, and tetrazolyl. Alternatively A2 is selected from the group consisting of cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, isoxazolyl, isothiazolyl, oxadiazolyl, thiadiazolyl, oxathiazolyl, pyrazolyl, triazolyl, and tetrazolyl. In a most preferred embodiment of formula (I) A2 is selected from the group consisting of pyrrolidinyl, tetrahydrofuranyl, dihydropyrrole, pyrrolidinonyl, cyclopentadienyl, isoxazolyl, isothiazolyl, oxadiazolyl, thiadiazolyl, oxathiazolyl, pyrazolyl, triazolyl, and tetrazolyl.

In another embodiment A2 is selected from 5-membered heterocyclyl. In an alternative embodiment A2 is selected from 5-membered heteroaryl. These 5-membered heterocyclyl or heteroaryl may each independently be as defined herein above.

In an alternative preferred embodiment A2 is —NHC(R⁴R⁵)—.

In a preferred embodiment of formula (I) A3 is C (carbon atom). In a specific embodiment of formula (I) A3 forms a heterocyclic ring together with R⁴.

In a preferred embodiment of formula (I) A4 is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂O—, —CH₂OCH₂—, and —OCH₂CH₂—. In an alternative embodiment of formula (I) A4 is selected from the group consisting of —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂O—, —CH₂OCH₂—, and —OCH₂CH₂—.

In a more preferred embodiment of formula (I) A4 is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHSO₂—, —SO₂NH—, —CH₂CH₂CH₂—, —CH₂CH₂O—, —CH₂OCH₂—, and —OCH₂CH₂—. It is an advantage of this embodiment that the A4 linker is selected so as to increase the stability of the compounds of formula (I), the compounds of the present invention having these specific A4 linkers are more stable compared to compounds having for example —NH—C(O)— or —C(O)—O— linkers. As the skilled person within the field will know peptide moieties such as —NH—C(O)— moieties may render compounds susceptible to for example proteolytic enzymes. In an even more preferred embodiment A4 is selected from the group consisting of —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, and —SO₂NH—. In an yet even more preferred embodiment A4 is selected from the group consisting of —CH₂—, —C(O)—, —NH—, —O—, —S—, and —SO₂—.

Alternatively, A4 is selected from the group consisting of single bond, —NH—, —O—, —S—, —SO₂—, —NHCH₂—, —CH₂NH—, —OCH₂—, —CH₂O—, —SCH₂—, —CH₂S—, —SO₂CH₂—, —CH₂SO₂—, —NHSO₂—, —SO₂NH—, —CH₂CH₂NH—, —CH₂CH₂S—, —CH₂CH₂SO₂—, —CH₂NHCH₂—, —CH₂OCH₂—, —CH₂SCH₂—, —CH₂SO₂CH₂—, —NHCH₂CH₂—, —OCH₂CH₂—, —SCH₂CH₂—, —SO₂CH₂CH₂—, —CH₂SO₂NH—, —CH₂NHSO₂—, —SO₂NHCH₂—, and —NHSO₂CH₂—.

In an alternative embodiment A4 is selected from the group consisting of —CH₂—, —CH₂CH₂—, and —CH₂CH₂CH₂—. In another alternative embodiment A4 is selected from the group consisting of —C(O)—, —C(O)CH₂—, —CH₂C(O)—, —OCH₂—, —CH₂O—, —CH₂CH₂O—, —CH₂OCH₂—, and —OCH₂CH₂—. In a further alternative embodiment A4 is selected from the group consisting of —NH—, —O—, —S—, —SO₂—, —NHCH₂—, —CH₂NH—, —SCH₂—, —CH₂S—, —SO₂CH₂—, —CH₂SO₂—, —NHSO₂—, and —SO₂NH—. In a particular embodiment A4 is a single bond.

In one preferred embodiment of formula (I) the moiety X is defined by the structure

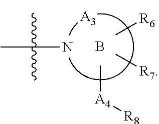

B may for example be selected from the group consisting of 2-azabicyclo[2.2.0]hexane, 6-azabicyclo[3.2.0]heptane, 7-azabicyclo[4.2.0]octane, 3-azabicyclo[3.2.0]heptane, 2-azabicyclo[3.2.0]heptane, octahydrocyclopenta[c]pyrrole, octahydrocyclopenta[b]pyrrole, octahydro-1H-isoindole, octahydro-1H-indole, decahydrocyclohepta[c]pyrrole, decahydrocyclohepta[b]pyrrole, octahydro-1,3-benzothiazole, octahydro-7H-pyrrolo[2,3-c]pyridin-7-one, octahydro-1H-pyrrolo[2,3-c]pyridine, octahydro-2H-cyclohepta[d][1,3]thiazole, octahydropyrrolo[2,3-c]azepin-8(1H)-one, decahydropyrrolo[2,3-c]azepine, 2,3-dihydro-1H-indole, 2,3-dihydro-1H-isoindole, octahydropyrrolo[3,4-b]pyrrole, octahydropyrrolo[2,3-e][1,3]oxazine, octahydro[1,3]oxazolo[4,5-c]pyridine, hexahydro-3aH-[1,3]oxazolo[4,5-e][1,3]oxazine, hexahydro-3aH-[1,3]oxazolo[4,5-e][1,3]oxazine, octahydropyrrolo[2,3-e][1,3]thiazine, hexahydro-3aH-[1,3]thiazolo[4,5-e][1,3]thiazine, hexahydro-3aH-[1,3]oxazolo[4,5-e][1,3]thiazine, octahydropyrrolo[3,4-b]pyrrole, hexahydropyrrolo[3,4-b]pyrrol-6(1H)-one, 3-azabicyclo[4.2.0]octane, 2-azabicyclo[4.2.0]octane, octahydro-1H-Cyclopenta[c]pyridine, octahydro-1H-cyclopenta[b]pyridine, decahydroisoquinoline, decahydroquinoline, decahydro-1H-cyclohepta[b]pyridine, decahydro-1H-cyclohepta[c]pyridine, octahydro-2H-1,3-benzothiazine, octahydro-2,7-naphthyridin-1(2H)-one, decahydro-2,7-naphthyridine, 1,2,3,4-tetrahydroisoquinoline, 1,2,3,4-tetrahydroquinoline, octahydro-1H-pyrrolo[3,2-c]pyridine, 2-azaspiro[3.3]heptane, 1-azaspiro[3.3]heptane, 1-azaspiro[3.4]octane, 2-azaspiro[3.4]octane, 2-azaspiro[3.5]nonane, 1-azaspiro[3.5]nonane, 2-azaspiro[3.6]decane, 1-azaspiro[3.6]decane, 6-azaspiro[3.4]octane, 5-azaspiro[3.4]octane, 1-azaspiro[4.4]nonane, 2-azaspiro[4.4]nonane, 2-azaspiro[4.5]decane, 1-azaspiro[4.5]decane, 2-azaspiro[4.6]undecane, 2-azaspiro[4.6]undecane, 2,7-diazaspiro[4.4]nonane, 1,7-diazaspiro[4.4]nonane, 2,7-diazaspiro[4.5]decane, 1,7-diazaspiro[4.5]decane, 2,9-diazaspiro[5.5]undecane, 2,8-diazaspiro[5.5]undecane, 1,8-diazaspiro[5.5]undecane, octahydropyrrolo[3,4-b]pyrrole, 2,7-diazaspiro[4.4]nonane, 1,7-diazaspiro[4.4]nonane, 2,7-diazaspiro[4.5]decane, 1,7-diazaspiro[4.5]decane, 7-azaspiro[3.5]nonane, 6-azaspiro[3.5]nonane, 5-azaspiro[3.5]nonane, 8-azaspiro[4.5]decane, 7-azaspiro[4.5]decane, 6-azaspiro[4.5]decane, 3-azaspiro[5.5]undecane, 2-azaspiro[5.5]undecane, 1-azaspiro[5.5]undecane, 3-azaspiro[5.6]dodecane, 2-azaspiro[5.6]dodecane, 1-azaspiro[5.6]dodecane, 2,9-diazaspiro[5.5]undecane, 2,8-diazaspiro[5.5]undecane, 1,8-diazaspiro[5.5]undecane, 2-azabicyclo[1.1.1]pentane, 5-azabicyclo[2.1.1]hexane, 6-azabicyclo[3.1.1]heptane, 7-azabicyclo[4.1.1]octane, 2-azabicyclo[2.1.1]hexane, 7-azabicyclo[2.2.1]heptane, 2-azabicyclo[2.2.1]heptane, 6-azabicyclo[3.2.1]octane, 2-azabicyclo[3.2.1]octane, 3-azabicyclo[3.2.1]octane, 7-azabicyclo[4.2.1]nonane, 9-azabicyclo[4.2.1]nonane, and 3-azabicyclo[4.2.1].

In a preferred embodiment of formula (I) B is selected from the group consisting of 4 membered, 5 membered, 6 membered, and 7 membered heterocyclic or heteroaromatic ring systems, wherein any of these ring systems optionally may be substituted by one or more substituents as defined herein above. Any of these heterocyclyl, and heteroaryl may each independently be as defined herein above, or more preferably be selected from the group consisting of 5 membered and 6 membered heterocyclic and heteroaromatic rings. More particularly B may be selected from the group consisting of azetidine, 1,2-diazetidine, 1,3-diazetidine, 1,2-oxazetidine, 1,3-oxazetidine, 1,2-thiazetidine, 1,3-thiazetidine, 1,2-dihydroazete, pyrrolidine, pyrazolidine, imidazolidine, isoxazolidine, 1,3-oxazolidine, isothiazolidine, 1,3-thiazolidine, 2,3-dihydro-1H-pyrrole, 2,5-dihydro-1H-pyrrole, 2,5-dihydroisoxazole, 2,3-dihydro-1,3-oxazole, 2,5-dihydroisothiazole, 2,3-dihydro-1,3-thiazole, 2,3-dihydroisoxazole, 2,3-dihydroisothiazole, piperidine, hexahydropyridazine, hexahydropyrimidine, piperazine, 1,2-oxazinane, 1,3-oxazinane, morpholine, 1,2-thiazinane, 1,3-thiazinane, thiomorpholine, 1,2,3,4-tetrahydropyridine, 1,2,3,6-tetrahydropyridine, 1,2,3,6-tetrahydropyridine, 1,2-dihydropyridine, 1,4-dihydropyridine, 1,2,3,4-tetrahydropyridazine, 1,2,3,4-tetrahydropyrimidine, 1,2,3,4-tetrahydropyrazine, 5,6-dihydro-2H-1,2-oxazine, 3,6-dihydro-2H-1,3-oxazine, 3,4-dihydro-2H-1,4-oxazine, 5,6-dihydro-2H-1,2-thiazine, 3,6-dihydro-2H-1,3-thiazine, 3,4-dihydro-2H-1,4-thiazine, 3,6-dihydro-2H-1,2-oxazine, 3,4-dihydro-2H-1,3-oxazine, 3,4-dihydro-2H-1,2-oxazine, 1,2-dihydropyridine, 1,4-dihydropyridine, tetrahydropyrimidin-4(1H)-one, piperazin-2-one, 1,3,5-triazinan-2-one, piperidin-4-one, piperidin-3-one, azepane, 1,2-diazepane, 1,3-diazepane, 1,4-diazepane, 1,2-oxazepane, 1,3-oxazepane, 1,4-oxazepane, 1,2-thiazepane, 1,3-thiazepane, 1,4-thiazepane, 2,3,4,5-tetrahydro-1H-azepine, 2,3,4,7-tetrahydro-1H-azepine, 2,3,6,7-tetrahydro-1H-azepine, 2,3-dihydro-1H-azepine, 1H-azepine, 4,5-dihydro-1H-azepine, 2,3,4,5-tetrahydro-1H-1,2-diazepine, 2,3,4,5-tetrahydro-1H-1,3-diazepine, 2,3,4,5-tetrahydro-1H-1,4-diazepine, 4,5,6,7-tetrahydro-1H-1,4-diazepine, 2,5,6,7-tetrahydro-1,2-oxazepine, 2,3,6,7-tetrahydro-1,3-oxazepine, 2,3,4,7-tetrahydro-1,4-oxazepine, 4,5,6,7-tetrahydro-1,4-oxazepine, 2,5,6,7-tetrahydro-1,2-thiazepine, 2,3,6,7-tetrahydro-1,3-thiazepine, 2,3,4,7-tetrahydro-1,4-thiazepine, 4,5,6,7-tetrahydro-1,4-thiazepine, 2,3,4,5-tetrahydro-1,2-oxazepine, 2,3,6,7-tetrahydro-1,2-oxazepine, 2,3,4,7-tetrahydro-1,3-oxazepine, and 2,3,4,5-tetrahydro-1,4-oxazepine.

In a more preferred embodiment of formula (I) B is selected from the group consisting of pyrrolidine, pyrazolidine, imidazolidine, isoxazolidine, 1,3-oxazolidine, isothiazolidine, 1,3-thiazolidine, 2,3-dihydro-1H-pyrrole, 2,5-dihydro-1H-pyrrole, 2,5-dihydroisoxazole, 2,3-dihydro-1,3-oxazole, 2,5-dihydroisothiazole, 2,3-dihydro-1,3-thiazole, 2,3-dihydroisoxazole, 2,3-dihydroisothiazole, piperidine, hexahydropyridazine, hexahydropyrimidine, piperazine, 1,2-oxazinane, 1,3-oxazinane, morpholine, 1,2-thiazinane, 1,3-thiazinane, thiomorpholine, 1,2,3,4-tetrahydropyridine, 1,2,3,6-tetrahydropyridine, 1,2,3,6-tetrahydropyridine, 1,2-dihydropyridine, 1,4-dihydropyridine, 1,2,3,4-tetrahydropyridazine, 1,2,3,4-tetrahydropyrimidine, 1,2,3,4-tetrahydropyrazine, 5,6-dihydro-2H-1,2-oxazine, 3,6-dihydro-2H-1,3-oxazine, 3,4-dihydro-2H-1,4-oxazine, 5,6-dihydro-2H-1,2-thiazine, 3,6-dihydro-2H-1,3-thiazine, 3,4-dihydro-2H-1,4-thiazine, 3,6-dihydro-2H-1,2-oxazine, 3,4-dihydro-2H-1,3-oxazine, 3,4-dihydro-2H-1,2-oxazine, 1,2-dihydropyridine, 1,4-dihydropyridine, tetrahydropyrimidin-4(1H)-one, piperazin-2-one, 1,3,5-triazinan-2-one, piperidin-4-one, and piperidin-3-one. More preferably B is selected from the group consisting of azetidin-1-yl, 1,2-diazetidin-1-yl, 1,3-diazetidin-1-yl, 1,2-oxazetidin-2-yl, 1,2-thiazetidin-2-yl, pyrrolidin-1-yl, imidazolidin-1-yl, 1,3-oxazolidin-3-yl, 1,3-thiazolidin-3-yl, piperidin-1-yl, 1,3-oxazinan-3-yl, morpholin-4-yl, and 3-oxopiperazin-1-yl, and 4-oxopiperidin-1-yl. Even more preferably B may be selected from the group consisting of azetidin-1-yl, pyrrolidin-1-yl, piperidin-1-yl, 2-oxo-piperazinyl, morpholin-4-yl, and piperazin-1-yl.

In a most preferred embodiment of formula (I) B is pyrrolidinyl.

In an alternative embodiment of formula (I) B is selected from the group consisting of bicyclic, fused or spiro-cyclic heterocyclyl, and bicyclic, fused or spiro-cyclic heteroaryl rings, any of which rings optionally may be substituted by one or more substituents as defined herein above. Any of these bicyclic, fused or spirocyclic heterocyclyl, and heteroaryl may each independently be as defined herein above, or more preferably be selected from the group consisting of 2,3-dihydro-1H-indol-1-yl, 1,3-dihydro-2H-isoindol-2-yl, hexahydropyrrolo[2,3-e][1,3]oxazin-5(2H)-yl, hexahydro[1,3]oxazolo[4,5-c]pyridin-3(2H)-yl, tetrahydro-3aH-[1,3]oxazolo[4,5-e][1,3]oxazin-1(2H)-yl, hexahydro[1,3]thiazolo[4,5-c]pyridin-3(2H)-yl, hexahydropyrrolo[2,3-e][1,3]thiazin-5(2H)-yl, tetrahydro-3aH-[1,3]thiazolo[4,5-e][1,3]thiazin-1(2H)-yl, tetrahydro-3aH-[1,3]thiazolo[4,5-e][1,3]oxazin-1(2H)-yl, tetrahydro-3aH-[1,3]oxazolo[4,5-e][1,3]thiazin-1(2H)-yl, 3,4-dihydroisoquinolin-2(1H)-yl, 3,4-dihydroquinolin-1(2H)-yl, hexahydropyrrolo[3,4-b]pyrrol-5(1H)-yl, octahydropyrrolo[2,3-c]azepin-1(2H)-yl, 7-oxooctahydro-1H-pyrrolo[2,3-c]pyridin-1-yl, 8-oxooctahydropyrrolo[2,3-c]azepin-1(2H)-yl, 6-oxohexahydropyrrolo[3,4-b]pyrrol-1(2H)-yl, octahydro-1H-pyrrolo[2,3-c]pyridin-1-yl, octahydro-1H-pyrrolo[3,2-c]pyridin-1-yl, and 2,7-diazaspiro[4.5]dec-2-yl. Even more preferably B may be selected from the group consisting of octahydro-1H-pyrrolo[2,3-c]pyridin-1-yl, octahydro-1H-pyrrolo[3,2-c]pyridin-1-yl, octahydropyrrolo[2,3-c]azepin-1(2H)-yl, octahydro-2,7-naphthyridin-2(1H)-yl, 3,4-dihydroisoquinolin-2(1H)-yl, 3,4-dihydroquinolin-1(2H)-yl, hexahydropyrrolo[3,4-b]pyrrol-5(1H)-yl, octahydropyrrolo[2,3-c]azepin-1(2H)-yl, 7-oxooctahydro-1H-pyrrolo[2,3-c]pyridin-1-yl, 8-oxooctahydropyrrolo[2,3-c]azepin-1(2H)-yl, 6-oxohexahydropyrrolo[3,4-b]pyrrol-1(2H)-yl, and 2,7-diazaspiro[4.5]dec-2-yl.

For the above-mentioned embodiments of B applies that the functional moieties A4, R⁶ and R⁷ may be attached to any chemically feasible position of the ring system B. In a preferred embodiment of formula (I) A4 is attached to B, via a ring atom next to the Nitrogen atom of B.

In one embodiment of formula (I) moiety X, wherein B, A4, R⁶, R⁷, and R⁸ are combined, is selected from the group consisting of

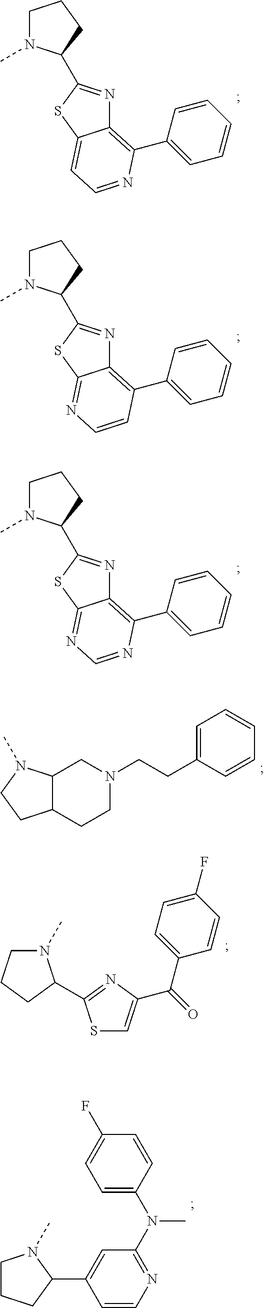

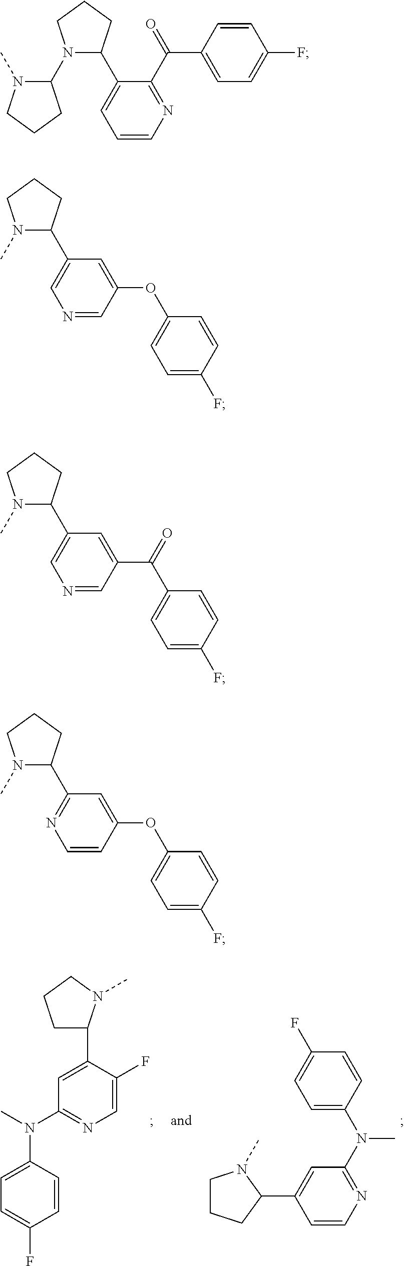

wherein the dotted line indicates the attachment point of X to the remaining part of formula (I).

In an alternative preferred embodiment of formula (I) the moiety X is defined by the structure

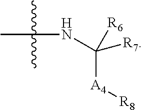

In a preferred embodiment of formula (I) R¹ is selected from the group consisting of H, C₁-C₆ alkyl, C₃-C₁₀ cycloalkyl, aryl, heterocyclyl, and heteroaryl, wherein any alkyl, cycloalkyl, aryl, heterocyclyl, and heteroaryl optionally are substituted. The substituents may be selected from substituents as defined herein above. More preferably R¹ may be selected from the group consisting of H and C₁-C₄ alkyl. Even more preferably R¹ may be H.

In a preferred embodiment of formula (I) R² is selected from the group consisting of H, C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(CH₂)₁₋₄₋heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, wherein the heterocyclic ring optionally is substituted. The here mentioned substitution may be by one or more substituents as mentioned herein above. More preferably R² may be selected from the group consisting of H, C₁-C₄ alkyl, C₁-C₄ alkoxy, C₂-C₄ alkenyl, C₂-C₄ alkynyl, wherein any alkyl, alkenyl and alkynyl optionally are substituted; or wherein R² together with R⁵ optionally may form a heterocyclic ring together with the nitrogen to which R² is attached, wherein the heterocyclic ring optionally is substituted. Even more preferably R² may be selected from the group consisting of H, C₁-C₄ alkyl, C₁-C₄ alkoxy, C₃-C₆ cycloalkyl, —(CH₂)₁₋₄-cycloalkyl, wherein any alkyl, cycloalkyl, optionally are substituted; or wherein R² together with R⁵ optionally may form a heterocyclic ring together with the nitrogen to which R² is attached, wherein the heterocyclic ring optionally is substituted. Most preferably R² may be methyl.

In an alternative embodiment of formula (I) R² is selected from the group consisting of C₃-C₁₀ cycloalkyl, aryl, heterocyclyl, heteroaryl, —(CH₂)₁₋₆-aryl, —(CH₂)₁₋₆-heterocyclyl, and —(CH₂)₁₋₆-heteroaryl, and wherein any cycloalkyl, aryl, heterocyclyl, and heteroaryl optionally are substituted. The substituent may be any substituent as defined herein above and the cycloalkyl, aryl, heterocyclyl, and heteroaryl may each independently be as defined herein above.

In a particular embodiment of formula (I) at least one of R¹ and R² is H. More preferably R² may be H.

In an alternative, preferred embodiment of formula (I), at least one of R¹ and R² is different from H. It has surprisingly been found, that the presence of at least one of R¹ and R² different from H, may improve the compounds cell permeability. To this end it is especially preferred that one of R¹ and R² are selected from the group consisting of C₁-C₄ alkyl, C₁-C₄ alkoxy, C₂-C₄ alkenyl, and C₂-C₄ alkynyl, wherein any alkyl, alkenyl and alkynyl optionally are substituted; more preferably selected from the group consisting of C₁-C₄ alkyl, and C₁-C₄ alkoxy; even more preferably methyl or ethyl; and yet even more preferably methyl. Accordingly in a preferred embodiment of formula (I) R¹ is H and R² is methyl.

In an alternative embodiment of formula (I) R² together with R⁵ forms a heterocyclic ring together with the nitrogen to which R² is attached, wherein the heterocyclic ring optionally is substituted. When such a heterocyclic ring is formed R² may be seen as a single bond or for example an alkyl moiety depending on what is relevant for the specific heterocyclic ring. Accordingly, in one embodiment R² together with R⁵ forms a heterocyclic ring together with the nitrogen to which R² is attached, wherein the heterocyclic ring optionally is substituted, and wherein R² is a single bond. The heterocyclic ring may be any ring as defined herein above, and preferably may be a 5-, 6- or 7-membered heterocyclic ring, more preferably a 5 or 6-membered heterocyclic ring. For this embodiment of the invention the heterocyclic ring may optionally be substituted with one or more substituents as defined herein above, and more preferably the heterocyclic ring may be substituted with one or more substituents selected from the group consisting of —F, —Cl, —OH, —CF₃, C₁-C₄ alkyl, —CN, and —NO₂

More preferably the heterocyclic ring formed by R² together with R⁵ may be selected from the group consisting of pyrrolidinyl, piperidinyl, azetidinyl, 1,2-diazetidinyl, 1,2-oxazetidinyl, 1,2-thiazetidinyl, pyrazolidinyl, isoxazolidinyl, imidazolidinyl, 1,3-oxazolidinyl, 1,3-thiazolidinyl, hexahydropyridazinyl, hexahydropyrimidinyl, piperazinyl, 1,2-oxazinanyl, 1,3-oxazinanyl, morpholinyl, 1,2-thiazinanyl, 1,3-thiazinanyl, and thiomorpholinyl, and wherein the ring optionally is substituted. Even more preferably the heterocyclic ring may be selected from the group consisting of azetidinyl, pyrrolidinyl, and piperidinyl, and wherein the ring optionally is substituted.

In an alternative embodiment of the compounds of formula (I) R¹ and R² are both H.

In a preferred embodiment of formula (I) R³ is selected from the group consisting of H, hydroxy, halogen, C₁-C₄ alkyl, C₁-C₄ alkoxy, C₂-C₄ alkenyl, C₂-C₄ alkynyl, and C₃-C₆ cycloalkyl, wherein any alkyl, alkenyl and alkynyl optionally are substituted. The substituents may be any one or more substituents as defined herein above. More preferably R³ may be selected from the group consisting of H, hydroxy, and C₁-C₄ alkyl. Even more preferably R³ may be H. In a particular embodiment of formula (I) R³ is selected from the group consisting of H, OH, methyl, ethyl, and —CH₂OH; more particularly R³ is selected from the group consisting of OH and —CH₂OH.

Alternatively R³ may be selected from the group consisting of fluoro and —CH₂F. In one embodiment of the invention R³ is not methyl.

In a preferred embodiment of formula (I) R⁴ and R⁵ each independently are selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —NH—(CH₂)ₙ—Z₂, —O—(CH₂)ₙ—Z₂, —CH₂—NH—(CH₂)ₙ—Z₂, —CH₂—O—(CH₂)ₙ—Z₂, and —(CH₂)ₙ—Z₂, wherein Z₂ is as defined herein above or below, and wherein any alkyl, alkenyl, alkynyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, and wherein any heterocyclic ring optionally is substituted. More preferably R⁴ and R⁵ each independently may be selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, and —(CH₂)ₙ—Z₂, wherein Z₂ is as defined herein above or below, and wherein any alkyl, alkenyl, alkynyl, cycloalkyl, aryl, heterocyclyl, and heteroaryl optionally are substituted. Even more preferably R⁴ and R⁵ each independently may be selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein n is 0 or an integer from 1 to 3; wherein Z₂ is as defined herein above or below, and wherein any alkyl, cycloalkyl, heterocyclyl, and heteroaryl optionally are substituted. Yet even more preferably R⁴ and R⁵ each independently may be selected from the group consisting of H, hydroxyl, —NH₂, —CN, —SO₂, —NO₂, halogen, C₁-C₃ alkyl, C₁-C₃ alkyl substituted with fluoro, C₁-C₃ alkoxy, C₃-C₆ cycloalkyl, C₃-C₆ heterocyclyl, C₃-C₆ heteroaryl and —(CH₂)ₙ—Z₂, wherein n is 0 or 1, Z₂ is as defined herein above or below, and wherein any alkyl, cycloalkyl, aryl, heterocyclyl, and heteroaryl optionally are substituted. The substitution referred to in relation to R⁴ and R⁵ may be by any one or more substituents as described herein above.

In another preferred embodiment of formula (I) R⁴ and R⁵ each independently are selected from the group consisting of C₂-C₆ alkyl, C₂-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein n is 0 or 1, Z₂ is as defined herein above and below, and wherein any alkyl, cycloalkyl, aryl, heterocyclyl, and heteroaryl optionally are substituted.

In one embodiment of formula (I) R⁴ does not form a heterocyclic ring together with A₃ and the nitrogen to which A3 is attached.

In a more preferred embodiment of formula (I) R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl. More preferably R⁴ and R⁵ each independently may be selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

In a specific embodiment of formula (I) R⁴ and R⁵ each independently are selected from an C₁-C₆ alkyl, and preferably R⁴ and R⁵ each independently may be selected from the group consisting of H, methyl, ethyl, propyl, isopropyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl. More preferably R⁴ and R⁵ each independently may be selected from the group consisting of H, methyl, ethyl, propyl, isopropyl, methoxy, and ethoxy.

In another specific embodiment of formula (I) R⁴ and R⁵ each independently are selected from the group consisting of H, methyl, ethyl, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl. More preferably R⁴ and R⁵ each independently may be selected from the group consisting of H, methyl, ethyl, methoxy, and ethoxy.

In a particular embodiment of formula (I) R⁴ and R⁵ each independently are selected from the group consisting of H, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, —CH₂CF₃, and —CF₂CF₃. In another particular embodiment of formula (I) R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —SO₂, and —NO₂.

In another particular embodiment of formula (I) R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, and tert-butyl butyl. Alternatively, R⁴ and R⁵ each independently may be selected from the group consisting of H, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, tert-butyl. These two embodiments are especially preferred for compounds of formula (I) wherein A2 is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems.

In an alternative embodiment of formula (I) R⁴ and R⁵ each independently are selected from the group consisting of C₃-C₁₀ cycloalkyl, aryl, heterocyclyl, and heteroaryl. More preferably R⁴ and R⁵ each independently may be selected from the group consisting of cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl.

Alternatively R⁴ and R⁵ each independently may be selected from the group consisting of bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, azetidinylmethyl, aziridinylmethyl, pyrrolidinylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, azetidinylethyl, aziridinylethyl, pyrrolidinylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl.

In a further embodiment of formula (I) R⁴ and R⁵ each independently are selected from the group consisting of cyclohexyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl. More preferably R⁴ and R⁵ each independently may be selected from the group consisting of tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl.

In a particular embodiment of formula (I) R⁴ together with A3 forms a heterocyclic ring together with the nitrogen to which A3 is attached, wherein the heterocyclic ring optionally is substituted.

In another particular embodiment of formula (I) R⁵ together with R² forms a heterocyclic ring together with the nitrogen to which R² is attached, wherein the heterocyclic ring optionally is substituted.

In a preferred embodiment of formula (I) Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}—heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —CO(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)_{q}-heterocyclyl, and —O—C(O)—(CH₂)_{q}-heteroaryl, and wherein any alkyl, cycloalkyl, aryl, heterocyclyl, and heteroaryl optionally are substituted. In an alternative embodiment of formula (I) Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}—heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, and wherein any alkyl, cycloalkyl, aryl, heterocyclyl, and heteroaryl optionally are substituted. More preferably Z₂ may be selected from the group consisting of H, —OH, —NH₂, —CN, —SO₂, —NO₂, halogen, C₁-C₆ alkoxy, C₃-C₁₀ cycloalkyl, C₃-C₁₀ heterocyclyl, and C₃-C₁₀ heteroaryl, wherein any alkyl, cycloalkyl, heterocyclyl, and heteroaryl optionally are substituted. Even more preferably Z₂ may be selected from the group consisting of H, —OH, —NH₂, —CN, —SO₂, —NO₂, halogen, C₁-C₃ alkoxy, C₃-C₆ cycloalkyl, C₃-C₆ heterocyclyl, and C₆-C₁₀ heteroaryl, and wherein any alkyl, cycloalkyl, heterocyclyl, and heteroaryl optionally are substituted.

The substitution referred to in relation to Z² may be by any one or more substituents as described herein above. In a preferred embodiment of formula (I) substituents for any alkyl, cycloalkyl, aryl, heterocyclyl, and heteroaryl of R⁴, R⁵, and Z₂ is one or more substituents each independently selected from the group consisting of chloro, fluoro, hydroxyl, —C(O)NH₂, C₁-C₆ alkyl, C₁-C₆ alkoxy, and —CN.

In a specific embodiment of formula (I) Z₂ is selected from the group consisting of —H, methyl, —OH, —NH₂, —CN, —F, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, wherein any alkyl, cycloalkyl, aryl, heterocyclyl, and heteroaryl optionally are substituted.

In a preferred embodiment of formula (I) R⁶ and R⁷ each independently are selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(C₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, and wherein any alkyl, cycloalkyl, aryl, heterocyclyl, and heteroaryl optionally are substituted; wherein Z₃ is selected from the group consisting of H, F, —OH, —NH₂, —NO₂, —CN, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂), —heteroaryl, —NH(R⁹), —N(R⁹)—SO₂—C₁-C₆ alkyl, —N(R⁹)—SO₂—(CH₂)ᵣ—C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂)ᵣ—C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—N(R¹⁰)(R¹¹), wherein any alkyl, cycloalkyl, aryl, heterocyclyl, and heteroaryl optionally are substituted; p is 0, or an integer from 1 to 2; and wherein r is 0, or an integer from 1 to 2.

In a preferred embodiment of formula (I) at least one of R⁶ and R⁷ are different from H. The present inventors have found that by attaching an additional substituent, besides the substituent defined as -A4-R⁸, to the B ring system an improved activity profile is seen.

Particularly, in one embodiment R⁶ and R⁷ each independently are selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(C₂)ₚ—Z₃, wherein Z₃ is as defined herein above or below, and wherein any alkyl, cycloalkyl, aryl, heterocyclyl, and heteroaryl optionally are substituted. Accordingly, in this embodiment both of R⁶ and R⁷ is different from H, hereby giving an even further improved activity profile.

More particularly, in one embodiment of formula (I) R⁶ and R⁷ each independently are selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —O—(CH₂)ₚ—Z₃, and —(CH₂)ₚ—Z₃, wherein p is 0 or an integer from 1 to 3; wherein Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂— (CH₂)ᵣ-heterocyclyl, —SO₂—(CH₂)ᵣ-heteroaryl, —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)ᵣ-heteroaryl, —OC(O)—C₁-C₁₀ alkyl, —O—C(O)—(CH₂)ᵣ—C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl; and wherein any alkyl, cycloalkyl, aryl, heterocyclyl, and heteroaryl optionally are substituted.

In a specific embodiment of formula (I) at least one of R⁶ and R⁷ each independently are selected from the group consisting of methyl, —OH, —NH₂, —CN, —F, —Cl, —Br, —CH₂OH, methoxy, —CH₂F, —CHF₂, —CF₃, —CH₂Cl, —CH₂CH₂OH, ethoxy, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

In one embodiment of formula (I) R⁶ and R⁷ each independently is C₁-C₆ alkyl, wherein the alkyl optionally is substituted. In an alternative embodiment at least one of R⁶ and R⁷ each independently are C₃-C₁₀ cycloalkyl, wherein the cycloalkyl optionally is substituted.

In a further alternative embodiment at least one of R⁶ and R⁷ each independently are aryl, wherein the aryl optionally is substituted. More preferably R⁶ and R⁷ each independently may be phenyl optionally substituted with one to three substitutents selected from the group consisting of hydroxy, fluoro, chloro, bromo, iodo, methoxy, ethoxy. Even more preferably R⁶ and R⁷ each independently may be phenyl, fluorophenyl, hydroxyphenyl, chlorophenyl, difluorophenyl, dichlorophenyl, trifluorophenyl, and trichlorophenyl.

In a further alternative embodiment at least one of R⁶ and R⁷ each independently are heterocyclyl, wherein the heterocyclyl optionally is substituted. In a further alternative embodiment at least one of R⁶ and R⁷ each independently are heteroaryl, wherein the heteroaryl optionally is substituted. The heterocyclyl and heteroaryl may be as define herein.

For some embodiments of formula (I) R⁶ and R⁷ are both H.

In a more preferred embodiment of formula (I) at least one of R⁶ and R⁷ each independently are selected from the group consisting of cyclopropyl, cyclobutyl, cyclopentyl, cyclohexyl, bicyclo[2.2.2]octanyl, azetidinyl, tetrahydro-2H-pyranyl, piperidinyl, tetrahydro-2H-thiopyranyl, morpholinyl, piperazinyl, thiomorpholinylaziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl, and wherein any of the ring system optionally are substituted. Even more preferably at least one of R⁶ and R⁷ each independently may be a ring system selected from the group consisting of cyclopropyl, cyclobutyl, cyclopentyl, cyclohexyl, bicyclo[2.2.2]octanyl, aziridinyl, azetidinyl, tetrahydro-2H-pyranyl, piperidinyl, tetrahydro-2H-thiopyranyl, morpholinyl, piperazinyl, thiomorpholinyl, pyrrolidinyl, and tetrahydrofuranyl, and wherein the ring system optionally is substituted.

In a preferred embodiment of formula (I) Z₃ is selected from the group consisting of —H, methyl, —OH, —NH₂, —CN, —F, —Cl, —Br, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl. More preferably Z₃ may be selected from the group consisting of —H, methyl, —OH, —NH₂, —CN, —F, —Cl, —Br, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, pyrazinyl, pyrrolyl, pyrazolyl, furanyl, thienyl, fluorophenyl, hydroxyphenyl, chlorophenyl, difluorophenyl, dichlorophenyl, trifluorophenyl, and trichlorophenyl.

The substitution referred to in relation to R⁶, R⁷ and Z³ may be by any one or more substituents as described herein above.

In a preferred embodiment of formula (I) R⁸ is selected from the group consisting of C₃-C₆ cycloalkyl, aryl, heterocyclyl, heteroaryl, aryl-C₁-C₆ alkyl, C₃-C₆ cycloalkyl-aryl, aryl-C₃-C₆ cycloalkyl, C₃-C₆ cycloalkyl-heterocyclyl, heterocyclyl-C₃-C₆ cycloalkyl, C₃-C₆ cycloalkyl-heteroaryl, heteroaryl-C₃-C₆ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₆ cycloalkyl-O-aryl, aryl-O—C₃-C₆ cycloalkyl, C₃-C₆ cycloalkyl-O-heterocyclyl, heterocyclyl-O—C₃-C₆ cycloalkyl, C₃-C₆ cycloalkyl-O-heteroaryl, heteroaryl-O—C₃-C₆ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₆ cycloalkyl-C(O)-aryl, aryl-C(O)—C₃-C₆ cycloalkyl, C₃-C₆ cycloalkyl-C(O)-heterocyclyl, heterocyclyl-C(O)—C₃-C₆ cycloalkyl, C₃-C₆ cycloalkyl-C(O)-heteroaryl, heteroaryl-C(O)—C₃-C₆ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₆ cycloalkyl-CH₂-aryl, aryl-CH₂—C₃-C₆ cycloalkyl, C₃-C₆ cycloalkyl-CH₂-heterocyclyl, heterocyclyl-CH₂—C₃-C₆ cycloalkyl, C₃-C₆ cycloalkyl-CH₂-heteroaryl, heteroaryl-CH₂—C₃-C₆ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₆ cycloalkyl-CH₂CH₂-aryl, aryl-CH₂CH₂—C₃-C₆ cycloalkyl, C₃-C₆ cycloalkyl-CH₂CH₂-heterocyclyl, heterocyclyl-CH₂CH₂—C₃-C₆ cycloalkyl, C₃-C₆ cycloalkyl-CH₂CH₂-heteroaryl, heteroaryl-CH₂CH₂—C₃-C₆ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₆ cycloalkyl-NH-aryl, aryl-NH—C₃-C₆ cycloalkyl, C₃-C₆ cycloalkyl-NH-heterocyclyl, heterocyclyl-NH—C₃-C₆ cycloalkyl, C₃-C₆ cycloalkyl-NH-heteroaryl, heteroaryl-NH—C₃-C₆ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₆ cycloalkyl-N(Me)-aryl, aryl-N(Me)-C₃-C₆ cycloalkyl, C₃-C₆ cycloalkyl-N(Me)-heterocyclyl, heterocyclyl-N(Me)-C₃-C₆ cycloalkyl, C₃-C₆ cycloalkyl-N(Me)-heteroaryl, heteroaryl-N(Me)-C₃-C₆ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₆ cycloalkyl-NHC(O)-aryl, aryl-NHC(O)—C₃-C₆ cycloalkyl, C₃-C₆ cycloalkyl-NHC(O)-heterocyclyl, heterocyclyl-NHC(O)—C₃-C₆ cycloalkyl, C₃-C₆ cycloalkyl-NHC(O)-heteroaryl, heteroaryl-NHC(O)—C₃-C₆ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₆ cycloalkyl-C(O)NH-aryl, aryl-C(O)NH—C₃-C₆ cycloalkyl, C₃-C₆ cycloalkyl-C(O)NH-heterocyclyl, heterocyclyl-C(O)NH—C₃-C₆ cycloalkyl, C₃-C₆ cycloalkyl-C(O)NH-heteroaryl, heteroaryl-C(O)NH—C₃-C₆ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₆ cycloalkyl-NHC(O)NH-aryl, aryl-NHC(O)NH—C₃-C₆ cycloalkyl, C₃-C₆ cycloalkyl-NHC(O)NH-heterocyclyl, heterocyclyl-NHC(O)NH—C₃-C₆ cycloalkyl, C₃-C₆ cycloalkyl-NHC(O)NH-heteroaryl, heteroaryl-NHC(O)NH—C₃-C₆ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted.

In a more preferred embodiment of formula (I) R⁸ is selected from the group consisting of C₃-C₁₀ cycloalkyl, heterocyclyl, heteroaryl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH—heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; and wherein any cycloalkyl, aryl, heterocyclyl, and heteroaryl optionally may be substituted.

In an alternative embodiment of formula (I) R⁸ is selected from the group consisting of C₃-C₁₀ cycloalkyl, aryl, heterocyclyl and heteroaryl; and wherein cycloalkyl, heterocyclyl, and heteroaryl optionally may be substituted.

In one particular embodiment of formula (I) R⁸ is selected from the group consisting of C₃-C₁₀ cycloalkyl, heterocyclyl, heteroaryl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, heterocyclyl-heteroaryl, heteroaryl-heterocyclyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, heterocyclyl-O-heteroaryl, heteroaryl-O-heterocyclyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, heterocyclyl-C(O)-heteroaryl, heteroaryl-C(O)-heterocyclyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, heterocyclyl-CH₂-heteroaryl, heteroaryl-CH₂-heterocyclyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, heterocyclyl-CH₂CH₂-heteroaryl, heteroaryl-CH₂CH₂-heterocyclyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, heterocyclyl-NH-heteroaryl, heteroaryl-NH-heterocyclyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, heterocyclyl-N(Me)-heteroaryl, heteroaryl-N(Me)-heterocyclyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, heterocyclyl-NHC(O)-heteroaryl, heteroaryl-NHC(O)-heterocyclyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, heterocyclyl-C(O)NH-heteroaryl, heteroaryl-C(O)NH-heterocyclyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, heterocyclyl-NHC(O)NH-heteroaryl, and heteroaryl-NHC(O)NH-heterocyclyl; wherein cycloalkyl, heterocyclyl, and heteroaryl optionally may be substituted.

In an alternative embodiment of formula (I) R⁸ is selected from the group consisting of aryl-C(O)—C₃-C₁₀ cycloalkyl, aryl-C(O)-heteroaryl, aryl-C(O)-heterocyclyl, aryl-C(O)NH—C₃-C₁₀ cycloalkyl, aryl-C(O)NH-heteroaryl, aryl-C(O)NH-heterocyclyl, aryl-C₁-C₆ alkyl, aryl-C₃-C₁₀ cycloalkyl, aryl-CH₂—C₃-C₁₀ cycloalkyl, aryl-CH₂CH₂—C₃-C₁₀ cycloalkyl, aryl-CH₂CH₂-heteroaryl, aryl-CH₂CH₂-heterocyclyl, aryl-CH₂-heteroaryl, aryl-CH₂-heterocyclyl, aryl-heteroaryl, aryl-heterocyclyl, aryl-N(Me)-C₃-C₁₀ cycloalkyl, aryl-N(Me)-heteroaryl, aryl-N(Me)-heterocyclyl, aryl-NHC(O)—C₃-C₁₀ cycloalkyl, aryl-NHC(O)-heteroaryl, aryl-NHC(O)-heterocyclyl, aryl-NHC(O)NH—C₃-C₁₀ cycloalkyl, aryl-NHC(O)NH-heteroaryl, aryl-NHC(O)NH-heterocyclyl, aryl-NH—C₃-C₁₀ cycloalkyl, aryl-NH-heteroaryl, aryl-NH-heterocyclyl, aryl-O—C₃-C₁₀ cycloalkyl, aryl-O-heteroaryl, and aryl-O-heterocyclyl.

In another particular embodiment of formula (I) R⁸ is selected from the group consisting of C₃-C₁₀ cycloalkyl-aryl, C₃-C₁₀ cycloalkyl-C(O)-aryl, C₃-C₁₀ cycloalkyl-C(O)-heteroaryl, C₃-C₁₀ cycloalkyl-C(O)-heterocyclyl, C₃-C₁₀ cycloalkyl-C(O)NH-aryl, C₃-C₁₀ cycloalkyl-C(O)NH-heteroaryl, C₃-C₁₀ cycloalkyl-C(O)NH-heterocyclyl, C₃-C₁₀ cycloalkyl-CH₂-aryl, C₃-C₁₀ cycloalkyl-CH₂CH₂-aryl, C₃-C₁₀ cycloalkyl-CH₂CH₂-heteroaryl, C₃-C₁₀ cycloalkyl-CH₂CH₂-heterocyclyl, C₃-C₁₀ cycloalkyl-CH₂-heteroaryl, C₃-C₁₀ cycloalkyl-CH₂-heterocyclyl, C₃-C₁₀ cycloalkyl-heteroaryl, C₃-C₁₀ cycloalkyl-heterocyclyl, C₃-C₁₀ cycloalkyl-N(Me)-aryl, C₃-C₁₀ cycloalkyl-N(Me)-heteroaryl, C₃-C₁₀ cycloalkyl-N(Me)-heterocyclyl, C₃-C₁₀ cycloalkyl-NH-aryl, C₃-C₁₀ cycloalkyl-NHC(O)-aryl, C₃-C₁₀ cycloalkyl-NHC(O)-heteroaryl, C₃-C₁₀ cycloalkyl-NHC(O)-heterocyclyl, C₃-C₁₀ cycloalkyl-NHC(O)NH-aryl, C₃-C₁₀ cycloalkyl-NHC(O)NH-heteroaryl, C₃-C₁₀ cycloalkyl-NHC(O)NH-heterocyclyl, C₃-C₁₀ cycloalkyl-NH-heteroaryl, C₃-C₁₀ cycloalkyl-NH-heterocyclyl, C₃-C₁₀ cycloalkyl-O-aryl, C₃-C₁₀ cycloalkyl-O-heteroaryl, and C₃-C₁₀ cycloalkyl-O-heterocyclyl.

In a further particular embodiment of formula (I) R⁸ is selected from the group consisting of heteroaryl-C(O)NH-aryl, heteroaryl-aryl, heteroaryl-C(O)-aryl, heteroaryl-C(O)—C₃-C₁₀ cycloalkyl, heteroaryl-C(O)-heterocyclyl, heteroaryl-C(O)NH—C₃-C₁₀ cycloalkyl, heteroaryl-C(O)NH-heterocyclyl, heteroaryl-C₃-C₁₀ cycloalkyl, heteroaryl-CH₂-aryl, heteroaryl-CH₂—C₃-C₁₀ cycloalkyl, heteroaryl-CH₂CH₂-aryl, heteroaryl-CH₂CH₂—C₃-C₁₀ cycloalkyl, heteroaryl-CH₂CH₂-heterocyclyl, heteroaryl-CH₂-heterocyclyl, heteroaryl-heterocyclyl, heteroaryl-N(Me)-aryl, heteroaryl-N(Me)-C₃-C₁₀ cycloalkyl, heteroaryl-N(Me)-heterocyclyl, heteroaryl-NH-aryl, heteroaryl-NHC(O)-aryl, heteroaryl-NHC(O)—C₃-C₁₀ cycloalkyl, heteroaryl-NHC(O)-heterocyclyl, heteroaryl-NHC(O)NH-aryl, heteroaryl-NHC(O)NH—C₃-C₁₀ cycloalkyl, heteroaryl-NHC(O)NH-heterocyclyl, heteroaryl-NH—C₃-C₁₀ cycloalkyl, heteroaryl-NH-heterocyclyl, heteroaryl-O-aryl, heteroaryl-O—C₃-C₁₀ cycloalkyl, and heteroaryl-O-heterocyclyl.

In a further particular embodiment of formula (I) R⁸ is selected from the group consisting of heterocyclyl-aryl, heterocyclyl-C(O)-aryl, heterocyclyl-C(O)—C₃-C₁₀ cycloalkyl, heterocyclyl-C(O)-heteroaryl, heterocyclyl-C(O)NH-aryl, heterocyclyl-C(O)NH—C₃-C₁₀ cycloalkyl, heterocyclyl-C(O)NH-heteroaryl, heterocyclyl-C₃-C₁₀ cycloalkyl, heterocyclyl-CH₂-aryl, heterocyclyl-CH₂C₃-C₁₀ cycloalkyl, heterocyclyl-CH₂CH₂-aryl, heterocyclyl-CH₂CH₂—C₃-C₁₀ cycloalkyl, heterocyclyl-CH₂CH₂-heteroaryl, heterocyclyl-CH₂-heteroaryl, heterocyclyl-heteroaryl, heterocyclyl-N(Me)-aryl, heterocyclyl-N(Me)-C₃-C₁₀ cycloalkyl, heterocyclyl-N(Me)-heteroaryl, heterocyclyl-NH-aryl, heterocyclyl-NHC(O)-aryl, heterocyclyl-NHC(O)—C₃-C₁₀ cycloalkyl, heterocyclyl-NHC(O)-heteroaryl, heterocyclyl-NHC(O)NH-aryl, heterocyclyl-NHC(O)NH—C₃-C₁₀ cycloalkyl, heterocyclyl-NHC(O)NH-heteroaryl, heterocyclyl-NH—C₃-C₁₀ cycloalkyl, heterocyclyl-NH-heteroaryl, heterocyclyl-O-aryl, heterocyclyl-O—C₃-C₁₀ cycloalkyl, and heterocyclyl-O-heteroaryl.

In a preferred embodiment of formula (I) R⁸ is selected from the group consisting of aryl-heterocyclyl and heteroaryl-heterocyclyl.

In a specific embodiment of formula (I) R⁸ is selected from the group consisting of azetidinyl, cyclopropanyl, cyclobutanyl, cyclopentanyl, cyclohexanyl, cyclohexanylcyclobutyl, cyclohexanylcyclopropyl, cyclohexylcyclohexyl, phenylcyclobutyl, phenylcyclobutyl, phenylcyclohexyl, phenoxycyclobutyl, phenoxycyclopentyl, phenoxycyclohexyl, benzylcyclobutyl, benzylcyclobutyl, benzylcyclohexyl, phenylaminocyclobutyl, phenylaminocyclobutyl, phenylaminocyclohexyl, 7-azabicyclo[4.2.0]octa-1,3,5-trienyl, 2,3-dihydro-1H-indolyl, 1,2,3,4-tetrahydroquinolinyl, 2,3-dihydro-1H-isoindolyl, 1,2,3,4-tetrahydroisoquinolinyl, phenylazetidinyl, phenylpyrrolidinyl, phenylpiperidinyl, phenylazetidinyl, phenylazetidinonyl, phenylpyrrolidinonyl, phenylpiperidinonyl, phenoxyazetidinyl, phenoxypyrrolidinyl, phenoxypiperidinyl, phenoxyazetidinyl, phenoxypyrrolidinyl, phenoxypiperidinyl, phenoxypiperidinyl, phenoxyazetidinonyl, phenoxypyrrolidinonyl, phenoxypiperidinonyl, benzylazetidinyl, benzylpyrrolidinyl, benzylpiperidinyl, benzylazetidinonyl, benzylpyrrolidinonyl, benzylpiperidinonyl, phenylaminoazetidinyl, phenylaminopyrrolidinyl, phenylaminopiperidinyl, phenylaminoazetidinyl, phenylaminoazetidinonyl, phenylaminopyrrolidinonyl, phenylaminopiperidinonyl, phenyl, phenylphenyl, benzylphenyl, phenoxyphenyl, phenylaminophenyl, phenylsulfanylphenyl, phenylcarbonylphenyl, naphtyl, phenalenyl, anthracenyl, phenylnaphtyl, 5-phenylnaphthalen-2-yl, phenylfuranyl, phenylpyrrolyl, phenylthiophenyl, phenylisoxazolyl, phenyloxazolyl, phenyloxadiazolyl, benzylisoxazolyl, benzyloxazolyl, benzyloxadiazolyl, thiazolyl, phenylthiazolyl, imidazolylthiazolyl, pyrazinylthiazolyl, phenylthiadiazolyl, [1,3]thiazolo[5,4-b]pyridinyl, [1,3]oxazolo[5,4-b]pyridinyl, 3H-imidazo[4,5-b]pyridinyl, [1,3]thiazolo[5,4-c]pyridinyl, [1,3]oxazolo[5,4-c]pyridinyl, 3H-imidazo[4,5-c]pyridinyl, [1,3]thiazolo[4,5-c]pyridinyl, [1,3]oxazolo[4,5-c]pyridinyl, 1H-imidazo[4,5-c]pyridinyl, [1,3]thiazolo[5,4-c]pyridazinyl, [1,3]oxazolo[5,4-c]pyridazinyl, 7H-imidazo[4,5-c]pyridazinyl, [1,3]thiazolo[5,4-d]pyrimidinyl, [1,3]oxazolo[5,4-d]pyrimidinyl, 9H-purinyl, [1,3]thiazolo[4,5-d]pyridazinyl, [1,3]oxazolo[4,5-d]pyridazinyl, 1H-imidazo[4,5-d]pyridazinyl, [1,3]thiazolo[5,4-d][1,2,3]triazinyl, [1,3]oxazolo[5,4-d][1,2,3]triazinyl, 7H-imidazo[4,5-d][1,2,3]triazinyl, phenylpyrazolyl, phenyltriazolyl, phenyltetrazolyl, benzylpyrazolyl, benzyltriazolyl, benzyltetrazolyl, naphatalenylcyclopropanyl, naphtalenylmethylcyclobutanyl, naphtalenylaminocyclopentanyl, napthalenyloxyazetidinyl, naphtalenylcarbonylpyrrolidinyl, naphatalenylpiperidinyl, naphtalenylmethylazetidinonyl, naphtalenylaminopyrrolidinonyl, napthalenyloxypiperidinonyl, naphtalenylcarbonylpyrazolyl, naphatalenyltriazolyl, naphtalenylmethyltetrazolyl, naphtalenylaminofuranyl, napthalenyloxypyrrolyl, naphtalenylcarbonylthienyl, and naphatalenyloxazolyl. More preferably R⁸ is selected from the group consisting of phenyl, phenylcyclopentyl, phenylpyrrolidine, benzylpyrrolidine, phenoxypyrrolidine, and phenylaminopyrrolidine.

The substitution referred to in relation to R⁸ may be by any one or more substituents as described herein above. Preferably the substituents for R⁸ may be one or more substituents selected from the group consisting of halogen, hydroxyl, C₁-C₆ alkyl, C₁-C₆ alkoxy, —CN, —NO₂, —NH₂, —SO₂—C₁-C₆ alkyl, —S(O)—C₁-C₆ alkyl, C₂-C₆ alkenyl, C₂-C₆ alkynyl, C₃-C₁₀ cycloalkyl, aryl, heterocyclyl, and heteroaryl. More preferably the one or more substituents may be selected from the group consisting of halogen, hydroxyl, C₁-C₆ alkyl, C₁-C₆ alkoxy, —CN, —NO₂, —SO₂—C₁-C₆ alkyl, —NH₂, —SO₂—C₁-C₆ alkyl, —S(O)—C₁-C₆ alkyl, C₂-C₆ alkenyl, and C₂-C₆ alkynyl. Even more preferably the one or more substituents may be selected from the group consisting of fluoro, chloro, hydroxy, methoxy, ethoxy, methyl, ethyl, propyl, isopropyl, tert-butyl, sec-butyl, cyano, nitro, sulfanyl, methylsulfanyl, sulfonyl, and methylsulfonyl.

The R⁹ moiety for use in connection with Z² and Z³ moieties may be as defined herein above, more preferably R⁹ may be selected from the group consisting of H, C₁-C₄ alkyl, trifluoromethyl, trifluoroethyl, C₁-C₄ alkoxy, halogen-C₁-C₄ alkyl, —(CH₂)₀₋₂-aryl, —(CH₂)₀₋₂-heterocyclyl, and —(C₂)₀₋₂-heteroaryl. Even more preferably R⁹ may be selected from the group consisting of H, methyl, ethyl, trifluoromethyl, —CH₂OH, —(CH₂)₀₋₁-aryl, and —(CH₂)₀₋₁-heteroaryl. Yet more preferably R⁹ may be selected from the group consisting of H, methyl, ethyl, trifluoromethyl, —CH₂OH, aryl, and heterocyclyl.

The R¹⁰ and/or R¹¹ moieties for use in connection with Z² and Z³ moieties may be as defined herein above, more preferably R¹⁰ and R¹¹ each independently may be selected from the group consisting of H, C₁-C₄ alkyl, C₃-C₇ cycloalkyl, aryl, —(CH₂)₁₋₂—C₃-C₇ cycloalkyl, —(CH₂)₁₋₂-aryl, wherein alkyl, cycloalkyl, and aryl optionally are substituted, or R¹⁰ together with R¹¹ may form a heterocyclyl ring together with the nitrogen to which they are attached. Even more preferably R¹⁰ and R¹¹ each independently may be selected from the group consisting of H, C₁-C₄ alkyl, C₃-C₇ cycloalkyl, aryl, —(C₂)₁₋₂—C₃-C₇ cycloalkyl, —(CH₂)₁₋₂-aryl, wherein alkyl, cycloalkyl, and aryl optionally are substituted.

More specifically R¹⁰ and R¹¹ may each independently be selected from the group consisting of —H, methyl, ethyl, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, pyridinyl, pyridazinyl, imidazolyl, imidazolidinyl, pyrimidinyl, pyrazolyl, triazolyl, pyrazinyl, pyrazolinyl, pyrazolidinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl, aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, 1,4-dioxaspiro[4.2]heptyl, 2-oxopiperazinyl, 2-oxopiperidinyl, 2,8-diazaspiro[4.5]decanyl and 8-azaspiro[4.5]decanyl.

Alternatively R¹⁰ together with R¹¹ may form a heterocyclyl ring together with the nitrogen to which they are attached.

In relation to the Z₁ moiety m specifies the chain length for —(CH₂)— chains, m may be as defined herein above, or more preferably m may be 0, or an integer from 1 to 3, such as e.g. 1, 2 or 2.

In relation to R⁴ and R⁵ n specifies the chain length for —(CH₂)— chains, n may be as defined herein above, or more preferably n may be 0, or an integer from 1 to 3, such as e.g. 1, 2 or 2.

In relation to the Z₂ moiety q specifies the chain length for —(CH₂)— chains, q may be as defined herein above, or more preferably q may be 0, or an integer from 1 to 3, such as e.g. 1, 2 or 2.

In relation to R⁶ and R⁷ r specifies the chain length for —(CH₂)— chains, r may be as defined herein above, or more preferably r may be 0, or an integer from 1 to 3, such as e.g. 1, 2 or 2.

Examples of preferred compounds of formula (I) are:
(5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone;
(6-(1-(methylamino)ethyl)pyridin-2-yl)((2S,4R)-4-phenyl-2-(((R)-3-phenylpyrrolidin-1-yl)methyl)pyrrolidin-1-yl)methanone;
(2S,4R)-1-((3R,5S)-1-(2-((S)-2-aminopropanamido)-3-(1H-1,2,4-triazol-1-yl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)butanoyl)-3-phenylpyrrolidine-5-carbonyl)-N-methyl-4-phenylpyrrolidine-2-carboxamide;
(2S,4R)-1-((S)-2-((R)-2-aminopropanamido)-3-(4-carbamoylphenyl)propanoyl)-N—((R)-2,3-dihydro-1H-inden-1-yl)-4-phenylpyrrolidine-2-carboxamide;
(2R,3R)-1-((S)-2-((S)-2-aminopropanamido)-3-(3-carbamoylphenyl)propanoyl)-N—((R)-2,3-dihydro-1H-inden-1-yl)-3-phenylazetidine-2-carboxamide;
(2S,4R)-1-((S)-2-((S)-2-aminopropanamido)-3-(3-cyanophenyl)propanoyl)-N—((R)-2,3-dihydro-1H-inden-1-yl)-4-phenylpyrrolidine-2-carboxamide;
(2S,4R)-1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)-3-(furan-2-yl)propanoyl)-3-phenylpyrrolidine-5-carbonyl)-N-methyl-4-phenylpyrrolidine-2-carboxamide;
(S)—N—((S)-3-(3-cyanophenyl)-1-oxo-1-((2S,4R)-4-phenyl-2-(((R)-3-phenylpyrrolidin-1-yl)methyl)pyrrolidin-1-yl)propan-2-yl)-2-(methylamino)butanamide;
(2S,4R)-1-((S)-2-((R)-2-aminopropanamido)-3-(3-carbamoylphenyl)propanoyl)-N—((R)-2,3-dihydro-1H-inden-1-yl)-4-phenylpyrrolidine-2-carboxamide;
(2S,3S)-1-((S)-2-((S)-2-aminopropanamido)-3-(3-carbamoylphenyl)propanoyl)-N—((S)-2,3-dihydro-1H-inden-1-yl)-2-phenylazetidine-3-carboxamide;
{(2S,4R)-4-(4-Fluoro-phenyl)-2-[3(R)-(4-fluoro-phenyl)-pyrrolidine-1-carbonyl]-pyrrolidin-1-yl}-[5-(1(S)-methylamino-ethyl)-furan-2-yl]-methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(R)-methylamino-ethyl)-furan-2-yl]-methanone;
(5-(1(S)-aminoethyl)furan-2-yl)((2S,4R)-4-phenyl-2-(((R)-3-phenylpyrrolidin-1-yl)methyl)pyrrolidin-1-yl)methanone;
(5-(1(R)-amino-ethyl)furan-2-yl)((2S,4R)-4-phenyl-2-(((R)-3-phenylpyrrolidin-1-yl)methyl)pyrrolidin-1-yl)methanone;
(3-(1(S)-(methylamino)ethyl)phenyl)((2S,4R)-4-phenyl-2-(((R)-3-phenylpyrrolidin-1-yl)methyl)pyrrolidin-1-yl)methanone;
(3-(1(R)-(methylamino)ethyl)phenyl)((2S,4R)-4-phenyl-2-(((R)-3-phenylpyrrolidin-1-yl)methyl)pyrrolidin-1-yl)methanone;
(2S,4S)-4-Cyclohexyl-1-(2,8-diaza-spiro[4.5]decane-3-carbonyl)-pyrrolidine-2-carboxylic acid (R)-indan-1-ylamide;
2,8-Diaza-spiro[4.5]decane-3-carboxylic acid [(S)-cyclohexyl-((R)-indan-1-ylcarbamoyl)-methyl]-amide;
(2R,4R)-4-Cyclohexyl-1-(2,8-diaza-spiro[4.5]decane-3-carbonyl)-pyrrolidine-2-carboxylicacid(S)-indan(R)-1-ylamide; and
(2R,4R)-4-Cyclohexyl-1-(2,8-diaza-spiro[4.5]decane-3-carbonyl)-pyrrolidine-2-carboxylicacid(R)-indan(R)-1-ylamide.

Further examples of compounds of formula (I):
1-[2-(2-Amino-propionylamino)-3-pyridin-3-yl-propionyl]-4-phenyl-pyrrolidine-2-carboxylic acid (2-methylcarbamoyl-indan-1-yl)-amide;
1-{1-[2-(2-Amino-propionylamino)-3-pyridin-3-yl-propionyl]-4-phenyl-pyrrolidine-2-carbonyl}-4-phenyl-pyrrolidine-2-carboxylic acid methylamide;
1-[2-(2-Amino-propionylamino)-3-methyl-butyryl]-3-phenyl-azetidine-2-carboxylic acid (2-methylcarbamoyl-indan-1-yl)-amide;
1-{1-[2-(2-Amino-propionylamino)-butyryl]-4-phenyl-pyrrolidine-2-carbonyl}-4-phenyl-pyrrolidine-2-carboxylic acid methylamide;
1-{1-[2-(2-Amino-propionylamino)-3-(3-cyano-phenyl)-propionyl]-4-phenyl-pyrrolidine-2-carbonyl}-4-phenyl-pyrrolidine-2-carboxylic acid methylamide;
1-[2-(2-Amino-propionylamino)-3-cyclopropyl-propionyl]-4-phenyl-pyrrolidine-2-carboxylic acid (2-methylcarbamoyl-indan-1-yl)-amide;
1-[2-(2-Amino-propionylamino)-3-(3-chloro-phenyl)-propionyl]-3-phenyl-azetidine-2-carboxylic acid (2-methylcarbamoyl-indan-1-yl)-amide;
1-[2-(2-Amino-propionylamino)-4-methanesulfonyl-butyryl]-4-phenyl-pyrrolidine-2-carboxylic acid benzyl-methylcarbamoylmethyl-amide;
1-[2-(2-Amino-propionylamino)-3-ureido-propionyl]-4-phenyl-pyrrolidine-2-carboxylic acid benzyl-methylcarbamoylmethyl-amide;
1-[2-(2-Amino-propionylamino)-3-(3-cyano-phenyl)-propionyl]-4-phenyl-pyrrolidine-2-carboxylic acid (2-methylcarbamoyl-indan-1-yl)-amide;
1-{1-[2-(2-Amino-propionylamino)-3-pyridin-3-yl-propionyl]-4-phenyl-pyrrolidine-2-carbonyl}-4-phenyl-pyrrolidine-2-carboxylic acid methylamide;
1-[2-(2-Amino-propionylamino)-3-pyrrolidin-2-yl-propionyl]-4-phenyl-pyrrolidine-2-carboxylic acid (2-methylcarbamoyl-indan-1-yl)-amide;
1-{1-[2-(2-Amino-propionylamino)-3-(3-cyano-phenyl)-propionyl]-4-phenyl-pyrrolidine-2-carbonyl}-4-phenyl-pyrrolidine-2-carboxylic acid methylamide;
1-{1-[2-(2-Amino-propionylamino)-3-[1,2,4]triazol-1-yl-propionyl]-4-phenyl-pyrrolidine-2-carbonyl}-4-phenyl-pyrrolidine-2-carboxylic acid methylamide;
1-[2-(2-Amino-propionylamino)-3-(3-carbamoyl-phenyl)-propionyl]-3-phenyl-azetidine-2-carboxylic acid (2-methylcarbamoyl-indan-1-yl)-amide;
1-{1-[2-(2-Amino-propionylamino)-3-pyridin-3-yl-propionyl]-4-phenyl-pyrrolidine-2-carbonyl}-3-phenyl-pyrrolidine-2-carboxylic acid methylamide;
1-[2-(2-Amino-propionylamino)-3-cyclopropyl-propionyl]-3-phenyl-azetidine-2-carboxylic acid (2-methylcarbamoyl-indan-1-yl)-amide;
1-[2-(2-Amino-propionylamino)-butyryl]-4-phenyl-pyrrolidine-2-carboxylic acid [1-methylcarbamoyl-2-(3-trifluoromethyl-phenyl)-ethyl]-amide;
2-[6-(1-Amino-ethyl)-pyridine-2-carbonyl]-1,2,3,4-tetrahydro-isoquinoline-3-carboxylic acid indan-1-ylamide;
1-(3-(1-aminoethyl)benzoyl)-N-(−2,3-dihydro-1H-inden-1-yl)-octahydro-1H-indole-2-carboxamide;
1-[6-(1-Amino-ethyl)-pyridine-2-carbonyl]-3-phenyl-azetidine-2-carboxylic acid indan-1-ylamide;
1-(3-(1-aminoethyl)benzoyl)-N-(-2,3-dihydro-1H-inden-1-yl)-3-phenylpyrrolidine-2-carboxamide;
1-[6-(1-Amino-ethyl)-pyridine-2-carbonyl]-4-phenyl-pyrrolidine-2-carboxylic acid indan-1-ylamide;
1-(3-(1-aminoethyl)benzoyl)-N-(-2,3-dihydro-1H-inden-1-yl)-5-phenylpyrrolidine-2-carboxamide;
1-(3-(1-aminoethyl)benzamido)-N-(-2,3-dihydro-1H-inden-1-yl)-2,3-dihydro-1H-indene-2-carboxamide;
1-[6-(1-Amino-ethyl)-pyridine-2-carbonyl]-4-(4-fluoro-phenyl)-pyrrolidine-2-carboxylic acid indan-1-ylamide;
1-(3-(1-aminoethyl)benzoyl)-4-(4-chlorophenyl)-N-(-2,3-dihydro-1H-inden-1-yl)pyrrolidine-2-carboxamide;
2-amino-N-(-4-methyl-1-oxo-1-(-4-phenyl-2-((3-phenylpyrrolidin-1-yl)methyl)pyrrolidin-1-yl)pentan-2-yl)propanamide;
2-amino-N-(-3-cyclohexyl-1-oxo-1-(-4-phenyl-2-((3-phenylpyrrolidin-1-yl)methyl)pyrrolidin-1-yl)propan-2-yl)propanamide;
2-amino-N-(-3-methyl-1-oxo-1-(-4-phenyl-2-((3-phenylpyrrolidin-1-yl)methyl)pyrrolidin-1-yl)butan-2-yl)propanamide;
2-amino-N-(-3-methyl-1-oxo-1-(-4-phenyl-2-((3-phenylpyrrolidin-1-yl)methyl)pyrrolidin-1-yl)butan-2-yl)propanamide;
2-amino-N-(-3-methyl-1-oxo-1-(-4-phenyl-2-((3-phenylpyrrolidin-1-yl)methyl)pyrrolidin-1-yl)pentan-2-yl)propanamide;
2-amino-N-(-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-4-(1H-tetrazol-5-yl)butan-2-yl)propanamide;
2-amino-N-(-3-(3-chlorophenyl)-1-oxo-1-(-4-phenyl-2-((3-phenylpyrrolidin-1-yl)methyl)pyrrolidin-1-yl)propan-2-yl)propanamide;
2-amino-N-(-3-(4-chlorophenyl)-1-oxo-1-(-4-phenyl-2-((3-phenylpyrrolidin-1-yl)methyl)pyrrolidin-1-yl)propan-2-yl)propanamide;
2-amino-N-(-3-(2,4-dichlorophenyl)-1-oxo-1-(-4-phenyl-2-((3-phenylpyrrolidin-1-yl)methyl)pyrrolidin-1-yl)propan-2-yl)propanamide;
2-amino-N-(-3-(3,4-dichlorophenyl)-1-oxo-1-(-4-phenyl-2-((3-phenylpyrrolidin-1-yl)methyl)pyrrolidin-1-yl)propan-2-yl)propanamide;
2-amino-N-(-3-(3,4-difluorophenyl)-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-3-(4-(trifluoromethyl)phenyl)propan-2-yl)propanamide;
2-amino-N-(-3-(3-cyanophenyl)-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-3-(pyridin-3-yl)propan-2-yl)propanamide;
2-amino-N-(-1-oxo-1-(-4-phenyl-2-((3-phenylpyrrolidin-1-yl)methyl)pyrrolidin-1-yl)butan-2-yl)propanamide;
2-amino-N-(-3-cyclopropyl-1-oxo-1-(-4-phenyl-2-((3-phenylpyrrolidin-1-yl)methyl)pyrrolidin-1-yl)propan-2-yl)propanamide;
3-(-2-(-2-aminopropanamido)-3-oxo-3-(-4-phenyl-2-((3-phenylpyrrolidin-1-yl)methyl)pyrrolidin-1-yl)propyl)benzamide;
4-(-2-(-2-aminopropanamido)-3-oxo-3-(-4-phenyl-2-((3-phenylpyrrolidin-1-yl)methyl)pyrrolidin-1-yl)propyl)benzamide;
2-amino-N-(-4,4-dimethyl-1-oxo-1-(-4-phenyl-2-((3-phenylpyrrolidin-1-yl)methyl)pyrrolidin-1-yl)pentan-2-yl)propanamide;
(4-(1-aminoethyl)-5-methylfuran-2-yl)(-4-phenyl-2-((3-phenylpyrrolidin-1-yl)methyl)pyrrolidin-1-yl)methanone;
(6-(1-aminoethyl)pyridin-2-yl)(-4-phenyl-2-((3-phenylpyrrolidin-1-yl)methyl)pyrrolidin-1-yl)methanone;
(3-(1-aminoethyl)phenyl)(-4-phenyl-2-((3-phenylpyrrolidin-1-yl)methyl)pyrrolidin-1-yl)methanone;
1-(3-(1-aminoethyl)-2-methylfuran-5-carbonyl)-N-(-2,3-dihydro-1H-inden-1-yl)-4-phenylpyrrolidine-2-carboxamide;
1-[6-(1-Amino-ethyl)-pyridine-2-carbonyl]-4-phenyl-pyrrolidine-2-carboxylic acid indan-1-ylamide;
1-(3-(1-aminoethyl)benzoyl)-N-(-2,3-dihydro-1H-inden-1-yl)-4-phenylpyrrolidine-2-carboxamide;
(6-(1-aminoethyl)pyridin-2-yl)(-2-((-3-(4-fluorophenyl)pyrrolidin-1-yl)methyl)-4-phenylpyrrolidin-1-yl)methanone;
(3-(1-aminoethyl)phenyl)(-2-((-3-(4-chlorophenyl)pyrrolidin-1-yl)methyl)-4-phenylpyrrolidin-1-yl)methanone;
(-4-(4-fluorobenzyl)-2-((3-phenylpyrrolidin-1-yl)methyl)pyrrolidin-1-yl)(5-(1-aminoethyl)furan-2-yl)methanone;
(2,8-Diaza-spiro[4.5]dec-3-yl)-[-4-phenyl-2-(3-phenyl-pyrrolidine-1-carbonyl)-pyrrolidin-1-yl]-methanone;
1-(2,8-Diaza-spiro[4.5]decane-3-carbonyl)-4-phenyl-pyrrolidine-2-carboxylic acid-indan-1-ylamide;
4-Cyclohexyl-1-(2,8-diaza-spiro[4.5]decane-3-carbonyl)-pyrrolidine-2-carboxylic acid-indan-1-ylamide;
2,8-Diaza-spiro[4.5]decane-3-carboxylic acid [-cyclohexyl-(-indan-1-ylcarbamoyl)-methyl]-amide;
[5-(1-Amino-ethyl)-furan-2-yl]-[4-(4-fluoro-benzyl)-2-(3-phenyl-pyrrolidine-1-carbonyl)-pyrrolidin-1-yl]-methanone;
4-Benzyl-1-(2,8-diaza-spiro[4.5]decane-3-carbonyl)-pyrrolidine-2-carboxylic acid (2-carbamoyl-indan-1-yl)-amide;
(5-(1-aminoethyl)furan-2-yl)(3-phenyl-2-((3-phenylazetidin-1-yl)methyl)azetidin-1-yl)methanone;
(6-(1-aminoethyl)piperidin-2-yl)(-4-phenyl-2-((3-phenylazetidin-1-yl)methyl)pyrrolidin-1-yl)methanone;
[3-(1-Amino-ethyl)-phenyl]-{-2-[3-(4-fluoro-benzyl)-pyrrolidine-1-carbonyl]-4-phenylpyrrolidin-1-yl}-methanone;
(5-(1-aminoethyl)furan-2-yl)(-2-((3-phenylpyrrolidin-1-yl)methyl)pyrrolidin-1-yl)methanone;
(6-(1-aminoethyl)piperidin-2-yl)(-3-((3-phenylpyrrolidin-1-yl)methyl)-3,4-dihydroisoquinolin-2(1H)-yl)methanone;
(6-(1-aminoethyl)pyridin-2-yl)(-2-((3-phenylpyrrolidin-1-yl)methyl)-octahydroindol-1-yl)methanone;
(3-(1-aminoethyl)phenyl)(-4-(benzyloxy)-2-((3-phenylpyrrolidin-1-yl)methyl)pyrrolidin-1-yl)methanone;
(5-(1-aminoethyl)furan-2-yl)(-4-fluoro-2-((3-phenylpyrrolidin-1-yl)methyl)pyrrolidin-1-yl)methanone;
6-(1-aminoethyl)-N-(3-(3-phenylpyrrolidin-1-yl)-1-(1H-1,2,4-triazol-1-yl)propan-2-yl)piperidine-2-carboxamide;
(6-(1-aminoethyl)pyridin-2-yl)(3-phenyl-2-((3-phenylpyrrolidin-1-yl)methyl)azetidin-1-yl)methanone;
(3-(1-aminoethyl)phenyl)(-3-phenyl-2-((3-phenylpyrrolidin-1-yl)methyl)pyrrolidin-1-yl)methanone;
(6-(1-aminoethyl)pyridin-2-yl)(-4-phenyl-2-((3-phenylpyrrolidin-1-yl)methyl)pyrrolidin-1-yl)methanone;
(3-(1-aminoethyl)phenyl)(-2-phenyl-5-((3-phenylpyrrolidin-1-yl)methyl)pyrrolidin-1-yl)methanone;
(5-(1-aminoethyl)furan-2-yl)(-4-((3-phenylpyrrolidin-1-yl)methyl)thiazolidin-3-yl)methanone;
3-(1-aminoethyl)-N-(-2-((3-phenylpyrrolidin-1-yl)methyl)-2,3-dihydro-1H-inden-1-yl)benzamide;
[5-(1-Amino-ethyl)-furan-2-yl]-[-4-methylamino-2-(-3-phenyl-pyrrolidine-1-carbonyl)-pyrrolidin-1-yl]-methanone;
(6-(1-aminoethyl)piperidin-2-yl)(-4-hydroxy-4-phenyl-2-((3-phenylpyrrolidin-1-yl)methyl)pyrrolidin-1-yl)methanone;
(6-(1-aminoethyl)pyridin-2-yl)(-4-(4-fluorophenyl)-2-((3-phenylpyrrolidin-1-yl)methyl)pyrrolidin-1-yl)methanone;
(3-(1-aminoethyl)phenyl)(-4-(4-chlorophenyl)-2-((3-phenylpyrrolidin-1-yl)methyl)pyrrolidin-1-yl)methanone;
[6-(1-Amino-ethyl)-piperidin-2-yl]-[-4-phenyl-2-(2-phenyl-pyrrolidine-1-carbonyl)-pyrrolidin-1-yl]-methanone;
4-(-1-(2-(1-aminoethyl)furan-5-carbonyl)-3-phenylpyrrolidine-5-carbonyl)-1,3-dimethylpiperazin-2-one;
(6-(1-aminoethyl)piperidin-2-yl)(-2-((-2,3-dihydro-1H-inden-1-ylamino)methyl)pyrrolidin-1-yl)methanone;
1-(2-(1-aminoethyl)furan-5-carbonyl)-4-(benzyloxy)-N-(-2,3-dihydro-1H-inden-1-yl)pyrrolidine-2-carboxamide;
(6-(1-aminoethyl)piperidin-2-yl)(-2-((-2,3-dihydro-1H-inden-1-ylamino)methyl)-4-fluoropyrrolidin-1-yl)methanone;
4-(4-fluorobenzyl)-1-(2-(1-aminoethyl)furan-5-carbonyl)-N-(-2,3-dihydro-1H-inden-1-yl)pyrrolidine-2-carboxamide;
(5-(1-aminoethyl)furan-2-yl)(-4-((-2,3-dihydro-1H-inden-1-ylamino)methyl)thiazolidin-3-yl)methanone;
2-(-4-(2-(1-aminoethyl)piperidine-6-carbonyl)-3-benzyl-2-oxopiperazin-1-yl)-N-(-2,3-dihydro-1H-inden-1-yl)acetamide;
1-(2-(1-aminoethyl)piperidine-6-carbonyl)-N-(-2,3-dihydro-1H-inden-1-yl)-4-hydroxy-4-phenylpyrrolidine-2-carboxamide;
(5-(1-aminoethyl)-2-methylfuran-3-yl)(-4-phenyl-2-((3-phenylpyrrolidin-1-yl)methyl)pyrrolidin-1-yl)methanone;
(6-(1-aminoethyl)piperidin-2-yl)(-4-phenyl-2-((3-phenylpyrrolidin-1-yl)methyl)pyrrolidin-1-yl)methanone;
(4-(aminomethyl)-5-isobutylfuran-2-yl)(-4-phenyl-2-((3-phenylpyrrolidin-1-yl)methyl)pyrrolidin-1-yl)methanone;
1-(2-(1-aminoethyl)-5-methylfuran-4-carbonyl)-N-(-2,3-dihydro-1H-inden-1-yl)-4-phenylpyrrolidine-2-carboxamide;
1-(2-(1-aminoethyl)furan-5-carbonyl)-N-(-2,3-dihydro-1H-inden-1-yl)-4-phenylpyrrolidine-2-carboxamide;
1-(2-(1-aminoethyl)piperidine-6-carbonyl)-N-(-2,3-dihydro-1H-inden-1-yl)-4-phenylpyrrolidine-2-carboxamide;
1-(3-(aminomethyl)-2-isobutylfuran-5-carbonyl)-N-(-2,3-dihydro-1H-inden-1-yl)-4-phenylpyrrolidine-2-carboxamide;
[5-(1-Amino-ethyl)-furan-2-yl]-{-4-(4-fluoro-phenyl)-2-[3-(4-fluoro-phenyl)-pyrrolidine-1-carbonyl]-pyrrolidin-1-yl}-methanone;
[6-(1-Amino-ethyl)-piperidin-2-yl]-{-4-(4-chloro-phenyl)-2-[3-(4-chloro-phenyl)-pyrrolidine-1-carbonyl]-pyrrolidin-1-yl}-methanone;
(6-(1-aminoethyl)pyridin-2-yl)(-2-((-3-(3-fluorophenyl)pyrrolidin-1-yl)methyl)-4-phenylpyrrolidin-1-yl)methanone;
(3-(1-aminoethyl)phenyl)(-2-((-3-(3,4-dichlorophenyl)pyrrolidin-1-yl)methyl)-4-phenylpyrrolidin-1-yl)methanone;
(-4-(4-fluorophenyl)-2-((-3-(3-fluorophenyl)pyrrolidin-1-yl)methyl)pyrrolidin-1-yl)(5-(1-(methylamino)propyl)furan-2-yl)methanone;
(5-(1-aminoethyl)furan-2-yl)(2-((2,3-dihydro-1H-inden-1-ylamino)methyl)-4-phenylpyrrolidin-1-yl)methanone;
2-amino-N-(-5-oxo-1-phenyl-3-((3-phenylpyrrolidin-1-yl)methyl)-octahydro-1H-pyrrolo[1,2-a]azepin-6-yl)propanamide;
(5-(-1-aminoethyl)furan-2-yl)(-2-(phenoxymethyl)-4-phenylpyrrolidin-1-yl)methanone;
(5-(-1-aminoethyl)furan-2-yl)(-2-((naphthalen-1-yloxy)methyl)-4-phenylpyrrolidin-1-yl)methanone;
(5-(-1-aminoethyl)furan-2-yl)(-2-((2,3-dihydro-1H-inden-1-ylamino)methyl)-4-phenylpyrrolidin-1-yl)methanone;
(5-(-1-aminoethyl)furan-2-yl)(-4-phenyl-2-((-1,2,3,4-tetrahydronaphthalen-1-ylamino)methyl)pyrrolidin-1-yl)methanone;
(5-(-1-aminoethyl)furan-2-yl)(-2-(2-benzyl-2H-tetrazol-5-yl)-4-phenylpyrrolidin-1-yl)methanone;
(5-(-1-aminoethyl)furan-2-yl)(-2-(4-benzyloxazol-2-yl)-4-phenylpyrrolidin-1-yl)methanone;
[5-(-1-Amino-ethyl)-furan-2-yl]-[-2-(5-benzoyl-[1,2,4]oxadiazol-3-yl)-4-phenyl-pyrrolidin-1-yl]-methanone;
1-(2-(-1-aminoethyl)furan-5-carbonyl)-4-phenyl-N-(4-phenyl-1,2,3-thiadiazol-5-yl)pyrrolidine-2-carboxamide;
1-(2-(-1-aminoethyl)furan-5-carbonyl)-4-phenyl-N-(1-phenyl-1H-pyrazol-5-yl)pyrrolidine-2-carboxamide;
1-(2-(-1-aminoethyl)furan-5-carbonyl)-4-phenyl-N-(5-phenyl-1H-tetrazol-1-yl)pyrrolidine-2-carboxamide;
(5-(-1-aminoethyl)furan-2-yl)(-2-((1-methyl-1H-indol-3-yl)methyl)-4-phenylpyrrolidin-1-yl)methanone;
1-(3-((-1-(2-(-1-aminoethyl)furan-5-carbonyl)-4-phenylpyrrolidin-2-yl)methyl)-1H-indol-1-yl)ethanone;
(5-(-1-aminoethyl)furan-2-yl)(-2-(benzofuran-3-ylmethyl)-4-phenylpyrrolidin-1-yl)methanone;
[5-(-1-Amino-ethyl)-2-methoxy-phenyl]-[-4-phenyl-2-(-3-phenyl-pyrrolidine-1-carbonyl)-pyrrolidin-1-yl]-methanone;
[5-(-1-Amino-ethyl)-2-benzyloxy-phenyl]-[-4-phenyl-2-(-3-phenyl-pyrrolidine-1-carbonyl)-pyrrolidin-1-yl]-methanone;
[5-(-1-Amino-ethyl)-4-ethoxy-2-piperidin-1-yl-phenyl]-[-4-phenyl-2-(-3-phenyl-pyrrolidine-1-carbonyl)-pyrrolidin-1-yl]-methanone;
[5-(-1-Amino-ethyl)-1H-pyrrol-2-yl]-[-4-phenyl-2-(-3-phenyl-pyrrolidine-1-carbonyl)-pyrrolidin-1-yl]-methanone;
[5-(-1-Amino-ethyl)-furan-2-yl]-[-4-phenyl-2-(-3-phenyl-pyrrolidine-1-carbonyl)-pyrrolidin-1-yl]-methanone;
[5-(-1-Amino-ethyl)-[1,2,4]oxadiazol-3-yl]-[-4-phenyl-2-(-3-phenyl-pyrrolidine-1-carbonyl)-pyrrolidin-1-yl]-methanone;
[3-(-1-Amino-ethyl)-[1,2,4]oxadiazol-5-yl]-[-4-phenyl-2-(-3-phenyl-pyrrolidine-1-carbonyl)-pyrrolidin-1-yl]-methanone;
[5-(-1-Amino-ethyl)-oxazol-2-yl]-[-4-phenyl-2-(-3-phenyl-pyrrolidine-1-carbonyl)-pyrrolidin-1-yl]-methanone;
[5-(-1-Amino-ethyl)-1H-imidazol-2-yl]-[-4-phenyl-2-(-3-phenyl-pyrrolidine-1-carbonyl)-pyrrolidin-1-yl]-methanone;
[4-(-1-Amino-ethyl)-1-methyl-1H-imidazol-2-yl]-[-4-phenyl-2-(-3-phenyl-pyrrolidine-1-carbonyl)-pyrrolidin-1-yl]-methanone;
[4-(-1-Amino-ethyl)-phenyl]-[-4-phenyl-2-(-3-phenyl-pyrrolidine-1-carbonyl)-pyrrolidin-1-yl]-methanone;
(6-Aminomethyl-pyridin-2-yl)-[-4-phenyl-2-(-3-phenyl-pyrrolidine-1-carbonyl)-pyrrolidin-1-yl]-methanone;
(5-Aminomethyl-thiazol-2-yl)-[-4-phenyl-2-(-3-phenyl-pyrrolidine-1-carbonyl)-pyrrolidin-1-yl]-methanone;
(5-Aminomethyl-thiophen-2-yl)-[-4-phenyl-2-(-3-phenyl-pyrrolidine-1-carbonyl)-pyrrolidin-1-yl]-methanone;
(5-Methylaminomethyl-thiophen-2-yl)-[-4-phenyl-2-(-3-phenyl-pyrrolidine-1-carbonyl)-pyrrolidin-1-yl]-methanone;
(5-Methylaminomethyl-furan-2-yl)-[-4-phenyl-2-(-3-phenyl-pyrrolidine-1-carbonyl)-pyrrolidin-1-yl]-methanone;
(5-Aminomethyl-furan-2-yl)-[-4-phenyl-2-(-3-phenyl-pyrrolidine-1-carbonyl)-pyrrolidin-1-yl]-methanone;
(2-Aminomethyl-1,5-dimethyl-1H-imidazol-4-yl)-[-4-phenyl-2-(-3-phenyl-pyrrolidine-1-carbonyl)-pyrrolidin-1-yl]-methanone;
(5-Methylaminomethyl-[1,2,4]oxadiazol-3-yl)-[-4-phenyl-2-(-3-phenyl-pyrrolidine-1-carbonyl)-pyrrolidin-1-yl]-methanone;
[2-(-1-Amino-ethyl)-5-methyl-oxazol-4-yl]-[-4-phenyl-2-(-3-phenyl-pyrrolidine-1-carbonyl)-pyrrolidin-1-yl]-methanone;
(5-Aminomethyl-[1,2,4]oxadiazol-3-yl)-[-4-phenyl-2-(-3-phenyl-pyrrolidine-1-carbonyl)-pyrrolidin-1-yl]-methanone;
(5-Aminomethyl-furan-3-yl)-[-4-phenyl-2-(-3-phenyl-pyrrolidine-1-carbonyl)-pyrrolidin-1-yl]-methanone;
(4-Aminomethyl-5-methyl-furan-2-yl)-[-4-phenyl-2-(-3-phenyl-pyrrolidine-1-carbonyl)-pyrrolidin-1-yl]-methanone;
(4-Aminomethyl-5-isobutyl-furan-2-yl)-[-4-phenyl-2-(-3-phenyl-pyrrolidine-1-carbonyl)-pyrrolidin-1-yl]-methanone;
(5-Aminomethyl-isoxazol-3-yl)-[-4-phenyl-2-(-3-phenyl-pyrrolidine-1-carbonyl)-pyrrolidin-1-yl]-methanone;
(5-Aminomethyl-thiophen-3-yl)-[-4-phenyl-2-(-3-phenyl-pyrrolidine-1-carbonyl)-pyrrolidin-1-yl]-methanone;
[2-(-1-Amino-ethyl)-oxazol-5-yl]-[-4-phenyl-2-(-3-phenyl-pyrrolidine-1-carbonyl)-pyrrolidin-1-yl]-methanone;
(6-Methyl-2,8-diaza-spiro[4.5]dec-3-yl)-[-4-phenyl-2-(3-phenyl-pyrrolidin-1-ylmethyl)-pyrrolidin-1-yl]-methanone;
(6-Ethyl-2,8-diaza-spiro[4.5]dec-3-yl)-[-4-phenyl-2-(3-phenyl-pyrrolidin-1-ylmethyl)-pyrrolidin-1-yl]-methanone;
(-4-(4-fluorophenyl)-2-((3-(4-fluorophenyl)cyclopentyl)methyl)pyrrolidin-1-yl)(5-(1-(methylamino)ethyl)furan-2-yl)methanone;
[6-(1-Methylamino-ethyl)-piperidin-2-yl]-[-4-phenyl-2-(-3-phenyl-pyrrolidine-1-carbonyl)-pyrrolidin-1-yl]-methanone;
(6-(1-(methylamino)ethyl)piperidin-2-yl)(-4-phenyl-2-((-3-phenylpyrrolidin-1-yl)methyl)pyrrolidin-1-yl)methanone;
(2,8-Diaza-spiro[4.5]dec-3-yl)-[-2-(4-phenyl-thiazolo[4,5-c]pyridin-2-yl)-pyrrolidin-1-yl]-methanone;
(2,8-Diaza-spiro[4.5]dec-3-yl)-[-2-(7-phenyl-thiazolo[5,4-b]pyridin-2-yl)-pyrrolidin-1-yl]-methanone;
(2,8-Diaza-spiro[4.5]dec-3-yl)-[-2-(7-phenyl-thiazolo[5,4-d]pyrimidin-2-yl)-pyrrolidin-1-yl]-methanone;
(2,8-Diaza-spiro[4.5]dec-3-yl)-(6-phenethyl-octahydro-pyrrolo[2,3-c]pyridin-1-yl)-methanone;
{2-[1-(2,8-Diaza-spiro[4.5]decane-3-carbonyl)-pyrrolidin-2-yl]-thiazol-4-yl}-(4-fluoro-phenyl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
{3-[1′-(2,8-Diaza-spiro[4.5]decane-3-carbonyl)-[1,2]bipyrrolidinyl-2-yl]-pyridin-2-yl}-(4-fluoro-phenyl)-methanone;
(2,8-Diaza-spiro[4.5]dec-3-yl)-{2-[5-(4-fluoro-phenoxy)-pyridin-3-yl]-pyrrolidin-1-yl}-methanone;
{5-[1-(2,8-Diaza-spiro[4.5]decane-3-carbonyl)-pyrrolidin-2-yl]-pyridin-3-yl}-(4-fluoro-phenyl)-methanone;
(2,8-Diaza-spiro[4.5]dec-3-yl)-{2-[4-(4-fluoro-phenoxy)-pyridin-2-yl]-pyrrolidin-1-yl}-methanone;
(2,8-Diaza-spiro[4.5]dec-3-yl)-(2-{5-fluoro-2-[(4-fluoro-phenyl)-methyl-amino]-pyridin-4-yl}-pyrrolidin-1-yl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
[5-(1-Methylamino-ethyl)-furan-2-yl]-[-2-(7-phenyl-thiazolo[5,4-b]pyridin-2-yl)-pyrrolidin-1-yl]-methanone;
(5-(1-(methylamino)ethyl)furan-2-yl)(-2-(4-phenylthiazolo[4,5-c]pyridin-2-yl)pyrrolidin-1-yl)methanone;
(5-(1-(methylamino)ethyl)furan-2-yl)(-2-(7-phenylthiazolo[5,4-d]pyrimidin-2-yl)pyrrolidin-1-yl)methanone;
(4-Fluoro-phenyl)-(3-{1′-[5-(1-methylamino-ethyl)-furan-2-carbonyl]-[1,2′]-Dipyrrolidinyl-2-yl}-pyridin-2-yl)-methanone;
(octahydro-6-phenethylpyrrolo[2,3-c]pyridin-1-yl)(5-(1-(methylamino)ethyl)furan-2-yl)methanone;
(4-Fluoro-phenyl)-(2-{1-[5-(1-methylamino-ethyl)-furan-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(5-(1-(methylamino)ethyl)furan-2-yl)methanone;
(2-(5-(4-fluorophenoxy)pyridin-3-yl)pyrrolidin-1-yl)(5-(1-(methylamino)ethyl)furan-2-yl)methanone;
(4-Fluoro-phenyl)-(5-{1-[5-(1-methylamino-ethyl)-furan-2-carbonyl]-pyrrolidin-2-yl}-pyridin-3-yl)-methanone;
(2-(4-(4-fluorophenoxy)pyridin-2-yl)pyrrolidin-1-yl)(5-(1-(methylamino)ethyl)furan-2-yl)methanone;
(2-(2-(N-(4-fluorophenyl)-N-methylamino)-5-fluoropyridin-4-yl)pyrrolidin-1-yl)(5-(1-(methylamino)ethyl)furan-2-yl)methanone;
(3-(1-(methylamino)ethyl)phenyl)(-2-(7-phenylthiazolo[5,4-b]pyridin-2-yl)pyrrolidin-1-yl)methanone;
(3-(1-(methylamino)ethyl)phenyl)(-2-(4-phenylthiazolo[4,5-c]pyridin-2-yl)pyrrolidin-1-yl)methanone;
(3-(1-(methylamino)ethyl)phenyl)(-2-(7-phenylthiazolo[5,4-d]pyrimidin-2-yl)pyrrolidin-1-yl)methanone;
(4-Fluoro-phenyl)-(3-{1′-[3-(1-methylamino-ethyl)-benzoyl]-[1,2′]-Dipyrrolidinyl-2-yl}-pyridin-2-yl)-methanone;
(octahydro-6-phenethylpyrrolo[2,3-c]pyridin-1-yl)(3-(1-(methylamino)ethyl)phenyl)methanone;
(4-Fluoro-phenyl)-(2-{1-[3-(1-methylamino-ethyl)-benzoyl]-pyrrolidin-2-yl}-thiazol-4-yl)-methanone;
(2-(2-(N-(4-fluorophenyl)-N-methylamino)pyridin-4-yl)pyrrolidin-1-yl)(3-(1-(methylamino)ethyl)phenyl)methanone;
(2-(5-(4-fluorophenoxy)pyridin-3-yl)pyrrolidin-1-yl)(3-(1-(methylamino)ethyl)phenyl)methanone;
(4-Fluoro-phenyl)-(5-{1-[3-(1-methylamino-ethyl)-benzoyl]-pyrrolidin-2-yl}-pyridin-3-yl)-methanone;
(2-(4-(4-fluorophenoxy)pyridin-2-yl)pyrrolidin-1-yl)(3-(1-(methylamino)ethyl)phenyl)methanone;

(2-(2-(N-(4-fluorophenyl)-N-methylamino)-5-fluoropyridin-4-yl)pyrrolidin-1-yl)(3-(1-(methylamino)ethyl)phenyl)methanone;
(2-(2-(N-(4-fluorophenyl)-N-methylamino)pyridin-4-yl)pyrrolidin-1-yl)(3-(1-(methylamino)ethyl)phenyl)methanone;
(6-(1-(methylamino)ethyl)piperidin-2-yl)(-2-(7-phenylthiazolo[5,4-b]pyridin-2-yl)pyrrolidin-1-yl)methanone;
(6-(1-(methylamino)ethyl)piperidin-2-yl)(-2-(4-phenylthiazolo[4,5-c]pyridin-2-yl)pyrrolidin-1-yl)methanone;
(6-(1-(methylamino)ethyl)piperidin-2-yl)(-2-(7-phenylthiazolo[5,4-d]pyrimidin-2-yl)pyrrolidin-1-yl)methanone;
(4-Fluoro-phenyl)-(3-{1′-[6-(1-methylamino-ethyl)-piperidine-2-carbonyl]-[1,2′]-Dipyrrolidinyl-2-yl}-pyridin-2-yl)-methanone;
(octahydro-6-phenethylpyrrolo[2,3-c]pyridin-1-yl)(6-(1-(methylamino)ethyl)piperidin-2-yl)methanone;
(4-Fluoro-phenyl)-(2-{1-[6-(1-methylamino-ethyl)-piperidine-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(6-(1-(methylamino)ethyl)piperidin-2-yl)methanone;
(2-(5-(4-fluorophenoxy)pyridin-3-yl)pyrrolidin-1-yl)(6-(1-(methylamino)ethyl)piperidin-2-yl)methanone;
(4-Fluoro-phenyl)-(5-{1-[6-(1-methylamino-ethyl)-piperidine-2-carbonyl]-pyrrolidin-2-yl}-pyridin-3-yl)-methanone;
(2-(4-(4-fluorophenoxy)pyridin-2-yl)pyrrolidin-1-yl)(6-(1-(methylamino)ethyl)piperidin-2-yl)methanone;
(2-(2-(N-(4-fluorophenyl)-N-methylamino)-5-fluoropyridin-4-yl)pyrrolidin-1-yl)(6-(1-(methylamino)ethyl)piperidin-2-yl)methanone;
(2-(2-(N-(4-fluorophenyl)-N-methylamino)pyridin-4-yl)pyrrolidin-1-yl)(6-(1-(methylamino)ethyl)piperidin-2-yl)methanone;
{-1-[5-(1-Amino-ethyl)-furan-2-carbonyl]-4-phenyl-pyrrolidin-2-yl}-[-3-(4-fluoro-phenyl)-pyrrolidin-1-yl]-methanone;
[-1-[5-(1-Amino-ethyl)-furan-2-carbonyl]-4-(-4-fluoro-phenyl)-pyrrolidin-2-yl]-(-3-phenyl-pyrrolidin-1-yl)-methanone; and
(-4-(4-fluorophenyl)-2-((-3-(4-fluorophenyl)pyrrolidin-1-yl)methyl)pyrrolidin-1-yl)(5-(1-(methylamino)ethyl)furan-2-yl)methanone.

For the above mentioned further compounds of formula (I) the following stereoisomers are preferred:
(2S,4R)-1-((S)-2-((S)-2-aminopropanamido)-3-(pyridin-3-yl)propanoyl)-N-((1R,2R)-2-(methylcarbamoyl)-2,3-dihydro-1H-inden-1-yl)-4-phenylpyrrolidine-2-carboxamide;
(2S,4R)-1-((3R,5S)-1-((S)-2-((S)-2-aminopropanamido)-3-(pyridin-3-yl)propanoyl)-3-phenylpyrrolidine-5-carbonyl)-N-methyl-4-phenylpyrrolidine-2-carboxamide;
1-((S)-2-((S)-2-aminopropanamido)-3-methylbutanoyl)-N-((1R,2R)-2-(methylcarbamoyl)-2,3-dihydro-1H-inden-1-yl)-3-phenylazetidine-2-carboxamide;
(2S,4R)-1-((3R,5S)-1-((S)-2-((S)-2-aminopropanamido)butanoyl)-3-phenylpyrrolidine-5-carbonyl)-N-methyl-4-phenylpyrrolidine-2-carboxamide;
1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide;
(2S,4R)-1-((S)-2-((S)-2-aminopropanamido)-3-cyclopropylpropanoyl)-N-((1R,2R)-2-(methylcarbamoyl)-2,3-dihydro-1H-inden-1-yl)-4-phenylpyrrolidine-2-carboxamide;
1-((S)-2-((S)-2-aminopropanamido)-3-(3-chlorophenyl)propanoyl)-N-((1R,2R)-2-(methylcarbamoyl)-2,3-dihydro-1H-inden-1-yl)-3-phenylazetidine-2-carboxamide;
(2S,4R)-1-((S)-2-((S)-2-aminopropanamido)-4-(methylsulfonyl)butanoyl)-N-benzyl-N-(2-(methylamino)-2-oxoethyl)-4-phenylpyrrolidine-2-carboxamide;
1-((S)-2-((S)-2-aminopropanamido)-3-((2S,4R)-2-(benzyl(2-(methylamino)-2-oxoethyl)carbamoyl)-4-phenylpyrrolidin-1-yl)-3-oxopropyl)urea;
(2S,4R)-1-((S)-2-((S)-2-aminopropanamido)-3-(3-cyanophenyl)propanoyl)-N-((1R,2R)-2-(methylcarbamoyl)-2,3-dihydro-1H-inden-1-yl)-4-phenylpyrrolidine-2-carboxamide;
1-((3R,5S)-1-((S)-2-((S)-2-aminopropanamido)-3-(pyridin-3-yl)propanoyl)-3-phenylpyrrolidine-5-carbonyl)-N-methyl-4-phenylpyrrolidine-2-carboxamide;
(2S,4R)-1-(2-((S)-2-aminopropanamido)-3-((S)-pyrrolidin-2-yl)propanoyl)-N-((1R,2R)-2-(methylcarbamoyl)-2,3-dihydro-1H-inden-1-yl)-4-phenylpyrrolidine-2-carboxamide;
1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide;
(2S,4R)-1-((3R,5S)-1-(2-((S)-2-aminopropanamido)-3-(1H-1,2,4-triazol-1-yl)propanoyl)-3-phenylpyrrolidine-5-carbonyl)-N-methyl-4-phenylpyrrolidine-2-carboxamide;
1-((S)-2-((S)-2-aminopropanamido)-3-(3-carbamoylphenyl)propanoyl)-N-((1R,2R)-2-(methylcarbamoyl)-2,3-dihydro-1H-inden-1-yl)-3-phenylazetidine-2-carboxamide;
(2S,3S)-1-((3R,5S)-1-((S)-2-((S)-2-aminopropanamido)-3-(pyridin-3-yl)propanoyl)-3-phenylpyrrolidine-5-carbonyl)-N-methyl-3-phenylpyrrolidine-2-carboxamide;
1-((S)-2-((S)-2-aminopropanamido)-3-cyclopropylpropanoyl)-N-((1R,2R)-2-(methylcarbamoyl)-2,3-dihydro-1H-inden-1-yl)-3-phenylazetidine-2-carboxamide;
(2S,4R)-1-((S)-2-((S)-2-aminopropanamido)butanoyl)-N-(1-(methylamino)-1-oxo-3-(3-(trifluoromethyl)phenyl)propan-2-yl)-4-phenylpyrrolidine-2-carboxamide;
(S)-2-(2-(1-aminoethyl)picolinoyl)-N—((R)-2,3-dihydro-1H-inden-1-yl)-1,2,3,4-tetrahydroisoquinoline-3-carboxamide;
(2S)-1-(3-(1-aminoethyl)benzoyl)-N—((R)-2,3-dihydro-1H-inden-1-yl)-octahydro-1H-indole-2-carboxamide;
1-(2-(1-aminoethyl)picolinoyl)-N—((R)-2,3-dihydro-1H-inden-1-yl)-3-phenylazetidine-2-carboxamide;
(2S,3S)-1-(3-(1-aminoethyl)benzoyl)-N—((R)-2,3-dihydro-1H-inden-1-yl)-3-phenylpyrrolidine-2-carboxamide;
(2R,4S)-1-(2-(1-aminoethyl)picolinoyl)-N—((R)-2,3-dihydro-1H-inden-1-yl)-4-phenylpyrrolidine-2-carboxamide;
(2R,5S)-1-(3-(1-aminoethyl)benzoyl)-N—((R)-2,3-dihydro-1H-inden-1-yl)-5-phenylpyrrolidine-2-carboxamide;
(1R,2R)-1-(3-(1-aminoethyl)benzamido)-N—((R)-2,3-dihydro-1H-inden-1-yl)-2,3-dihydro-1H-indene-2-carboxamide;
(2S,4R)-1-(2-(1-aminoethyl)picolinoyl)-N—((R)-2,3-dihydro-1H-inden-1-yl)-4-(4-fluorophenyl)pyrrolidine-2-carboxamide;
(2S,4R)-1-(3-(1-aminoethyl)benzoyl)-4-(4-chlorophenyl)-N—((R)-2,3-dihydro-1H-inden-1-yl)pyrrolidine-2-carboxamide;
(S)-2-amino-N—((S)-4-methyl-1-oxo-1-((2S,4R)-4-phenyl-2-((3-phenylpyrrolidin-1-yl)methyl)pyrrolidin-1-yl)pentan-2-yl)propanamide;
(S)-2-amino-N—((S)-3-cyclohexyl-1-oxo-1-((2S,4R)-4-phenyl-2-((3-phenylpyrrolidin-1-yl)methyl)pyrrolidin-1-yl)propan-2-yl)propanamide;
(S)-2-amino-N—((R)-3-methyl-1-oxo-1-((2S,4R)-4-phenyl-2-((3-phenylpyrrolidin-1-yl)methyl)pyrrolidin-1-yl)butan-2-yl)propanamide;
(S)-2-amino-N—((S)-3-methyl-1-oxo-1-((2S,4R)-4-phenyl-2-((3-phenylpyrrolidin-1-yl)methyl)pyrrolidin-1-yl)butan-2-yl)propanamide;
(S)-2-amino-N-((2R,3S)-3-methyl-1-oxo-1-((2S,4R)-4-phenyl-2-((3-phenylpyrrolidin-1-yl)methyl)pyrrolidin-1-yl)pentan-2-yl)propanamide;
(S)-2-amino-N—((S)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-4-(1H-tetrazol-5-yl)butan-2-yl)propanamide;
(S)-2-amino-N—((S)-3-(3-chlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(4-chlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(2,4-dichlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(3,4-dichlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(3,4-difluorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-3-(4-(trifluoromethyl)phenyl)propan-2-yl)propanamide;
(S)-2-amino-N—((S)-3-(3-cyanophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-3-(pyridin-3-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)butan-2-yl)propanamide;
(S)-2-amino-N—((S)-3-cyclopropyl-1-oxo-1-((2S,4R)-4-phenyl-2-((3-phenylpyrrolidin-1-yl)methyl)pyrrolidin-1-yl)propan-2-yl)propanamide;
3-((S)-2-((S)-2-aminopropanamido)-3-oxo-3-((2S,4R)-4-phenyl-2-((3-phenylpyrrolidin-1-yl)methyl)pyrrolidin-1-yl)propyl)benzamide;
4-((S)-2-((S)-2-aminopropanamido)-3-oxo-3-((2S,4R)-4-phenyl-2-((3-phenylpyrrolidin-111)methyl)pyrrolidin-1-yl)propyl)benzamide;
(S)-2-amino-N—((R)-4,4-dimethyl-1-oxo-1-((2S,4R)-4-phenyl-2-((3-phenylpyrrolidin-1-yl)methyl)pyrrolidin-1-yl)pentan-2-yl)propanamide;
(4-(1-aminoethyl)-5-methylfuran-2-yl)((2S,4R)-4-phenyl-2-((3-phenylpyrrolidin-1-yl)methyl)pyrrolidin-1-yl)methanone;
(6-(1-aminoethyl)pyridin-2-yl)((2S,4R)-4-phenyl-2-((3-phenylpyrrolidin-1-yl)methyl)pyrrolidin-1-yl)methanone;
(3-(1-aminoethyl)phenyl)((2S,4R)-4-phenyl-2-((3-phenylpyrrolidin-1-yl)methyl)pyrrolidin-1-yl)methanone;
(2S,4R)-1-(3-(1-aminoethyl)-2-methylfuran-5-carbonyl)-N—((R)-2,3-dihydro-1H-inden-1-yl)-4-phenylpyrrolidine-2-carboxamide;
(2S,4R)-1-(2-(1-aminoethyl)picolinoyl)-N—((R)-2,3-dihydro-1H-inden-1-yl)-4-phenylpyrrolidine-2-carboxamide;
(2S,4R)-1-(3-(1-aminoethyl)benzoyl)-N—((R)-2,3-dihydro-1H-inden-1-yl)-4-phenylpyrrolidine-2-carboxamide;
(6-(1-aminoethyl)pyridin-2-yl)((2S,4R)-2-(((R)-3-(4-fluorophenyl)pyrrolidin-1-yl)methyl)-4-phenylpyrrolidin-1-yl)methanone;
(3-(1-aminoethyl)phenyl)((2S,4R)-2-(((R)-3-(4-chlorophenyl)pyrrolidin-1-yl)methyl)-4-phenylpyrrolidin-1-yl)methanone;
((2S)-4-(4-fluorobenzyl)-2-((3-phenylpyrrolidin-1-yl)methyl)pyrrolidin-1-yl)(5-(1-aminoethyl)furan-2-yl)methanone;
(2,8-Diaza-spiro[4.5]dec-3-yl)-[(2S,4R)-4-phenyl-2-(3-phenyl-pyrrolidine-1-carbonyl)-pyrrolidin-1-yl]-methanone;
(2S,4R)-1-(2,8-Diaza-spiro[4.5]decane-3-carbonyl)-4-phenyl-pyrrolidine-2-carboxylic acid (R)-indan-1-ylamide;
(2S,4S)-4-Cyclohexyl-1-(2,8-diaza-spiro[4.5]decane-3-carbonyl)-pyrrolidine-2-carboxylic acid (R)-indan-1-ylamide;
2,8-Diaza-spiro[4.5]decane-3-carboxylic acid [(S)-cyclohexyl-((R)-indan-1-ylcarbamoyl)-methyl]-amide;
[5-(1-Amino-ethyl)-furan-2-yl]-[(S)-4-(4-fluoro-benzyl)-2-(3-phenyl-pyrrolidine-1-carbonyl)-pyrrolidin-1-yl]-methanone;
(2S,4R)-4-Benzyl-1-(2,8-diaza-spiro[4.5]decane-3-carbonyl)-pyrrolidine-2-carboxylic acid ((1R,2R)-2-carbamoyl-indan-1-yl)-amide;
(5-(1-aminoethyl)furan-2-yl)(3-phenyl-2-((3-phenylazetidin-1-yl)methyl)azetidin-1-yl)methanone;
(6-(1-aminoethyl)piperidin-2-yl)((2S,4R)-4-phenyl-2-((3-phenylazetidin-1-yl)methyl)pyrrolidin-1-yl)methanone;
[3-(1-Amino-ethyl)-phenyl]-{(2S,4R)-2-[3-(4-fluoro-benzyl)-pyrrolidine-1-carbonyl]-4-phenylpyrrolidin-1-yl}-methanone;
(5-(1-aminoethyl)furan-2-yl)((S)-2-((3-phenylpyrrolidin-1-yl)methyl)pyrrolidin-1-yl)methanone;
(6-(1-aminoethyl)piperidin-2-yl)((S)-3-((3-phenylpyrrolidin-1-yl)methyl)-3,4-dihydroisoquinolin-2(1H)-yl)methanone;
(6-(1-aminoethyl)pyridin-2-yl)((2S)-2-((3-phenylpyrrolidin-1-yl)methyl)-octahydroindol-1-yl)methanone;
(3-(1-aminoethyl)phenyl)((2S)-4-(benzyloxy)-2-((3-phenylpyrrolidin-1-yl)methyl)pyrrolidin-1-yl)methanone;
(5-(1-aminoethyl)furan-2-yl)((2S,4R)-4-fluoro-2-((3-phenylpyrrolidin-1-yl)methyl)pyrrolidin-1-yl)methanone;
6-(1(S)-aminoethyl)-N-(3-(3-phenylpyrrolidin-1-yl)-1-(1H-1,2,4-triazol-1-yl)propan-2-yl)piperidine-2-carboxamide;
(6-(1-aminoethyl)pyridin-2-yl)(3-phenyl-2-((3-phenylpyrrolidin-1-yl)methyl)azetidin-1-yl)methanone;
(3-(1-aminoethyl)phenyl)((2S,3S)-3-phenyl-2-((3-phenylpyrrolidin-1-yl)methyl)pyrrolidin-1-yl)methanone;
(6-(1-aminoethyl)pyridin-2-yl)((2S,4S)-4-phenyl-2-((3-phenylpyrrolidin-1-yl)methyl)pyrrolidin-1-yl)methanone;
(3-(1-aminoethyl)phenyl)((2S,5R)-2-phenyl-5-((3-phenylpyrrolidin-1-yl)methyl)pyrrolidin-1-yl)methanone;
(5-(1-aminoethyl)furan-2-yl)((R)-4-((3-phenylpyrrolidin-1-yl)methyl)thiazolidin-3-yl)methanone;
3-(1-aminoethyl)-N-((1R,2S)-2-((3-phenylpyrrolidin-1-yl)methyl)-2,3-dihydro-1H-inden-1-yl)benzamide;
[5-(1-Amino-ethyl)-furan-2-yl]-[(S)-4-methylamino-2-((S)—(R)-3-phenyl-pyrrolidine-1-carbonyl)-pyrrolidin-1-yl]-methanone;
(6-(1-aminoethyl)piperidin-2-yl)((2S,4S)-4-hydroxy-4-phenyl-2-((3-phenylpyrrolidin-1-yl)methyl)pyrrolidin-1-yl)methanone;
(6-(1-aminoethyl)pyridin-2-yl)((2S,4R)-4-(4-fluorophenyl)-2-((3-phenylpyrrolidin-1-yl)methyl)pyrrolidin-1-yl)methanone;
(3-(1-aminoethyl)phenyl)((2S,4R)-4-(4-chlorophenyl)-2-((3-phenylpyrrolidin-1-yl)methyl)pyrrolidin-1-yl)methanone;
[6-(1-Amino-ethyl)-piperidin-2-yl]-[(2S,4R)-4-phenyl-2-(2-phenyl-pyrrolidine-1-carbonyl)-pyrrolidin-1-yl]-methanone;
4-((3R,5S)-1-(2-(1-aminoethyl)furan-5-carbonyl)-3-phenylpyrrolidine-5-carbonyl)-1,3-dimethylpiperazin-2-one;
(6-(1-aminoethyl)piperidin-2-yl)((S)-2-(((R)-2,3-dihydro-1H-inden-1-ylamino)methyl)pyrrolidin-1-yl)methanone;
(2S)-1-(2-(1-aminoethyl)furan-5-carbonyl)-4-(benzyloxy)-N—((R)-2,3-dihydro-1H-inden-1-yl)pyrrolidine-2-carboxamide;
(6-(1-aminoethyl)piperidin-2-yl)((2S,4R)-2-(((R)-2,3-dihydro-1H-inden-1-ylamino)methyl)-4-fluoropyrrolidin-1-yl)methanone;
(2S,4R)-4-(4-fluorobenzyl)-1-(2-(1-aminoethyl)furan-5-carbonyl)-N—((R)-2,3-dihydro-1H-inden-1-yl)pyrrolidine-2-carboxamide;
(5-(1-aminoethyl)furan-2-yl)((R)-4-(((R)-2,3-dihydro-1H-inden-1-ylamino)methyl)thiazolidin-3-yl)methanone;
2-((S)-4-(2-(1-aminoethyl)piperidine-6-carbonyl)-3-benzyl-2-oxopiperazin-1-yl)-N—((R)-2,3-dihydro-1H-inden-1-yl)acetamide;
(2S,4S)-1-(2-(1-aminoethyl)piperidine-6-carbonyl)-N—((R)-2,3-dihydro-1H-inden-1-yl)-4-hydroxy-4-phenylpyrrolidine-2-carboxamide;
(5-(1-aminoethyl)-2-methylfuran-3-yl)((2S,4R)-4-phenyl-2-((3-phenylpyrrolidin-1-yl)methyl)pyrrolidin-1-yl)methanone;
(6-(1-aminoethyl)piperidin-2-yl)((2S,4R)-4-phenyl-2-((3-phenylpyrrolidin-1-yl)methyl)pyrrolidin-1-yl)methanone;
(4-(aminomethyl)-5-isobutylfuran-2-yl)((2S,4R)-4-phenyl-2-((3-phenylpyrrolidin-1-yl)methyl)pyrrolidin-1-yl)methanone;
(2S,4R)-1-(2-(1-aminoethyl)-5-methylfuran-4-carbonyl)-N—((R)-2,3-dihydro-1H-inden-1-yl)-4-phenylpyrrolidine-2-carboxamide;
(2S,4R)-1-(2-(1-aminoethyl)furan-5-carbonyl)-N—((R)-2,3-dihydro-1H-inden-1-yl)-4-phenylpyrrolidine-2-carboxamide;
(2S,4R)-1-(2-(1-aminoethyl)piperidine-6-carbonyl)-N—((R)-2,3-dihydro-1H-inden-1-yl)-4-phenylpyrrolidine-2-carboxamide;
(2S,4R)-1-(3-(aminomethyl)-2-isobutylfuran-5-carbonyl)-N—((R)-2,3-dihydro-1H-inden-1-yl)-4-phenylpyrrolidine-2-carboxamide;
[5-(1-Amino-ethyl)-furan-2-yl]-{(2S,4R)-4-(4-fluoro-phenyl)-2-[(R)-3-(4-fluoro-phenyl)-pyrrolidine-1-carbonyl]-pyrrolidin-1-yl}-methanone;
[6-(1-Amino-ethyl)-piperidin-2-yl]-{(2S,4R)-4-(4-chloro-phenyl)-2-[(R)-3-(4-chloro-phenyl)-pyrrolidine-1-carbonyl]-pyrrolidin-1-yl}-methanone;
(6-(1-aminoethyl)pyridin-2-yl)((2S,4R)-2-(((R)-3-(3-fluorophenyl)pyrrolidin-1-yl)methyl)-4-phenylpyrrolidin-1-yl)methanone;
(3-(1-aminoethyl)phenyl)((2S,4R)-2-(((R)-3-(3,4-dichlorophenyl)pyrrolidin-1-yl)methyl)-4-phenylpyrrolidin-1-yl)methanone;
((2S,4R)-4-(4-fluorophenyl)-2-(((R)-3-(3-fluorophenyl)pyrrolidin-1-yl)methyl)pyrrolidin-1-yl)(5-(1-(methylamino)propyl)furan-2-yl)methanone;
(5-(1-aminoethyl)furan-2-yl)(2-((2,3-dihydro-1H-inden-1-ylamino)methyl)-4-phenylpyrrolidin-1-yl)methanone;
(S)-2-amino-N-((1R,3S,6S)-5-oxo-1-phenyl-3-((3-phenylpyrrolidin-1-yl)methyl)-octahydro-1H-pyrrolo[1,2-a]azepin-6-yl)propanamide;
(5-((S)-1-aminoethyl)furan-2-yl)((2S,4R)-2-(phenoxymethyl)-4-phenylpyrrolidin-1-yl)methanone;
(5-((S)-1-aminoethyl)furan-2-yl)((2S,4R)-2-((naphthalen-1-yloxy)methyl)-4-phenylpyrrolidin-1-yl)methanone;
(5-((S)-1-aminoethyl)furan-2-yl)((2S,4R)-2-((2,3-dihydro-1H-inden-1-ylamino)methyl)-4-phenylpyrrolidin-1-yl)methanone;
(5-((S)-1-aminoethyl)furan-2-yl)((2S,4R)-4-phenyl-2-(((R)-1,2,3,4-tetrahydronaphthalen-1-ylamino)methyl)pyrrolidin-1-yl)methanone;
(5-((S)-1-aminoethyl)furan-2-yl)((2S,4R)-2-(2-benzyl-2H-tetrazol-5-yl)-4-phenylpyrrolidin-1-ylmethanone;
(5-((S)-1-aminoethyl)furan-2-yl)((2S,4R)-2-(4-benzyloxazol-2-yl)-4-phenylpyrrolidin-1-yl)methanone;
[5-((S)-1-Amino-ethyl)-furan-2-yl]-[(2S,4R)-2-(5-benzoyl-[1,2,4]oxadiazol-3-yl)-4-phenyl-pyrrolidin-1-yl]-methanone;
(2S,4R)-1-(2-((S)-1-aminoethyl)furan-5-carbonyl)-4-phenyl-N-(4-phenyl-1,2,3-thiadiazol-5-yl)pyrrolidine-2-carboxamide;
(2S,4R)-1-(2-((S)-1-aminoethyl)furan-5-carbonyl)-4-phenyl-N-(1-phenyl-1H-pyrazol-5-yl)pyrrolidine-2-carboxamide;
(2S,4R)-1-(2-((S)-1-aminoethyl)furan-5-carbonyl)-4-phenyl-N-(5-phenyl-1H-tetrazol-1-yl)pyrrolidine-2-carboxamide;
(5-((S)-1-aminoethyl)furan-2-yl)((2S,4R)-2-((1-methyl-1H-indol-3-ylmethyl)-4-phenylpyrrolidin-1-ylmethanone;
1-(3-(((2S,4R)-1-(2-((S)-1-aminoethyl)furan-5-carbonyl)-4-phenylpyrrolidin-2-ylmethyl)-1H-indol-1-yl)ethanone;
(5-((S)-1-aminoethyl)furan-2-yl)((2S,4R)-2-(benzofuran-3-ylmethyl)-4-phenylpyrrolidin-1-ylmethanone;
[5-((S)-1-Amino-ethyl)-2-methoxy-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-2-benzyloxy-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-4-ethoxy-2-piperidin-1-yl-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-1H-pyrrol-2-yl]-[(2S,4R)-4-phenyl-2-((R)-3-phenyl-pyrrolidine-1-carbonyl)-pyrrolidin-1-yl]-methanone;
[5-((S)-1-Amino-ethyl)-furan-2-yl]-[(2S,4R)-4-phenyl-2-((R)-3-phenyl-pyrrolidine-1-carbonyl)-pyrrolidin-1-yl]-methanone;
[5-((S)-1-Amino-ethyl)-[1,2,4]oxadiazol-3-yl]-[(2S,4R)-4-phenyl-2-((R)-3-phenyl-pyrrolidine-1-carbonyl)-pyrrolidin-1-yl]-methanone;
[3-((S)-1-Amino-ethyl)-[1,2,4]oxadiazol-5-yl]-[(2S,4R)-4-phenyl-2-((R)-3-phenyl-pyrrolidine-1-carbonyl)-pyrrolidin-1-yl]-methanone;
[5-((S)-1-Amino-ethyl)-oxazol-2-yl]-[(2S,4R)-4-phenyl-2-((R)-3-phenyl-pyrrolidine-1-carbonyl)-pyrrolidin-1-yl]-methanone;
[5-((S)-1-Amino-ethyl)-1H-imidazol-2-yl]-[(2S,4R)-4-phenyl-2-((R)-3-phenyl-pyrrolidine-1-carbonyl)-pyrrolidin-1-yl]-methanone;
[4-((S)-1-Amino-ethyl)-1-methyl-1H-imidazol-2-yl]-[(2S,4R)-4-phenyl-2-((R)-3-phenyl-pyrrolidine-1-carbonyl)-pyrrolidin-1-yl]-methanone;
[4-((S)-1-Amino-ethyl)-phenyl]-[(2S,4R)-4-phenyl-2-((R)-3-phenyl-pyrrolidine-1-carbonyl)-pyrrolidin-1-yl]-methanone;
(6-Aminomethyl-pyridin-2-yl)-[(2S,4R)-4-phenyl-2-((R)-3-phenyl-pyrrolidine-1-carbonyl)-pyrrolidin-1-yl]-methanone;
(5-Aminomethyl-thiazol-2-yl)-[(2S,4R)-4-phenyl-2-((R)-3-phenyl-pyrrolidine-1-carbonyl)-pyrrolidin-1-yl]-methanone;
(5-Aminomethyl-thiophen-2-yl)-[(2S,4R)-4-phenyl-2-((R)-3-phenyl-pyrrolidine-1-carbonyl)-pyrrolidin-1-yl]-methanone;
(5-Methylaminomethyl-thiophen-2-yl)-[(2S,4R)-4-phenyl-2-((R)-3-phenyl-pyrrolidine-1-carbonyl)-pyrrolidin-1-yl]-methanone;
(5-Methylaminomethyl-furan-2-yl)-[(2S,4R)-4-phenyl-2-((R)-3-phenyl-pyrrolidine-1-carbonyl)-pyrrolidin-1-yl]-methanone;
(5-Aminomethyl-furan-2-yl)-[(2S,4R)-4-phenyl-2-((R)-3-phenyl-pyrrolidine-1-carbonyl)-pyrrolidin-1-yl]-methanone;
(2-Aminomethyl-1,5-dimethyl-1H-imidazol-4-yl)-[(2S,4R)-4-phenyl-2-((R)-3-phenyl-pyrrolidine-1-carbonyl)-pyrrolidin-1-yl]-methanone;
(5-Methylaminomethyl-[1,2,4]oxadiazol-3-yl)-[(2S,4R)-4-phenyl-2-((R)-3-phenyl-pyrrolidine-1-carbonyl)-pyrrolidin-1-yl]-methanone;
[2-((S)-1-Amino-ethyl)-5-methyl-oxazol-4-yl]-[(2S,4R)-4-phenyl-2-((R)-3-phenyl-pyrrolidine-1-carbonyl)-pyrrolidin-1-yl]-methanone;
(5-Aminomethyl-[1,2,4]oxadiazol-3-yl)-[(2S,4R)-4-phenyl-2-((R)-3-phenyl-pyrrolidine-1-carbonyl)-pyrrolidin-1-yl]-methanone;
(5-Aminomethyl-furan-3-yl)-[(2S,4R)-4-phenyl-2-((R)-3-phenyl-pyrrolidine-1-carbonyl)-pyrrolidin-1-yl]-methanone;
(4-Aminomethyl-5-methyl-furan-2-yl)-[(2S,4R)-4-phenyl-2-((R)-3-phenyl-pyrrolidine-1-carbonyl)-pyrrolidin-1-yl]-methanone;
(4-Aminomethyl-5-isobutyl-furan-2-yl)-[(2S,4R)-4-phenyl-2-((R)-3-phenyl-pyrrolidine-1-carbonyl)-pyrrolidin-1-yl]-methanone;
(5-Aminomethyl-isoxazol-3-yl)-[(2S,4R)-4-phenyl-2-((R)-3-phenyl-pyrrolidine-1-carbonyl)-pyrrolidin-1-yl]-methanone;
(5-Aminomethyl-thiophen-3-yl)-[(2S,4R)-4-phenyl-2-((R)-3-phenyl-pyrrolidine-1-carbonyl)-pyrrolidin-1-yl]-methanone;
[2-((S)-1-Amino-ethyl)-oxazol-5-yl]-[(2S,4R)-4-phenyl-2-((R)-3-phenyl-pyrrolidine-1-carbonyl)-pyrrolidin-1-yl]-methanone;
(6-Methyl-2,8-diaza-spiro[4.5]dec-3-yl)-[(2S,4R)-4-phenyl-2-(3-phenyl-pyrrolidin-1-ylmethyl)-pyrrolidin-1-yl]-methanone;
(6-Ethyl-2,8-diaza-spiro[4.5]dec-3-yl)-[(2S,4R)-4-phenyl-2-(3-phenyl-pyrrolidin-1-ylmethyl)-pyrrolidin-1-yl]-methanone;
((2R,4R)-4-(4-fluorophenyl)-2-((3-(4-fluorophenyl)cyclopentyl)methyl)pyrrolidin-1-yl)(5-(1-(methylamino)ethyl)furan-2-yl)methanone;
[6-(1-Methylamino-ethyl)-piperidin-2-yl]-[(2S,4R)-4-phenyl-2-((R)-3-phenyl-pyrrolidine-1-carbonyl)-pyrrolidin-1-yl]-methanone;
(6-(1-(methylamino)ethyl)piperidin-2-yl)((2S,4R)-4-phenyl-2-(((R)-3-phenylpyrrolidin-1-yl)methyl)pyrrolidin-1-yl)methanone;
(2,8-Diaza-spiro[4.5]dec-3-yl)-[(S)-2-(4-phenyl-thiazolo[4,5-c]pyridin-2-yl)-pyrrolidin-1-yl]-methanone;
(2,8-Diaza-spiro[4.5]dec-3-yl)-[(S)-2-(7-phenyl-thiazolo[5,4-b]pyridin-2-yl)-pyrrolidin-1-yl]-methanone;
(2,8-Diaza-spiro[4.5]dec-3-yl)-[(S)-2-(7-phenyl-thiazolo[5,4-d]pyrimidin-2-yl)-pyrrolidin-1-yl]-methanone;
(2,8-Diaza-spiro[4.5]dec-3-yl)-(6-phenethyl-octahydro-pyrrolo[2,3-c]pyridin-1-yl)-methanone;
{2-[1-(2,8-Diaza-spiro[4.5]decane-3-carbonyl)-pyrrolidin-2-yl]-thiazol-4-yl}-(4-fluoro-phenyl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
{3-[1′-(2,8-Diaza-spiro[4.5]decane-3-carbonyl)-[1,2]bipyrrolidinyl-2-yl]-pyridin-2-yl}-(4-fluoro-phenyl)-methanone;
(2,8-Diaza-spiro[4.5]dec-3-yl)-{2-[5-(4-fluoro-phenoxy)-pyridin-3-yl]-pyrrolidin-1-yl}-methanone;
{5-[1-(2,8-Diaza-spiro[4.5]decane-3-carbonyl)-pyrrolidin-2-yl]-pyridin-3-yl}-(4-fluoro-phenyl)-methanone;
(2,8-Diaza-spiro[4.5]dec-3-yl)-{2-[4-(4-fluoro-phenoxy)-pyridin-2-yl]-pyrrolidin-1-yl}-methanone;
(2,8-Diaza-spiro[4.5]dec-3-yl)-(2-{5-fluoro-2-[(4-fluoro-phenyl)-methyl-amino]-pyridin-4-yl}-pyrrolidin-1-yl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
[5-(1-Methylamino-ethyl)-furan-2-yl]-[(S)-2-(7-phenyl-thiazolo[5,4-b]pyridin-2-yl)-pyrrolidin-1-yl]-methanone;
(5-(1-(methylamino)ethyl)furan-2-yl)((S)-2-(4-phenylthiazolo[4,5-c]pyridin-2-yl)pyrrolidin-1-yl)methanone;
(5-(1-(methylamino)ethyl)furan-2-yl)((S)-2-(7-phenylthiazolo[5,4-d]pyrimidin-2-yl)pyrrolidin-1-yl)methanone;
(4-Fluoro-phenyl)-(3-{1′-[5-(1-methylamino-ethyl)-furan-2-carbonyl]-[1,2′]-Dipyrrolidinyl-2-yl}-pyridin-2-yl)-methanone;
(octahydro-6-phenethylpyrrolo[2,3-c]pyridin-1-yl)(5-(1-(methylamino)ethyl)furan-2-yl)methanone;
(4-Fluoro-phenyl)-(2-{1-[5-(1-methylamino-ethyl)-furan-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(5-(1-(methylamino)ethyl)furan-2-yl)methanone;
(2-(5-(4-fluorophenoxy)pyridin-3-yl)pyrrolidin-1-yl)(5-(1-(methylamino)ethyl)furan-2-yl)methanone;
(4-Fluoro-phenyl)-(5-{1-[5-(1-methylamino-ethyl)-furan-2-carbonyl]-pyrrolidin-2-yl}-pyridin-3-yl)-methanone;
(2-(4-(4-fluorophenoxy)pyridin-2-yl)pyrrolidin-1-yl)(5-(1-(methylamino)ethyl)furan-2-yl)methanone;
(2-(2-(N-(4-fluorophenyl)-N-methylamino)-5-fluoropyridin-4-yl)pyrrolidin-1-yl)(5-(1-(methylamino)ethyl)furan-2-yl)methanone;
(3-(1-(methylamino)ethyl)phenyl)((S)-2-(7-phenylthiazolo[5,4-b]pyridin-2-yl)pyrrolidin-1-yl)methanone;
(3-(1-(methylamino)ethyl)phenyl)((S)-2-(4-phenylthiazolo[4,5-c]pyridin-2-yl)pyrrolidin-1-yl)methanone;
(3-(1-(methylamino)ethyl)phenyl)((S)-2-(7-phenylthiazolo[5,4-d]pyrimidin-2-yl)pyrrolidin-1-yl)methanone;
(4-Fluoro-phenyl)-(3-{1′-[3-(1-methylamino-ethyl)-benzoyl]-[1,2′]-Dipyrrolidinyl-2-yl}-pyridin-2-yl)-methanone;
(octahydro-6-phenethylpyrrolo[2,3-c]pyridin-1-yl)(3-(1-(methylamino)ethyl)phenyl)methanone;
(4-Fluoro-phenyl)-(2-{1-[3-(1-methylamino-ethyl)-benzoyl]-pyrrolidin-2-yl}-thiazol-4-yl)-methanone;
(2-(2-(N-(4-fluorophenyl)-N-methylamino)pyridin-4-yl)pyrrolidin-1-yl)(3-(1-(methylamino)ethyl)phenyl)methanone;
(2-(5-(4-fluorophenoxy)pyridin-3-yl)pyrrolidin-1-yl)(3-(1-(methylamino)ethyl)phenyl)methanone;
(4-Fluoro-phenyl)-(5-{1-[3-(1-methylamino-ethyl)-benzoyl]-pyrrolidin-2-yl}-pyridin-3-yl)-methanone;
(2-(4-(4-fluorophenoxy)pyridin-2-yl)pyrrolidin-1-yl)(3-(1-(methylamino)ethyl)phenyl)methanone;
(2-(2-(N-(4-fluorophenyl)-N-methylamino)-5-fluoropyridin-4-yl)pyrrolidin-1-yl)(3-(1-(methylamino)ethyl)phenyl)methanone;
(2-(2-(N-(4-fluorophenyl)-N-methylamino)pyridin-4-yl)pyrrolidin-1-yl)(3-(1-(methylamino)ethyl)phenyl)methanone;
(6-(1-(methylamino)ethyl)piperidin-2-yl)((S)-2-(7-phenylthiazolo[5,4-b]pyridin-2-yl)pyrrolidin-1-yl)methanone;
(6-(1-(methylamino)ethyl)piperidin-2-yl)((S)-2-(4-phenylthiazolo[4,5-c]pyridin-2-yl)pyrrolidin-1-yl)methanone;
(6-(1-(methylamino)ethyl)piperidin-2-yl)((S)-2-(7-phenylthiazolo[5,4-d]pyrimidin-2-yl)pyrrolidin-1-yl)methanone;
(4-Fluoro-phenyl)-(3-{1′-[6-(1-methylamino-ethyl)-piperidine-2-carbonyl]-[1,2′]-Dipyrrolidinyl-2-yl}-pyridin-2-yl)-methanone;
(octahydro-6-phenethylpyrrolo[2,3-c]pyridin-1-yl)(6-(1-(methylamino)ethyl)piperidin-2-yl)methanone;
(4-Fluoro-phenyl)-(2-{1-[6-(1-methylamino-ethyl)-piperidine-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(6-(1-(methylamino)ethyl)piperidin-2-yl)methanone;
(2-(5-(4-fluorophenoxy)pyridin-3-yl)pyrrolidin-1-yl)(6-(1-(methylamino)ethyl)piperidin-2-yl)methanone;
(4-Fluoro-phenyl)-(5-{1-[6-(1-methylamino-ethyl)-piperidine-2-carbonyl]-pyrrolidin-2-yl}-pyridin-3-yl)-methanone;
(2-(4-(4-fluorophenoxy)pyridin-2-yl)pyrrolidin-1-yl)(6-(1-(methylamino)ethyl)piperidin-2-yl)methanone;
(2-(2-(N-(4-fluorophenyl)-N-methylamino)-5-fluoropyridin-4-yl)pyrrolidin-1-yl)(6-(1-(methylamino)ethyl)piperidin-2-yl)methanone;
(2-(2-(N-(4-fluorophenyl)-N-methylamino)pyridin-4-yl)pyrrolidin-1-yl)(6-(1-(methylamino)ethyl)piperidin-2-yl)methanone;
{(2S,4R)-1-[5-(1-Amino-ethyl)-furan-2-carbonyl]-4-phenyl-pyrrolidin-2-yl}-[(R)-3-(4-fluoro-phenyl)-pyrrolidin-1-yl]-methanone;
[(S)-1-[5-(1-Amino-ethyl)-furan-2-carbonyl]-4-((R)-4-fluoro-phenyl)-pyrrolidin-2-yl]-((R)-3-phenyl-pyrrolidin-1-yl)-methanone; and
((2S,4R)-4-(4-fluorophenyl)-2-(((R)-3-(4-fluorophenyl)pyrrolidin-1-yl)methyl)pyrrolidin-1-yl)(5-(1-(methylamino)ethyl)furan-2-yl)methanone.

The term “linker”, as used herein, refers to a moiety which covalently links separate parts of a molecule or separate molecules, such as the linker A₄ of formula I, or the linker L of formula VI or VII, respectively. Accordingly, one point of the linker may be attached to one part of the molecule of formula I and another point of the linker may be attached to another part of said molecule; or alternatively, one point of the linker may be attached to one point of a molecule of formula I and another point of the linker may be attached to one point of a separate molecule of formula I. See furthermore the particular description of linkers L for formula VI and VII, herein below.

The terms “treating” and “treatment”, as used herein, refers to reversing, alleviating, inhibiting the progress of, or preventing the disorder or condition to which such term applies, or one or more symptoms of such disorder or condition.

The term “protecting group”, as used herein, means a hydroxy or amino protecting group which is selected from typical hydroxy or amino protecting groups described in Protective Groups in Organic Synthesis edited by T. W. Greene et al. (John Wiley & Sons, 1991);

It will be apparent to one of skill in the art when a compound of the invention can exist as a salt form, especially as an acid addition salt or a base addition salt. When a compound can exist in a salt form, such salt forms are included within the scope of the invention. Although any salt form may be useful in chemical manipulations, such as purification procedures, only pharmaceutically acceptable salts are useful for pharmaceutically products. The term “pharmaceutically acceptable salts, solvates or prodrugs” as used herein refers to those acid and base additions salts, solvates, and prodrugs of the compounds of the present invention which are, within the scope of sound medical judgment, suitable for use without undue toxicity, irritation, allergic response, and the like, commensurate with a reasonable benefit/risk ratio, and effective for their intended use, as well as the zwitterionic forms, where possible, of the compounds of the invention.

Certain acidic or basic compounds of the present invention may exist as zwitterions. It is well known in the art that compounds containing both amino and carboxyl groups often exist in equilibrium with their zwitterionic forms. Thus, any of the compounds described herein throughout that contain, for example, both amino and carboxyl groups, also include their corresponding zwitterions.

Pharmaceutically acceptable acid and base addition salts refers to the relatively non-toxic, inorganic and organic addition salts of compounds of the present invention. These salts can be prepared in situ during the final isolation and purification of the compounds or by separately reacting the purified compound in its free acid or base form with a suitable organic or inorganic compound and isolating the salt thus formed. In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt.

The pharmaceutically acceptable acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Salts may be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. (See, for example, Berge S. M. et al., “Pharmaceutical Salts,” J. Pharm. Sci., 1977; 66:1-19 which is incorporated herein by reference.)

The compounds of the present invention can exist in unsolvated forms as well as solvated forms, including hydrated forms. In general, the solvated forms, including hydrated forms, are equivalent to unsolvated forms and are intended to be encompassed within the scope of the present invention.

The term “prodrug” refers to compounds that are rapidly transformed in vivo to yield the parent compound of the above formulae, for example, by hydrolysis in blood. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Since prodrugs are known to enhance numerous desirable qualities of pharmaceuticals (e.g., solubility, bioavailability, manufacturing, etc.) the compounds of the present invention and employed in the present methods may, if desired, be delivered in prodrug form. Examples of prodrugs include pharmaceutically acceptable, non-toxic esters of the compounds of the present invention, including C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred, such as e.g. methyl, ethyl, n-propyl, iso-propyl, butyl, isobutyl, sec-butyl, and tert-butyl. Esters of the compounds of the present invention may be prepared according to conventional methods “March's Advanced Organic Chemistry, 5^{th} Edition”. M. B. Smith & J. March, John Wiley & Sons, 2001. A preferred class of prodrugs are compounds in which a nitrogen atom in an amino, amidino, aminoalkyleneamino, iminoalkyleneamino or guanidino group is substituted with a hydroxy (OH) group, an alkylcarbonyl (—CO—R) group, an alkoxycarbonyl (—CO—OR), an acyloxyalkyl-alkoxycarbonyl (—CO—O—R—O—CO—R) group where R is a monovalent or divalent group and as defined above or a group having the formula —C(O)—O—CPIP2-haloalkyl, where P1 and P2 are the same or different and are H, lower alkyl, lower alkoxy, cyano, halo lower alkyl or aryl. Preferably the nitrogen atom is one of the nitrogen atoms of the amidino group of the compounds of the invention. These prodrug compounds are prepared by reacting the compounds of the invention described above with an activated acyl compound to bond a nitrogen atom in the compound of the invention to the carbonyl of the activated acyl compound. Suitable activated carbonyl compounds contain a good leaving group bonded to the carbonyl carbon and include acyl halides, acyl amines, acyl pyridinium salts, acyl alkoxides, in particular acyl phenoxides such as p-nitrophenoxy acyl, dinitrophenoxy acyl, fluorophenoxy acyl, and difluorophenoxy acyl. The reactions are generally exothermic and are earned out in inert solvents at reduced temperatures such as −78° C. to about 5° C. The reactions are usually also carried out in the presence of an inorganic base such as potassium carbonate or sodium bicarbonate, or an organic base such as an amine, including pyridine, triethylamine, etc. One manner of preparing prodrugs is described in U.S. Ser. No. 08/843,369 filed Apr. 15, 1997 (corresponding to PCT publication WO9846576) the contents of which are incorporated herein by reference in their entirety.

Compounds of formula (I) may contain chiral centers and therefore may exist in different enantiomeric and diastereomeric forms. This invention relates to all optical isomers and all stereoisomers of compounds of the formula I, both as racemic mixtures and as individual enantiomers and diastereoisomers ((+)- and (−)-optically active forms) of such compounds, and mixtures thereof, and to all pharmaceutical compositions and methods of treatment defined herein that contain or employ them, respectively. Individual isomers can be obtained by known methods, such as optical resolution, optically selective reaction, or chromatographic separation in the preparation of the final product or its intermediate.

A preferred embodiment of the invention comprises compounds of formula (I), wherein the carbon atom with R³ attached is in the S-configuration.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the formula I, is to be understood as referring also to the corresponding tautomers, tautomeric mixtures, and salts of these compounds, unless otherwise stated.

The present invention also includes isotopically-labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I. Compounds of the present invention and pharmaceutically acceptable salts of said compounds that contain the aforementioned isotopes and/or other isotopes of other atoms are within the scope of this invention. Isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of the present invention can generally be prepared by carrying out the procedures disclosed in the Schemes and/or in the Examples below, by substituting a readily available isotopically labeled reagent for a non-isotopically labeled reagent.

### Compounds of Formula (I) Having Formulas (II), (III), (IV), and (V)

In one embodiment of the invention the compounds of formula (I) are of formula (II)

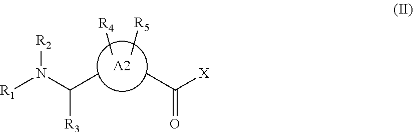

or a pharmaceutically acceptable salt, solvate or prodrug thereof,
wherein R¹, R², R³, R⁴, R⁵, R⁶, R⁷, R⁸, B, A₁, A₃, A₄, and X are as defined for formula (I) herein above, and A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems.

In a preferred embodiment of formula (II) X is

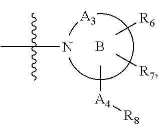

and the compounds are accordingly of formula (IIa):

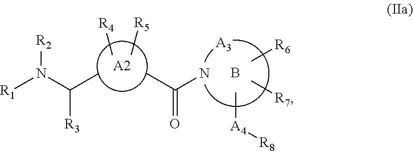

or a pharmaceutically acceptable salt, solvate or prodrug thereof,
wherein
R¹, R², R³, R⁴, R⁵, R⁶, R⁷, R⁸, B, A₁, A₃, A₄, and X are as defined for formula (I) herein above, and A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems; and with the proviso that when A₁ is a single bond, A₂ is an oxazol ring, B is a pyrrolidinyl, R¹ and R² is H, R³ is selected from H or methyl, R⁴ and R⁵ is selected from H or methyl, and R⁸ is phenyl, 4-hydroxy-1-phenyl, or 3-indolyl, then at least one of R⁶ and R⁷ is different from H.

In a preferred embodiment of formula (IIa), at least one of R¹ and R² is different from H.

It has surprisingly been found, that the presence of at least one of R¹ and R² different from H, may improve the compounds cell permeability. To this end it is especially preferred that one of R¹ and R² are selected from the group consisting of C₁-C₄ alkyl, C₁-C₄ alkoxy, C₂-C₄ alkenyl, and C₂-C₄ alkynyl, wherein any alkyl, alkenyl and alkynyl optionally are substituted; more preferably selected from the group consisting of C₁-C₄ alkyl, and C₁-C₄ alkoxy; even more preferably methyl and ethyl; and yet even more preferably methyl. Accordingly in a preferred embodiment of formula (IIa) R¹ is H and R² is methyl.

In an alternative embodiment of the compounds of formula (IIa) R¹ and R² are both H.

The present inventors have found that the compounds of formula (II) and formula (IIa) comprising a R⁶ and/or R⁷ substituent have an improved activity profile compared to compounds without said R⁶ and/or R⁷ group. Accordingly, in a preferred embodiment of formula (IIa) at least one of R⁶ and R⁷ is not H, and R¹, R², R³, R⁴, R⁵, R⁸, B, A₂, A₃, and A₄ are as defined for formula (I) herein above. More preferably at least one of R⁶ and R⁷ each independently may be selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein Z₃ and p is as defined herein above, and wherein any alkyl, cycloalkyl, aryl, heterocyclyl, and heteroaryl optionally are substituted, as specified for formula (I).

In an alternative embodiment of formula (IIa) at least one of R⁶ and R⁷ each independently are C₃-C₁₀ cycloalkyl, wherein the cycloalkyl optionally is substituted. In a further alternative embodiment at least one of R⁶ and R⁷ each independently are aryl, wherein the aryl optionally is substituted. More preferably R⁶ and R⁷ each independently may be phenyl optionally substituted with one to three substitutents selected from the group consisting of hydroxy, fluoro, chloro, bromo, iodo, methoxy, ethoxy. Even more preferably R⁶ and R⁷ each independently may be phenyl, fluorophenyl, hydroxyphenyl, chlorophenyl, difluorophenyl, dichlorophenyl, trifluorophenyl, and trichlorophenyl. In a further alternative embodiment at least one of R⁶ and R⁷ each independently are heterocyclyl, wherein the heterocyclyl optionally is substituted. In a further alternative embodiment at least one of R⁶ and R⁷ each independently are heteroaryl, wherein the heteroaryl optionally is substituted. The heterocyclyl and heteroaryl may be as define herein.

In a more preferred embodiment of formula (IIa) at least one of R⁶ and R⁷ each independently are selected from the group consisting of cyclopropyl, cyclobutyl, cyclopentyl, cyclohexyl, bicyclo[2.2.2]octanyl, azetidinyl, tetrahydro-2H-pyranyl, piperidinyl, tetrahydro-2H-thiopyranyl, morpholinyl, piperazinyl, thiomorpholinylaziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl, and wherein any of the ring system optionally are substituted.

It has furthermore been found that compounds of formula (IIa) having substituted ring structures as the R⁸ moiety has an improved activity profile, preferably the ring structures are substituted with a further ring structure giving a bulky group. Accordingly, in a further embodiment of formula (IIa), R⁸ is selected from the group consisting of substituted C₃-C₁₀ cycloalkyl, aryl, heterocyclyl, and heteroaryl, heterocyclyl and substituted heteroaryl; and R¹, R², R³, R⁴, R⁵, R⁶, R⁷, B, A₂, A₃, and A₄ are as defined for formula (I) herein above. More preferably R⁸ may be selected from the group consisting of aryl-C₁-C₆ alkyl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted.

For this embodiment it is even more preferred that R⁸ is selected from the group consisting of C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; and wherein any cycloalkyl, aryl, heterocyclyl, and heteroaryl optionally may be substituted.

Furthermore for this embodiment it is yet even more preferred that R⁸ is selected from the group consisting heterocyclyl-C₃-C₁₀ cycloalkyl, heteroaryl-C₃-C₁₀ cycloalkyl, heterocyclyl-heteroaryl, heteroaryl-heterocyclyl, heterocyclyl-O—C₃-C₁₀ cycloalkyl, heteroaryl-O—C₃-C₁₀ cycloalkyl, heterocyclyl-O-heteroaryl, heteroaryl-O-heterocyclyl, heterocyclyl-C(O)—C₃-C₁₀ cycloalkyl, heteroaryl-C(O)—C₃-C₁₀ cycloalkyl, heterocyclyl-C(O)-heteroaryl, heteroaryl-C(O)-heterocyclyl, heterocyclyl-CH₂—C₃-C₁₀ cycloalkyl, heteroaryl-CH₂—C₃-C₁₀ cycloalkyl, heterocyclyl-CH₂-heteroaryl, heteroaryl-CH₂-heterocyclyl, heterocyclyl-CH₂CH₂—C₃-C₁₀ cycloalkyl, heteroaryl-CH₂CH₂—C₃-C₁₀ cycloalkyl, heterocyclyl-CH₂CH₂-heteroaryl, heteroaryl-CH₂CH₂-heterocyclyl, heterocyclyl-NH—C₃-C₁₀ cycloalkyl, heteroaryl-NH—C₃-C₁₀ cycloalkyl, heterocyclyl-NH-heteroaryl, heteroaryl-NH-heterocyclyl, heterocyclyl-N(Me)-C₃-C₁₀ cycloalkyl, heteroaryl-N(Me)-C₃-C₁₀ cycloalkyl, heterocyclyl-N(Me)-heteroaryl, heteroaryl-N(Me)-heterocyclyl, heterocyclyl-NHC(O)—C₃-C₁₀ cycloalkyl, heteroaryl-NHC(O)—C₃-C₁₀ cycloalkyl, heterocyclyl-NHC(O)-heteroaryl, heteroaryl-NHC(O)-heterocyclyl, heterocyclyl-C(O)NH—C₃-C₁₀ cycloalkyl, heteroaryl-C(O)NH—C₃-C₁₀ cycloalkyl, heterocyclyl-C(O)NH-heteroaryl, heteroaryl-C(O)NH-heterocyclyl, heterocyclyl-NHC(O)NH—C₃-C₁₀ cycloalkyl, heteroaryl-NHC(O)NH—C₃-C₁₀ cycloalkyl, heterocyclyl-NHC(O)NH-heteroaryl, and heteroaryl-NHC(O)NH-heterocyclyl; wherein cycloalkyl, heterocyclyl, and heteroaryl optionally may be substituted.

Compounds of formula (IIa) having A₄ linkers different from groups containing an amide or ester moiety, have been found to have an improved stability compared to compounds having A₄ linkers with these moieties. Accordingly, in a further embodiment of formula (IIa), the A₄ linker is selected from the group consisting of —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂O—, —CH₂OCH₂—, and —OCH₂CH₂—; and R¹, R², R³, R⁴, R⁵, R⁶, R⁷, B, A₁, A₂, and A₃, are as defined for formula (I) herein above. More preferably R⁸ may be selected from the group consisting of heterocyclyl and heteroaryl, wherein the heterocyclyl and heteroaryl optionally may be substituted. Furthermore, for this embodiment of formula (IIa) it is especially preferred that R⁸ may be selected from the group consisting of heterocyclyl-C₃-C₁₀ cycloalkyl, heteroaryl-C₃-C₁₀ cycloalkyl, heterocyclyl-heteroaryl, heteroaryl-heterocyclyl, heterocyclyl-O—C₃-C₁₀ cycloalkyl, heteroaryl-O—C₃-C₁₀ cycloalkyl, heterocyclyl-O-heteroaryl, heteroaryl-O-heterocyclyl, heterocyclyl-C(O)—C₃-C₁₀ cycloalkyl, heteroaryl-C(O)—C₃-C₁₀ cycloalkyl, heterocyclyl-C(O)-heteroaryl, heteroaryl-C(O)-heterocyclyl, heterocyclyl-CH₂—C₃-C₁₀ cycloalkyl, heteroaryl-CH₂—C₃-C₁₀ cycloalkyl, heterocyclyl-CH₂-heteroaryl, heteroaryl-CH₂-heterocyclyl, heterocyclyl-CH₂CH₂—C₃-C₁₀ cycloalkyl, heteroaryl-CH₂CH₂—C₃-C₁₀ cycloalkyl, heterocyclyl-CH₂CH₂-heteroaryl, heteroaryl-CH₂CH₂-heterocyclyl, heterocyclyl-NH—C₃-C₁₀ cycloalkyl, heteroaryl-NH—C₃-C₁₀ cycloalkyl, heterocyclyl-NH-heteroaryl, heteroaryl-NH-heterocyclyl, heterocyclyl-N(Me)-C₃-C₁₀ cycloalkyl, heteroaryl-N(Me)-C₃-C₁₀ cycloalkyl, heterocyclyl-N(Me)-heteroaryl, heteroaryl-N(Me)-heterocyclyl, heterocyclyl-NHC(O)—C₃-C₁₀ cycloalkyl, heteroaryl-NHC(O)—C₃-C₁₀ cycloalkyl, heterocyclyl-NHC(O)-heteroaryl, heteroaryl-NHC(O)-heterocyclyl, heterocyclyl-C(O)NH—C₃-C₁₀ cycloalkyl, heteroaryl-C(O)NH—C₃-C₁₀ cycloalkyl, heterocyclyl-C(O)NH-heteroaryl, heteroaryl-C(O)NH-heterocyclyl, heterocyclyl-NHC(O)NH—C₃-C₁₀ cycloalkyl, heteroaryl-NHC(O)NH—C₃-C₁₀ cycloalkyl, heterocyclyl-NHC(O)NH-heteroaryl, and heteroaryl-NHC(O)NH-heterocyclyl; wherein cycloalkyl, heterocyclyl, and heteroaryl optionally may be substituted. Even more preferably the A₄ linker may be selected from the group consisting of single bond, —NH—, —O—, —S—, —SO₂—, —NHCH₂—, —CH₂NH—, —OCH₂—, —CH₂O—, —SCH₂—, —CH₂S—, —SO₂CH₂—, —CH₂SO₂—, —NHSO₂—, —SO₂NH—, —CH₂CH₂NH—, —CH₂CH₂S—, —CH₂CH₂SO₂—, —CH₂NHCH₂—, —CH₂OCH₂—, —CH₂SCH₂—, —CH₂SO₂CH₂—, —NHCH₂CH₂—, —OCH₂CH₂—, —SCH₂CH₂—, —SO₂CH₂CH₂—, —CH₂SO₂NH—, —CH₂NHSO₂—, —SO₂NHCH₂—, and —NHSO₂CH₂—.

In relation to the above mentioned embodiments of formula (IIa) the substituents of R⁸, if any, may be any substituent as defined herein above, more preferably the one or more substituents are selected from the group consisting of halogen, hydroxyl, C₁-C₆ alkyl, C₁-C₆ alkoxy, —CN, —NO₂, —NH₂, —SO₂—C₁-C₆ alkyl, —S(O)—C₁-C₆ alkyl, C₂-C₆ alkenyl, C₂-C₆ alkynyl, C₃-C₁₀ cycloalkyl, aryl, heterocyclyl, and heteroaryl.

A further embodiment of the invention relates to compounds of formula (IIa) wherein R¹ is H; and R², R³, R⁴, R⁵, R⁶, R⁷, R⁸, B, A₁, A₂, A₃ and A₄, are as defined for formula (I) herein above.

More preferably R¹ may be H and R² may be selected from the group consisting of H, C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(CH₂)₁₋₄-heteroaryl, wherein any alkyl, alkenyl, alkynyl, cycloalkyl, aryl, heterocyclyl, and heteroaryl. Even more preferably R¹ may be H and R² may be methyl.

Examples of specific preferred compounds of formula (IIa):
(5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone;
(6-(1-(methylamino)ethyl)pyridin-2-yl)((2S,4R)-4-phenyl-2-(((R)-3-phenylpyrrolidin-1-yl)methyl)pyrrolidin-1-yl)methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(S)-methylamino-ethyl)-furan-2-yl]-methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(R)-methylamino-ethyl)-furan-2-yl]-methanone;
(5-(1(S)-aminoethyl)furan-2-yl)((2S,4R)-4-phenyl-2-(((R)-3-phenylpyrrolidin-1-yl)methyl)pyrrolidin-1-yl)methanone;
(5-(1(R)-amino-ethyl)furan-2-yl)((2S,4R)-4-phenyl-2-(((R)-3-phenylpyrrolidin-1-yl)methyl)pyrrolidin-1-yl)methanone;
(3-(1(S)-(methylamino)ethyl)phenyl)((2S,4R)-4-phenyl-2-(((R)-3-phenylpyrrolidin-1-yl)methyl)pyrrolidin-1-yl)methanone; and
(3-(1(R)-(methylamino)ethyl)phenyl)((2S,4R)-4-phenyl-2-(((R)-3-phenylpyrrolidin-1-yl)methyl)pyrrolidin-1-yl)methanone.

Further examples of specific preferred compounds of formula (IIa):
2-[6-(1-Amino-ethyl)-pyridine-2-carbonyl]-1,2,3,4-tetrahydro-isoquinoline-3-carboxylic acid indan-1-ylamide;
1-(3-(1-aminoethyl)benzoyl)-N-(-2,3-dihydro-1H-inden-1-yl)-octahydro-1H-indole-2-carboxamide;
1-[6-(1-Amino-ethyl)-pyridine-2-carbonyl]-3-phenyl-azetidine-2-carboxylic acid indan-1-ylamide;
1-(3-(1-aminoethyl)benzoyl)-N-(-2,3-dihydro-1H-inden-1-yl)-3-phenylpyrrolidine-2-carboxamide;
1-[6-(1-Amino-ethyl)-pyridine-2-carbonyl]-4-phenyl-pyrrolidine-2-carboxylic acid indan-1-ylamide;
1-(3-(1-aminoethyl)benzoyl)-N-(-2,3-dihydro-1H-inden-1-yl)-5-phenylpyrrolidine-2-carboxamide;
1-(3-(1-aminoethyl)benzamido)-N-(-2,3-dihydro-1H-inden-1-yl)-2,3-dihydro-1H-indene-2-carboxamide;
1-[6-(1-Amino-ethyl)-pyridine-2-carbonyl]-4-(4-fluoro-phenyl)-pyrrolidine-2-carboxylic acid indan-1-ylamide;
1-(3-(1-aminoethyl)benzoyl)-4-(4-chlorophenyl)-N-(-2,3-dihydro-1H-inden-1-yl)pyrrolidine-2-carboxamide;
(4-(1-aminoethyl)-5-methylfuran-2-yl)(-4-phenyl-2-((3-phenylpyrrolidin-1-yl)methyl)pyrrolidin-1-yl)methanone;
(6-(1-aminoethyl)pyridin-2-yl)(-4-phenyl-2-((3-phenylpyrrolidin-1-yl)methyl)pyrrolidin-1-yl)methanone;
(3-(1-aminoethyl)phenyl)(-4-phenyl-2-((3-phenylpyrrolidin-1-yl)methyl)pyrrolidin-1-yl)methanone;
1-(3-(1-aminoethyl)-2-methylfuran-5-carbonyl)-N-(-2,3-dihydro-1H-inden-1-yl)-4-phenylpyrrolidine-2-carboxamide;
1-[6-(1-Amino-ethyl)-pyridine-2-carbonyl]-4-phenyl-pyrrolidine-2-carboxylic acid indan-1-ylamide;
1-(3-(1-aminoethyl)benzoyl)-N-(-2,3-dihydro-1H-inden-1-yl)-4-phenylpyrrolidine-2-carboxamide;
(6-(1-aminoethyl)pyridin-2-yl)(-2-((-3-(4-fluorophenyl)pyrrolidin-1-yl)methyl)-4-phenylpyrrolidin-1-yl)methanone;
(3-(1-aminoethyl)phenyl)(-2-((-3-(4-chlorophenyl)pyrrolidin-1-yl)methyl)-4-phenylpyrrolidin-1-yl)methanone;
(-4-(4-fluorobenzyl)-2-((3-phenylpyrrolidin-1-yl)methyl)pyrrolidin-1-yl)(5-(1-aminoethyl)furan-2-yl)methanone;
[5-(1-Amino-ethyl)-furan-2-yl]-[4-(4-fluoro-benzyl)-2-(3-phenyl-pyrrolidine-1-carbonyl)-pyrrolidin-1-yl]-methanone;
(5-(1-aminoethyl)furan-2-yl)(3-phenyl-2-((3-phenylazetidin-1-yl)methyl)azetidin-1-yl)methanone;
(6-(1-aminoethyl)piperidin-2-yl)(-4-phenyl-2-((3-phenylazetidin-1-yl)methyl)pyrrolidin-1-yl)methanone;
[3-(1-Amino-ethyl)-phenyl]-{-2-[3-(4-fluoro-benzyl)-pyrrolidine-1-carbonyl]-4-phenylpyrrolidin-1-yl}-methanone;
(5-(1-aminoethyl)furan-2-yl)(-2-((3-phenylpyrrolidin-1-yl)methyl)pyrrolidin-1-yl)methanone;
(6-(1-aminoethyl)piperidin-2-yl)(-3-((3-phenylpyrrolidin-1-yl)methyl)-3,4-dihydroisoquinolin-2(1H)-yl)methanone;
(6-(1-aminoethyl)pyridin-2-yl)(-2-((3-phenylpyrrolidin-1-yl)methyl)-octahydroindol-1-yl)methanone;
(3-(1-aminoethyl)phenyl)(-4-(benzyloxy)-2-((3-phenylpyrrolidin-1-yl)methyl)pyrrolidin-1-yl)methanone;
(5-(1-aminoethyl)furan-2-yl)(-4-fluoro-2-((3-phenylpyrrolidin-1-yl)methyl)pyrrolidin-1-yl)methanone;
(6-(1-aminoethyl)pyridin-2-yl)(3-phenyl-2-((3-phenylpyrrolidin-1-yl)methyl)azetidin-1-yl)methanone;
(3-(1-aminoethyl)phenyl)(-3-phenyl-2-((3-phenylpyrrolidin-1-yl)methyl)pyrrolidin-1-yl)methanone;
(6-(1-aminoethyl)pyridin-2-yl)(-4-phenyl-2-((3-phenylpyrrolidin-1-yl)methyl)pyrrolidin-1-yl)methanone;
(3-(1-aminoethyl)phenyl)(-2-phenyl-5-((3-phenylpyrrolidin-1-yl)methyl)pyrrolidin-1-yl)methanone;
(5-(1-aminoethyl)furan-2-yl)(-4-((3-phenylpyrrolidin-1-yl)methyl)thiazolidin-3-yl)methanone;
3-(1-aminoethyl)-N-(-2-((3-phenylpyrrolidin-1-yl)methyl)-2,3-dihydro-1H-inden-1-yl)benzamide;
[5-(1-Amino-ethyl)-furan-2-yl]-[-4-methylamino-2-(-3-phenyl-pyrrolidine-1-carbonyl)-pyrrolidin-1-yl]-methanone;
(6-(1-aminoethyl)piperidin-2-yl)(-4-hydroxy-4-phenyl-2-((3-phenylpyrrolidin-1-yl)methyl)pyrrolidin-1-yl)methanone;
(6-(1-aminoethyl)pyridin-2-yl)(-4-(4-fluorophenyl)-2-((3-phenylpyrrolidin-1-yl)methyl)pyrrolidin-1-yl)methanone;
(3-(1-aminoethyl)phenyl)(-4-(4-chlorophenyl)-2-((3-phenylpyrrolidin-1-yl)methyl)pyrrolidin-1-yl)methanone;
[6-(1-Amino-ethyl)-piperidin-2-yl]-[-4-phenyl-2-(2-phenyl-pyrrolidine-1-carbonyl)-pyrrolidin-1-yl]-methanone;
4-(-1-(2-(1-aminoethyl)furan-5-carbonyl)-3-phenylpyrrolidine-5-carbonyl)-1,3-dimethylpiperazin-2-one;
(6-(1-aminoethyl)piperidin-2-yl)(-2-((-2,3-dihydro-1H-inden-1-ylamino)methyl)pyrrolidin-1-yl)methanone;
1-(2-(1-aminoethyl)furan-5-carbonyl)-4-(benzyloxy)-N-(-2,3-dihydro-1H-inden-1-yl)pyrrolidine-2-carboxamide;
(6-(1-aminoethyl)piperidin-2-yl)(-2-((-2,3-dihydro-1H-inden-1-ylamino)methyl)-4-fluoropyrrolidin-1-yl)methanone;
4-(4-fluorobenzyl)-1-(2-(1-aminoethyl)furan-5-carbonyl)-N-(-2,3-dihydro-1H-inden-1-yl)pyrrolidine-2-carboxamide;
(5-(1-aminoethyl)furan-2-yl)(-4-((-2,3-dihydro-1H-inden-1-ylamino)methyl)thiazolidin-3-yl)methanone;
2-(-4-(2-(1-aminoethyl)piperidine-6-carbonyl)-3-benzyl-2-oxopiperazin-1-yl)-N-(-2,3-dihydro-1H-inden-1-yl)acetamide;
1-(2-(1-aminoethyl)piperidine-6-carbonyl)-N-(-2,3-dihydro-1H-inden-1-yl)-4-hydroxy-4-phenylpyrrolidine-2-carboxamide;
(5-(1-aminoethyl)-2-methylfuran-3-yl)(-4-phenyl-2-((3-phenylpyrrolidin-1-yl)methyl)pyrrolidin-1-yl)methanone;
(6-(1-aminoethyl)piperidin-2-yl)(-4-phenyl-2-((3-phenylpyrrolidin-1-yl)methyl)pyrrolidin-1-yl)methanone;
(4-(aminomethyl)-5-isobutylfuran-2-yl)(-4-phenyl-2-((3-phenylpyrrolidin-1-yl)methyl)pyrrolidin-1-yl)methanone;
1-(2-(1-aminoethyl)-5-methylfuran-4-carbonyl)-N-(-2,3-dihydro-1H-inden-1-yl)-4-phenylpyrrolidine-2-carboxamide;
1-(2-(1-aminoethyl)furan-5-carbonyl)-N-(-2,3-dihydro-1H-inden-1-yl)-4-phenylpyrrolidine-2-carboxamide;
1-(2-(1-aminoethyl)piperidine-6-carbonyl)-N-(-2,3-dihydro-1H-inden-1-yl)-4-phenylpyrrolidine-2-carboxamide;
1-(3-(aminomethyl)-2-isobutylfuran-5-carbonyl)-N-(-2,3-dihydro-1H-inden-1-yl)-4-phenylpyrrolidine-2-carboxamide;
[5-(1-Amino-ethyl)-furan-2-yl]-{-4-(4-fluoro-phenyl)-2-[3-(4-fluoro-phenyl)-pyrrolidine-1-carbonyl]-pyrrolidin-1-yl}-methanone;
[6-(1-Amino-ethyl)-piperidin-2-yl]-{-4-(4-chloro-phenyl)-2-[3-(4-chloro-phenyl)-pyrrolidine-1-carbonyl]-pyrrolidin-1-yl}-methanone;
(6-(1-aminoethyl)pyridin-2-yl)(-2-((-3-(3-fluorophenyl)pyrrolidin-1-yl)methyl)-4-phenylpyrrolidin-1-yl)methanone;
(3-(1-aminoethyl)phenyl)(-2-((-3-(3,4-dichlorophenyl)pyrrolidin-1-yl)methyl)-4-phenylpyrrolidin-1-yl)methanone;
(-4-(4-fluorophenyl)-2-((-3-(3-fluorophenyl)pyrrolidin-1-yl)methyl)pyrrolidin-1-yl)(5-(1-(methylamino)propyl)furan-2-yl)methanone;
(5-(1-aminoethyl)furan-2-yl)(2-((2,3-dihydro-1H-inden-1-ylamino)methyl)-4-phenylpyrrolidin-1-yl)methanone;
(5-(-1-aminoethyl)furan-2-yl)(-2-(phenoxymethyl)-4-phenylpyrrolidin-1-yl)methanone;
(5-(-1-aminoethyl)furan-2-yl)(-2-((naphthalen-1-yloxy)methyl)-4-phenylpyrrolidin-1-yl)methanone;
(5-(-1-aminoethyl)furan-2-yl)(-2-((2,3-dihydro-1H-inden-1-ylamino)methyl)-4-phenylpyrrolidin-1-yl)methanone;
(5-(-1-aminoethyl)furan-2-yl)(-4-phenyl-2-((-1,2,3,4-tetrahydronaphthalen-1-ylamino)methyl)pyrrolidin-1-yl)methanone;
(5-(-1-aminoethyl)furan-2-yl)(-2-(2-benzyl-2H-tetrazol-5-yl)-4-phenylpyrrolidin-1-yl)methanone;
(5-(-1-aminoethyl)furan-2-yl)(-2-(4-benzyloxazol-2-yl)-4-phenylpyrrolidin-1-yl)methanone;
[5-(-1-Amino-ethyl)-furan-2-yl]-[-2-(5-benzoyl-[1,2,4]oxadiazol-3-yl)-4-phenyl-pyrrolidin-111]-methanone;
1-(2-(-1-aminoethyl)furan-5-carbonyl)-4-phenyl-N-(4-phenyl-1,2,3-thiadiazol-5-yl)pyrrolidine-2-carboxamide;
1-(2-(-1-aminoethyl)furan-5-carbonyl)-4-phenyl-N-(1-phenyl-1H-pyrazol-5-yl)pyrrolidine-2-carboxamide;
1-(2-(-1-aminoethyl)furan-5-carbonyl)-4-phenyl-N-(5-phenyl-1H-tetrazol-1-yl)pyrrolidine-2-carboxamide;
(5-(-1-aminoethyl)furan-2-yl)(-2-((1-methyl-1H-indol-3-yl)methyl)-4-phenylpyrrolidin-1-yl)methanone;
1-(3-((-1-(2-(-1-aminoethyl)furan-5-carbonyl)-4-phenylpyrrolidin-2-yl)methyl)-1H-indol-1-yl)ethanone;
(5-(-1-aminoethyl)furan-2-yl)(-2-(benzofuran-3-ylmethyl)-4-phenylpyrrolidin-1-yl)methanone;
[5-(-1-Amino-ethyl)-2-methoxy-phenyl]-[-4-phenyl-2-(-3-phenyl-pyrrolidine-1-carbonyl)-pyrrolidin-1-yl]-methanone;
[5-(-1-Amino-ethyl)-2-benzyloxy-phenyl]-[-4-phenyl-2-(-3-phenyl-pyrrolidine-1-carbonyl)-pyrrolidin-1-yl]-methanone;
[5-(-1-Amino-ethyl)-4-ethoxy-2-piperidin-1-yl-phenyl]-[-4-phenyl-2-(-3-phenyl-pyrrolidine-1-carbonyl)-pyrrolidin-1-yl]-methanone;
[5-(-1-Amino-ethyl)-1H-pyrrol-2-yl]-[-4-phenyl-2-(-3-phenyl-pyrrolidine-1-carbonyl)-pyrrolidin-1-yl]-methanone;
[5-(-1-Amino-ethyl)-[1,2,4]oxadiazol-3-yl]-[-4-phenyl-2-(-3-phenyl-pyrrolidine-1-carbonyl)-pyrrolidin-1-yl]-methanone;
[3-(-1-Amino-ethyl)-[1,2,4]oxadiazol-5-yl]-[-4-phenyl-2-(-3-phenyl-pyrrolidine-1-carbonyl)-pyrrolidin-1-yl]-methanone;
[5-(-1-Amino-ethyl)-oxazol-2-yl]-[-4-phenyl-2-(-3-phenyl-pyrrolidine-1-carbonyl)-pyrrolidin-1-yl]-methanone;
[5-(-1-Amino-ethyl)-1H-imidazol-2-yl]-[-4-phenyl-2-(-3-phenyl-pyrrolidine-1-carbonyl)-pyrrolidin-1-yl]-methanone;
[4-(-1-Amino-ethyl)-1-methyl-1H-imidazol-2-yl]-[-4-phenyl-2-(-3-phenyl-pyrrolidine-1-carbonyl)-pyrrolidin-1-yl]-methanone;
[4-(-1-Amino-ethyl)-phenyl]-[-4-phenyl-2-(-3-phenyl-pyrrolidine-1-carbonyl)-pyrrolidin-1-yl]-methanone;
(6-Aminomethyl-pyridin-2-yl)-[-4-phenyl-2-(-3-phenyl-pyrrolidine-1-carbonyl)-pyrrolidin-1-yl]-methanone;
(5-Aminomethyl-thiazol-2-yl)-[-4-phenyl-2-(-3-phenyl-pyrrolidine-1-carbonyl)-pyrrolidin-1-yl]-methanone;
(5-Aminomethyl-thiophen-2-yl)-[-4-phenyl-2-(-3-phenyl-pyrrolidine-1-carbonyl)-pyrrolidin-1-yl]-methanone;
(5-Methylaminomethyl-thiophen-2-yl)-[-4-phenyl-2-(-3-phenyl-pyrrolidine-1-carbonyl)-pyrrolidin-1-yl]-methanone;
(5-Methylaminomethyl-furan-2-yl)-[-4-phenyl-2-(-3-phenyl-pyrrolidine-1-carbonyl)-pyrrolidin-1-yl]-methanone;
(5-Aminomethyl-furan-2-yl)-[-4-phenyl-2-(-3-phenyl-pyrrolidine-1-carbonyl)-pyrrolidin-1-yl]-methanone;
(2-Aminomethyl-1,5-dimethyl-1H-imidazol-4-yl)-[-4-phenyl-2-(-3-phenyl-pyrrolidine-1-carbonyl)-pyrrolidin-1-yl]-methanone;
(5-Methylaminomethyl-[1,2,4]oxadiazol-3-yl)-[-4-phenyl-2-(-3-phenyl-pyrrolidine-1-carbonyl)-pyrrolidin-1-yl]-methanone;
[2-(-1-Amino-ethyl)-5-methyl-oxazol-4-yl]-[-4-phenyl-2-(-3-phenyl-pyrrolidine-1-carbonyl)-pyrrolidin-1-yl]-methanone;
(5-Aminomethyl-[1,2,4]oxadiazol-3-yl)-[-4-phenyl-2-(-3-phenyl-pyrrolidine-1-carbonyl)-pyrrolidin-1-yl]-methanone;
(5-Aminomethyl-furan-3-yl)-[-4-phenyl-2-(-3-phenyl-pyrrolidine-1-carbonyl)-pyrrolidin-1-yl]-methanone;
(4-Aminomethyl-5-methyl-furan-2-yl)-[-4-phenyl-2-(-3-phenyl-pyrrolidine-1-carbonyl)-pyrrolidin-1-yl]-methanone;
(4-Aminomethyl-5-isobutyl-furan-2-yl)-[-4-phenyl-2-(-3-phenyl-pyrrolidine-1-carbonyl)-pyrrolidin-1-yl]-methanone;
(5-Aminomethyl-isoxazol-3-yl)-[-4-phenyl-2-(-3-phenyl-pyrrolidine-1-carbonyl)-pyrrolidin-1-yl]-methanone;
(5-Aminomethyl-thiophen-3-yl)-[-4-phenyl-2-(-3-phenyl-pyrrolidine-1-carbonyl)-pyrrolidin-1-yl]-methanone;
[2-(-1-Amino-ethyl)-oxazol-5-yl]-[-4-phenyl-2-(-3-phenyl-pyrrolidine-1-carbonyl)-pyrrolidin-1-yl]-methanone;
[6-(1-Methylamino-ethyl)-piperidin-2-yl]-[-4-phenyl-2-(-3-phenyl-pyrrolidine-1-carbonyl)-pyrrolidin-1-yl]-methanone;
(6-(1-(methylamino)ethyl)piperidin-2-yl)(-4-phenyl-2-((-3-phenylpyrrolidin-1-yl)methyl)pyrrolidin-1-yl)methanone;
[5-(1-Methylamino-ethyl)-furan-2-yl]-[-2-(7-phenyl-thiazolo[5,4-b]pyridin-2-yl)-pyrrolidin-1-yl]-methanone;
(5-(1-(methylamino)ethyl)furan-2-yl)(-2-(4-phenylthiazolo[4,5-c]pyridin-2-yl)pyrrolidin-1-yl)methanone;
(5-(1-(methylamino)ethyl)furan-2-yl)(-2-(7-phenylthiazolo[5,4-d]pyrimidin-2-yl)pyrrolidin-1-yl)methanone;
(4-Fluoro-phenyl)-(3-{1′-[5-(1-methylamino-ethyl)-furan-2-carbonyl]-[1,2′]-Dipyrrolidinyl-2-yl}-pyridin-2-yl)-methanone;
(octahydro-6-phenethylpyrrolo[2,3-c]pyridin-1-yl)(5-(1-(methylamino)ethyl)furan-2-yl)methanone;
(4-Fluoro-phenyl)-(2-{1-[5-(1-methylamino-ethyl)-furan-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(5-(1-(methylamino)ethyl)furan-2-yl)methanone;
(2-(5-(4-fluorophenoxy)pyridin-3-yl)pyrrolidin-1-yl)(5-(1-(methylamino)ethyl)furan-2-yl)methanone;
(4-Fluoro-phenyl)-(5-{1-[5-(1-methylamino-ethyl)-furan-2-carbonyl]-pyrrolidin-2-yl}-pyridin-3-yl)-methanone;
(2-(4-(4-fluorophenoxy)pyridin-2-yl)pyrrolidin-1-yl)(5-(1-(methylamino)ethyl)furan-2-yl)methanone;
(2-(2-(N-(4-fluorophenyl)-N-methylamino)-5-fluoropyridin-4-yl)pyrrolidin-1-yl)(5-(1-(methylamino)ethyl)furan-2-yl)methanone;
(3-(1-(methylamino)ethyl)phenyl)(-2-(7-phenylthiazolo[5,4-b]pyridin-2-yl)pyrrolidin-1-yl)methanone;
(3-(1-(methylamino)ethyl)phenyl)(-2-(4-phenylthiazolo[4,5-c]pyridin-2-yl)pyrrolidin-1-yl)methanone;
(3-(1-(methylamino)ethyl)phenyl)(-2-(7-phenylthiazolo[5,4-d]pyrimidin-2-yl)pyrrolidin-1-yl)methanone;
(4-Fluoro-phenyl)-(3-{1′-[3-(1-methylamino-ethyl)-benzoyl]-[1,2′]-Dipyrrolidinyl-2-yl}-pyridin-2-yl)-methanone;
(octahydro-6-phenethylpyrrolo[2,3-c]pyridin-1-yl)(3-(1-(methylamino)ethyl)phenyl)methanone;
(4-Fluoro-phenyl)-(2-{1-[3-(1-methylamino-ethyl)-benzoyl]-pyrrolidin-2-yl}-thiazol-4-yl)-methanone;
(2-(2-(N-(4-fluorophenyl)-N-methylamino)pyridin-4-yl)pyrrolidin-1-yl)(3-(1-(methylamino)ethyl)phenyl)methanone;
(2-(5-(4-fluorophenoxy)pyridin-3-yl)pyrrolidin-1-yl)(3-(1-(methylamino)ethyl)phenyl)methanone;
(4-Fluoro-phenyl)-(5-{1-[3-(1-methylamino-ethyl)-benzoyl]-pyrrolidin-2-yl}-pyridin-3-yl)-methanone;
(2-(4-(4-fluorophenoxy)pyridin-2-yl)pyrrolidin-1-yl)(3-(1-(methylamino)ethyl)phenyl)methanone;
(2-(2-(N-(4-fluorophenyl)-N-methylamino)-5-fluoropyridin-4-yl)pyrrolidin-1-yl)(3-(1-(methylamino)ethyl)phenyl)methanone;
(2-(2-(N-(4-fluorophenyl)-N-methylamino)pyridin-4-yl)pyrrolidin-1-yl)(3-(1-(methylamino)ethyl)phenyl)methanone;
(6-(1-(methylamino)ethyl)piperidin-2-yl)(-2-(7-phenylthiazolo[5,4-b]pyridin-2-yl)pyrrolidin-1-yl)methanone;
(6-(1-(methylamino)ethyl)piperidin-2-yl)(-2-(4-phenylthiazolo[4,5-c]pyridin-2-yl)pyrrolidin-1-yl)methanone;
(6-(1-(methylamino)ethyl)piperidin-2-yl)(-2-(7-phenylthiazolo[5,4-d]pyrimidin-2-yl)pyrrolidin-1-yl)methanone;
(4-Fluoro-phenyl)-(3-{1′-[6-(1-methylamino-ethyl)-piperidine-2-carbonyl]-[1,2]bipyrrolidinyl-2-yl}-pyridin-2-yl)-methanone;
(octahydro-6-phenethylpyrrolo[2,3-c]pyridin-1-yl)(6-(1-(methylamino)ethyl)piperidin-2-yl)methanone;
(4-Fluoro-phenyl)-(2-{1-[6-(1-methylamino-ethyl)-piperidine-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(6-(1-(methylamino)ethyl)piperidin-2-yl)methanone;
(2-(5-(4-fluorophenoxy)pyridin-3-yl)pyrrolidin-1-yl)(6-(1-(methylamino)ethyl)piperidin-2-yl)methanone;
(4-Fluoro-phenyl)-(5-{1-[6-(1-methylamino-ethyl)-piperidine-2-carbonyl]-pyrrolidin-2-yl}-pyridin-3-yl)-methanone;
(2-(4-(4-fluorophenoxy)pyridin-2-yl)pyrrolidin-1-yl)(6-(1-(methylamino)ethyl)piperidin-2-yl)methanone;
(2-(2-(N-(4-fluorophenyl)-N-methylamino)-5-fluoropyridin-4-yl)pyrrolidin-1-yl)(6-(1-(methylamino)ethyl)piperidin-2-yl)methanone;
(2-(2-(N-(4-fluorophenyl)-N-methylamino)pyridin-4-yl)pyrrolidin-1-yl)(6-(1-(methylamino)ethyl)piperidin-2-yl)methanone;
{-1-[5-(1-Amino-ethyl)-furan-2-carbonyl]-4-phenyl-pyrrolidin-2-yl}-[-3-(4-fluoro-phenyl)-pyrrolidin-1-yl]-methanone;
[-1-[5-(1-Amino-ethyl)-furan-2-carbonyl]-4-(-4-fluoro-phenyl)-pyrrolidin-2-yl]-(-3-phenyl-pyrrolidin-1-yl)-methanone; and
(-4-(4-fluorophenyl)-2-((-3-(4-fluorophenyl)pyrrolidin-1-yl)methyl)pyrrolidin-1-yl)(5-(1-(methylamino)ethyl)furan-2-yl)methanone.

For the above mentioned further compounds of formula (IIa) the following stereoisomers are preferred:
(S)-2-(2-(1-aminoethyl)picolinoyl)-N—((R)-2,3-dihydro-1H-inden-1-yl)-1,2,3,4-tetrahydroisoquinoline-3-carboxamide;
(2S)-1-(3-(1-aminoethyl)benzoyl)-N—((R)-2,3-dihydro-1H-inden-1-yl)-octahydro-1H-indole-2-carboxamide;
1-(2-(1-aminoethyl)picolinoyl)-N—((R)-2,3-dihydro-1H-inden-1-yl)-3-phenylazetidine-2-carboxamide;
(2S,3S)-1-(3-(1-aminoethyl)benzoyl)-N—((R)-2,3-dihydro-1H-inden-1-yl)-3-phenylpyrrolidine-2-carboxamide;
(2R,4S)-1-(2-(1-aminoethyl)picolinoyl)-N—((R)-2,3-dihydro-1H-inden-1-yl)-4-phenylpyrrolidine-2-carboxamide;
(2R,5S)-1-(3-(1-aminoethyl)benzoyl)-N—((R)-2,3-dihydro-1H-inden-1-yl)-5-phenylpyrrolidine-2-carboxamide;
(1R,2R)-1-(3-(1-aminoethyl)benzamido)-N—((R)-2,3-dihydro-1H-inden-1-yl)-2,3-dihydro-1H-indene-2-carboxamide;
(2S,4R)-1-(2-(1-aminoethyl)picolinoyl)-N—((R)-2,3-dihydro-1H-inden-1-yl)-4-(4-fluorophenyl)pyrrolidine-2-carboxamide;
(2S,4R)-1-(3-(1-aminoethyl)benzoyl)-4-(4-chlorophenyl)-N—((R)-2,3-dihydro-1H-inden-1-yl)pyrrolidine-2-carboxamide;
(4-(1-aminoethyl)-5-methylfuran-2-yl)((2S,4R)-4-phenyl-2-((3-phenylpyrrolidin-1-yl)methyl)pyrrolidin-1-yl)methanone;
(6-(1-aminoethyl)pyridin-2-yl)((2S,4R)-4-phenyl-2-((3-phenylpyrrolidin-1-yl)methyl)pyrrolidin-1-yl)methanone;
(3-(1-aminoethyl)phenyl)((2S,4R)-4-phenyl-2-((3-phenylpyrrolidin-1-yl)methyl)pyrrolidin-1-yl)methanone;
(2S,4R)-1-(3-(1-aminoethyl)-2-methylfuran-5-carbonyl)-N—((R)-2,3-dihydro-1H-inden-1-yl)-4-phenylpyrrolidine-2-carboxamide;
(2S,4R)-1-(2-(1-aminoethyl)picolinoyl)-N—((R)-2,3-dihydro-1H-inden-1-yl)-4-phenylpyrrolidine-2-carboxamide;
(2S,4R)-1-(3-(1-aminoethyl)benzoyl)-N—((R)-2,3-dihydro-1H-inden-1-yl)-4-phenylpyrrolidine-2-carboxamide;
(6-(1-aminoethyl)pyridin-2-yl)((2S,4R)-2-(((R)-3-(4-fluorophenyl)pyrrolidin-1-yl)methyl)-4-phenylpyrrolidin-1-yl)methanone;
(3-(1-aminoethyl)phenyl)((2S,4R)-2-(((R)-3-(4-chlorophenyl)pyrrolidin-1-yl)methyl)-4-phenylpyrrolidin-1-yl)methanone;
((2S)-4-(4-fluorobenzyl)-2-((3-phenylpyrrolidin-1-yl)methyl)pyrrolidin-1-yl)(5-(1-aminoethyl)furan-2-yl)methanone;
[5-(1-Amino-ethyl)-furan-2-yl]-[(S)-4-(4-fluoro-benzyl)-2-(3-phenyl-pyrrolidine-1-carbonyl)-pyrrolidin-1-yl]-methanone;
(5-(1-aminoethyl)furan-2-yl)(3-phenyl-2-((3-phenylazetidin-1-yl)methyl)azetidin-1-yl)methanone;
(6-(1-aminoethyl)piperidin-2-yl)((2S,4R)-4-phenyl-2-((3-phenylazetidin-1-yl)methyl)pyrrolidin-1-yl)methanone;
[3-(1-Amino-ethyl)-phenyl]-{(2S,4R)-2-[3-(4-fluoro-benzyl)-pyrrolidine-1-carbonyl]-4-phenylpyrrolidin-1-yl}-methanone;
(5-(1-aminoethyl)furan-2-yl)((S)-2-((3-phenylpyrrolidin-1-yl)methyl)pyrrolidin-1-yl)methanone;
(6-(1-aminoethyl)piperidin-2-yl)((S)-3-((3-phenylpyrrolidin-1-yl)methyl)-3,4-dihydroisoquinolin-2(1H)-yl)methanone;
(6-(1-aminoethyl)pyridin-2-yl)((2S)-2-((3-phenylpyrrolidin-1-yl)methyl)-octahydroindol-1-yl)methanone;
(3-(1-aminoethyl)phenyl)((2S)-4-(benzyloxy)-2-((3-phenylpyrrolidin-1-yl)methyl)pyrrolidin-1-yl)methanone;
(5-(1-aminoethyl)furan-2-yl)((2S,4R)-4-fluoro-2-((3-phenylpyrrolidin-1-yl)methyl)pyrrolidin-1-yl)methanone;
(6-(1-aminoethyl)pyridin-2-yl)(3-phenyl-2-((3-phenylpyrrolidin-1-yl)methyl)azetidin-1-yl)methanone;
(3-(1-aminoethyl)phenyl)((2S,3S)-3-phenyl-2-((3-phenylpyrrolidin-1-yl)methyl)pyrrolidin-1-yl)methanone;
(6-(1-aminoethyl)pyridin-2-yl)((2S,4S)-4-phenyl-2-((3-phenylpyrrolidin-1-yl)methyl)pyrrolidin-1-yl)methanone;
(3-(1-aminoethyl)phenyl)((2S,5R)-2-phenyl-5-((3-phenylpyrrolidin-1-yl)methyl)pyrrolidin-1-yl)methanone;
(5-(1-aminoethyl)furan-2-yl)((R)-4-((3-phenylpyrrolidin-1-yl)methyl)thiazolidin-3-yl)methanone;
3-(1-aminoethyl)-N-((1R,2S)-2-((3-phenylpyrrolidin-1-yl)methyl)-2,3-dihydro-1H-inden-1-yl)benzamide;
[5-(1-Amino-ethyl)-furan-2-yl]-[(S)-4-methylamino-2-((S)—(R)-3-phenyl-pyrrolidine-1-carbonyl)-pyrrolidin-1-yl]-methanone;
(6-(1-aminoethyl)piperidin-2-yl)((2S,4S)-4-hydroxy-4-phenyl-2-((3-phenylpyrrolidin-1-yl)methyl)pyrrolidin-1-yl)methanone;
(6-(1-aminoethyl)pyridin-2-yl)((2S,4R)-4-(4-fluorophenyl)-2-((3-phenylpyrrolidin-1-yl)methyl)pyrrolidin-1-yl)methanone;
(3-(1-aminoethyl)phenyl)((2S,4R)-4-(4-chlorophenyl)-2-((3-phenylpyrrolidin-1-yl)methyl)pyrrolidin-1-yl)methanone;
[6-(1-Amino-ethyl)-piperidin-2-yl]-[(2S,4R)-4-phenyl-2-(2-phenyl-pyrrolidine-1-carbonyl)-pyrrolidin-1-yl]-methanone;
4-((3R,5S)-1-(2-(1-aminoethyl)furan-5-carbonyl)-3-phenylpyrrolidine-5-carbonyl)-1,3-dimethylpiperazin-2-one;
(6-(1-aminoethyl)piperidin-2-yl)((S)-2-(((R)-2,3-dihydro-1H-inden-1-ylamino)methyl)pyrrolidin-1-yl)methanone;
(2S)-1-(2-(1-aminoethyl)furan-5-carbonyl)-4-(benzyloxy)-N—((R)-2,3-dihydro-1H-inden-1-yl)pyrrolidine-2-carboxamide;
(6-(1-aminoethyl)piperidin-2-yl)((2S,4R)-2-(((R)-2,3-dihydro-1H-inden-1-ylamino)methyl)-4-fluoropyrrolidin-1-yl)methanone;
(2S,4R)-4-(4-fluorobenzyl)-1-(2-(1-aminoethyl)furan-5-carbonyl)-N—((R)-2,3-dihydro-1H-inden-1-yl)pyrrolidine-2-carboxamide;
(5-(1-aminoethyl)furan-2-yl)((R)-4-(((R)-2,3-dihydro-1H-inden-1-ylamino)methyl)thiazolidin-3-yl)methanone;
2-((S)-4-(2-(1-aminoethyl)piperidine-6-carbonyl)-3-benzyl-2-oxopiperazin-1-yl)-N—((R)-2,3-dihydro-1H-inden-1-yl)acetamide;
(2S,4S)-1-(2-(1-aminoethyl)piperidine-6-carbonyl)-N—((R)-2,3-dihydro-1H-inden-1-yl)-4-hydroxy-4-phenylpyrrolidine-2-carboxamide;
(5-(1-aminoethyl)-2-methylfuran-3-yl)((2S,4R)-4-phenyl-2-((3-phenylpyrrolidin-1-yl)methyl)pyrrolidin-1-yl)methanone;
(6-(1-aminoethyl)piperidin-2-yl)((2S,4R)-4-phenyl-2-((3-phenylpyrrolidin-1-yl)methyl)pyrrolidin-1-yl)methanone;
(4-(aminomethyl)-5-isobutylfuran-2-yl)((2S,4R)-4-phenyl-2-((3-phenylpyrrolidin-1-yl)methyl)pyrrolidin-1-yl)methanone;
(2S,4R)-1-(2-(1-aminoethyl)-5-methylfuran-4-carbonyl)-N—((R)-2,3-dihydro-1H-inden-1-yl)-4-phenylpyrrolidine-2-carboxamide;
(2S,4R)-1-(2-(1-aminoethyl)furan-5-carbonyl)-N—((R)-2,3-dihydro-1H-inden-1-yl)-4-phenylpyrrolidine-2-carboxamide;
(2S,4R)-1-(2-(1-aminoethyl)piperidine-6-carbonyl)-N—((R)-2,3-dihydro-1H-inden-1-yl)-4-phenylpyrrolidine-2-carboxamide;
(2S,4R)-1-(3-(aminomethyl)-2-isobutylfuran-5-carbonyl)-N—((R)-2,3-dihydro-1H-inden-1-yl)-4-phenylpyrrolidine-2-carboxamide;
[5-(1-Amino-ethyl)-furan-2-yl]-{(2S,4R)-4-(4-fluoro-phenyl)-2-[(R)-3-(4-fluoro-phenyl)-pyrrolidine-1-carbonyl]-pyrrolidin-1-yl}-methanone;
[6-(1-Amino-ethyl)-piperidin-2-yl]-{(2S,4R)-4-(4-chloro-phenyl)-2-[(R)-3-(4-chloro-phenyl)-pyrrolidine-1-carbonyl]-pyrrolidin-1-yl}-methanone;
(6-(1-aminoethyl)pyridin-2-yl)((2S,4R)-2-(((R)-3-(3-fluorophenyl)pyrrolidin-1-yl)methyl)-4-phenylpyrrolidin-1-yl)methanone;
(3-(1-aminoethyl)phenyl)((2S,4R)-2-(((R)-3-(3,4-dichlorophenyl)pyrrolidin-1-yl)methyl)-4-phenylpyrrolidin-1-yl)methanone;
((2S,4R)-4-(4-fluorophenyl)-2-(((R)-3-(3-fluorophenyl)pyrrolidin-1-yl)methyl)pyrrolidin-1-yl)(5-(1-(methylamino)propyl)furan-2-yl)methanone;
(5-(1-aminoethyl)furan-2-yl)(2-((2,3-dihydro-1H-inden-1-ylamino)methyl)-4-phenylpyrrolidin-1-yl)methanone; (5-((S)-1-aminoethyl)furan-2-yl)((2S,4R)-2-(phenoxymethyl)-4-phenylpyrrolidin-1-yl)methanone; (5-((S)-1-aminoethyl)furan-2-yl)((2S,4R)-2-((naphthalen-1-yloxy)methyl)-4-phenylpyrrolidin-1-yl)methanone;
(5-((S)-1-aminoethyl)furan-2-yl)((2S,4R)-2-((2,3-dihydro-1H-inden-1-ylamino)methyl)-4-phenylpyrrolidin-1-yl)methanone;
(5-((S)-1-aminoethyl)furan-2-yl)((2S,4R)-4-phenyl-2-(((R)-1,2,3,4-tetrahydronaphthalen-1-ylamino)methyl)pyrrolidin-1-yl)methanone;
(5-((S)-1-aminoethyl)furan-2-yl)((2S,4R)-2-(2-benzyl-2H-tetrazol-5-yl)-4-phenylpyrrolidin-1-yl)methanone;
(5-((S)-1-aminoethyl)furan-2-yl)((2S,4R)-2-(4-benzyloxazol-2-yl)-4-phenylpyrrolidin-1-yl)methanone;
[5-((S)-1-Amino-ethyl)-furan-2-yl]-[(2S,4R)-2-(5-benzoyl-[1,2,4]oxadiazol-3-yl)-4-phenyl-pyrrolidin-1-yl]-methanone;
(2S,4R)-1-(2-((S)-1-aminoethyl)furan-5-carbonyl)-4-phenyl-N-(4-phenyl-1,2,3-thiadiazol-5-yl)pyrrolidine-2-carboxamide;
(2S,4R)-1-(2-((S)-1-aminoethyl)furan-5-carbonyl)-4-phenyl-N-(1-phenyl-1H-pyrazol-5-yl)pyrrolidine-2-carboxamide;
(2S,4R)-1-(2-((S)-1-aminoethyl)furan-5-carbonyl)-4-phenyl-N-(5-phenyl-1H-tetrazol-1-yl)pyrrolidine-2-carboxamide;
(5-((S)-1-aminoethyl)furan-2-yl)((2S,4R)-2-((1-methyl-1H-indol-3-yl)methyl)-4-phenylpyrrolidin-1-yl)methanone;
1-(3-(((2S,4R)-1-(2-((S)-1-aminoethyl)furan-5-carbonyl)-4-phenylpyrrolidin-2-yl)methyl)-1H-indol-1-yl)ethanone;
(5-((S)-1-aminoethyl)furan-2-yl)((2S,4R)-2-(benzofuran-3-ylmethyl)-4-phenylpyrrolidin-1-yl)methanone;
[5-((S)-1-Amino-ethyl)-2-methoxy-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-2-benzyloxy-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-4-ethoxy-2-piperidin-1-yl-phenyl]-[(2S,4R)-4-phenyl-2-((R)-3-phenyl-pyrrolidine-1-carbonyl)-pyrrolidin-1-yl]-methanone;
[5-((S)-1-Amino-ethyl)-1H-pyrrol-2-yl]-[(2S,4R)-4-phenyl-2-((R)-3-phenyl-pyrrolidine-1-carbonyl)-pyrrolidin-1-yl]-methanone;
[5-((S)-1-Amino-ethyl)-[1,2,4]oxadiazol-3-yl]-[(2S,4R)-4-phenyl-2-((R)-3-phenyl-pyrrolidine-1-carbonyl)-pyrrolidin-1-yl]-methanone;
[3-((S)-1-Amino-ethyl)-[1,2,4]oxadiazol-5-yl]-[(2S,4R)-4-phenyl-2-((R)-3-phenyl-pyrrolidine-1-carbonyl)-pyrrolidin-1-yl]-methanone;
[5-((S)-1-Amino-ethyl)-oxazol-2-yl]-[(2S,4R)-4-phenyl-2-((R)-3-phenyl-pyrrolidine-1-carbonyl)-pyrrolidin-1-yl]-methanone;
[5-((S)-1-Amino-ethyl)-1H-imidazol-2-yl]-[(2S,4R)-4-phenyl-2-((R)-3-phenyl-pyrrolidine-1-carbonyl)-pyrrolidin-1-yl]-methanone;
[4-((S)-1-Amino-ethyl)-1-methyl-1H-imidazol-2-yl]-[(2S,4R)-4-phenyl-2-((R)-3-phenyl-pyrrolidine-1-carbonyl)-pyrrolidin-1-yl]-methanone;
[4-((S)-1-Amino-ethyl)-phenyl]-[(2S,4R)-4-phenyl-2-((R)-3-phenyl-pyrrolidine-1-carbonyl)-pyrrolidin-1-yl]-methanone;
(6-Aminomethyl-pyridin-2-yl)-[(2S,4R)-4-phenyl-2-((R)-3-phenyl-pyrrolidine-1-carbonyl)-pyrrolidin-1-yl]-methanone;
(5-Aminomethyl-thiazol-2-yl)-[(2S,4R)-4-phenyl-2-((R)-3-phenyl-pyrrolidine-1-carbonyl)-pyrrolidin-1-yl]-methanone;
(5-Aminomethyl-thiophen-2-yl)-[(2S,4R)-4-phenyl-2-((R)-3-phenyl-pyrrolidine-1-carbonyl)-pyrrolidin-1-yl]-methanone;
(5-Methylaminomethyl-thiophen-2-yl)-[(2S,4R)-4-phenyl-2-((R)-3-phenyl-pyrrolidine-1-carbonyl)-pyrrolidin-1-yl]-methanone;
(5-Methylaminomethyl-furan-2-yl)-[(2S,4R)-4-phenyl-2-((R)-3-phenyl-pyrrolidine-1-carbonyl)-pyrrolidin-1-yl]-methanone;
(5-Aminomethyl-furan-2-yl)-[(2S,4R)-4-phenyl-2-((R)-3-phenyl-pyrrolidine-1-carbonyl)-pyrrolidin-1-yl]-methanone;
(2-Aminomethyl-1,5-dimethyl-1H-imidazol-4-yl)-[(2S,4R)-4-phenyl-2-((R)-3-phenyl-pyrrolidine-1-carbonyl)-pyrrolidin-1-yl]-methanone;
(5-Methylaminomethyl-[1,2,4]oxadiazol-3-yl)-[(2S,4R)-4-phenyl-2-((R)-3-phenyl-pyrrolidine-1-carbonyl)-pyrrolidin-1-yl]-methanone;
[2-((S)-1-Amino-ethyl)-5-methyl-oxazol-4-yl]-[(2S,4R)-4-phenyl-2-((R)-3-phenyl-pyrrolidine-1-carbonyl)-pyrrolidin-1-yl]-methanone;
(5-Aminomethyl-[1,2,4]oxadiazol-3-yl)-[(2S,4R)-4-phenyl-2-((R)-3-phenyl-pyrrolidine-1-carbonyl)-pyrrolidin-1-yl]-methanone;
(5-Aminomethyl-furan-3-yl)-[(2S,4R)-4-phenyl-2-((R)-3-phenyl-pyrrolidine-1-carbonyl)-pyrrolidin-1-yl]-methanone;
(4-Aminomethyl-5-methyl-furan-2-yl)-[(2S,4R)-4-phenyl-2-((R)-3-phenyl-pyrrolidine-1-carbonyl)-pyrrolidin-1-yl]-methanone;
(4-Aminomethyl-5-isobutyl-furan-2-yl)-[(2S,4R)-4-phenyl-2-((R)-3-phenyl-pyrrolidine-1-carbonyl)-pyrrolidin-1-yl]-methanone;
(5-Aminomethyl-isoxazol-3-yl)-[(2S,4R)-4-phenyl-2-((R)-3-phenyl-pyrrolidine-1-carbonyl)-pyrrolidin-1-yl]-methanone;
(5-Aminomethyl-thiophen-3-yl)-[(2S,4R)-4-phenyl-2-((R)-3-phenyl-pyrrolidine-1-carbonyl)-pyrrolidin-1-yl]-methanone;
[2-((S)-1-Amino-ethyl)-oxazol-5-yl]-[(2S,4R)-4-phenyl-2-((R)-3-phenyl-pyrrolidine-1-carbonyl)-pyrrolidin-1-yl]-methanone;
[6-(1-Methylamino-ethyl)-piperidin-2-yl]-[(2S,4R)-4-phenyl-2-((R)-3-phenyl-pyrrolidine-1-carbonyl)-pyrrolidin-1-yl]-methanone;
(6-(1-(methylamino)ethyl)piperidin-2-yl)((2S,4R)-4-phenyl-2-(((R)-3-phenylpyrrolidin-1-yl)methyl)pyrrolidin-1-yl)methanone;
[5-(1-Methylamino-ethyl)-furan-2-yl]-[(S)-2-(7-phenyl-thiazolo[5,4-b]pyridin-2-yl)-pyrrolidin-1-yl]-methanone;
(5-(1-(methylamino)ethyl)furan-2-yl)((S)-2-(4-phenylthiazolo[4,5-c]pyridin-2-yl)pyrrolidin-1-yl)methanone;
(5-(1-(methylamino)ethyl)furan-2-yl)((S)-2-(7-phenylthiazolo[5,4-d]pyrimidin-2-yl)pyrrolidin-1-yl)methanone;
(4-Fluoro-phenyl)-(3-{1′-[5-(1-methylamino-ethyl)-furan-2-carbonyl]-[1,2]bipyrrolidinyl-2-yl}-pyridin-2-yl)-methanone;
(octahydro-6-phenethylpyrrolo[2,3-c]pyridin-1-yl)(5-(1-(methylamino)ethyl)furan-2-yl)methanone;
(4-Fluoro-phenyl)-(2-{1-[5-(1-methylamino-ethyl)-furan-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(5-(1-(methylamino)ethyl)furan-2-yl)methanone;
(2-(5-(4-fluorophenoxy)pyridin-3-yl)pyrrolidin-1-yl)(5-(1-(methylamino)ethyl)furan-2-yl)methanone;
(4-Fluoro-phenyl)-(5-{1-[5-(1-methylamino-ethyl)-furan-2-carbonyl]-pyrrolidin-2-yl}-pyridin-3-yl)-methanone;
(2-(4-(4-fluorophenoxy)pyridin-2-yl)pyrrolidin-1-yl)(5-(1-(methylamino)ethyl)furan-2-yl)methanone;
(2-(2-(N-(4-fluorophenyl)-N-methylamino)-5-fluoropyridin-4-yl)pyrrolidin-1-yl)(5-(1-(methylamino)ethyl)furan-2-yl)methanone;
(3-(1-(methylamino)ethyl)phenyl)((S)-2-(7-phenylthiazolo[5,4-b]pyridin-2-yl)pyrrolidin-1-yl)methanone;
(3-(1-(methylamino)ethyl)phenyl)((S)-2-(4-phenylthiazolo[4,5-c]pyridin-2-yl)pyrrolidin-1-yl)methanone;
(3-(1-(methylamino)ethyl)phenyl)((S)-2-(7-phenylthiazolo[5,4-d]pyrimidin-2-yl)pyrrolidin-1-yl)methanone;
(4-Fluoro-phenyl)-(3-{1′-[3-(1-methylamino-ethyl)-benzoyl]-[1,2′]-Dipyrrolidinyl-2-yl}-pyridin-2-yl)-methanone;
(octahydro-6-phenethylpyrrolo[2,3-c]pyridin-1-yl)(3-(1-(methylamino)ethyl)phenyl)methanone;
(4-Fluoro-phenyl)-(2-{1-[3-(1-methylamino-ethyl)-benzoyl]-pyrrolidin-2-yl}-thiazol-4-yl)-methanone;
(2-(2-(N-(4-fluorophenyl)-N-methylamino)pyridin-4-yl)pyrrolidin-1-yl)(3-(1-(methylamino)ethyl)phenyl)methanone;
(2-(5-(4-fluorophenoxy)pyridin-3-yl)pyrrolidin-1-yl)(3-(1-(methylamino)ethyl)phenyl)methanone;
(4-Fluoro-phenyl)-(5-{1-[3-(1-methylamino-ethyl)-benzoyl]-pyrrolidin-2-yl}-pyridin-3-yl)-methanone;
(2-(4-(4-fluorophenoxy)pyridin-2-yl)pyrrolidin-1-yl)(3-(1-(methylamino)ethyl)phenyl)methanone;
(2-(2-(N-(4-fluorophenyl)-N-methylamino)-5-fluoropyridin-4-yl)pyrrolidin-1-yl)(3-(1-(methylamino)ethyl)phenyl)methanone;
(2-(2-(N-(4-fluorophenyl)-N-methylamino)pyridin-4-yl)pyrrolidin-1-yl)(3-(1-(methylamino)ethyl)phenyl)methanone;
(6-(1-(methylamino)ethyl)piperidin-2-yl)((S)-2-(7-phenylthiazolo[5,4-b]pyridin-2-yl)pyrrolidin-1-yl)methanone;
(6-(1-(methylamino)ethyl)piperidin-2-yl)((S)-2-(4-phenylthiazolo[4,5-c]pyridin-2-yl)pyrrolidin-1-yl)methanone;
(6-(1-(methylamino)ethyl)piperidin-2-yl)((S)-2-(7-phenylthiazolo[5,4-d]pyrimidin-2-yl)pyrrolidin-1-yl)methanone;
(4-Fluoro-phenyl)-(3-{1′-[6-(1-methylamino-ethyl)-piperidine-2-carbonyl]-[1,2]bipyrrolidinyl-2-yl}-pyridin-2-yl)-methanone;
(octahydro-6-phenethylpyrrolo[2,3-c]pyridin-1-yl)(6-(1-(methylamino)ethyl)piperidin-2-yl)methanone;
(4-Fluoro-phenyl)-(2-{1-[6-(1-methylamino-ethyl)-piperidine-2-carbonyl]-pyrrolidin-2-yl}-thiazol-4-yl)-methanone;
(2-(2-(N-(4-fluorophenyl)-N-methylamino)pyridin-4-yl)pyrrolidin-1-yl)(6-(1-(methylamino)ethyl)piperidin-2-yl)methanone;
(2-(5-(4-fluorophenoxy)pyridin-3-yl)pyrrolidin-1-yl)(6-(1-(methylamino)ethyl)piperidin-2-yl)methanone;
(4-Fluoro-phenyl)-(5-{1-[6-(1-methylamino-ethyl)-piperidine-2-carbonyl]-pyrrolidin-2-yl}-pyridin-3-yl)-methanone;
(2-(4-(4-fluorophenoxy)pyridin-2-yl)pyrrolidin-1-yl)(6-(1-(methylamino)ethyl)piperidin-2-yl)methanone;
(2-(2-(N-(4-fluorophenyl)-N-methylamino)-5-fluoropyridin-4-yl)pyrrolidin-1-yl)(6-(1-(methylamino)ethyl)piperidin-2-yl)methanone;
(2-(2-(N-(4-fluorophenyl)-N-methylamino)pyridin-4-yl)pyrrolidin-1-yl)(6-(1-(methylamino)ethyl)piperidin-2-yl)methanone;
{(2S,4R)-1-[5-(1-Amino-ethyl)-furan-2-carbonyl]-4-phenyl-pyrrolidin-2-yl}-[(R)-3-(4-fluoro-phenyl)-pyrrolidin-1-yl]-methanone;
[(S)-1-[5-(1-Amino-ethyl)-furan-2-carbonyl]-4-((R)-4-fluoro-phenyl)-pyrrolidin-2-yl]-((R)-3-phenyl-pyrrolidin-1-yl)-methanone; and
((2S,4R)-4-(4-fluorophenyl)-2-(((R)-3-(4-fluorophenyl)pyrrolidin-1-yl)methyl)pyrrolidin-1-yl)(5-(1-(methylamino)ethyl)furan-2-yl)methanone.

One specific embodiment of the invention relates to compounds of formula (IIa) selected from the group consisting of: (5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone; and
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone.

Another specific embodiment of the present invention relates to compounds selected from the group consisting of [5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone;
(6-(1-(methylamino)ethyl)pyridin-2-yl)((2S,4R)-4-phenyl-2-(((R)-3-phenylpyrrolidin-1-yl)methyl)pyrrolidin-1-yl)methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(S)-methylamino-ethyl)-furan-2-yl]-methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(R)-methylamino-ethyl)-furan-2-yl]-methanone;
(5-(1(S)-aminoethyl)furan-2-yl)((2S,4R)-4-phenyl-2-(((R)-3-phenylpyrrolidin-1-yl)methyl)pyrrolidin-1-yl)methanone;
(5-(1(R)-amino-ethyl)furan-2-yl)((2S,4R)-4-phenyl-2-(((R)-3-phenylpyrrolidin-1-yl)methyl)pyrrolidin-1-yl)methanone;
(3-(1(S)-(methylamino)ethyl)phenyl)((2S,4R)-4-phenyl-2-(((R)-3-phenylpyrrolidin-1-yl)methyl)pyrrolidin-1-yl)methanone; and
(3-(1(R)-(methylamino)ethyl)phenyl)((2S,4R)-4-phenyl-2-(((R)-3-phenylpyrrolidin-1-yl)methyl)pyrrolidin-1-yl)methanone.

In a preferred embodiment of formula (II) X is

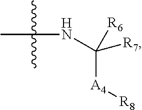

and the compounds are accordingly of formula (IIb):

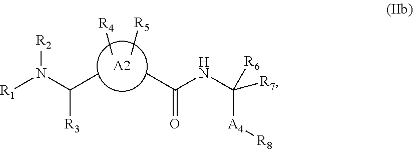

or a pharmaceutically acceptable salt, solvate or prodrug thereof,
wherein
R¹, R², R³, R⁴, R⁵, R⁶, R⁷, R⁸, A₁, A₃, and A₄, are as defined for formula (I) herein above, and A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems.

A preferred embodiment of the invention relates to compounds of formula (IIb) wherein R¹ is H; and R², R³, R⁴, R⁵, R⁶, R⁷, R⁸, A₁, A₂, A₃ and A₄, are as defined for formula (I) herein above. More preferably R¹ may be H and R² may be selected from the group consisting of H, C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(CH₂)₁₋₄-heteroaryl, wherein any alkyl, alkenyl, alkynyl, cycloalkyl, aryl, heterocyclyl, and heteroaryl optionally are substituted. Even more preferably R¹ may be H and R² may be may be selected from the group consisting of C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(CH₂)₁₋₄-heteroaryl, wherein any alkyl, alkenyl, alkynyl, cycloalkyl, aryl, heterocyclyl, and heteroaryl optionally are substituted. Even more preferably R¹ may be H and R² may be methyl. Compounds of formula (IIb) within said embodiment have been found to have an improved activity profile compared to compounds with R¹ different from H.

In an alternative embodiment of the compounds of formula (IIb) R¹ and R² are both H.

Example of a specific preferred compound of formula (IIb):
6-(1-aminoethyl)-N-(3-(3-phenylpyrrolidin-1-yl)-1-(1H-1,2,4-triazol-1-yl)propan-2-yl)piperidine-2-carboxamide; and more preferably 6-(1(S)-aminoethyl)-N-(3-(3-phenylpyrrolidin-1-yl)-1-(1H-1,2,4-triazol-1-yl)propan-2-yl)piperidine-2-carboxamide.

In one embodiment of the invention the compounds of formula (I) are of formula (III)

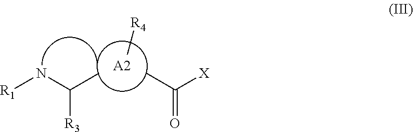

or a pharmaceutically acceptable salt, solvate or prodrug thereof,
wherein R¹, R³, R⁴, R⁶, R⁷, R⁸, B, A₁, A₃, A₄, and X are as defined for formula (I) herein above, A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems, and R² together with R⁵ forms a heterocyclic ring together with the nitrogen to which R² is attached, wherein the heterocyclic ring optionally is substituted. When such a heterocyclic ring is formed R² may be seen as a single bond or for example an alkyl moiety depending on what is relevant for the specific heterocyclic ring. Accordingly, in one embodiment R² together with R⁵ forms a heterocyclic ring together with the nitrogen to which R² is attached, wherein the heterocyclic ring optionally is substituted, and wherein R² is a single bond. The heterocyclic ring may be any ring as defined herein above, and preferably may be a 5-, 6- or 7-membered heterocyclic ring, more preferably a 5 or 6-membered heterocyclic ring. For this embodiment of the invention the heterocyclic ring may optionally be substituted with one or more substituents as defined herein above, and more preferably the heterocyclic ring may be substituted with one or more substituents selected from the group consisting of —F, —Cl, —OH, —CF₃, C₁-C₄ alkyl, —CN, and —NO₂.

In a preferred embodiment of formula (III) R¹, R², R³, R⁴, R⁵, A₁, and A₂ combined forms a spirocycle and is selected from the group consisting of

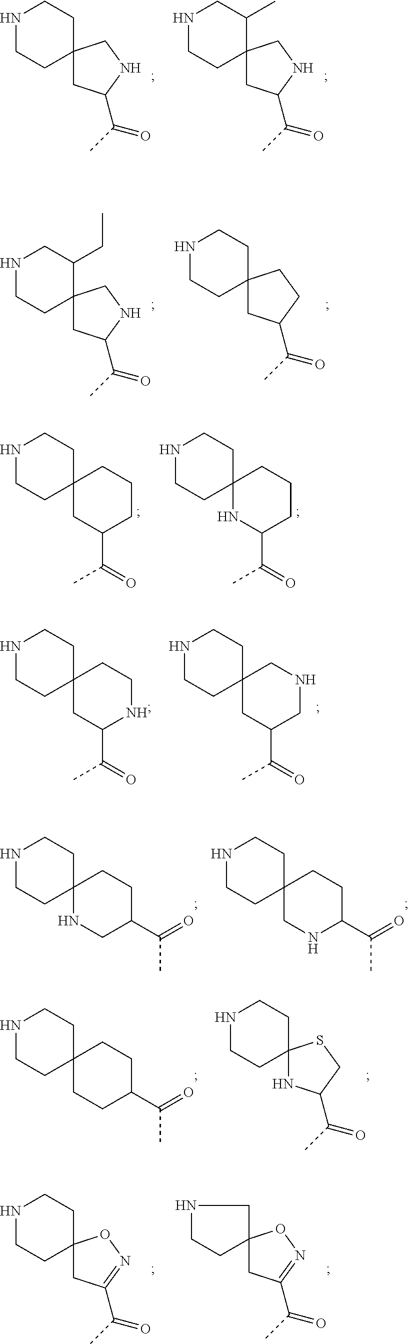

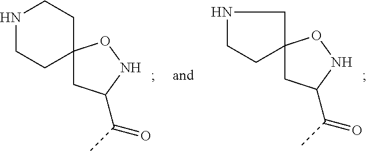

wherein the dotted line indicates the attachment point to X of formula (III). More preferably the spirocycle may be selected from the group consisting of

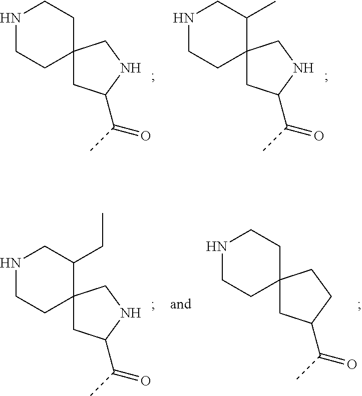

wherein the dotted line indicates the attachment point to X of formula (III).

In a preferred embodiment of formula (III) X is

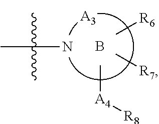

and the compounds are accordingly of formula (IIIa):

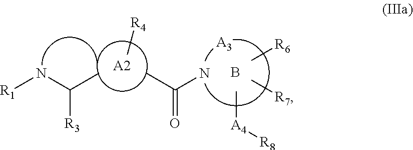

or a pharmaceutically acceptable salt, solvate or prodrug thereof,
wherein
R¹, R³, R⁴, R⁶, R⁷, R⁸, B, A₁, A₃, and A₄ are as defined for formula (I) herein above, A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems, and R² together with R⁵ forms a heterocyclic ring together with the nitrogen to which R² is attached, wherein the heterocyclic ring optionally is substituted, as further described herein above for formula (I) and formula (III).

A preferred embodiment of the invention relates to compounds of formula (IIIa) wherein R¹ is H; and R³, R⁴, R⁶, R⁷, R⁸, A₁, A₂, A₃ and A₄, are as defined for formula (I) herein above. Compounds of formula (IIIa) within said embodiment have been found to have an improved activity profile compared to compounds with R¹ different from H.

It has been found that the compounds of formula (III) and formula (IIIa) comprising a R⁶ and/or R⁷ substituent have an improved activity profile compared to compounds without said R⁶ and/or R⁷ group. Accordingly, in a preferred embodiment of formula (IIIa) at least one of R⁶ and R⁷ is not H, and R¹, R³, R⁴, R⁸, B, A₂, A₃, and A₄ are as defined for formula (I) herein above. More preferably at least one of R⁶ and R⁷ is selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(C₂)ₚ—Z₃, wherein Z₃ and p is as defined herein above for formula (I), and wherein any alkyl, cycloalkyl, aryl, heterocyclyl, and heteroaryl optionally are substituted.

Examples of specific preferred compounds of formula (IIIa):
(2S,4S)-4-Cyclohexyl-1-(2,8-diaza-spiro[4.5]decane-3-carbonyl)-pyrrolidine-2-carboxylic acid (R)-indan-1-ylamide;
2,8-Diaza-spiro[4.5]decane-3-carboxylic acid [(S)-cyclohexyl-((R)-indan-1-ylcarbamoyl)-methyl]-amide;
(2R,4R)-4-Cyclohexyl-1-(2,8-diaza-spiro[4.5]decane-3-carbonyl)-pyrrolidine-2-carboxylicacid(S)-indan(R)-1-ylamide; and
(2R,4R)-4-Cyclohexyl-1-(2,8-diaza-spiro[4.5]decane-3-carbonyl)-pyrrolidine-2-carboxylicacid(R)-indan(R)-1-ylamide.

Further examples of specific preferred compounds of formula (IIIa):
(2,8-Diaza-spiro[4.5]dec-3-yl)-[-4-phenyl-2-(3-phenyl-pyrrolidine-1-carbonyl)-pyrrolidin-1-yl]-methanone;
1-(2,8-Diaza-spiro[4.5]decane-3-carbonyl)-4-phenyl-pyrrolidine-2-carboxylic acid-indan-1-ylamide;
4-Cyclohexyl-1-(2,8-diaza-spiro[4.5]decane-3-carbonyl)-pyrrolidine-2-carboxylic acid-indan-1-ylamide;
2,8-Diaza-spiro[4.5]decane-3-carboxylic acid [-cyclohexyl-(-indan-1-ylcarbamoyl)-methyl]-amide;
4-Benzyl-1-(2,8-diaza-spiro[4.5]decane-3-carbonyl)-pyrrolidine-2-carboxylic acid (2-carbamoyl-indan-1-yl)-amide;
(6-Methyl-2,8-diaza-spiro[4.5]dec-3-yl)-[-4-phenyl-2-(3-phenyl-pyrrolidin-1-ylmethyl)-pyrrolidin-1-yl]-methanone;
(6-Ethyl-2,8-diaza-spiro[4.5]dec-3-yl)-[-4-phenyl-2-(3-phenyl-pyrrolidin-1-ylmethyl)-pyrrolidin-1-yl]-methanone;
(2,8-Diaza-spiro[4.5]dec-3-yl)-[-2-(4-phenyl-thiazolo[4,5-c]pyridin-2-yl)-pyrrolidin-1-yl]-methanone;
(2,8-Diaza-spiro[4.5]dec-3-yl)-[-2-(7-phenyl-thiazolo[5,4-b]pyridin-2-yl)-pyrrolidin-1-yl]-methanone;
(2,8-Diaza-spiro[4.5]dec-3-yl)-[-2-(7-phenyl-thiazolo[5,4-d]pyrimidin-2-yl)-pyrrolidin-1-yl]-methanone;
(2,8-Diaza-spiro[4.5]dec-3-yl)-(6-phenethyl-octahydro-pyrrolo[2,3-c]pyridin-1-yl)-methanone;
(2-[1-(2,8-Diaza-spiro[4.5]decane-3-carbonyl)-pyrrolidin-2-yl]-thiazol-4-yl)-(4-fluoro-phenyl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
{3-[1′-(2,8-Diaza-spiro[4.5]decane-3-carbonyl)-[1,2]bipyrrolidinyl-2-yl]-pyridin-2-yl}-(4-fluoro-phenyl)-methanone;
(2,8-Diaza-spiro[4.5]dec-3-yl)-{2-[5-(4-fluoro-phenoxy)-pyridin-3-yl]-pyrrolidin-1-yl}-methanone;
{5-[1-(2,8-Diaza-spiro[4.5]decane-3-carbonyl)-pyrrolidin-2-yl]-pyridin-3-yl}-(4-fluoro-phenyl)-methanone;
(2,8-Diaza-spiro[4.5]dec-3-yl)-{2-[4-(4-fluoro-phenoxy)-pyridin-2-yl]-pyrrolidin-1-yl}-methanone;
(2,8-Diaza-spiro[4.5]dec-3-yl)-(2-{5-fluoro-2-[(4-fluoro-phenyl)-methyl-amino]-pyridin-4-yl}-pyrrolidin-1-yl)-methanone; and
(2,8-Diaza-spiro[4.5]dec-3-yl)-(2-{2-[(4-fluoro-phenyl)-methyl-amino]-pyridin-4-yl}-pyrrolidin-1-yl)-methanone.

For the above mentioned further compounds of formula (IIIa) the following stereoisomers are preferred:
(2,8-Diaza-spiro[4.5]dec-3-yl)-[(2S,4R)-4-phenyl-2-(3-phenyl-pyrrolidine-1-carbonyl)-pyrrolidin-1-yl]-methanone;
(2S,4R)-1-(2,8-Diaza-spiro[4.5]decane-3-carbonyl)-4-phenyl-pyrrolidine-2-carboxylic acid (R)-indan-1-ylamide;
(2S,4S)-4-Cyclohexyl-1-(2,8-diaza-spiro[4.5]decane-3-carbonyl)-pyrrolidine-2-carboxylic acid (R)-indan-1-ylamide;
2,8-Diaza-spiro[4.5]decane-3-carboxylic acid [(S)-cyclohexyl-((R)-indan-1-ylcarbamoyl)-methyl]-amide;
(2S,4R)-4-Benzyl-1-(2,8-diaza-spiro[4.5]decane-3-carbonyl)-pyrrolidine-2-carboxylic acid ((1R,2R)-2-carbamoyl-indan-1-yl)-amide;
(6-Methyl-2,8-diaza-spiro[4.5]dec-3-yl)-[(2S,4R)-4-phenyl-2-(3-phenyl-pyrrolidin-1-ylmethyl)-pyrrolidin-1-yl]-methanone;
(6-Ethyl-2,8-diaza-spiro[4.5]dec-3-yl)-[(2S,4R)-4-phenyl-2-(3-phenyl-pyrrolidin-1-ylmethyl)-pyrrolidin-1-yl]-methanone;
(2,8-Diaza-spiro[4.5]dec-3-yl)-[(S)-2-(4-phenyl-thiazolo[4,5-c]pyridin-2-yl)-pyrrolidin-1-yl]-methanone;
(2,8-Diaza-spiro[4.5]dec-3-yl)-[(S)-2-(7-phenyl-thiazolo[5,4-b]pyridin-2-yl)-pyrrolidin-1-yl]-methanone;
(2,8-Diaza-spiro[4.5]dec-3-yl)-[(S)-2-(7-phenyl-thiazolo[5,4-d]pyrimidin-2-yl)-pyrrolidin-1-yl]-methanone;
(2,8-Diaza-spiro[4.5]dec-3-yl)-(6-phenethyl-octahydro-pyrrolo[2,3-c]pyridin-1-yl)-methanone;
{2-[1-(2,8-Diaza-spiro[4.5]decane-3-carbonyl)-pyrrolidin-2-yl]-thiazol-4-yl}-(4-fluoro-phenyl)-methanone;
(2,8-Diaza-spiro[4.5]dec-3-yl)-(2-{2-[(4-fluoro-phenyl)-methyl-amino]-pyridin-4-yl}-pyrrolidin-1-yl)-methanone;
{3-[1′-(2,8-Diaza-spiro[4.5]decane-3-carbonyl)-[1,2]bipyrrolidinyl-2-yl]-pyridin-2-yl}-(4-fluoro-phenyl)-methanone;
(2,8-Diaza-spiro[4.5]dec-3-yl)-{2-[5-(4-fluoro-phenoxy)-pyridin-3-yl]-pyrrolidin-1-yl}-methanone;
{5-[1-(2,8-Diaza-spiro[4.5]decane-3-carbonyl)-pyrrolidin-2-yl]-pyridin-3-yl}-(4-fluoro-phenyl)-methanone;
(2,8-Diaza-spiro[4.5]dec-3-yl)-{2-[4-(4-fluoro-phenoxy)-pyridin-2-yl]-pyrrolidin-1-yl}-methanone;
(2,8-Diaza-spiro[4.5]dec-3-yl)-(2-{5-fluoro-2-[(4-fluoro-phenyl)-methyl-amino]-pyridin-4-yl}-pyrrolidin-1-yl)-methanone; and
(2,8-Diaza-spiro[4.5]dec-3-yl)-(2-{2-[(4-fluoro-phenyl)-methyl-amino]-pyridin-4-yl}-pyrrolidin-1-yl)-methanone.

In a preferred embodiment of formula (III) X is

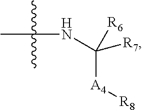

and the compounds are accordingly of formula (IIIb):

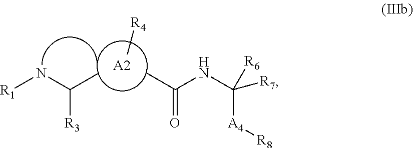

or a pharmaceutically acceptable salt, solvate or prodrug thereof,
wherein
R¹, R³, R⁴, R⁶, R⁷, R⁸, B, A₁, A₃, and A₄ are as defined for formula (I) herein above, A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems, and R² together with R⁵ forms a heterocyclic ring together with the nitrogen to which R² is attached, wherein the heterocyclic ring optionally is substituted, as further described herein above for formula (I) and formula (III).

A preferred embodiment of the invention relates to compounds of formula (IIIb) wherein R¹ is H; and R³, R⁴, R⁶, R⁷, R⁸, A₁, A₂, A₃ and A₄, are as defined for formula (I) herein above. Compounds of formula (IIIb) within said embodiment have been found to have an improved activity profile compared to compounds with R¹ different from H. Alternatively, R¹ may be H and R² may be selected from the group consisting of H, C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(CH₂)₁₋₄-heteroaryl, wherein any alkyl, alkenyl, alkynyl, cycloalkyl, aryl, heterocyclyl, and heteroaryl. More preferably R¹ may be H and R² may be methyl.

It has been found that the compounds of formula (III) and formula (IIIb) comprising a R⁶ and/or R⁷ substituent have an improved activity profile compared to compounds without said R⁶ and/or R⁷ group. Accordingly, in a preferred embodiment of formula (IIIb) at least one of R⁶ and R⁷ is not H, and R¹, R³, R⁴, R⁸, B, A₂, A₃, and A₄ are as defined for formula (I) herein above. More preferably at least one of R⁶ and R⁷ is selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(C₂)ₚ—Z₃, wherein Z₃ and p is as defined herein above for formula (I), and wherein any alkyl, cycloalkyl, aryl, heterocyclyl, and heteroaryl optionally are substituted.

In one embodiment of the invention the compounds of formula (I) are of formula (IV)

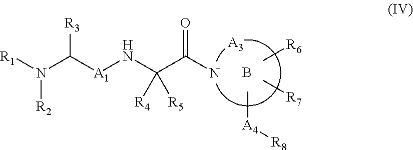

or a pharmaceutically acceptable salt, solvate or prodrug thereof,
wherein R¹, R², R³, R⁴, R⁵, R⁶, R⁷, R⁸, B, A₁, A₃, and A₄ are as defined for formula (I) herein above, and A₂ is —NHC(R⁴R⁵)—, with the proviso that
with the proviso that when A₂ is —NHC(R⁴R⁵)—, then X is not

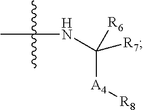

with the proviso that when A₁ is a —C(O)—, A₂ is —NHC(R⁴R⁵)—, B is pyrrolidinyl, R¹ is H,
R² is methyl, R³ is methyl or ethyl, and one of R⁴ and R⁵ is isopropyl, tert-butyl or cyclohexyl, then at least one of R⁶ and R⁷ is not H;
with the proviso that when A1 is a —C(O)—, A₂ is —NHC(R⁴R⁵)—, A₄ is a single bond, B is pyrrolidinyl, R¹ is H, R² is methyl, R³ is methyl, one of R⁴ and R⁵ is cyclohexyl, and one of R⁶ and R⁷ is H, then the other of R⁶ and R⁷ is not benzyloxy;
with the proviso that when A₁ is a —C(O)—, A₂ is —NHC(R⁴R⁵)—, B is octahydro-1H-pyrrolo[2,3-c]pyridin-1-yl, 7-oxooctahydro-1H-pyrrolo[2,3-c]pyridin-1-yl, octahydropyrrolo[2,3-c]azepin-1(2H)-yl, 8-oxooctahydropyrrolo[2,3-c]azepin-1(2H)-yl hexahydropyrrolo[3,4-b]pyrrol-1(2H)-yl, or 6-oxohexahydropyrrolo[3,4-b]pyrrol-1(2H)-yl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, B is 7-oxooctahydro-1H-pyrrolo[2,3-c]pyridinyl, A₄ is —CH₂CH₂—, R¹ is H, R² is methyl, R³ is methyl, one of R⁴ and R⁵ is isopropyl, R⁸ is phenyl, and one of R⁶ and R⁷ is H, then the other of R⁶ and R⁷ is not benzyloxy;
with the proviso that when A₁ is a —C(O)—, A₂ is —NHC(R⁴R⁵)—, A4 contains a —NHC(O)-fragment or is —CH₂—O—, B is pyrrolidinyl, R¹ and R² is H, R³ is methyl, ethyl, propyl or isopropyl, and R⁴ forms a heterocyclic ring with A₃, then at least one of R⁶ and R⁷ is not H; and
with the proviso that when A₁ is a —C(O)—, A₂ is —NHC(R⁴R⁵)—, A₄ contains a —NHC(O)-fragment, B is pyrrolidinyl, R³ is methyl, ethyl, propyl or isopropyl, and R⁴ forms a heterocyclic ring with A₃, then at least one of R⁶ and R⁷ is not H.

In a preferred embodiment of formula (IV) A₁ is —C(O)—, and R¹, R², R³, R⁴, R⁵, R⁶, R⁷, R⁸, B, A₃, and A₄ are as defined for formula (I) herein above. Compounds of formula (IV) within said embodiment have been found to have an improved activity profile compared to compounds with A₁ other than —C(O)—. For this embodiment it is furthermore preferred that at least one of R⁶ and R⁷ is not H. More preferably at least one of R⁶ and R⁷ may be selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein Z₃ and p is as defined herein above for formula (I), and wherein any alkyl, cycloalkyl, aryl, heterocyclyl, and heteroaryl optionally are substituted.

It has been found that the compounds of formula (IV) comprising a R⁶ and/or R⁷ substituent have an improved activity profile compared to compounds without said R⁶ and/or R⁷ group. Accordingly, in a preferred embodiment of formula (IV) at least one of R⁶ and R⁷ is not H, and R¹, R², R³, R⁴, R⁵, R⁸, B, A₃, and A₄ are as defined for formula (I) herein above. More preferably at least one of R⁶ and R⁷ is selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(C₂)ₚ—Z₃, wherein Z₃ and p is as defined herein above for formula (I), and wherein any alkyl, cycloalkyl, aryl, heterocyclyl, and heteroaryl optionally are substituted.

In a more preferred embodiment of formula (IV) A₁ is —C(O)—, B is heterocyclyl, at least one of R⁶ and R⁷ is different from H, R⁸ is a ring structure selected from substituted aryl, heteroaryl or heterocyclyl, and R¹, R², R³, R⁴, R⁵, R⁸, A₃, and A₄ are as defined for formula (I) herein above. More preferably the ring structure is substituted with a further ring structure giving a bulky group. Accordingly, it is furthermore preferred for this embodiment formula (IV), that R⁸ is selected from the group consisting of substituted C₃-C₁₀ cycloalkyl, aryl, heterocyclyl, and heteroaryl and one or more substituents are each independently selected from the group consisting of C₃-C₁₀ cycloalkyl, aryl, heterocyclyl, and heteroaryl. More preferably R⁸ is aryl-heterocyclyl.

In a further preferred embodiment of formula (IV) A₁ is —C(O)—, B is pyrrolidinyl, at least one of R⁶ and R⁷ is optionally substituted phenyl, A₄ is —CH₂—, R⁸ is selected from the group consisting of C₃-C₁₀ cycloalkyl-pyrrolidinyl, heterocyclyl-pyrrolidinyl, aryl-pyrrolidinyl and heteroaryl-pyrrolidinyl, and R¹, R², R³, R⁴, R⁵, R⁸, and A₃ are as defined for formula (I) herein above. Compounds of formula (IV) within said embodiment have been found to have an improved activity profile compared to compounds with A₄ containing a —C(O)—, such as for example —NHC(O)— and —C(O)—O—.

In a preferred embodiment of formula (IV), at least one of R¹ and R² is different from H. It has surprisingly been found, that the presence of at least one of R¹ and R² different from H, may improve the compounds cell permeability. To this end it is especially preferred that one of R¹ and R² are selected from the group consisting of C₁-C₄ alkyl, C₁-C₄ alkoxy, C₂-C₄ alkenyl, and C₂-C₄ alkynyl, wherein any alkyl, alkenyl and alkynyl optionally are substituted; more preferably selected from the group consisting of C₁-C₄ alkyl, and C₁-C₄ alkoxy; even more preferably methyl and ethyl; and yet even more preferably methyl. Accordingly in a preferred embodiment of formula (IV) R¹ is H and R² is methyl.

In an alternative embodiment of the compounds of formula (IV) R¹ and R² are both H.

Examples of specific preferred compounds of formula (IV):
(2S,4R)-1-((3R,5S)-1-(2-((S)-2-aminopropanamido)-3-(1H-1,2,4-triazol-1-yl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)butanoyl)-3-phenylpyrrolidine-5-carbonyl)-N-methyl-4-phenylpyrrolidine-2-carboxamide;
(2S,4R)-1-((S)-2-((R)-2-aminopropanamido)-3-(4-carbamoylphenyl)propanoyl)-N—((R)-2,3-dihydro-1H-inden-1-yl)-4-phenylpyrrolidine-2-carboxamide;
(2R,3R)-1-((S)-2-((S)-2-aminopropanamido)-3-(3-carbamoylphenyl)propanoyl)-N—((R)-2,3-dihydro-1H-inden-1-yl)-3-phenylazetidine-2-carboxamide;
(2S,4R)-1-((S)-2-((S)-2-aminopropanamido)-3-(3-cyanophenyl)propanoyl)-N—((R)-2,3-dihydro-1H-inden-1-yl)-4-phenylpyrrolidine-2-carboxamide;
(2S,4R)-1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)-3-(furan-2-yl)propanoyl)-3-phenylpyrrolidine-5-carbonyl)-N-methyl-4-phenylpyrrolidine-2-carboxamide;
(S)—N—((S)-3-(3-cyanophenyl)-1-oxo-1-((2S,4R)-4-phenyl-2-(((R)-3-phenylpyrrolidin-1-yl)methyl)pyrrolidin-1-yl)propan-2-yl)-2-(methylamino)butanamide;
(2S,4R)-1-((S)-2-((R)-2-aminopropanamido)-3-(3-carbamoylphenyl)propanoyl)-N—((R)-2,3-dihydro-1H-inden-1-yl)-4-phenylpyrrolidine-2-carboxamide; and
(2S,3S)-1-((S)-2-((S)-2-aminopropanamido)-3-(3-carbamoylphenyl)propanoyl)-N—((S)-2,3-dihydro-1H-inden-1-yl)-2-phenylazetidine-3-carboxamide.

Further examples of specific preferred compounds of formula (IV):
2-amino-N-(-4-methyl-1-oxo-1-(-4-phenyl-2-((3-phenylpyrrolidin-1-yl)methyl)pyrrolidin-1-yl)pentan-2-yl)propanamide;
2-amino-N-(-3-cyclohexyl-1-oxo-1-(-4-phenyl-2-((3-phenylpyrrolidin-1-yl)methyl)pyrrolidin-1-yl)propan-2-yl)propanamide;
2-amino-N-(-3-methyl-1-oxo-1-(-4-phenyl-2-((3-phenylpyrrolidin-1-yl)methyl)pyrrolidin-1-yl)butan-2-yl)propanamide;
2-amino-N-(-3-methyl-1-oxo-1-(-4-phenyl-2-((3-phenylpyrrolidin-1-yl)methyl)pyrrolidin-1-yl)butan-2-yl)propanamide;
2-amino-N-(-3-methyl-1-oxo-1-(-4-phenyl-2-((3-phenylpyrrolidin-1-yl)methyl)pyrrolidin-1-yl)pentan-2-yl)propanamide;
2-amino-N-(-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-4-(1H-tetrazol-5-yl)butan-2-yl)propanamide;
2-amino-N-(-3-(3-chlorophenyl)-1-oxo-1-(-4-phenyl-2-((3-phenylpyrrolidin-1-yl)methyl)pyrrolidin-1-yl)propan-2-yl)propanamide;
2-amino-N-(-3-(4-chlorophenyl)-1-oxo-1-(-4-phenyl-2-((3-phenylpyrrolidin-1-yl)methyl)pyrrolidin-1-yl)propan-2-yl)propanamide;
2-amino-N-(-3-(2,4-dichlorophenyl)-1-oxo-1-(-4-phenyl-2-((3-phenylpyrrolidin-1-yl)methyl)pyrrolidin-1-yl)propan-2-yl)propanamide;
2-amino-N-(-3-(3,4-dichlorophenyl)-1-oxo-1-(-4-phenyl-2-((3-phenylpyrrolidin-1-yl)methyl)pyrrolidin-1-yl)propan-2-yl)propanamide;
2-amino-N-(-3-(3,4-difluorophenyl)-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-3-(4-(trifluoromethyl)phenyl)propan-2-yl)propanamide;
2-amino-N-(-3-(3-cyanophenyl)-1-oxo-1-(-4-phenyl-2-((3-phenylpyrrolidin-1-yl)methyl)pyrrolidin-1-yl)propan-2-yl)propanamide;
2-amino-N-(-1-oxo-1-(-4-phenyl-2-((3-phenylpyrrolidin-1-yl)methyl)pyrrolidin-1-yl)-3-(pyridin-3-yl)propan-2-yl)propanamide;
2-amino-N-(-1-oxo-1-(-4-phenyl-2-((3-phenylpyrrolidin-1-yl)methyl)pyrrolidin-1-yl)butan-2-yl)propanamide;
2-amino-N-(-3-cyclopropyl-1-oxo-1-(-4-phenyl-2-((3-phenylpyrrolidin-1-yl)methyl)pyrrolidin-1-yl)propan-2-yl)propanamide;
3-(-2-(-2-aminopropanamido)-3-oxo-3-(-4-phenyl-2-((3-phenylpyrrolidin-1-yl)methyl)pyrrolidin-1-yl)propyl)benzamide;
4-(-2-(-2-aminopropanamido)-3-oxo-3-(-4-phenyl-2-((3-phenylpyrrolidin-1-yl)methyl)pyrrolidin-1-yl)propyl)benzamide; and
2-amino-N-(-4,4-dimethyl-1-oxo-1-(-4-phenyl-2-((3-phenylpyrrolidin-1-yl)methyl)pyrrolidin-1-yl)pentan-2-yl)propanamide.

For the above mentioned further compounds of formula (IV) the following stereoisomers are preferred:
(S)-2-amino-N—((S)-4-methyl-1-oxo-1-((2S,4R)-4-phenyl-2-((3-phenylpyrrolidin-1-yl)methyl)pyrrolidin-1-yl)pentan-2-yl)propanamide;
(S)-2-amino-N—((S)-3-cyclohexyl-1-oxo-1-((2S,4R)-4-phenyl-2-((3-phenylpyrrolidin-1-yl)methyl)pyrrolidin-1-yl)propan-2-yl)propanamide;
(S)-2-amino-N—((R)-3-methyl-1-oxo-1-((2S,4R)-4-phenyl-2-((3-phenylpyrrolidin-1-yl)methyl)pyrrolidin-1-yl)butan-2-yl)propanamide;
(S)-2-amino-N—((S)-3-methyl-1-oxo-1-((2S,4R)-4-phenyl-2-((3-phenylpyrrolidin-1-yl)methyl)pyrrolidin-1-yl)butan-2-yl)propanamide;
(S)-2-amino-N-((2R,3S)-3-methyl-1-oxo-1-((2S,4R)-4-phenyl-2-((3-phenylpyrrolidin-1-yl)methyl)pyrrolidin-1-yl)pentan-2-yl)propanamide;
(S)-2-amino-N—((S)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-4-(1H-tetrazol-5-yl)butan-2-yl)propanamide;
(S)-2-amino-N—((S)-3-(3-chlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(4-chlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(2,4-dichlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(3,4-dichlorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-3-(3,4-difluorophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-3-(4-(trifluoromethyl)phenyl)propan-2-yl)propanamide;
(S)-2-amino-N—((S)-3-(3-cyanophenyl)-1-oxo-1-((2S,4R)-4-phenyl-2-((3-phenylpyrrolidin-1-yl)methyl)pyrrolidin-1-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)-3-(pyridin-3-yl)propan-2-yl)propanamide;
(S)-2-amino-N—((S)-1-oxo-1-((2S,4R)-4-phenyl-2-((3-phenylpyrrolidin-1-yl)methyl)pyrrolidin-1-yl)butan-2-yl)propanamide;
(S)-2-amino-N—((S)-3-cyclopropyl-1-oxo-1-((2S,4R)-4-phenyl-2-((3-phenylpyrrolidin-1-yl)methyl)pyrrolidin-1-yl)propan-2-yl)propanamide;
3-((S)-2-((S)-2-aminopropanamido)-3-oxo-3-((2S,4R)-4-phenyl-2-((3-phenylpyrrolidin-1-yl)methyl)pyrrolidin-1-yl)propyl)benzamide;
4-((S)-2-((S)-2-aminopropanamido)-3-oxo-3-((2S,4R)-4-phenyl-2-((3-phenylpyrrolidin-1-yl)methyl)pyrrolidin-1-yl)propyl)benzamide; and
(S)-2-amino-N—((R)-4,4-dimethyl-1-oxo-1-((2S,4R)-4-phenyl-2-((3-phenylpyrrolidin-1-yl)methyl)pyrrolidin-1-yl)pentan-2-yl)propanamide.

In one embodiment of the invention the compounds of formula (I) are of formula (V)

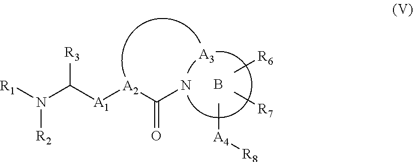

or a pharmaceutically acceptable salt, solvate or prodrug thereof,
wherein R¹, R², R³, R⁵, R⁶, Fr, R⁸, B, A₁, and A₄ are as defined for formula (I) herein above, and A₃ forms a heterocyclic ring together with R⁴. Preferably A₃ may be C (carbon atom).

It has been found that the compounds of formula (V) comprising a R⁶ and/or R⁷ groups have an improved activity profile compared to compounds without said R⁶ and/or R⁷ group. Accordingly, in a preferred embodiment of formula (V) at least one of R⁶ and R⁷ is not H, and R¹, R², R³, R⁴, R⁵, R⁸, B, A₁, A₂ and A₄ are as defined for formula (I) herein above. More preferably at least one of R⁶ and R⁷ is selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein Z₃ and p is as defined herein above for formula (I), and wherein any alkyl, cycloalkyl, aryl, heterocyclyl, and heteroaryl optionally are substituted.

In a preferred embodiment of formula (V), at least one of R¹ and R² is different from H. It has surprisingly been found, that the presence of at least one of R¹ and R² different from H, may improve the compounds cell permeability. To this end it is especially preferred that one of R¹ and R² are selected from the group consisting of C₁-C₄ alkyl, C₁-C₄ alkoxy, C₂-C₄ alkenyl, and C₂-C₄ alkynyl, wherein any alkyl, alkenyl and alkynyl optionally are substituted; more preferably selected from the group consisting of C₁-C₄ alkyl, and C₁-C₄ alkoxy; even more preferably methyl and ethyl; and yet even more preferably methyl. Accordingly in a preferred embodiment of formula (V) R¹ is H and R² is methyl.

In an alternative embodiment of the compounds of formula (V) R¹ and R² are both H.

An example of a specific preferred compound of formula (V):
2-amino-N-(-5-oxo-1-phenyl-3-((3-phenylpyrrolidin-1-yl)methyl)-octahydro-1H-pyrrolo[1,2-a]azepin-6-yl)propanamide.

For the above mentioned compound of formula (V) the following stereoisomer is preferred:
(S)-2-amino-N-((1R,3S,6S)-5-oxo-1-phenyl-3-((3-phenylpyrrolidin-1-yl)methyl)-octahydro-1H-pyrrolo[1,2-a]azepin-6-yl)propanamide.

### Compounds of Formula (VI) and (VII)

A further aspect of the invention relates to polymeric compounds, such as e.g. homodimers, heterodimers, homomultimers or heteromultimers, wherein compounds of formula (I) forms a polymeric compound of formula (VI)

Y-(L)ₘ-[Y-(L)ₘ]ₙ-Y (VI)

or a pharmaceutically acceptable salt, solvate or prodrug thereof,
wherein
Y is a monomeric unit of formula (I), wherein the first and the second or further monomeric units are the same or different and independently are selected from the compounds of formula (I) as defined herein;
L is the same or different and is a covalent linker, linking any part of one monomeric unit of formula (I), to any part of a second or further monomeric unit of formula (I);
m is an integer of 1 to 4; and
n is an integer of 0 to 5. Preferably m is 1; and n is an integer of 0 to 2.

A further aspect of the invention relates to compounds which comprise at least one monomer of formula (I), such as e.g., one monomer, two monomers, one homodimer, or two homodimers, linked to an entity E. These entity linked compounds are described by formula (VII)

Z-Lₘ-E (vii)

or a pharmaceutically acceptable salt, solvate or prodrug thereof,
wherein
Z is a compound of formula (I) as defined in herein or a polymeric compound of formula (VI) as defined herein;
L is a linker linking any part of Z to any part of E;
E is an entity selected from the group consisting of an affinity tag, such as e.g. a hexahistidine tag or biotin, a dye, such as e.g. fluorescein, an oligonucleotide, such as e.g. DNA or RNA, a protein, such as e.g. an antibody or biotin-binding protein, and a solid support; and
m is an integer of 1 to 4; preferably m is 1.

A linker L connecting two separate molecules of formula (I), as in formula (VI), or connecting a compound of formula (I) or a polymeric compound of formula VI with an entity E, as in formula (VII), may comprise two attachment groups (AG) and one or more Linker Elements (LE). Thus the linker L may be represented by the general structure

-AG-(LE)ₕ-AG- (L)

wherein
each AG independently is selected from the group consisting of single bond, H, —NH—, —NHC(O)—, —C(O)NH—, —SO₂—, —NHC(O)NH—, C₁-C₆ alkyl, C₁-C₆ alkoxy, C₂-C₆ alkenyl, C₂-C₆ alkynyl, C₃-C₁₀ cycloalkyl, aryl, heterocyclyl, and heteroaryl; wherein any alkyl, alkoxy, alkenyl, alkynyl, cycloalkyl, aryl, heterocyclyl, and heteroaryl optionally are substituted;
each linker element LE independently is selected from the group consisting of single bond, —O—, —NH—, —CH₂—, —NHC(O)—, —C(O)NH—, —SO₂—, —NH—C(O)—NH—, heterocyclyl, heteroaryl, and aryl; wherein any aryl, heterocyclyl, and heteroaryl optionally are substituted; and
h is 0, or an integer from 1 to 6, preferably 0 or an integer from 1 to 3, such as e.g. 1 or 2.

In a preferred embodiment of the invention, the attachment groups AG are each independently selected from the group consisting of single bond, H, —NH—, —NHC(O)—, —C(O)NH—, —SO₂—, —NHC(O)NH—, aryl, heterocyclyl, and heteroaryl; wherein any aryl, heterocyclyl, and heteroaryl optionally are substituted.

In a preferred embodiment of the invention the linker elements LE is selected from the group consisting of single bond, —O—, —NH—, —CH₂—, benzyl, naphtyl, biphenyl,

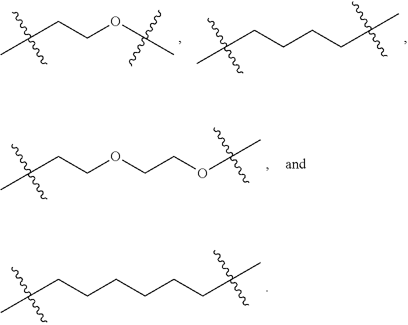

Preferably the linker L is selected from the group consisting of

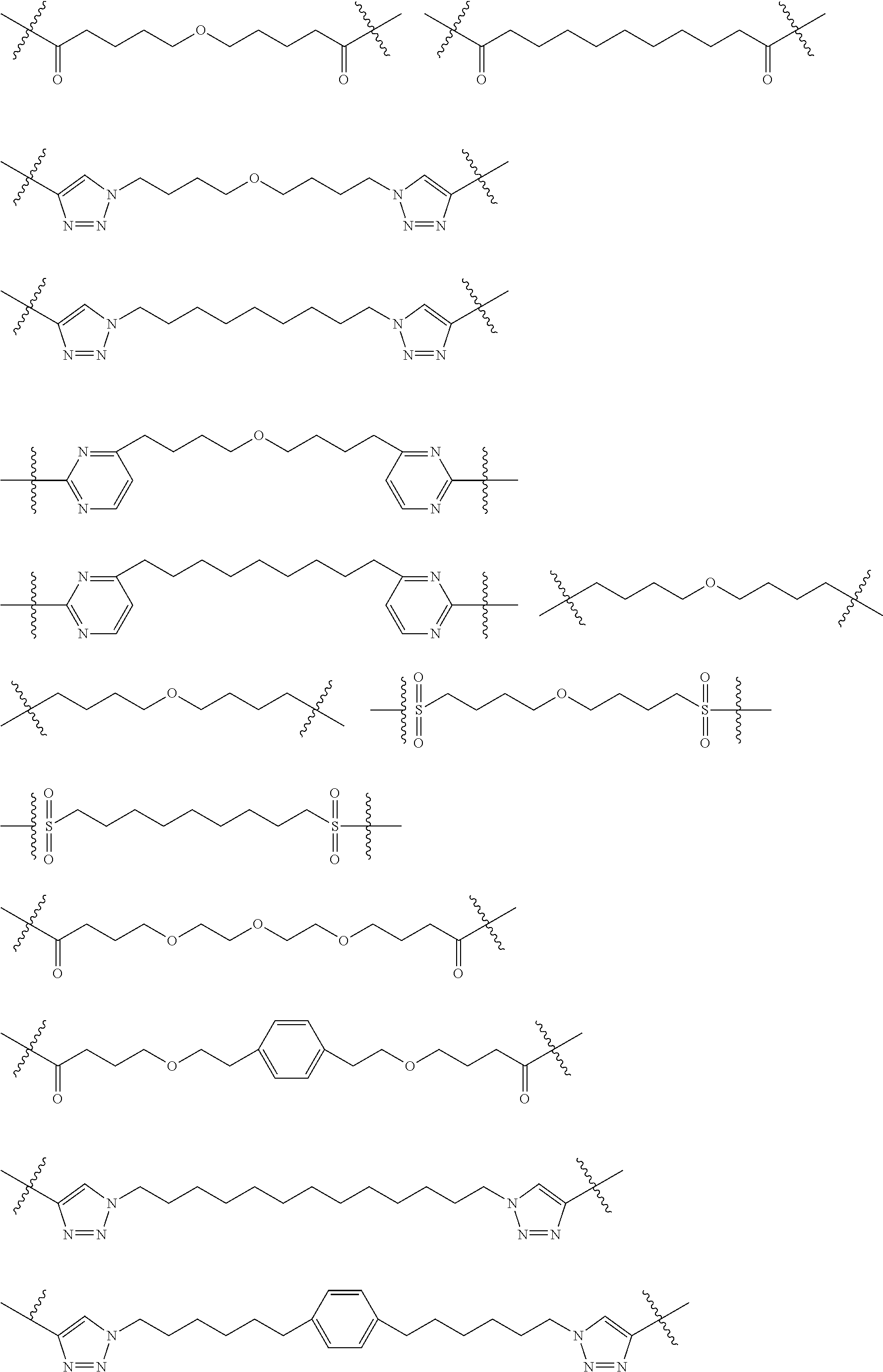

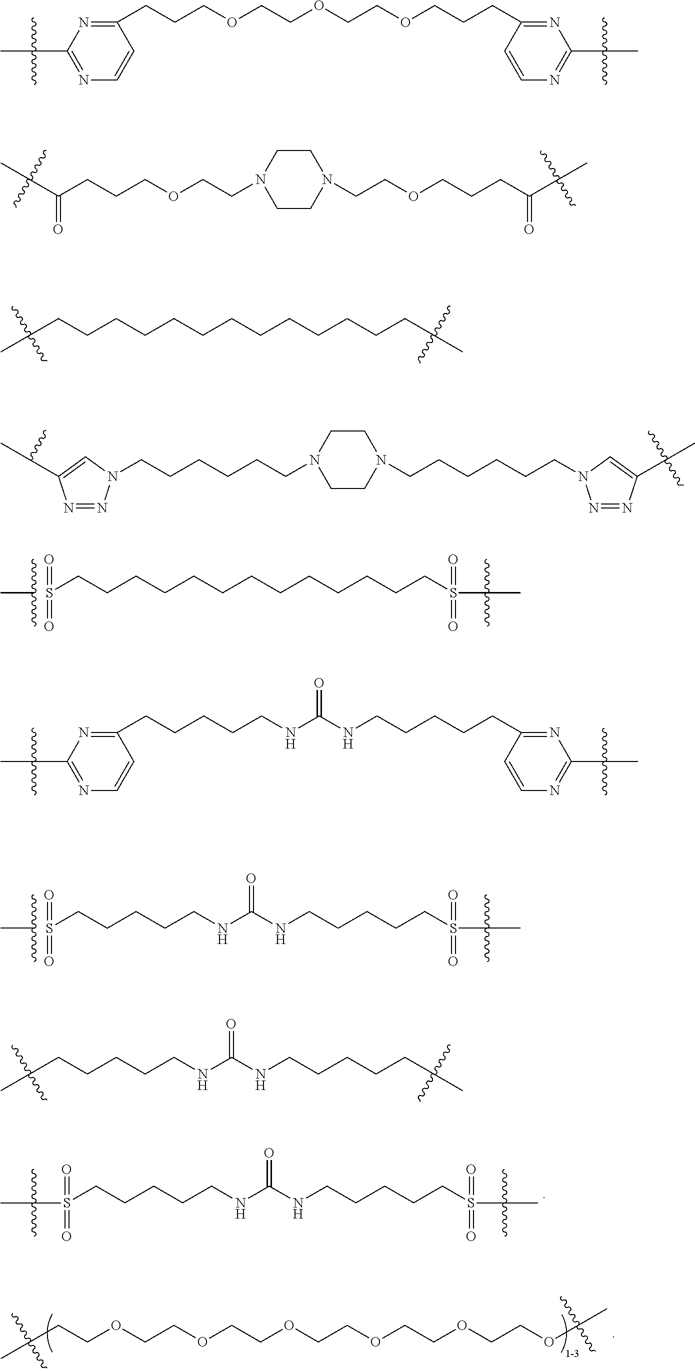

The linker L may furthermore be

### Treatment of Diseases

A further aspect of the present invention relates to compounds of formulas (I), (VI) and (VII), as defined herein, for use as a medicament.

A further aspect of the invention relates to compounds of formulas (I), (VI) and (VII) for treating proliferative diseases, such as e.g. malignant and benign tumors, cancer, metastases and other benign proliferative diseases. The present invention further relates to use of compounds of formulas (I), (VI) and (VII) for the preparation of a medicament for the treatment of proliferative diseases.

In one embodiment of the invention, the proliferative disease is cancer including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

Where a tumor, a tumor disease, a carcinoma, or a cancer is mentioned, also metastasis in the original organ or tissue and/or in any other location are implied. Accordingly, in one embodiment of the invention the cancer being treated is metastatic.

In another embodiment, the disease being treated is a proliferative disease resistant to conventional anticancer therapies, such as e.g. chemoresistant, radiant resistant, and hormone resistant diseases. Preferably the disease being treated is a proliferative disease that is refractory to the treatment with other chemotherapeutics, and more preferably the disease being treated is a proliferative disease that is refractory to treatment with other chemotherapeutics due to multidrug resistance, such as e.g. a benign or malignant tumor; more preferably the disease is a cancer that is refractory to treatment with other chemotherapeutics due to multidrug resistance.

In a further embodiment of the invention the proliferative disease is a hyperproliferative condition, such as e.g., leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

In a specific embodiment of the invention the proliferative disease is a benign proliferative disease, including, but not limited to, psoriasis, benign prostatic hypertrophy or restinosis.

In a further embodiment of the invention the proliferative disease is myeloma, preferably multiple myeloma. The term “myeloma” as used herein relates to a tumor composed of cells of the type normally found in the bone marrow. The term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia. Multiple myeloma is considered to be incurable by the use of conventional and high dose chemotherapies.

Another aspect of the present invention relates to use of compounds of formulas I, VI and VII for the treatment, amelioration or prevention of diseases and/disorders responsive to induction of apoptosis, such as e.g. disorders characterized by a dysregulation of apoptosis through a pathway involving IAPs, and preferably a pathway involving binding of IAPs to Smac and/or Caspases, such as e.g. Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14 or a structural or functional homolog thereof, and particularly XIAP binding interaction with caspases 3 and 7. More preferably the disorders are characterized by a dysregulation of apoptosis through a pathway involving IAPs, and preferably a pathway involving binding of XIAP and/or cIAP proteins to Smac and/or Caspases. The compounds of formula I induce apoptosis and/or potentiate the induction of apoptosis in response to apoptosis induction signals. Without being bound by theory it is therefore contemplated that the compounds of formula I, VI and VII sensitize cells to inducers of apoptosis, including cells that are resistant to such inducers. The IAP inhibitors of the present invention can thus be used to induce apoptosis in any disorder that can be treated, ameliorated, or prevented by the induction of apoptosis. Accordingly, in one embodiment of the invention the compounds of formulas I, VI and VII are used for promoting apoptosis in proliferating cells. In another embodiment of the invention the compounds of formulas I, VI and VII is used for sensitizing cells to inducers of apoptosis.

The present invention relates to the use of compounds of formula (I), (VI), and (VII) for the preparation of a medicament for promoting apoptosis in proliferating cells. The present invention furthermore relates to the use of compounds of formula (I), (VI), and (VII) for the preparation of a medicament for sensitizing cells to inducers of apoptosis.

The compounds of the invention inhibit the binding of IAP proteins to caspases, in particular XIAP binding interaction with caspases 3 and 7. The compounds also inhibit the binding of ML-IAP to Smac protein. Accordingly, the compounds of the invention are useful for inducing apoptosis in cells or sensitizing cells to apoptotic signals, in particular cancer cells.

In a further embodiment of the invention, the disorder to be treated is characterized by an overexpression of IAP proteins, accordingly, in this embodiment the cells (e.g., cancer cells) show elevated expression levels of IAP proteins as compared to non-pathological samples (e.g., non-cancerous cells). Alternatively, in another embodiment, the cells operationally manifest elevated expression levels of IAP proteins by virtue of executing the apoptosis program and dying in response to an effective amount of a compound of Formula I, said response occurring, at least in part, due to the dependence in such cells on IAP protein function for their survival.

As described herein compounds of the present invention are useful for inducing apoptosis in cells in which the mitochondrial apoptotic pathway is disrupted such that release of Smac from ML-IAP proteins is inhibited, for example by up regulation of Bcl-2 or down regulation of Bax/Bak. Accordingly, the compounds may be used for the treatment of all cancer types which fail to undergo apoptosis. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

As the compounds of the present invention are useful for sensitizing cells to apoptotic signals, the compounds may be administered prior to, concomitantly with, or following administration of radiation therapy or cytostatic or antineoplastic chemotherapy. Suitable cytostatic chemotherapy compounds include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (Vi) topoisomerase I poisons, such as camptothecin or topotecan; (vii) topoisomerasellpoisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, STI571 (CPG 57148B) or UCN-OI (7-hydroxystaurosporine); (x) miscellaneous investigational agents such as thioplatin, PS-341, phenylbutyrate, ET-18—OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists. In a preferred embodiment, compounds of the present invention the compounds according to the invention are co-administered with a cytostatic compound selected from the group consisting of cisplatin, doxorubicin, taxol, taxotere and mitomycin C. Most preferred, the cytostatic compound is doxorubicin. Further examples of combination treatment is described herein below in the section “combination therapy”.

The compounds of formula I according to the invention have a significant antiproliferative effect and promote differentiation, e.g. cell cycle arrest and apoptosis. Accordingly, the compounds of the invention are selectively toxic, or more toxic to rapidly proliferating cells, than to normal cells, particularly human cancer cells, such as e.g., cancerous tumors.

Other aspects of the present invention relates to use of compounds of formula I for the treatment, amelioration or prevention of diseases and/disorders such as e.g. T and B cell mediated autoimmune diseases; inflammatory diseases; infections, including, but are not limited to, infections caused by viruses, bacteria, fungi, mycoplasma, prions, and the like; hyperproliferative diseases; AIDS; degenerative conditions, vascular diseases, and the like.

The compounds and pharmaceutical compositions of the invention may be administered to any animal in need of such treatment. In a preferred embodiment of the invention the animal is a mammal, such as e.g. humans and veterinary animals (cows, sheep, pigs, horses, dogs, cats and the like). In a more preferred embodiment the animal is a human, and in an alternative embodiment the animals include veterinary animals, such as e.g. cows, sheep, pigs, horses, dogs, cats and the like.

The composition of the invention will be formulated, dosed, and administered in a fashion consistent with good medical practice. Factors for consideration in this context include the particular disorder being treated, the particular subject, such as a human, being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to treat a proliferative disorder, inhibit IAP interaction with for example caspases, induce apoptosis or sensitize a malignant cell to an apoptotic signal. Such amount is preferably below the amount that is toxic to normal cells, or the mammal as a whole.

### Pharmaceutical Compositions

In a further aspect the present invention relates to pharmaceutical compositions comprising the compounds of the present invention, i.e. compounds of formula (I), (VI), and (VII), as defined herein, and optionally one or more pharmaceutically acceptable excipients, diluents or carriers.

In one embodiment of the invention the pharmaceutical composition further comprises one or more additional active substances. The one or more additional active substances may be any additional active substance or an active substance as mentioned herein in the section “Combination therapy”. Preferably said one or more additional active substances are selected from anticancer agents, antineoplastic agents, cytotoxic drugs, and anti-tumor antibiotics. More preferably said one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

A compound of this invention may be administered alone or in combination with pharmaceutically acceptable carriers, diluents, or excipient in either single or multiple doses. Suitable pharmaceutical acceptable carriers, diluents and excipients include inert solid diluents or fillers, sterile aqueous solutions and various organic solvents. The pharmaceutical compositions formed by combining a compound of formula (I), or a pharmaceutically acceptable salt, solvate or prodrug thereof, with pharmaceutical acceptable carriers, diluents or excipients can be readily administered in a variety of dosage forms such as tablets, powders, lozenges, syrups, suppositories, injectable solutions and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. A preferred form for oral use are capsules, which include the formulation of the active compound with encapsulating material as a carrier providing a capsule in which the active component with or without other carriers, is surrounded by a carrier, which is thus in association with it. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

Possible pharmaceutical preparations which can be used rectally include, for example, suppositories, which consist of a combination of one or more of the active compounds with a suppository base. For preparing suppositories a suppository base e.g. in the form of a low melting wax, such as a mixture of fatty acid glycerides or cocoa butter, is first melted and the active component is dispersed homogeneously therein, as by stirring. The molten homogenous mixture is then poured into convenient size molds, allowed to cool, and thereby to solidify. Suitable suppository bases are, for example, natural or synthetic glycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

For parenteral administration, solutions containing a compound of this invention or a pharmaceutically acceptable salt, solvate or prodrug thereof, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. Aqueous injection suspensions may contain substances which increase the viscosity of the suspension include, for example, sodium carboxymethyl cellulose, sorbitol, and/or dextran. Optionally, the suspension may also contain stabilizers. The oily solutions/suspensions are suitable for intra-articular, intra-muscular and subcutaneous injection purposes. Suitable lipophilic solvents or vehicles include fatty oils, for example, sesame oil, or synthetic fatty acid esters, for example, ethyl oleate or triglycerides or polyethylene glycol-400. The preparation of all these solutions under sterile conditions is readily accomplished by standard pharmaceutical techniques well known to those skilled in the art.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil. Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

A compound of formula (I), (VI) and (VII), or a pharmaceutically acceptable salt thereof can be administered orally, transdermally (e.g., through the use of a patch), parenterally (e.g. intravenously), rectally, or topically. Pharmaceutical composition according to the present invention include all compositions wherein the compounds of the present invention are contained in an amount which is effective to achieve its intended purpose. In general, the daily dosage for treating a proliferative disease, or treating a disorders responsive to induction of apoptosis, will generally range from about 0.0001 to about 50.0 mg/kg body weight of the patient to be treated, preferably from about 0.0001 to about 40 mg/kg body weight of the patient to be treated, such as e.g., from about 0.0002 to about 30 mg/kg, from about 0.001 to about 20 mg/kg, from about 0.0015 to about 15 mg/kg, from about 0.01 to about 10 mg/kg, from about 0.1 to about 10 mg/kg, from about 0.5 to about 20 mg/kg, and from about 0.5 to about 20 mg/kg, or an equivalent amount of the pharmaceutically acceptable salt, solvate or prodrug thereof. Preferably, about 0.01 to about 10 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, and most preferably, from about 0.01 to about 5 mg/kg. In a topical formulation, the compound may preferably be present at a concentration of about 0.01 to 100 mg per gram of carrier. In a preferred embodiment, the compound is present at a concentration of about 0.07-1.0 mg/ml, more preferably about 0.1-0.5 mg/ml, most preferably about 0.4 mg/ml.

As an example, a compound of the formula (I) or a pharmaceutically acceptable salt, solvate or prodrug thereof, can be administered for treatment of a proliferative disease, or for treatment of a disorders responsive to induction of apoptosis, to an adult human of average weight (about 70 kg) in a dose ranging from about 0.01 mg up to about 2000 mg per day, preferably from about 0.1 to about 1000 mg per day, such as e.g., from about 0.1 to about 500 mg per day, and from about 0.1 to about 100 mg per day, or such as e.g., from about 1 to about 1000 mg per day, from about 10 to about 1000 mg per day, from about 100 to about 1000 mg per day, from about 200 to about 1000 mg per day, and from about 500 to about 1000 mg per day, in single or divided (i.e., multiple) portions, or an equivalent amount of the pharmaceutically acceptable salt, solvate or prodrug thereof. The dosage may be given as a single dosage or be divided into several doses for administration during a day. Usually, children receive half of the adult dose.

In general, the therapeutically-effective compounds of this invention (i.e. compounds of formula I) are present in pharmaceutical compositions at concentration levels ranging from about 0.01 to 99% by weight, such as from about 0.25 to 95% by weight, preferably from about 5% to about 95% by weight, more preferably from 10% to 95% by weight, such as e.g., from 20% to 95% by weight, from 30% to 95% by weight, from 40% to 95% by weight, more preferably from 50% to 95% by weight, such as e.g., from 60% to 95% by weight, and from 70% to 95% by weight.

Variations based on the aforementioned dosage ranges may be made by a physician of ordinary skill taking into account known considerations such as the weight, age, and condition of the person being treated, the severity of the affliction, and the particular route of administration chosen.

The pharmaceutical preparations of the invention are preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. A unit dosage for oral administration may comprise from about 0.01 to about 50 mg, preferably about 0.1 to about 10 mg of the compound. The unit dose may be administered one or more times daily as one or more tablets or capsules each containing from about 0.1 to about 10 mg, conveniently about 0.25 to 50 mg of the compound or its salts, solvates or prodrugs.

In general a tablet formulation could typically contain between about 5 mg to about 1500 mg of a compound according to the present invention (or a salt, solvate or prodrug thereof) whilst tablet fill weights may for example range from 50 mg to 3000 mg. Examples of formulation for tablets and capsules are illustrated below here in tables 1-5.

TABLE 1
Example of tablet comprising 10% active substance

| Ingredient | % w/w |
|---|---|
| A compound of formula I, or a salt, solvate or prodrug thereof | 10.000* |
| Lactose | 64.125 |
| Starch | 21.375 |
| Croscarmellose Sodium | 3.000 |
| Magnesium Stearate | 1.500 |

*This quantity is typically adjusted in accordance with the desired dosage.

TABLE 2
Example of tablet comprising 100 mg active substance

| Ingredient | Amount, mg |
|---|---|
| A compound of formula I, or a salt, solvate or prodrug | 100* |
| thereof | |
| Starch | 259 |
| Lactose | 259 |
| Magnesium stearate | 3.3 |
| Talc | 29.7 |

*This quantity is typically adjusted in accordance with the desired dosage

The above example formulations in tables 1 and 2 may further contain e.g. colour, flavour or a coating in order to e.g. disguise an unpleasant taste, or for example an enterocoating in order to protect the active compound from gastric acid.

TABLE 3
Example of tablet comprising 50 mg active compound

| Ingredient: | Amount, mg | |
|---|---|---|
| A compound of formula (I), or a salt, solvate or prodrug | 50 | mg |
| thereof | | |
| Wheat starch | 60 | mg |
| Lactose | 50 | mg |
| Colloidal silica | 5 | mg |
| Talcum | 9 | mg |
| Magnesium stearate | 1 | mg |
| Total | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed. The dry granules are pressed through a sieve having a mesh size of 3 mm, mixed with a pre-sieved mixture (1 mm sieve) of the remaining corn starch, magnesium stearate and talcum and compressed to form slightly biconvex tablets.

TABLE 4
Example of tablet comprising 100 mg active compound

| Ingredients: | Amount, mg | |
|---|---|---|
| A compound of formula (I), or a salt, solvate or prodrug | 100 | mg |
| thereof | | |
| Crystalline lactose | 240 | mg |
| Avicel | 80 | mg |
| PVPPXL | 20 | mg |
| Aerosil | 2 | mg |
| Magnesium stearate | 5 | mg |
| Total | 447 | mg |

Manufacture: The active ingredient is mixed with the carrier materials and compressed by means of a tabletting machine (Korsch EKO, Stempeldurchmesser 10 mm).

TABLE 5
Example of capsule comprising 100 mg active compound

| Ingredient: | Amount, mg | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| Total | 318. | mg |

Manufacturing is done by mixing the components and filling them into hard gelatine capsules, size 1.

The pharmaceutical compositions of the present invention may be prepared in a manner known per se, for example by means of conventional dissolving, lyophilizing, mixing, granulating or confectioning processes.

### Combination Therapy

The compounds and pharmaceutical compositions of the present invention may be administered alone or in combination with one or more additional active substances. The active substance may be comprised in a pharmaceutical composition together with the compound of the invention, or be administered individually. In one embodiment of the invention the further active substance is selected from the group consisting of antiproliferative agents, anti-hyperproliferative agents, anticancer agents, chemotherapeutic agents, antineoplastics agents, antimicrobial agents, antiviral agents, antifungal agents, anti-inflammatory agents, and natural products (such as e.g., plant and/or animal derived products). Preferably the one or more additional active substances are selected from anti-hyperproliferative agents, and antineoplastic agents; more preferably alkylating agents, antimetabolites, and natural products. More preferably the one or more additional active substances are selected from anticancer agents, antineoplastic agents, cytotoxic drugs, and anti-tumor antibiotics.

In a preferred embodiment of the invention the compounds of the present invention are administered in combination with one or more antiproliferative agents, including, but are not limited to, aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

In a more preferred embodiment of the invention the compounds of the present invention are administered in combination with one or more anticancer agents, including, but are not limited to, compounds that inhibit tumor angiogenesis, such as e.g. protease inhibitors, epidermal growth factor receptor kinase inhibitors, vascular endothelial growth factor receptor kinase inhibitors and the like; cytotoxic drugs, such as e.g. antimetabolites, like purine and pyrimidine analog antimetabolites; antimitotic agents like microtubule stabilizing drugs and antimitotic alkaloids; platinum coordination complexes; anti-tumor antibiotics; alkylating agents, such as e.g. nitrogen mustards and nitrosoureas; endocrine agents, such as e.g. adrenocorticosteroids, androgens, anti-androgens, estrogens, anti-estrogens, aromatase inhibitors, gonadotropin-releasing hormone agonists and somatostatin analogues; and compounds that target an enzyme or receptor that is overexpressed and/or otherwise involved a specific metabolic pathway that is upregulated in the tumor cell, for example ATP and GTP phosphodiesterase inhibitors, histone deacetylase inhibitors, protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; methionine aminopeptidase inhibitors, proteasome inhibitors, and cyclooxygenase inhibitors, such as e.g. cyclooxygenase-1 or -2 inhibitors.

In a preferred embodiment of the invention the one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

In an alternative embodiment of the invention the compounds of the present invention are contemplated to be administered in combination with one or more anticancer agents or antiproliferative agents, including, but not limited to, agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-a) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like.

Numerous other examples of chemotherapeutic compounds and anticancer therapies suitable for co-administration with the compounds of the present invention are known to those skilled in the art.

In a preferred embodiment the one or more anticancer or antiproliferative agentsare selected from the group consisting of radiation (e.g., X-rays, gamma rays, UV); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; molecules which cause RNA interference (RNAi); antibodies (e.g., HERCEPTIN, RITUXAN, ZEVALIN, ERBITUX, Abagovomab, OvaRex (oregovomab), zalutumumab, Proxinium/VB4-845, Removab, (catumaxomab), Rencarex/WX-G250, Tarceva (erlotinib), Vectibix (panitumumab) and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

Alkylating agents suitable for use in a combination therapy according to the present invention include, but are not limited to:

1) nitrogen mustards (e.g., mechlorethamine, cyclophosphamide, ifosfamide, melphalan (L-sarcolysin); and chlorambucil);
2) ethylenimines and methylmelamines (e.g, hexamethylmelamine and thiotepa);
3) alkyl sulfonates (e.g., busulfan);
4) nitrosoureas (e.g., carmustine (BCNU); lomustine (CCNU); semustine (methyl-CCNU); and streptozocin (streptozotocin)); and
5) triazenes (e.g, dacarbazine (DTIC; dimethyltriazenoimid-azolecarboxamide).

Antimetabolites suitable for use in a combination therapy according to the present invention include, but are not limited to:

1) folic acid analogs (e.g., methotrexate (amethopterin));
2) pyrimidine analogs (e.g., fluorouracil (5-fluorouracil; 5-FU), floxuridine (fluorode-oxyuridine; FudR), and cytarabine (cytosin arabinoside)); and
3) purine analogs (e.g., mercaptopurine (6-mercaptopurine; 6-MP), thioguanine (6-thioguanine; TG), and pentostatin (2′-deoxycoformycin)).

Chemotherapeutic agents suitable for use in a combination therapy according to the present invention include, but are not limited to:

1) vinca alkaloids (e.g., vinblastine (VLB), vincristine);
2) epipodophyllotoxins (e.g., etoposide and teniposide);
3) antibiotics (e.g., dactinomycin (actinomycin D), daunorubicin (daunomycin; mbidomycin), doxorubicin, bleomycin, plicamycin (mithramycin), and mitomycin (mitomycin C));
4) enzymes (e.g., L-asparaginase);
5) biological response modifiers (e.g., interferon-alfa);
6) platinum coordinating complexes (e.g., cisplatin (cis-DDP) and carboplatin);
7) anthracenediones (e.g., mitoxantrone);
8) substituted ureas (e.g., hydroxyurea);
9) methylhydrazine derivatives (e.g., procarbazine (N-methylhydrazine; MIH));
10) adrenocoltical suppressants (e.g., mitotane (o, p′-DDD) and aminoglutethimide);
11) adrenocorticosteroids (e.g., prednisone);
12) progestins (e.g., hydroxyprogesterone caproate, medroxyprogesterone acetate, and megestrol acetate);
13) estrogens (e.g., diethylstilbestrol and ethinyl estradiol);
14) antiestrogens (e.g., tamoxifen);
15) androgens (e.g., testosterone propionate and fluoxymesterone);
16) antiandrogens (e.g., flutamide); and
17) gonadotropin-releasing hormone analogs (e.g., leuprolide).

Another class of active substances which may be used in a combination therapy according to the present invention are those which are able to sensitize for or induce apoptosis by binding to death receptors (“death receptor agonists”). Such agonists of death receptors include death receptor ligands such as tumor necrosis factor a (TNF-(X), tumor necrosis factor β (TNF-β, lymphotoxin-α), LT-β (lymphotoxin-β, TRAIL (Apo2L, DR4 ligand), CD95 (Fas, APO-I) ligand, TRAMP (DR3, Apo-3) ligand, DR6 ligand as well as fragments and derivatives of any of said ligands. Preferably, the death receptor ligand is TNF-α. More preferably the death receptor ligand is Apo2L/TRAIL. Furthermore, death receptors agonists comprise agonistic antibodies to death receptors such as anti-CD95 antibody, anti-TRAIL-R1 (DR4) antibody, anti-TRAIL-R2 (DR5) antibody, anti-TRAIL-R3 antibody, anti-TRAIL-R4 antibody, anti-DR6 antibody, anti-TNF-RI antibody and anti-TRAMP (DR3) antibody as well as fragments and derivatives of any of said antibodies.

Any oncolytic agent that is routinely used in a cancer therapy context may be used in a combination therapy according to the present invention. For example, the U.S. Food and Drug Administration maintain a formulary of oncolytic agents approved for use in the United States. International counterpart agencies to the U.S. F. D. A. maintain similar formularies. Those skilled in the art will appreciate that the “product labels” required on all U.S. approved chemotherapeutics describe approved indications, dosing information, toxicity data, and the like, for the exemplary agents. For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” ninth edition, Eds. Hardman et al., 1996.

In a preferred embodiment of the invention, the one or more anticancer agents for use in combination with compounds according to the present invention include adriamycin, 5-fluorouracil, etoposide, camptothecin, actinomycin D, mitomycin C, cisplatin, docetaxel, gemcitabine, carboplatin, oxaliplatin, bortezomib, gefitinib, and bevacizumab. These agents can be prepared and used singularly, in combined therapeutic compositions, in kits, or in combination with immunotherapeutic agents, and the like.

For the treatment of acute myeloid leukemia (AML), it is preferred that compounds of the present invention is used in combination with standard leukemia therapies, more preferably in combination with therapies used for the treatment of AML. In particular, compounds of formula I, VI and VII can be administered in combination with one or more farnesyl transferase inhibitors and/or other active substances useful for the treatment of AML, more particularly active substances selected from Daunorubicin, Adriamycin, Ara-C, VP-16, Teniposide, Mitoxantrone, Idarubicin, Carboplatinum and PKC412.

In another embodiment of the invention the compounds of the present invention are administered in combination with one or more antimicrobial agents. As an antimicrobial agent may be used any agent that can kill, inhibit, or otherwise attenuate the function of microbial organisms, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like. The antimicrobial agent is preferably selected from antibacterial agents, antiviral agents, antifungal agents, and the like.

By combination therapy is meant administration of a combination of a compound of the present invention in combination with one or more active substances, wherein the administration may be simultaneously, separately or sequentially. Accordingly, the combined administration may be in one unit dosage form, or individually in separate unit dosages forms. When separate unit dosage forms are used, then the administration may be substantially at the same time or sequentially, for example over a time interval that especially allow for an improved effect such as e.g. a synergistic effect.

In one embodiment of the invention the compound of the present invention is administered prior to the one or more additional active substances, such as an additional anticancer agent, for example 0.5, 1, 2, 3, 4, 5, 10, 12, or 18 hours prior to administration of the additional active substance, for example 1, 2, 3, 4, 5, or 6 days prior to administration of the additional active substance, for example 1, 2, 3, or 4 weeks prior to administration of the additional active substance, for example. In another embodiment the compound of the present invention is administered after the one or more additional active substances, for example 0.5, 1, 2, 3, 4, 5, 10, 12, or 18 hours after the administration of the additional active substance, for example 1, 2, 3, 4, 5, or 6 days after the administration of the additional active substance, or for example 1, 2, 3, or 4 weeks after the administration of the additional active substance such as e.g. the additional anticancer agent. In some embodiments, the compound and the therapeutic or anticancer agent are administered concurrently but on different schedules, e.g., the compound is administered daily while the one or more additional active substances is administered once a week, once every two weeks, once every three weeks, or once every four weeks. In other embodiments, the compound is administered once a week while the one or more additional active substances is administered daily, once a week, once every two weeks, once every three weeks, or once every four weeks.

The term “aromatase inhibitor”, as used herein, relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A preferred embodiment of the invention relates to the administration of a compound according to the invention in combination with an aromatase inhibitor, and particularly for the treatment of hormone receptor positive tumors, such as e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A preferred embodiment of the invention relates to the administration of a compound according to the invention in combination with an antiestrogen, and particularly for the treatment of estrogen receptor positive tumors, such as e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO00/31247, and hereby incorporated by reference. A preferred embodiment of the invention relates to the administration of a compound according to the invention in combination with a microtubule active agent, and particularly Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes, but is not limited to, compounds disclosed in WO 02/22577, which is hereby incorporated by reference, especially N-hydroxy-3-[4-[[(2-hydroxyethyl)[2-(1H-indol-3-yl)ethyl]-amino]methyl]phenyl]-2E-2o propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN. A preferred embodiment of the invention the invention relates to the administration of a compound according to the invention in combination with an antineoplastic antimetabolite.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity and further anti-angiogenic compounds” as used herein includes, but is not limited to, protein tyrosine kinase and/or serine and/or threonine kinase inhibitors or lipid kinase inhibitors, e.g.:

a) compounds targeting, decreasing or inhibiting the activity of the fibroblast growth factor-receptors (FGF-Rs);
b) compounds targeting, decreasing or inhibiting the activity of the insulin-like growth factor receptor I (IGF-IR), such as compounds which target, decrease or inhibit the activity of IGF-IR, especially compounds which inhibit the IGF-IR receptor, such as those compounds disclosed in WO 02/092599;
c) compounds targeting, decreasing or inhibiting the activity of the Trk receptor tyrosine kinase family;
d) compounds targeting, decreasing or inhibiting the activity of the Axl receptor tyrosine kinase family;
e) compounds targeting, decreasing or inhibiting the activity of the c-Met receptor;
f) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of thecyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a P13K inhibitor);
g) compounds targeting, decreasing or inhibiting the activity of a protein-tyrosine kinase, such as imatinib mesylate (GLIVEC/GLEEVEC) or tyrphostin. A tyrphostin is preferably a low molecular weight (Mw<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); and
h) compounds targeting, decreasing or inhibiting the activity of the epidermal growth factor family of receptor tyrosine kinases (EGF-R, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HERCEPTIN), cetuximab, Iressa, Tarceva, CI-1033, EKB-569, GW-2016, E1.1, E2.4, E2.5, E6.2, E6.4, E2.11, E6.3 or E7.6.3, and 7H-pyrrolo-[2,3-d]pyrimidine which are disclosed in WO 03/013541.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, [alpha]-[gamma]- or -tocopherol or a- y- or 5-tocotrienol.

The term “cyclooxygenase inhibitor” as used herein includes, but is not limited to, e.g., Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-aryl-aminophenylacetic acid, e.g. 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican®), CCI-779 and ABT578.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA®. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferony.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor”, e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MM1270B or AAJ996.

The term “agents used in the treatment of hematologic malignancies” as used herein includes, but is not limited to FMS-like tyrosine kinase inhibitors e.g. compounds targeting, decreasing or inhibiting the activity of Flt-3; interferon, 1-b-D-arabinofuransylcytosine (ara-c) and bisulfan; and ALK inhibitors e.g. compounds which target, decrease or inhibit anaplastic lymphoma kinase.

The term “compounds which target, decrease or inhibit the activity of Flt-3” are especially compounds, proteins or antibodies which inhibit Flt-3, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to trastuzumab (Herceptin®), Trastuzumab-DM1, erlotinib (Tarceva®), bevacizumab (Avastin®), rituximab (Rituxan®), PR064553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

Compounds of the present invention may furthermore be used to advantage in combination with known therapeutic processes such as for example radiation therapy. In particular the compounds of the present invention may be used as radiosensitizers, and more particularly for the treatment of tumors which exhibit poor sensitivity to radiotherapy.

The phrase “radiation therapy” refers to the use of electromagnetic or particulate radiation in the treatment of neoplasia. Radiation therapy is based on the principle that high-dose radiation delivered to a target area will result in the death of reproducing cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. Examples of radiotherapeutic agents are provided in, but not limited to, radiation therapy and is known in the art (Hellman, Principles of Radiation Therapy, Cancer, in Principles I and Practice of Oncology, 24875 (Devita et al., 4th ed., vol 1, 1993). Recent advances in radiation therapy include three-dimensional conformal external beam radiation, intensity modulated radiation therapy (MRT), stereotactic radiosurgery and brachytherapy (interstitial radiation therapy), the latter placing the source of radiation directly into the tumor as implanted “seeds”. These newer treatment modalities deliver greater doses of radiation to the tumor, which accounts for their increased effectiveness when compared to standard external beam radiation therapy.

Accordingly, the present invention relates to a method of treating proliferative diseases by administering a compound of the present invention with radiation therapy to a subject in need of such treatment. Any type, amount, or delivery and administration systems may be used to deliver the therapeutic dose of radiation to a subject such as an animal. For example, the subject may receive photon radiotherapy, particle beam radiation therapy, other types of radiotherapies, and combinations thereof. In one embodiment the radiation is delivered to the subject using a linear accelerator. In another embodiment the radiation is delivered using a gamma knife. The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

Ionizing radiation with beta-emitting radionuclides is considered the most useful for radiotherapeutic applications because of the moderate linear energy transfer (LET) of the ionizing particle (electron) and its intermediate range (typically several millimeters in tissue). Gamma rays deliver dosage at lower levels over much greater distances.

Alpha particles represent the other extreme, they deliver very high LET dosage, but have an extremely limited range and must, therefore, be in intimate contact with the cells of the tissue to be treated. In addition, alpha emitters are generally heavy metals, which limits the possible chemistry and presents undue hazards from leakage of radionuclide from the area to be treated. Depending on the tumor to be treated all kinds of emitters are conceivable within the scope of the present invention.

Accordingly, any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity. The total dose of radiation administered to a subject is about 1 Gray (Gy) to about 100 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the subject to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Furthermore, the present invention encompasses types of non-ionizing radiation like e.g. ultraviolet (UV) radiation, high energy visible light, microwave radiation (hyperthermia therapy), infrared (IR) radiation and lasers. In a particular embodiment of the present invention UV radiation is applied.

In the method of treating proliferative diseases by administering a compound of the present invention with radiation therapy, one or more additional radiosentiziers may be administered such as e.g., metronidazole, misonidazole, intra-arterial Budr, intravenous iododeoxyuridine (ludR), nitroimidazole, 5-substituted-4-nitroimidazoles, 2H-isoindolediones, [[(2-bromoethyl)-aminoamethyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells, whereas radioprotectors protect healthy tissue from the harmful effects of radiation.

The structure of the active agents identified by code numbers, generic or trade names may be taken from the actual edition of the standard compendium “The Merck Index” or from databases, e.g. Patents International (e.g. IMS World Publications). The above-mentioned compounds, which may be used in combination with a compound of the present invention, can be prepared and administered as described in the art such as in the documents cited above, which is hereby incorporated by reference.

### Method of Treatment

In a further aspect the present invention relates to a method of treating diseases in a subject, said method comprises administering to said subject a therapeutically effective amount of a compound of formulas (I), (VI), or (VII), or pharmaceutically acceptable salts, solvates or prodrugs thereof, as defined herein, to a subject in need of such treatment. The disease may be any disease or disorder as mentioned herein, such as for example mentioned in the section “Treatment of diseases”, and the compound may be administered alone or in pharmaceutical composition such as for example mention in the section “Pharmaceutical compositions”.

In a preferred embodiment of this aspect of the invention the method is a method of treating a proliferative disease in a subject, said method comprises administering to said subject a therapeutically effective amount of a compound of formulas (I), (VI), or (VII), or pharmaceutically acceptable salts, solvates or prodrugs thereof, as defined herein, to a subject in need of such treatment. The proliferative disease may be any proliferative disease as described herein above. Preferably the proliferative disease is cancer.

In one embodiment of the method according to the invention, the compound of formulas (I), (VI), or (VII), or pharmaceutically acceptable salts, solvates or prodrugs thereof, as defined herein, is administered in combination with one or more additional active substances. The active substances may be any active substances, and preferably an active substance as described herein above. More preferably the one or more additional active substances are selected from anticancer agents, antineoplastic agents, cytotoxic drugs, and anti-tumor antibiotics. Even more preferably the one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

The present invention further relates to a method of promoting apoptosis in proliferating cells, which comprises contacting the proliferating cells with an effective apoptosis promoting amount of a compound according to the invention. Preferably the compound according to the invention binds to the Smac binding site of XIAP and/or cIAP proteins.

As described herein above, the compound of the present invention may be used for sensitizing cells to inducers of apoptosis. Accordingly, the invention further relates to a method of sensitizing cells to inducers of apoptosis, said method comprises contacting the cells with an effective amount of a compound according to the invention.

In one embodiment of the methods of treatment according to the invention the subject is an animal in need of such treatment. In a preferred embodiment of the invention the animal is a mammal, such as e.g. humans and veterinary animals (cows, sheep, pigs, horses, dogs, cats and the like). In a more preferred embodiment the animal is a human, and in an alternative embodiment the animals include veterinary animals, such as e.g. cows, sheep, pigs, horses, dogs, cats and the like.

The features mentioned above for the compounds of formula (I), or pharmaceutically acceptable salt, solvates or prodrugs thereof, for pharmaceutical compositions according to the invention, and for the use of compounds of formula (I) for treating diseases, apply mutatis mutandis for the methods of treatment and methods of sensitizing cells to inducers of apoptosis or methods of promoting apoptosis in proliferating cells, according to the present invention.

### General Synthesis

The compounds employed in the methods of the present invention may be prepared in a number of ways. The compounds can be synthesized, for example, by the methods described below, or variations thereon as appreciated by the person skilled in the art. All processes disclosed in association with the present invention are contemplated to be practiced on any scale, including milligram, gram, multi-gram, kilogram, multi-kilogram or commercial industrial scale.

The compounds of the present invention may be prepared by the procedures described in the general methods presented below or by the specific methods described in the example section and the preparation section, or by routine modifications thereof. The present invention also encompasses any one or more of these processes for preparing the compounds of formula (I), (VI), and (VII), in addition to any novel intermediates used therein.

### General Synthesis: Compounds of Formula (I), (IIa), (IIb), (IIIa), (IIIB), (IV), and (V)

The compounds of formula (I) of the present invention may be prepared by a variety of processes, for example as shown in the following Methods A to G, together with routine modifications thereof, which will be well known to persons skilled in the art. The following Methods A to G illustrate the preparation of compounds of formula (IIa), (IIb), (IIIa), (IIIb), (IV) and (V), respectively. Methods H through AS illustrate the preparation of intermediates. Unless otherwise indicated, R¹, R², R³, R⁴, R⁵, R⁶, R⁷, R⁸, A₁, A₂, A³, A⁴ and B in the following methods are as defined herein above.

The term “protecting group”, as used hereinafter, means a hydroxy, carboxy or amino-protecting group which is selected from typical hydroxy, carboxy or amino-protecting groups described in Protective Groups in Organic Synthesis edited by T. W. Greene et al. (John Wiley & Sons, 1999). Examples of suitable amino protecting groups include, but are not limited to t-butyloxycarbonyl (Boc), 9-fluorenylmethoxycarbonyl (Fmoc), benzyloxycarbonyl (Cbz), allyloxycarbonyl (Alloc), trifluoroacetyl (TFAc), methylsulfonylethyl carbamate (Msec) and 2-nitrobenzenesulfonamide (Ns). If the coupling reaction described in other sections is synthesised via solid support, Fmoc, Alloc and Ns are preferred as protecting groups were as if a reaction is performed in solution Boc, Cbz and Ns are preferred as protecting groups. Examples of suitable carboxy protection groups include, but are not limited to methyl (Me), ethyl (Et), t-butyl (t-Bu) and benzyl (Bn). Examples of suitable hydroxy protection groups include, but are not limited to t-Bu, Bn, trimethylsilyl (TMS) and t-butyldimethylsilyl (TBDMS).

The illustrated compounds in Methods A to G may be protected during the synthesis. If the amino group in (IIa), (IIb), (IIIa), (IIIb), (IV) and (V) and the corresponding starting material 2 is primary or secondary, the amino group is protected (e.g. R¹ or R² is a protecting group). The protecting group is chosen according to the procedures used, and is preferably selected from Fmoc, Alloc and Ns when 1 is attached to a solid support. If the reaction is performed in solution; Boc, Cbz and Ns are preferably selected as protecting groups for 2.

However the final compounds of formula (I) are unprotected and all illustrated products in Methods A to G, which are protected with the protecting groups previously mentioned, may be deprotected as described in Protective Groups in Organic Synthesis edited by T. W. Greene et al. (John Wiley & Sons, 1999) and Protecting Group edited by P. J. kocieński (Thieme, 2004, 3. Ed), the disclosures of which hereby are incorporated by reference.

The term “leaving group”, as used herein, signifies a group capable of being substituted by nucleophilic groups, such as a hydroxyl, mercapto group, amines or carboanions and examples of such leaving groups include halogen atoms, an alkylsulfonyl group and an arylsulfonyl group. Of these, a bromine atom, a chlorine atom and a methylsulfonyl and an arylsulfonyl group are preferred.

The term “reaction inert solvent”, as used herein, means a solvent that will not react with any starting material or reagent in a reaction.

The coupling reaction for an amide coupling in the different synthesis step below may be performed under any suitable reaction conditions well-known to a person skilled in the art. The coupling reaction is preferably carried out in the presence of a base, a coupling reagent, and a reaction inert solvent that at least partly dissolves the reagents. Examples of suitable solvents include, but are not limited to halogenated hydrocarbons, such as dichloromethane (DCM), chloroform, carbon tetrachloride and 1,2-dichloroethane (DCE); ethers, such as diethyl ether, diisopropyl ether, t-butyl methyl ether, dimethoxyethane, tetrahydrofuran (THF) and dioxane; and amides, such as N,N-dimethylformamide (DMF), N-methyl-2-pyrrolidone (NMP) and N,N-dimethylacetamide (DMA). Of these solvents, DCM and DMF are preferred. Any base commonly used in reactions of this type may be used here. Examples of suitable bases include amines, such as 1,8-diazabicyclo[5.4.0]undec-7-ene (DBU), pyridine, triethylamine (TEA), N-methylmorpholine (NMM) and diisopropylethylamine (DIPEA); alkali metal carbonates, such as sodium hydrogencarbonat, sodium carbonate and cesium carbonate. Of these, DIPEA is preferred.

Likewise, any coupling reagent and coupling additive commonly used in an amide coupling reactions may be used in amide coupling reaction described below here. Examples of suitable coupling reagents include, but are not limited to uronium-based derivatives, such as 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 2-(6-chloro-1H-benzotriazole-1-yl)-1,1,3,3-tetramethyluronium hexafluorophosphate (HCTU); phosphonium-based derivatives, such as benzotriazole-1-yl-oxy-tris-(dimethylamino)-phosphonium hexafluorophosphate (BOP), benzotriazole-1-yl-oxy-tris-pyrrolidino-phosphonium hexafluorophosphate (PyBOP) and bromo-tris-pyrrolidino-phosphonium hexafluorophosphate (PyBrOP); carbodiimide derivative, such as diisopropylcarbodiimide (DIC), dicyclohexylcarbodiimide (DCC) and 1-Ethyl-3-(3-dimethyllaminopropyl)carbodiimide hydrochloride (EDC×HCl), or a mixture of one of the carbodiimide derivative together with a coupling additive. Examples of suitable coupling additives includes, but are not limited to, N-hydroxy-5-norbornene-endo-2,3-dicarboximide (HONB), N-hydroxysuccinimide (HOSu), N-hydroxybenzotriazole (HOBt), 1-hydroxy-7-azabenzotriazole (HOAt), 3,4-dihydro-3-hydroxy-4-oxo-1,2,3-benzotriazine (DhbtOH) and 4-dimethylaminopyridine (DMAP). Examples of other coupling reagents and additives are described in Activating Agents and Protecting Groups (Handbook of Reagents for Organic Synthesis) edited by A. J. Pearson and W. R. Roush (John Wiley & Sons, 1999) and the disclosures of which are incorporated herein by references. Of the listed coupling agents, PyBOP, DhbtOH, HATU, DIC and EDC×HCl are preferred. The amide coupling reaction can take place over a wide range of temperatures, and the precise reaction temperature is not critical to the invention. The preferred reaction temperature will depend upon such factors as the nature of the solvent, and the starting materials and the amount of one coupling partner compared to the other. However, in general, it is convenient to carry out the reaction at a temperature of from about 0° C. to about 60° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 24 hours will usually suffice.

The amide coupling reaction can either be carried out in solution or on solid support with the preferred solvents or mixtures thereof. There is no particular restriction on the nature of the solid support as long as the support is inert towards the reaction conditions and is capable of binding one of the starting materials to the support via a cleavable linkage.

Examples of suitable supports include, but are not limited to: 2-Chlorotritylchloride resin, Fmoc Rink amide resin and 3-Formylindol-1-yl methyl resin, 3-Formylindol-1-yl acetic acid AMS resin and {3-[(Methyl-Fmoc-amino)-methyl]indol-1-yl}acetyl AM resin.

The amide coupling reaction may furthermore be carried out in solution with solid supported reagents and scavenger which are commercially available from different suppliers such as Varian, Novabiochem and Biotage. Either the base, or the coupling reagent and additive can be supported in these kinds of amide coupling reactions, such as e.g. Method A. After the reaction, solid supported scavenger can be used to scavenge any excess of base, starting material, coupling reagents and additives. Examples of suitable reagents supports for coupling reaction include, but are not limited to: PL-TEA Resin, PL-DIPAM Resin, PL-DMAP Resin, PL-DCC Resin, PL-HOBt Resin, PL-IIDQ Resin and PL-Mukaiyama Resin. Examples of suitable scavenger supports for amide coupling reactions include, but are not limited to, PL-SO₃H Resin, PL-NCO Resin and PL-MIA Resin.

### Method A

Compounds of formula (IIa) may be prepared by the method illustrated in Scheme A:

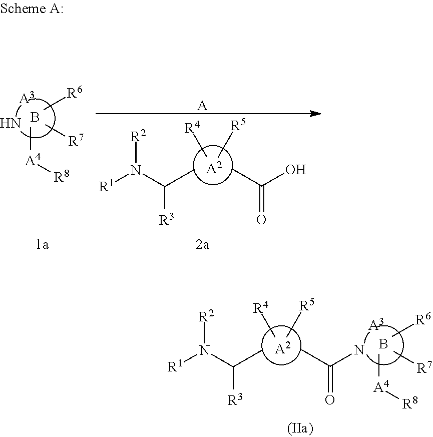

Synthesis A: In this step the compound of formula (IIa) is prepared by an amide coupling reaction between the compounds of formula 1a with the compound of formula 2a. The preferred conditions for amide coupling reactions are described under general synthesis and can involve both solution and solid supported procedures. The compounds of formula 1a and 2a are either commercially available or can be prepared according to the Methods AA to AQ and H to T set forth below.

### Method B

Compounds of formula (IIb) may be prepared by the method illustrated in Scheme B:

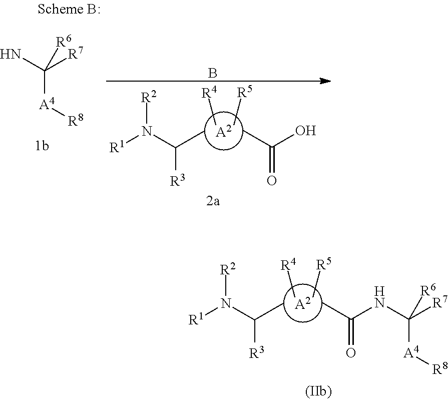

Synthesis B: In this step the compound of formula (IIb) is prepared by an amide coupling reaction between the compounds of formula 1b with the compound of formula 2a. The preferred conditions for amide coupling reactions are described under general synthesis and can involve both solution and solid supported procedures. The compounds of formula 1b and 2a are either commercially available or can be prepared according to the Methods in AR and H to T set forth below.

### Method C

Compounds of formula (IIIa) may be prepared by the method illustrated in Scheme C:

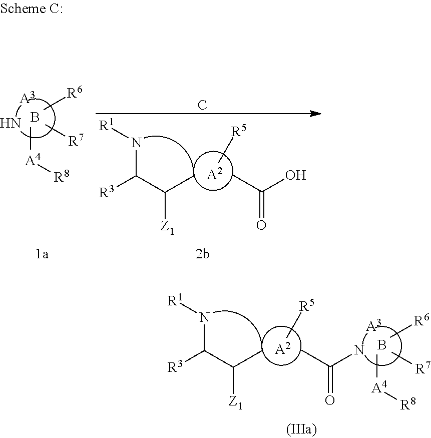

Synthesis C: In this step the compound of formula (IIIa) is prepared by an amide coupling reaction between the compounds of formula 1a with the compound of formula 2b. The preferred conditions for amide coupling reactions are described under general synthesis and can involve both solution and solid supported procedures. The compounds of formula 1a and 2b are either commercially available or can be prepared according to the Methods AA to AQ and U to W set forth below.

### Method D

Compounds of formula (IIIb) may be prepared by the method illustrated in Scheme D:

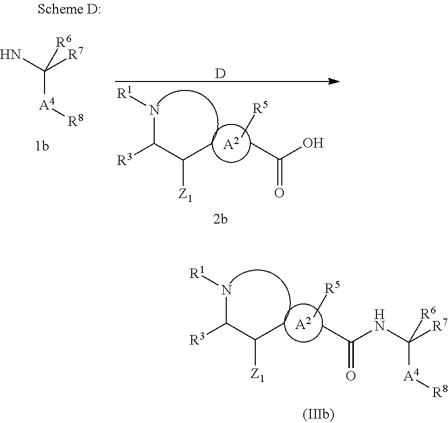

Synthesis D: In this step the compound of formula (IIIb) is prepared by an amide coupling reaction between the compounds of formula 1a with the compound of formula 2b. The preferred conditions for amide coupling reactions are described under general synthesis and can involve both solution and solid supported procedures. The compounds of formula 1b and 2b are either commercially available or can be prepared according to the Methods in AR and U to W set forth below.

### Method E

Compounds of formula (IV) may be prepared by the method illustrated in Scheme E:

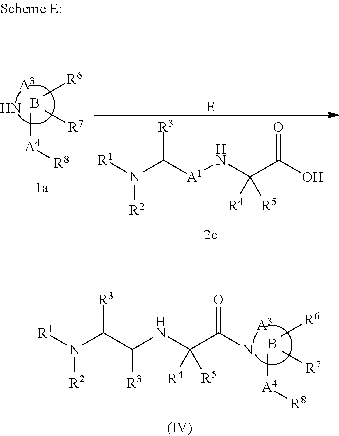

Synthesis E: In this step the compound of formula (IV) is prepared by an amide coupling reaction between the compounds of formula 1a with the compound of formula 2c. The preferred conditions for amide coupling reactions are described under general synthesis and can involve both solution and solid supported procedures. The compounds of formula 1a and 2c are either commercially available or can be prepared according to the Methods AA to AQ and X to Z set forth below.

### Method F

Compounds of formula (IV) wherein A² is a —NHCR⁴R⁵— moiety and A¹ is a carbonyl may be prepared according to procedures illustrated in Scheme F:

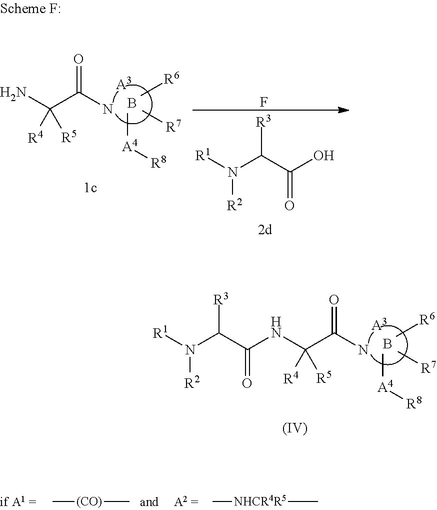

Synthesis F: In this step the compound of formula (IV) is prepared by an amide coupling reaction between the compounds of formula 1c with the compound of formula 2d. The preferred conditions for amide coupling reactions are described under general synthesis and can involve both solution and solid supported procedures. The compounds of formula 1c and 2d are either commercially available or can be prepared according to the methods in Method AS set forth below.

### Method G

Compounds of formula (V) wherein A² is a —NHCR⁴R⁵— moiety and A¹ is carbonyl may be prepared according to procedures may be prepared by the method illustrated in Scheme G:

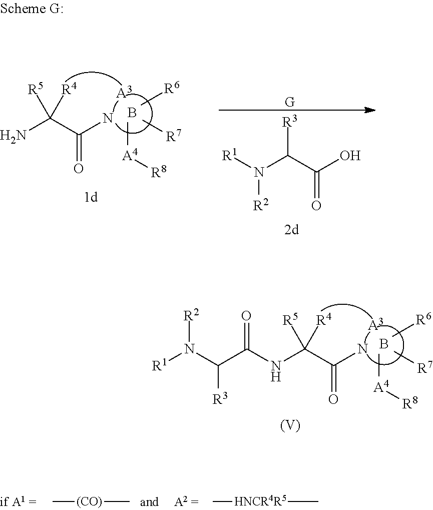

Synthesis G: In this step the compound of formula (V) is prepared by an amide coupling reaction between the compounds of formula 1d with the compound of formula 2d. The preferred conditions for amide coupling reactions are described under general synthesis and can involve both solution and solid supported procedures. The compounds of formula 1d and 2d are either commercially available or can be prepared according to the method in Method AT set forth below.

### Preparations of Intermediates 2

The prepared structures of Intermediates 2 are amino esters. Protection is therefore necessary for intermediates 2 with primary and secondary amino groups. The amino protecting group is chosen according to solution or solid phase strategies described under general synthesis. The protection can be performed either on the starting material or the on intermediate itself according to protocols described in Protective Groups in Organic Synthesis edited by T. W. Greene et al. (John Wiley & Sons, 1999) and Protecting Group edited by P. J. kocieński (Thieme, 2004, 3. Ed) and the disclosures of which are incorporated herein by references. The ester group on the intermediates would have to be cleaved to the acid before use in Methods A to G. Depending on the type of protection group the amino functionality carries; the ester group will be cleaved orthogonally to the amino protecting group and would involve either basic or acidic conditions.

Cleavage of esters under basic conditions may be performed in a mixture of organic solvent and water at temperatures of about 0° C. to about 100° C. for about 1 to 24 hours to give the corresponding acid. Suitable bases include but are not limited to alkali hydroxides and alkali carbonates. Suitable organic solvents include but are not limited to ethanol, methanol, and tetrahydrofurane. Cleavage of esters under acidic conditions may be performed in a pure organic solvent or in a mixture of organic solvent and water at temperatures of about 0° C. to about 100° C. for about 1 to 24 hours to give the corresponding acid. Suitable acids include but are not limited to trifluoroacetic acid, sulphuric acid and hydrochloric acid. Suitable organic solvents include but are not limited to dioxane, tetrahydrofuran, dichloromethane and diethyl ether. The starting materials for the intermediates are either commercially available or obtained by conventional methods known to those skilled in the art.

### Preparations of Intermediates 2a

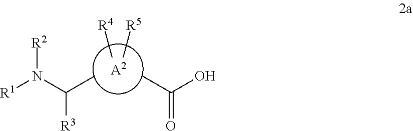

Intermediates of formula 2a are used for preparing compounds of the invention in which A² is cycloalkyl, aryl, heterocyclyl, or heteroaryl, such as compounds of formula (IIa) and (IIb). These intermediates may be prepared by various methods depending on the specific type of ring structure. The following Methods H to Y describes the methods for synthesis of different types of ring structures.

### Method H

Intermediate of formula 2a, wherein A² is an 1,2,4-oxadiazole, may be prepared according to the procedures described by Wang et al. Org. Lett. 2005, 7, 925-928 or in US20040019215, both of which hereby are incorporated by reference, as illustrated in Scheme H.

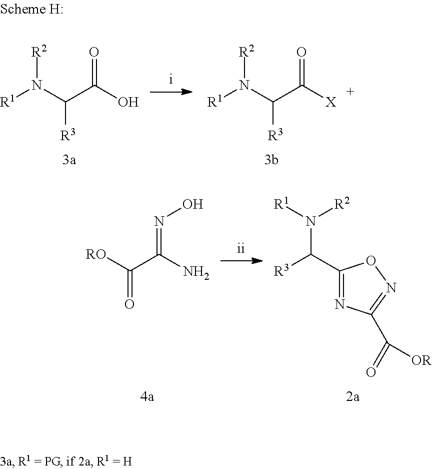

Step i: The amino acid 3a is converted to the activated acid 3b where x is a chloride, activated ester or an anhydride.

Step ii: The activated acid 3b is coupled to 4a and ring-closed to form 2a in which A² is a 1,2,4-oxadiazole.

### Method I

Intermediates of formula 2a, wherein A² is an 1,3,4-oxadiazole may be prepared according to the procedures described in US20040019215, which is hereby incorporated by reference, as illustrated in Scheme I.

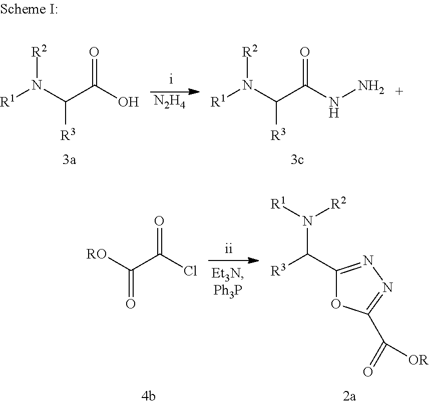

Step i: Using hydrazine hydrate the amino acid ester 3a is converted to 3c.

Step ii: 3c is coupled to 4b in the presence of a base such as sodium hydrogencarbonate. After purification the coupling product is treated with triethylamine and triphenylphosphine at elevated temperature to form 2a in which A² is a 1,3,4-oxadiazole.

### Method J

Intermediates of formula 2a, wherein A² is an oxazole may be prepared according to the procedures described by Trukhin et al. Synlett 2005, 2072-2076, the disclosures of which hereby is incorporated by references. A representative method is illustrated in Scheme J.

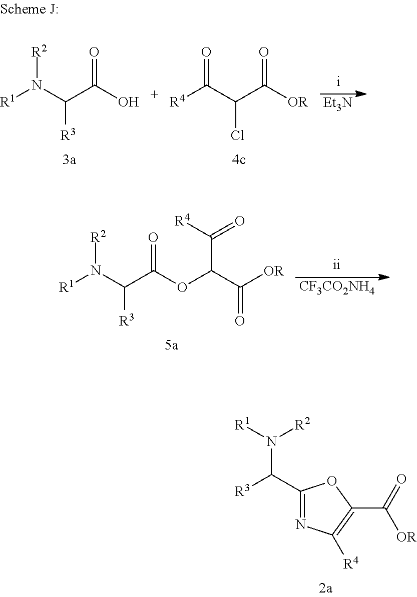

Step i: 3a is coupled to 4c to give 5a in the presence of a base such as triethylamine.

Step ii: 5a is ring-closed at high temperature in the presence of an ammonium salt such as ammonium trifluoroacetate to form 2a in which A² is an oxazole.

### Method K

Intermediates of formula 2a, wherein A² is an oxazole may be obtained from conventional methods known to those skilled in the art, such as Wist et al. Bioorg. Med. Chem. 2007, 2935-2943; Riedrich et al. Angew. Chem. Int. Ed. 2007, 46, 2701-2703; You et al. J. Org. Chem. 2003, 68, 9506-9509; Bagley et al. Synlett 1996, 825-826; Falorni et al. Eur. J. Org. Chem. 2000, 3217-3222, the disclosures of which are incorporated herein by references. A representative method is illustrated in Scheme K.

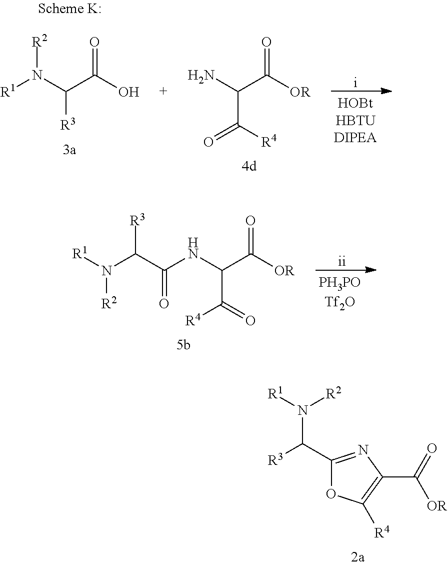

Step i: 3a is coupled to 4d to give 5b in the presence of a base such as DIPEA and coupling reagents and additives such as HBTU and HOBt.

Step ii: 5b is ring-closed in the presence of triphenylphosphine oxide and triflic anhydride to form 2a in which A² is an oxazole.

### Method L

Intermediates of formula 2a, wherein A² is an imidazole may be obtained from conventional methods known to those skilled in the art, such as Haberhauer et al. Eur. J. Org. Chem. 2003, 3209-3218; Haberhauer et al. Chem. Eur. J. 2005, 11, 6718-6726; the disclosures of which are incorporated herein by references. A representative method is illustrated in Scheme L.

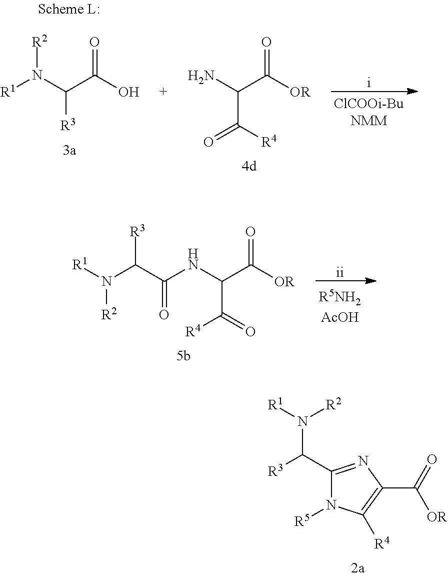

Step i: 3a is coupled to 4d to give 5b in the presence of a base such NMM and a coupling reagent such as isobutyl chloroformate.

Step ii: 5b is ring closed in present of a primary amine and acetic acid to form 2a in which A² is an imidazole.

### Method M

Intermediates of formula 2a, wherein A² is a thiazole may be prepared according to the procedures described in WO20005097766, which is hereby incorporated by reference, as illustrated in Scheme M.

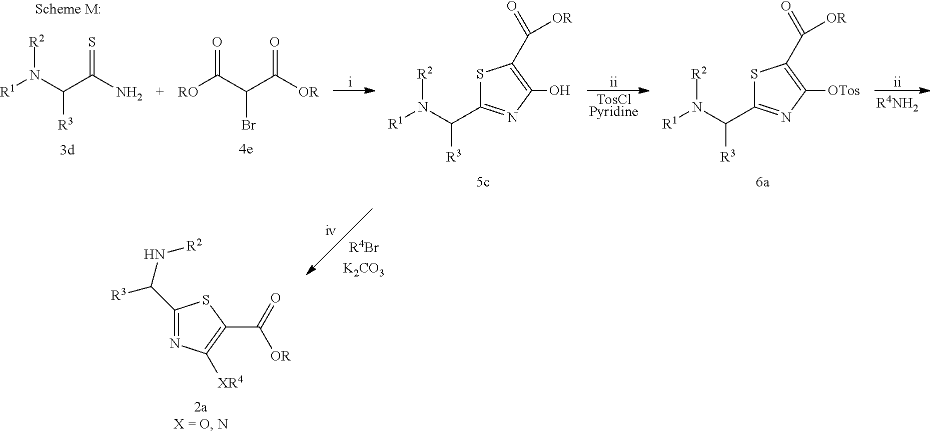

Step i: 5c may be prepared by a reaction between 3d and 4e in an inert organic solvent at temperatures of about 50 to about 200° C., preferably at about 100 to 150° C., for about 0.5 to about 24 hours. Suitable solvents include benzene, toluene, xylene, and DMF.

Step ii: 5c may be converted to 6a using para-tosyl chloride in the presence of an organic base and an organic solvent at a temperature of about −20° C. to about 100° C. for 1 to 48 hours. Suitable bases include TEA, DIPEA, pyridine, 2,6-lutidine. Suitable organic solvents include DCM, DCE, chloroform, THF and diethyl ether.

Step iii: In present of a primary amine and at elevated temperature of about 50° C. to 200° C., preferably at about 100° C. to 150° C., for 5 to 48 hours compound 6a may be converted to 2a in which X=N.

Step iv: 5c may be converted to 2a where X=O in a nucleophilic substitution using any alkylating reagent with a leaving group on such as bromide or an alkylsulfonyl group. The reaction should be performed in the presence of a base and an organic solvent at a temperature of about −20° C. to about 100° C. for 1 to 48 hours. Suitable bases include potassium carbonate and sodium hydride. Suitable organic solvents include acetone, NMP and THF.

### Method N

Intermediates of formula 2a, wherein A² is a thiazole may be obtained from conventional methods known to those skilled in the art, such as Bagley et al. Synthesis 2007, 3535-3541 and Riedrich et al. Angew. Chem. Int. Ed. 2007, 46, 2701-2703; the disclosures of which are incorporated herein by references. A representative method is illustrated in Scheme N.

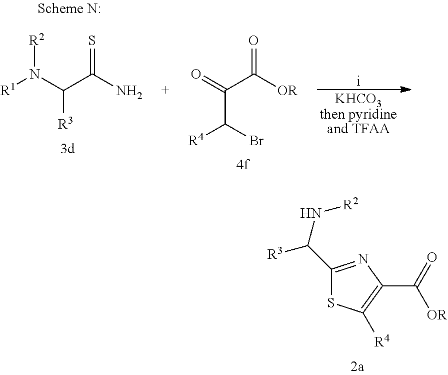

Step i: 3d is ring-closed with 4f in the presence of potassium carbonate to give a hydroxythiazoline intermediate which eliminate water upon treatment with pyridine and trifluoroacetic anhydride (TFAA) to give 2a in which A² is a thiazole.

### Method O

Intermediates of formula 2a, wherein A² is a 1,2,4-oxadiazole, may be obtained from conventional methods known to those skilled in the art, such as Borg et al. J. Org. Chem. 1995, 60, 3112-3120; which is hereby incorporated by references. A representative method is illustrated in Scheme O.

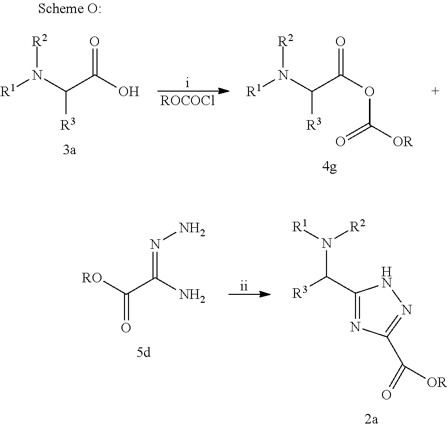

Step i: The amino acid 3a is converted to the activated acid 4g.

Step ii: The activated acid 4g is coupled to 5d and ring-closed to form 2a in which A² is a 1,2,4-triazole.

### Method P

Intermediates of formula 2a, wherein A² is a benzene or pyridine ring, may be prepared according to the procedures described by Bhattacharyya et al. Synlett. 1999, 1781-1783 or Peterson et al. Synth. Comm. 2002, 32, 443-448; the disclosures of which are incorporated herein by references. A representative method is illustrated in Scheme P.

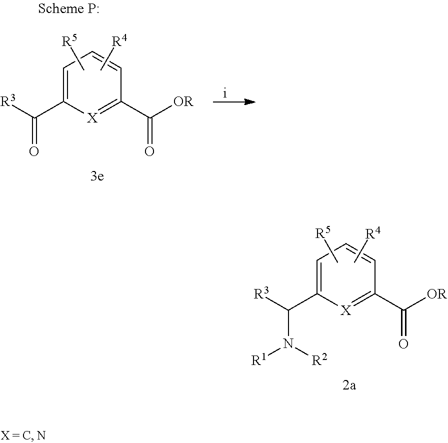

Step i: The reductive amination is performed in the presence of amine, with or without a Lewis acid and a hydride source. The reaction is performed at a temperature of about −78° C. to 100° C., preferably at about 0 to 50° C., for 5 to 48 hours to convert compounds 3e to 2a. Examples of Lewis acids include, but are not limited to: titanium isopropoxide, titanium methoxide, titanium ethoxide and borotrifluoride. Examples of hydride source include, but are not limited to: sodium borohydride, sodium cyanoborohydride, sodium triacetoxyborohydride, ammonium acetate/rhodium(III) or borane. Suitable solvents include, but are not limited to: dioxane, THF, NMP, DMF, DCM, diethyl ether and ethyl acetate.

### Method Q

Intermediates of formula 2a, wherein A² is a benzene, heterocycle, carbocycle or pyridine ring, may be prepared according to procedures described in WO9426779; Yanagisawa et al. J. Med. Chem. 1988, 31, 422-428; or Vaccaro et al J. Med. Chem. 1996, 39, 1704-1719, each of which hereby are incorporated by reference, as illustrated in Scheme Q.

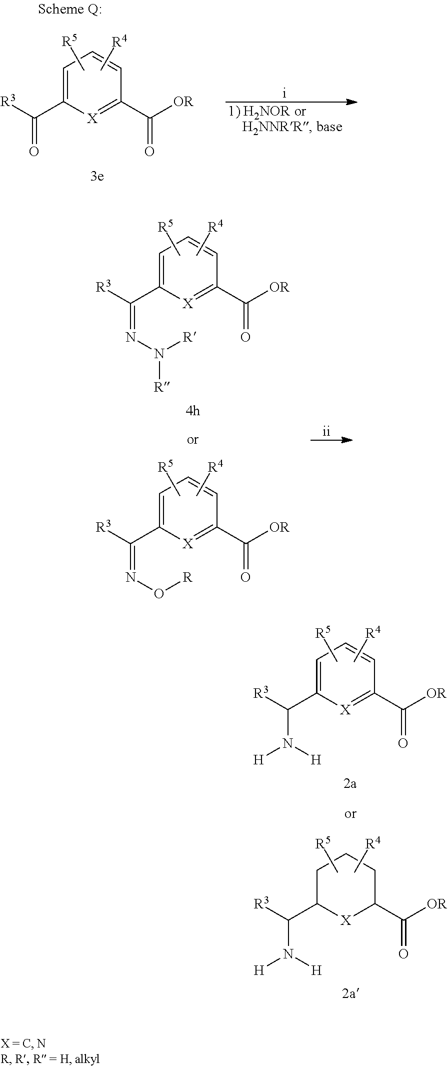

Step i: In the presence of nucleophiles such as O-alkyl hydroxylamine, hydroxylamine hydrochloride, N-alkyl, N,N-dialkyl hydrazine and base such as pyridine, sodium acetate, sodium hydrogencarbonate or sodium carbonate the oximes or hydrazones 4h may be formed at temperatures about 0 to 60° C. from 3e. Suitable solvents for the reaction include, but are not limited to: dioxane, THF, DCM, methanol, ethanol and ethyl acetate.

Step ii: The oxime or hydrazone is reduced in presence of a catalyst and hydrogen to form 2a. Suitable catalyst include, but are not limited to: palladium, rhodium, Raney nickel, zinc. Examples of hydrogen sources include, but are not limited to: hydrogen, zinc/acetic acid, zinc/hydrochloric acid, ammoniumformiate. Suitable solvents for the reaction include, but are not limited to: dioxane, THF, DCM, ethyl acetate, methanol and ethanol.

### Method R

Intermediates of formula 2a, wherein A² is a furan or a pyrrole, may be prepared according to procedures described by Ercoli et al. J. Org. Chem. 1967, 32, 2917-2918, the disclosures of which hereby is incorporated by references. A representative example is illustrated in Scheme R.

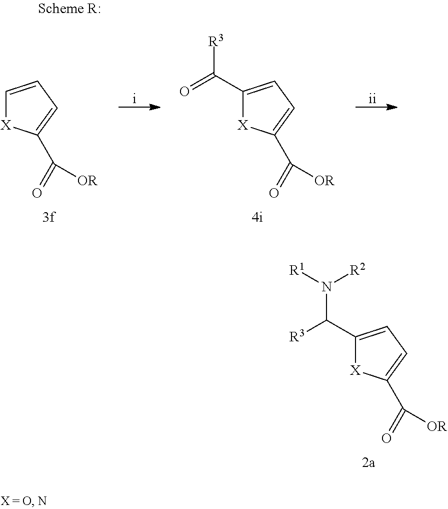

Step i: The Fiedel-Crafts acylation is performed in the presence of an acylating agent and a Lewis acid and at temperatures of about 0° C. to 100° C., preferably at about 0 to 50° C., for 5 to 48 hours to convert compound 3f to 41. Suitable acylating agent include, but are not limited to: anhydrides and acid chlorides. Suitable Lewis acids include, but are not limited to: tin tetrachloride, borotrifluoride, titanium tetrachloride, aluminium trichloride, sulphuric acid and hydrogen chloride. The solvent are usually the acylating agent however solvents like benzene, toluene and diethyl ether may be applied.

Step ii: The Methods from P or Q are used.

### Method S

Intermediates of formula 2a which are enantiomeric pure and wherein A² is a furan, may be prepared according to procedures described by Chakraborty et al. Synlett, 2004, 2484-2488, which is hereby incorporated by reference. See intermediate 2a in Scheme S.

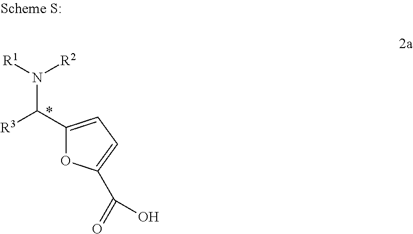

### Method T

If the amino group described in Methods H to S is a primary amine it may be possible to transform the amino group into a secondary amine according to procedures described by Bowman et al. Tetrahedron, 1997, 53, 15787-15798; Miller et al. J. Am. Chem. Soc. 1998, 120, 2690-2691 and Fukuyama et al. Chem. Commun. 2004, 353-359; the disclosures of which are incorporated herein by references. A representative example is illustrated in Scheme T.

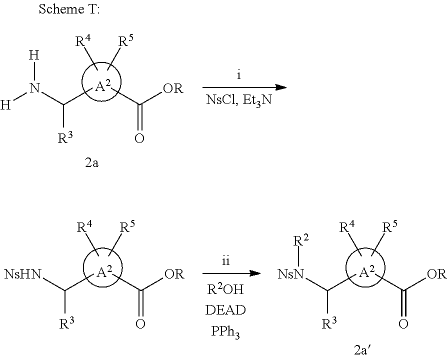

Step i: In the presence of a base the amino group in 2a is protected which a protecting group which gives rise to a pKa of 9-11 for the amine proton. Suitable protecting groups include but are not limited to trifluoroacetyl and 2-nitrobenzenesulfonyl. Suitable bases include but are not limited to DIPEA, pyridine, triethylamine, sodium carbonate, sodium hydrogencarbonate and sodium hydroxide. Suitable organic solvents include but are not limited to pure organic solvents such as DCM, dioxan, DMF, tetrahydrofuran and a mixture of water and the previous mentioned organic solvents. Reactions may be performed at temperatures about 0° C. to about 60° C. for about 1 to 24 hours.

Step ii: The Mitsunobu reaction involves, besides Ns protected amin and an alcohol, a phosphine and a dialkyl azodicarboxylate to form 2a′. Suitable phosphines include but are not limited to triphenyl phosphine, tributyl phosphine (TBP). Suitable dialkyl azodicarboxylate include but are not limited to 1,1′-(azadicarbonyl)dipiperidine (ADDP) or diethylazadicarboxylate (DEAD). Suitable organic solvent include but are not limited to DCM, dioxan, DMF, and tetrahydrofuran. Reaction may be performed at temperatures of about 0° C. to about 100° C. for about 1 to 24 hours. The reaction can take place in solution as well as on solid support.

### Preparations of Intermediate 2b

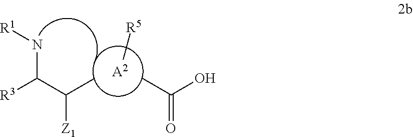

Intermediates of formula 2b are used for preparing spiro compounds of the invention in which two rings are joined at a single atom, such as compounds of formula (IIIa) and (IIIb). One of the rings is the A² whereas the second ring contains the terminal amino group and is formed by R² and R⁵, which together form a heterocyclic ring. A² can be cycloalkyl, and heterocyclyl whereas the second ring can be a heterocyclyl. These intermediates may be prepared by variety of processes well known for persons skilled in the art.

### Method U

Intermediates of formula 2b, wherein the spiro compound contains two heterocycles in which one is an isoxazolidine may be prepared in according to procedures described by Grigg et al. Tetrahedron 1991, 47, 4477-4494, which is hereby incorporated by reference, as illustrated in Scheme U.

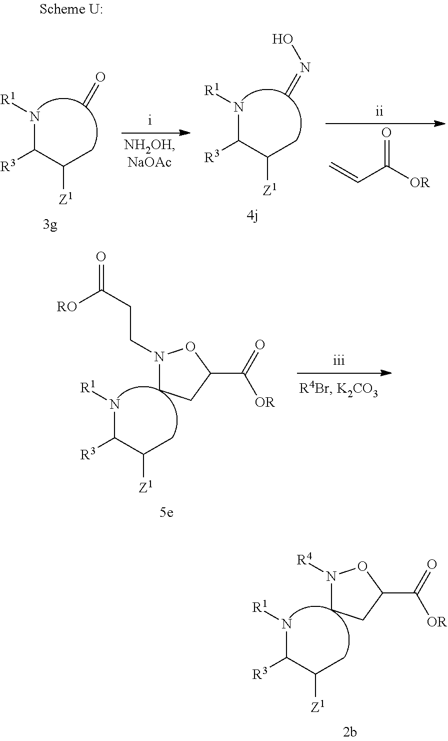

Step i: The keton 3g is transformed to the oxime derivate 4j.

Step ii: The oxime 4j reacts with alkyl acrylate in a 1,3-dipolar cycloaddition to form spiro compound 5e.

Step iii: The compound 5e is alkylated in presence of base to form 2b. Examples of bases include, but are not limited to: alkali carbonates, alkali hydroxides, DBU, DIPEA, alkali t-butoxyl. Examples of solvents include, but are not limited to DCM, chloroform, carbon tetrachloride and DCE; ethers, such as diethyl ether, diisopropyl ether, t-butyl methyl ether, dimethoxyethane, THF and dioxane; and amides, such as DMF, NMP and DMA.

### Method V

Intermediates of formula 2b, wherein the spiro compound contains two amine heterocycles may be prepared in according to procedures described by Teetz et al. Tetrahedron Lett. 1981, 25, 4483-4486, which is hereby incorporated by reference, as illustrated in Scheme V.

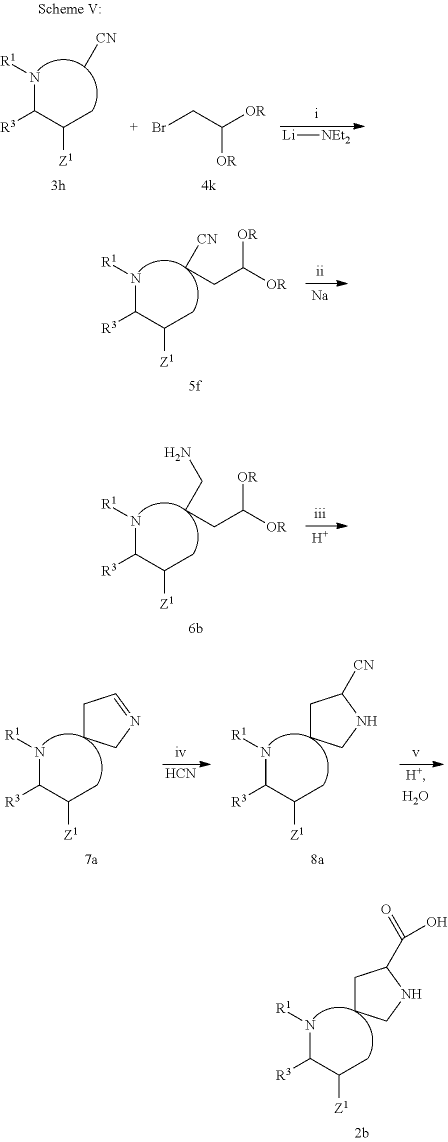

Step i: 3h is alkylated with 4j in presence of lithium diethylamine to give compound 5f.

Step ii: The cyano group in compound 5f is reduced with sodium to give compound 6b.

Step iii: In presence of acid the 6b is ring closed to give 7a.

Step iv: Addition of hydrogencyanide to 7a gives 8a.

Step v: The cyano group is hydrolyzed to give the acid derivate 2b.

### Method W

Intermediates of formula 2b, wherein the spiro compound contains two heterocycles in which one is a thiazol may be prepared in according to procedures described by Refouvelet et al. Chem. Pharm. Bull. 1994, 42, 1076-1083, which is hereby incorporated by reference, as illustrated in Scheme W.

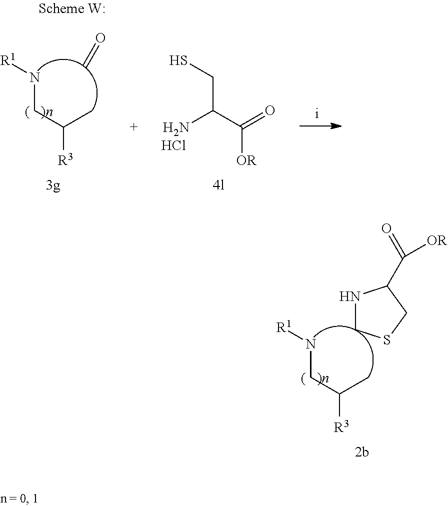

Step i: At elevated temperature compound 3g is ring-closed with 41 to give 2b.

### Preparations of Intermediates 2c

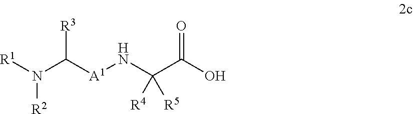

Intermediates of formula 2c are used for preparing peptide like compounds of the invention with formula (IV). The A² is defined as —NHCR⁴R⁵— whereas A¹ can be carbonyl or methylene, (—CH₂—). These intermediates may be prepared by various methods depending on the specific 2c structure. The following Methods X to Z describes methods for synthesis of different 2c structures.

### Method X

Intermediates of formula 2c, wherein A² is a —NHCR⁴R⁵— moiety and A¹ is a carbonyl, may be prepared by the method illustrated in Scheme X.

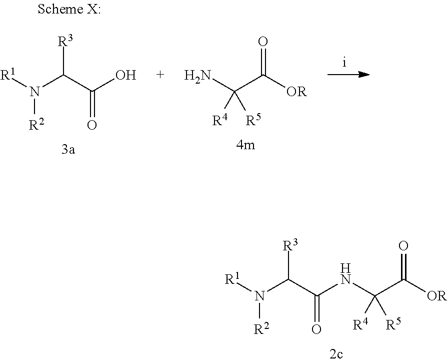

Step i: The compound 2c is prepared by an amide coupling reaction between 3a and 4m. The preferred conditions for amide coupling reactions are described under general synthesis and can involve both solution and solid supported procedures.

### Method Y

Intermediates of formula 2c′, wherein A² is a —NHCR⁴R⁵— moiety and A¹ is an methylene may be prepared according to procedures described by Brown et al. J. Org. Chem. 1982, 47, 3153-3163 and Hall et al. J. Org. Chem. 1999, 64, 698-699; the disclosures of which are incorporated herein by references. A representative example is illustrated in Scheme Y.

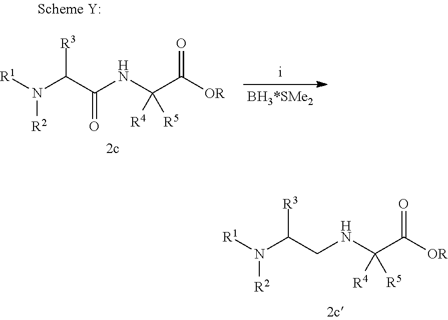

Step i: In presence of borane the secondary amide in 2c is reduced to 2c′.

### Method Z

Intermediate of formula 2c, wherein A² is a —NHCR⁴R⁵— moiety and A¹ is an methylene may be prepared according to procedures described by Bowman et al. Tetrahedron, 1997, 53, 15787-15798; Miller et al. J. Am. Chem. Soc. 1998, 120, 2690-2691, and Fukuyama et al. Chem. Commun. 2004, 353-359; the disclosures of which are incorporated herein by references. A representative example is illustrated in Scheme Z.

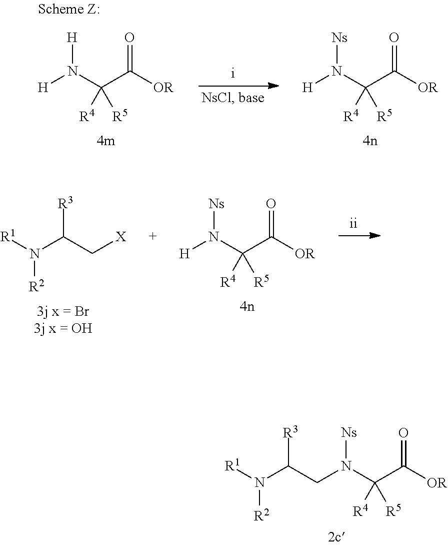

Step i: In presence of a base the amino group in 4m is protected which a protecting group which gives rise to a pKa of 9-11 for the amine proton in 4n. Suitable protecting groups include but are not limited to trifluoroacetyl and 2-nitrobenzenesulfonyl. Suitable bases include but are not limited to DIPEA, pyridine, triethylamine, sodium carbonate, sodium hydrogencarbonate and sodium hydroxide. Suitable organic solvent include but are not limited to pure organic solvent such as DCM, dioxan, DMF, tetrahydrofuran and a mixture of water and the previous mentioned organic solvent. Reaction may be performed at temperatures of about 0° C. to about 60° C. for about 1 to 24 hours.

Step ii: 4n is alkylated using base and 31 or using Mitsunobu conditions together with 3j. Suitable bases together with 3i and 4n include but are not limited to sodium carbonate, potassium carbonate, cesium carbonate and DBU. Suitable organic solvent include but are not limited to DCM, dioxan, DMF, and tetrahydrofuran. Reaction may be performed at temperatures of about 0° C. to about 100° C. for about 1 to 24 hours. The Mitsunobu reaction involves beside the two starting materials 3j and 4n, a phosphine and a dialkyl azodicarboxylate to form 2c′. Suitable phosphines include but are not limited to triphenyl phosphine, tributyl phosphine (TBP). Suitable dialkyl azodicarboxylate include but are not limited to 1,1′-(azadicarbonyl)dipiperidine (ADDP) or diethylazadicarboxylate (DEAD). Suitable organic solvent include but are not limited to DCM, dioxan, DMF, and tetrahydrofuran. Reaction may be performed at temperatures of about 0° C. to about 100° C. for about 1 to 24 hours. The reaction can take place in solution as well as on solid support.

### Preparations of Intermediates 2d

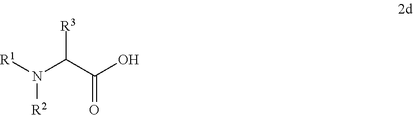

Intermediates of formula 2d are used for preparing peptide like compounds of the invention with formula (IV) and (V). These intermediates are commercial available or protected analogues thereof, which may be obtained from conventional methods known to those skilled in the art.

### Preparations of Intermediates 1

The prepared representatives of intermediates 1 are amines and protection is necessary for the starting material 3. The protecting group is chosen according to the procedures used. For some reactions such as amide formation and reductive amination, the methods can be applied in solution as well on solid phase. In solution Boc, Cbz and Ns are preferably selected as protecting groups for 3. On solid support Fmoc, Alloc and Ns are preferably selected as protecting groups for 3 when 4 is linked to a solid support. The amino group of 4 may be protected before coupling to a solid support and Fmoc, Ns and Alloc are the preferred choice of protection group. 1 is deprotected before use in Methods A to G. Protection of 3 and 4 and deprotection of 1 and 4 are in according to protocols described in Protective Groups in Organic Synthesis edited by T. W. Greene et al. (John Wiley & Sons, 1999) and Protecting Group edited by P. J. kocieński (Thieme, 2004, 3. Ed), the disclosures of which are incorporated herein by references.

The starting materials for the intermediates are either commercially available or obtained by conventional methods known to those skilled in the art.

### Preparations of Intermediates 1a

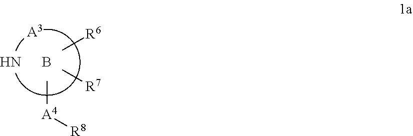

Intermediates of formula 1a are used for preparing compounds of formula (IIa), (IIIa) and (IV). These intermediates may be prepared by various methods depending on the specific type of linkage A⁴ between B and R⁸. The following Methods AA to AT describes methods for coupling of B with R⁸.

### Method AA

Intermediate of formula 1a, wherein A⁴ is an amide moiety may be prepared by the method illustrated in Scheme AA.

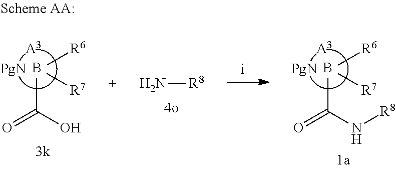

Step i: In this step the compound 2c is prepared by an amide coupling reaction between 3k and 4o. The preferred condition for amide coupling reaction is described under general synthesis and can involve both solution and solid supported procedures.

### Method AB

Intermediates of formula 1a′, wherein A⁴ is a (—CH₂NH—) moiety may be prepared according to procedures described by Brown et al. J. Org. Chem. 1982, 47, 3153-3163 and Hall et al. J. Org. Chem. 1999, 64, 698-699, the disclosures of which are incorporated herein by references. A representative example is illustrated in Scheme AB.

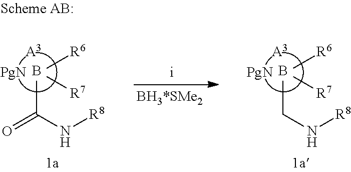

Step i: In presence of borane the amide 1a is reduced to 1a′.

### Method AC

Intermediates of formula 1a, wherein A⁴ is an amide moiety may be prepared by the method illustrated in Scheme AC.

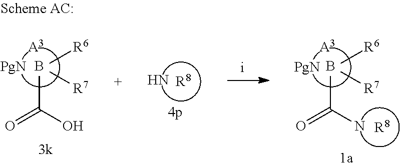

Step i: In this step the compound 1a is prepared by an amide coupling reaction between 3k and 4p. The preferred conditions for the amide coupling reaction is described under general synthesis and can involve both solution and solid supported procedures.

### Method AD

Intermediate of formula 1a′, wherein A⁴ is a (—CH₂NH—) moiety may be prepared as described in Method AA. A representative example is illustrated in Scheme AD.

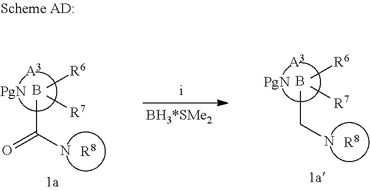

Step i: In presence of borane the amide 1a is reduced to 1a′

### Method AE

Intermediates of formula 1a, wherein A⁴ is a single bound between B and R⁸, may be prepared according to scheme AE wherein R⁸ is a thiazole.

Q represents, but is not limited to: H, halogen, hydroxyl, alkoxy, alkyl, cycloalkyl, aryl and heterocyclyl optionally substituted with halogen, hydroxyl, alkoxyl and alkyl. Z⁴, Z⁵ and Z⁶ are independently CQ or N

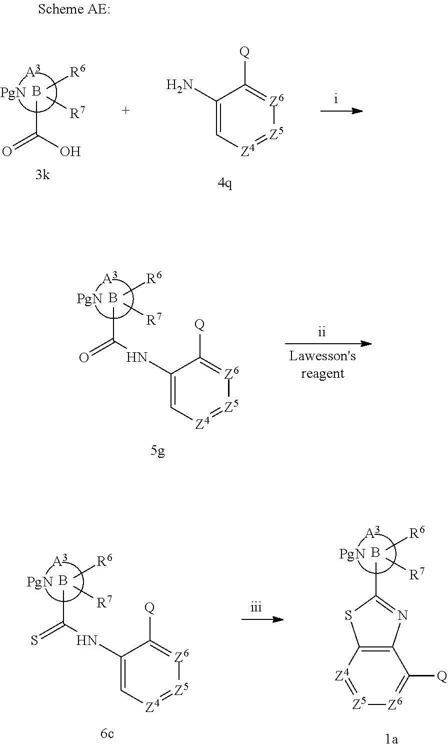

Step i: Amine 4q is coupled with 3k using standard amide formation procedures, to form amide 5g.

Step ii: 5g is converted to the corresponding thioamide 6c by reacting with Lawesson's reagent.

Step iii: Thioamide 6c is cyclized, for example with K₃Fe(CN)₆ in ethanol to form 1a.

### Method AF

Alternatively, 1a thiazole intermediates may be prepared according to Scheme AF.

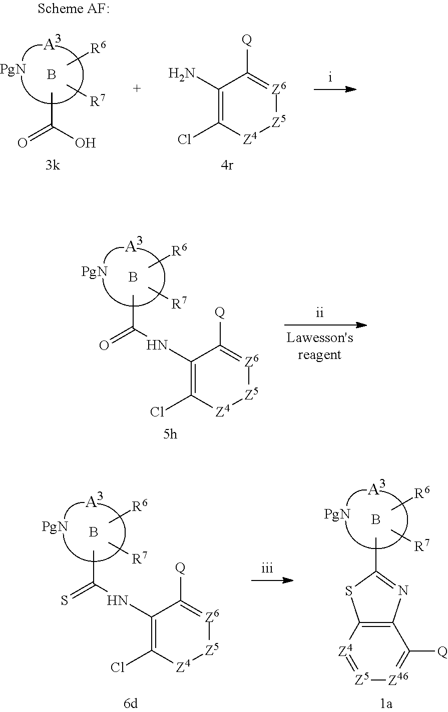

Step i: Acid 3k is coupled to chloro-substituted amine 4r using HATU and DIPEA to give amide 5h.

Step ii: 5h is reacted with Lawesson's reagent to give 6d.

Step iii: 6d is heated to give cyclized compound 1a.

### Method AG

Oxazole intermediates 1a may be prepared according to the procedures described by Wang et al. (Bioorganic & Medicinal Chemistry (2004), 12(1):17-21) as illustrated in Scheme AG.

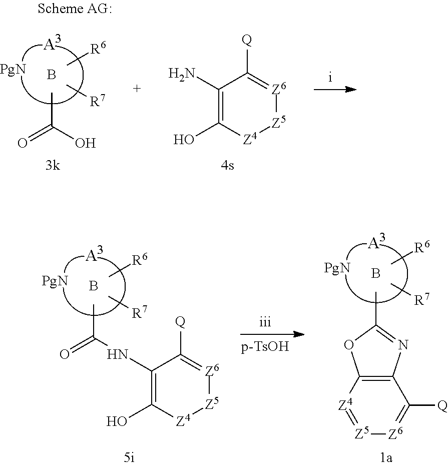

Step i: Similar to schemes AE and AF, an acid 3k is coupled with amine 4s to give amide 51.

Step ii: Amide 51 is treated with p-toluenesulfonic acid in refluxing toluene to give 1a.

### Method AH

Alternatively, oxazole intermediates 1a may be prepared according to the procedures described by Kauffman al. (Journal of Heterocyclic Chemistry (2002), 39(5), 981-988), which is hereby incorporated by reference, illustrated in Scheme AH.

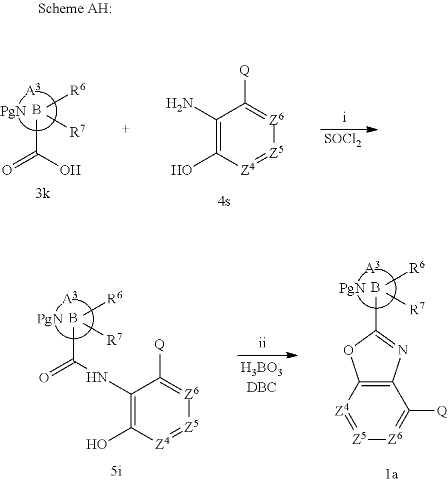

Step i: A mixture of acid 3k, thionylchloride and N-methylpyrrolidinone is refluxed in dioxane under an inert gas and the resulting acid chloride is coupled with the hydroxy amine 4s to give amide 51.

Step ii: 51 is then heated with boronic acid in dibutylcarbinol to give 1a.

### Method AI

Imidazole intermediates 1a may be prepared according to the procedures described by Kumar et al. (Bioorganic & Medicinal Chemistry (2002), 10(12), 3997-4004), which is hereby incorporated by reference, as illustrated in Scheme AI.

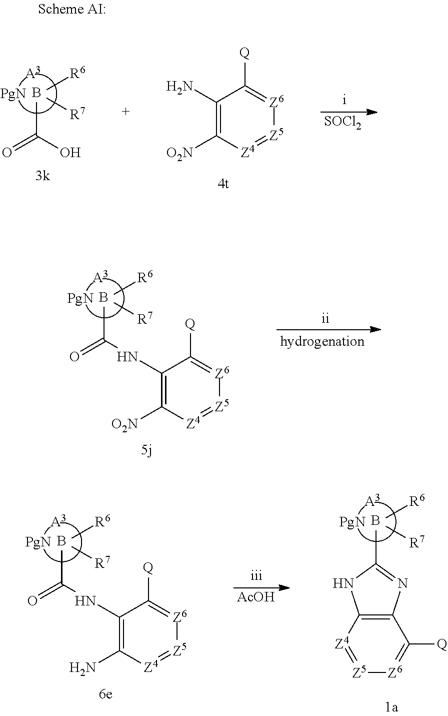

Step i: Acid chloride 3k is coupled with nitro/amine 4t to give amide 5j.

Step ii: The nitro group of amide 5j is reduced to the corresponding amine 6e, for example with iron or Pd/H₂.

Step iii: 6e is cyclised by heating with acetic acid to give 1a.

### Method AJ

Intermediate of formula 1a, wherein A⁴ is an ethylene unit between B and R⁸ may be prepared in according to procedures described in WO 2005/097791 and WO2006/107964, or as illustrated in Scheme AJ.

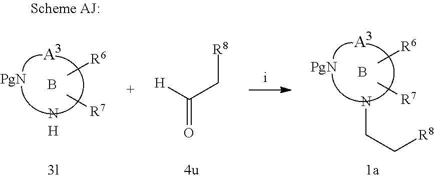

Step i: The reductive amination is performed in presence of amine 31, aldehyde 4u, acetic acid and a hydride source. The reaction is performed at a temperature of about 0° C. to 100° C., preferably at about 0 to 50° C., for 5 to 48 hours to convert compound 31 to 1a. Examples of hydride source include, but are not limited to: sodium borohydride, sodium cyanoborohydride and sodium triacetoxyborohydride. The solvent include, but are not limited to: dioxane, THF, NMP, DMF, DCM, diethyl ether and ethyl acetate

### Method AK

Tetrazole intermediates of formula 1a, wherein A⁴ is a bound between B and R⁸ may be prepared in according to procedures described in WO2005/097791, which is hereby incorporated by reference, as illustrated in Scheme AK.

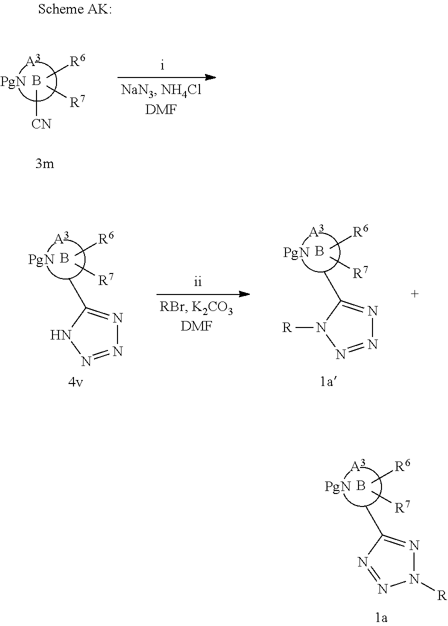

Synthesis i: 3m is converted to the tetrazole 4v in presence of sodium azide and ammonium chloride.

Synthesis ii: A mixture of 1a and 1a′ are generated by alkylation of 4v in presence of potassium carbonate. 1a can be isolated from 1a′ and used further on.

### Method AL

1,2,4-Oxadiazole intermediates of formula 1a, wherein A⁴ is a bound between B and R⁸ may be prepared in according to procedures described by Wang et al. Org. Lett. 2005, 7, 925-928 or in US20040019215, which is hereby incorporated by reference, as illustrated in Scheme AL.

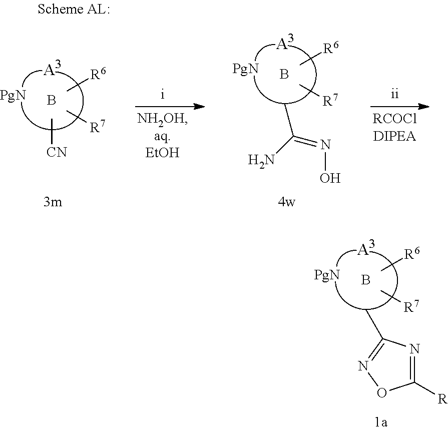

Step i: 3m is converted to the amideoxime 4w using hydroxylamine.

Step ii: 4w is ring closed to form 1a in presence of acid chloride and DIPEA.

### Method AM

Intermediate of formula 1a, wherein A⁴ is an (—CH₂O—) unit between B and R⁸ may be prepared by the method illustrated in Scheme AM.

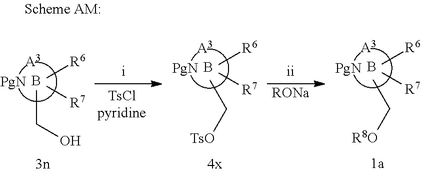

Synthesis i: Compound 3n is converted to 4x using tosyl chloride and pyridine.

Synthesis ii: Compound 4x is transformed to 1a by a substitution of an alkoxide for a tosyl leaving group.

### Method AN

Oxazole intermediates of formula 1a, wherein A⁴ is a bound between B and R⁸ may be prepared in according to procedures described by Wist al. Bioorg. Med. Chem. 2007, 15, 2935-2943; WO2004007529; the disclosures of which are incorporated herein by references. A representative example is illustrated in Scheme AN.

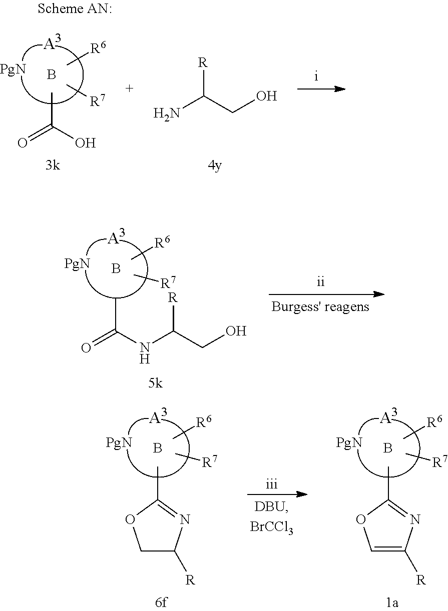

Synthesis i: 3k and 4y are coupled together in an amide coupling reaction to form 5k.

Synthesis ii: In presence of Burgess reagens (methyl(carboxysulfamoyl)-triethylammonium hydroxide) 5k is ring closed to 6f.

Synthesis iii: 6f is oxidized to 1a in presence of DBU and trichlorobromomethan.

### Method AO

Oxazole intermediates of formula 1a, wherein A⁴ is a bound between B and R⁸ may be prepared in according to procedures described by Trukin et al. Synlett 2005, 2072-2076; the disclosures of which are incorporated herein by references. A representative example is illustrated in Scheme AO.

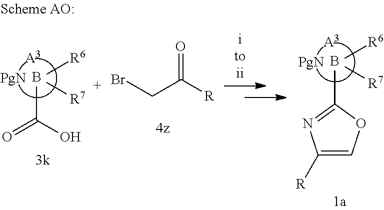

Step i to ii: Using 3k and 4z compound 1a is synthesised with the same method in Method J.

### Method AP

Thiazole intermediates of formula 1a, wherein A⁴ is a bound between B and R⁸ may be obtained from conventional methods known to those skilled in the art, such as Bagley et al. Synthesis 2007, 3535-3541 and Riedrich et al. Angew. Chem. Int. Ed. 2007, 46, 2701-2703; the disclosures of which are incorporated herein by references. A representative method is illustrated in Scheme AP.

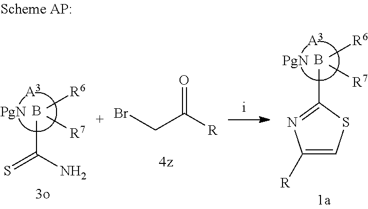

Step i: Using 3o and 4z compound 1a is synthesised in according to step i in Method N.

### Method AQ

Intermediate of formula 1a, wherein A⁴ is a bound between B and R⁸ may be prepared in according to procedures described in WO2005/097791, which is hereby incorporated by reference, as illustrated in Scheme AQ.

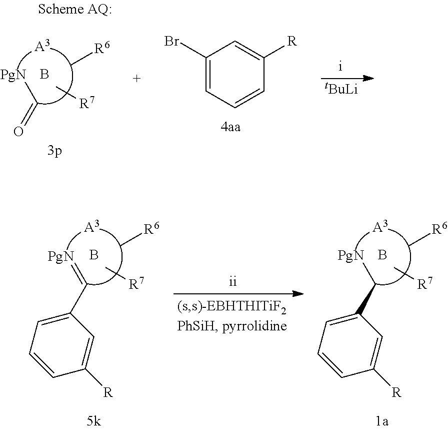

Step i: 4aa was added to 3p in presence of tert-butyl lithium to give the imine 5k.

Step ii: The imine 5k was reduced with a chrial lewis acid such as (ethylenebistetrahydroindenyl)titanium fluoride (EBHTHITiF₂) and a reducing agent such as phenylsilane to give the (s) enantiomer of 1a.

### Preparations of Intermediates 1b

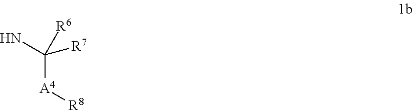

Intermediates of formula 1b are used for preparing compounds of formula (IIb) and (IIIb). These intermediates are acyclic analogues of 1a and may therefore be prepared using the same methods for the preparation of 1a. Examples of acyclic analogues 1b that may be prepared according to the above described methods for 1a, can be seen in scheme AR.

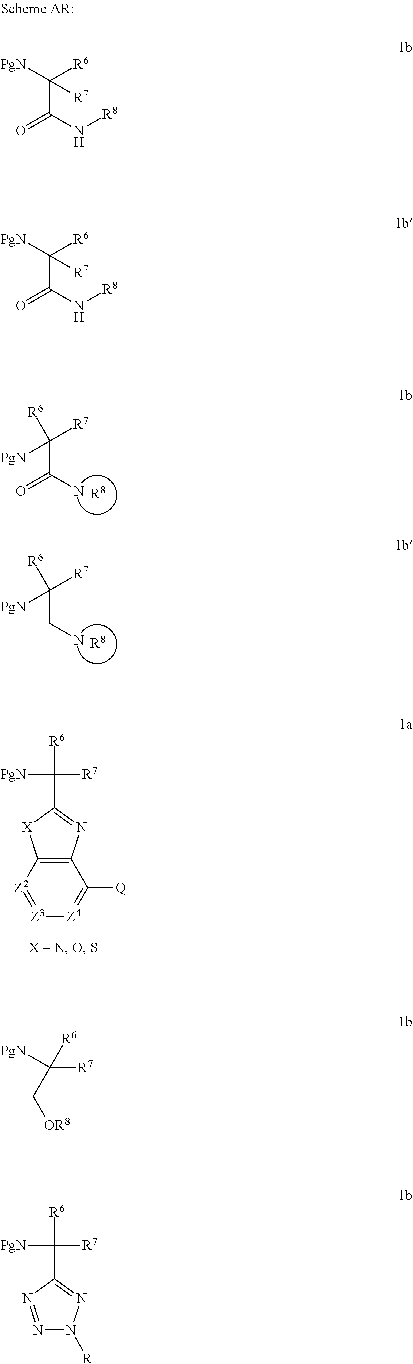

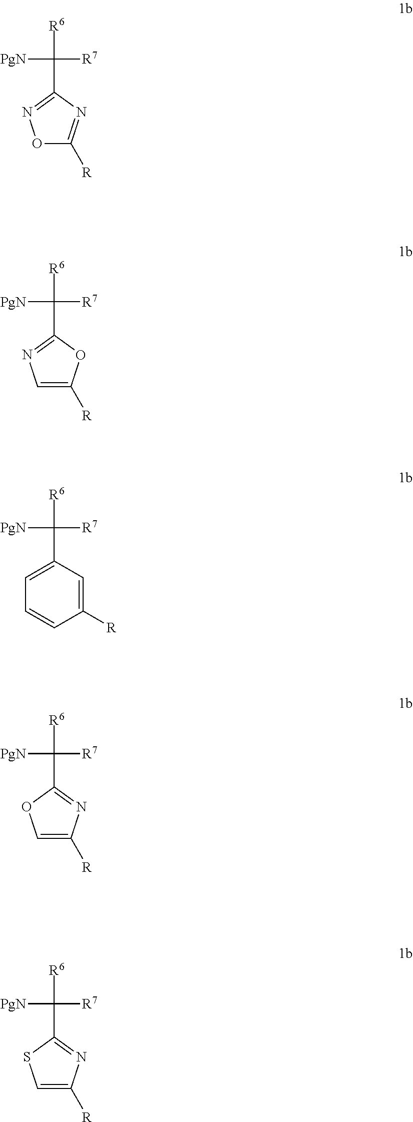

### Preparations of Intermediates 1c

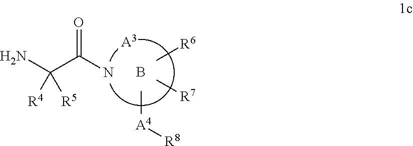

Intermediates of formula 1c, wherein A² is a (—NHCHR⁴R⁵—) moiety are used for preparing compounds of formula (IV) and may be obtained from an amide coupling reaction between starting materials already generated via described methods or commercial available compounds. See scheme AS.

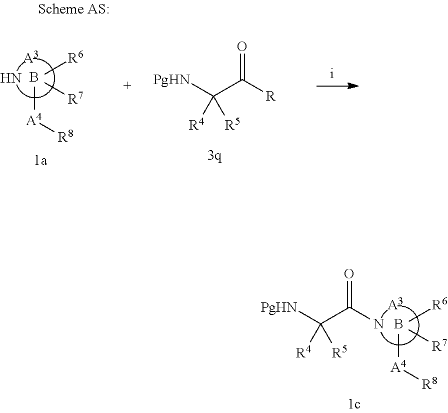

Step i: In this step the compound of formula 1c is prepared by an amide coupling reaction between the compounds of formula 1a with the compound of formula 3q. The preferred conditions for amide coupling reactions are described under general synthesis and can involve both solution and solid supported procedures if 1a is linked to a solid support.

### Preparations of Intermediate 1d

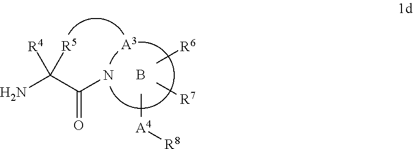

Intermediates of formula 1d are used for preparing compounds of formula (V). These intermediates are analogues of 1a and may therefore be prepared using the same methods for the preparation of 1a when A⁴ is an amide moiety. See scheme AT. The starting material 3r may be prepared according to procedures described by Angiolini et al. Eur. J. Org. Chem. 2000, 2571-2581; Sun et al. J. Med. Chem. 2004, 47, 4147-4150; J. Am. Soc. Chem. 2004, 126, 16686-16687; WO2005/069894; the disclosures of which are incorporated herein by references.

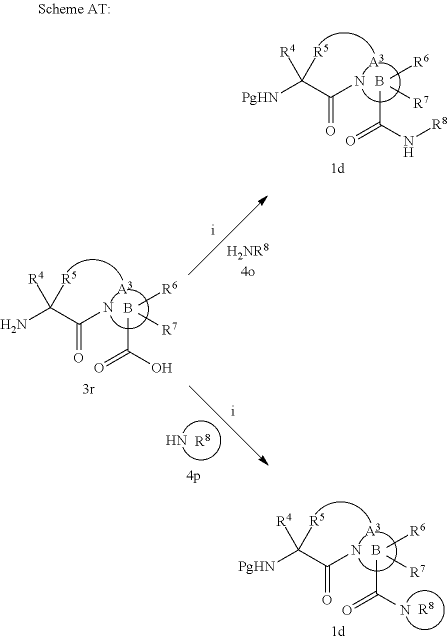

Step i: The compound of formula 1d is prepared by an amide coupling reaction between the compound of formula 3r with the compounds of formula 4o or 4p. The preferred conditions for amide coupling reactions are described under general synthesis and can involve both solution and solid supported procedures if 4o or 4p are linked to a solid support.

### General Synthesis: Polymeric Compounds (VI) and Compounds (VII)

A general procedure for preparation of a homodimer of compounds of formula (I), i.e. a compound of formula (VI) comprising to identical Y and wherein n is 0, is treatment of an intermediate of formula (I) with a suitable reactive handle such as a primary or secondary amine, a —C(O)H, a —COOH or a —OH group with a reactive linker.

A general procedure for preparation of a homomultimer of compounds of formula (I), i.e. a compound of formula (VI) comprising to identical Y and wherein n is an integer from 1-5, is treatment of an intermediate of formula (I) with a suitable reactive handle such as a primary or secondary amine, a —C(O)H, a —COOH or a —OH group; and a protecting group protected suitable reactive handle such as a primary or secondary amine, a —C(O)H, a —COOH or a —OH group with a protecting group with a reactive linker. Then the protecting group is removed and the dimer is treated with a reactive linker providing a trimer of compounds of formula (I), i.e. a compound of formula (VI). Similarly higher-order multimers may be prepared.

A general procedure for preparation of a heterodimer of compounds of formula (I), i.e. a compound of formula (VI) comprising to different Y and wherein n is 0, is treatment of a mixture of intermediates of formula (I) each with a suitable reactive handle chosen from the group consisting of a primary or secondary amine, a —C(O)H, a —COOH or a —OH group with a suitable reactive linker.

A general procedure for preparation of compounds of formula (I) linked to an entity (E), i.e. a compound of formula (VII), is treatment of an entity (E) carrying a suitable reactive handle such as a primary or secondary amine, a —C(O)H, a —COOH or a —OH group with a reactive linker (L) and then treating the E-L compound thus provided with an intermediate of a compound of formula (I) in one or more steps.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form or to any specific optical; or geometric isomer, except where such stereochemistry is clearly defined.

Various scientific articles, patents and other publications are referred to throughout the specification. Each of these publications is incorporated by reference herein in its entirety.

The following Examples illustrate the present invention. It is to be understood, however, that the invention, as fully described herein and as recited in the claims, is not intended to be limited by the details of the following Examples.

### EXAMPLES

In the following examples and preparations, unless stated otherwise, all operations were carried out at room or ambient temperature, that is, in the range of 18-25° C., evaporation of solvent was carried out using a rotary evaporator under reduced pressure with a bath temperature of up to 60° C., reactions were monitored by thin layer chromatography (TLC) and reaction times are given for illustration only; All melting points (mp) were determined in open capillary tubes using a Büchi B-540 Melting Point instrument and are uncorrected (polymorphism may result in different melting points); the structure and purity of all isolated compounds were assured by at least one of the following techniques: TLC (Merck silica gel 60 F₂₅₄ precoated TLC plates), mass spectrometry or nuclear magnetic resonance spectra (NMR). Yields are given for illustrative purposes only. Workup with a cation-exchange column was carried out using MP-TsOH cartridge (Argonaut), which was preconditioned with dichloromethane. Flash column chromatography was carried out using Merck silica gel 60 (63-200 μm) or pre-packed silicagel gel columns (Silicycle) on a combiflash Companion system (Teledyne-Isco). Low-resolution mass spectral data (ESI) were obtained on Waters Micromass HPLC mass analyser. ¹H and ¹³C NMR spectra were obtained at 300 MHz and 75 MHz, respectively, on a Bruker UltraShield 300 instrument. Chemical shifts are reported in part per million (ppm) on the δ scale relatively to the chemical shift of the deuterated solvent; conventional abbreviations used are: s=singlet, d=doublet, t=triplet, q=quartet, quint=quintet, m=multiplet, bs=broad singlet, etc.

All solvents and commercially available compounds were used as received. Parallel synthesis on solid phase was performed in polypropylene filtration tubes with polyethylene frits. Reaction vessels were agitated on a shaker (IKA® KS130 BASIC shaker). The used solid supports are commercially available from Varian, Novabiochem and Biotage

### Synthetic Procedure Abbreviations Used are as Follows:

Boc: tert-butoxycarbonyl;
Bp: boiling point;
n-BuLi: butyl lithium;
DCM: methylene chloride;
DBU: 1,8-diazabicyclo[5.4.0]unddec-7-ene;
DEAD: Diethyl azodicarboxylate;
DhbtOH: 3,4-dihydro-3-hydroxy-4-oxo-1,2,3-benzotriazine;
DIPEA: diisopropylethylamine;
DIC: diisopropylcarbodiimide;

### DMF: N,N-dimethylformamide;

S,S-EBTHITiF₂: ethylenebis(tetrahydroindenyl) titanium fluoride;
EtOAc: ethyl acetate;
eq.: equivalent(s);
Fmoc: 9-fluorenylmethoxycarbonyl;
g: gram(s);
HATU: 2-(7-aza-1H-benzotriazole-1-yl)-1,1,3,3-tetramethyluronium hexafluorophosphate;
LC: liquid chromatography;
MeOH: methanol;
mL: milliliter(s);

### NMM: N-methylmorpholine;

mmol: millimoles;

### NMP: N-methylpyrrolidone;

NMR: nuclear magnetic resonance;
Mp: melting point;
2-NsCl: 2-nitro-benzenesulfonyl chloride;
Ns: 2-nitro-benzenesulfonyl;
PyBOP: benzotriazole-1-yl-oxy-tris-pyrrolidino-phosphonium hexafluorophosphate;
quant.: quantitative yield;
TEA: triethylamine;
TFA: trifluoroacetic acid;
THF: tetrahydrofuran;
TMOF: trimethyl orthoformate;

### Preparation of Intermediates 2a

### Preparation 1

### 6-(1-(2-nitrophenylsulfonamido)ethyl)piperidine-2-carboxylic acid

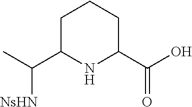

### Step 1. 6-(1-Amino-ethyl)-piperidine-2-carboxylic acid ethyl ester

Synthesised according to Method Q, Step i and ii:

To 6-acetyl-pyridine-2-carboxylic acid methyl ester (0.11 g, 0.63 mmol) dissolved in ethanol-water (1.5 mL, 2:1) was added hydroxylamine hydrochloride (0.043 g, 0.63 mmol) and sodium acetate (0.051 g, 0.63 mmol). The reaction mixture was stirred overnight at 55° C. The reaction was concentrated under reduced pressure and redissolved in DCM (10 mL). The organic phase was washed with water, dried over sodium sulfate, filtered and evaporated under reduced pressure. The residue was dissolved in ethanol (4 mL) followed by the addition of concentrated sulphuric acid (0.1 mL) and 5% rhodium on carbon (0.01 g). The mixture was hydrogenated at 15 bars for two days at room temperature. The reaction mixture was filtered through celite and the solvent evaporated under reduced pressure to afford 94 mg the title compound.

### Step 2. 6-(1-(2-nitrophenylsulfonamido)ethyl)piperidine-2-carboxylic acid

Synthesised according to Method T, Step i:

The amino acid ester (94 mg, 0.47 mmol) was dissolved in dioxan-water (2:1, 15 mL). Sodium carbonate (0.53 g, 5.0 mmol) and 2-nitro-benzenesulfonyl chloride (0.28 g, 1.25 mmol) were added and the temperature kept at 0° C. for 2 hours and at room temperature for 16 hours. The reaction mixture was acidified with 1M sodium hydrogensulfate and diluted with DCM (30 mL). The aqueous phase was collected and rendered alkaline with sodium carbonate and extracted twice with DCM (15 mL). The combined organic layers were dried over sodium sulfate, filtered and evaporated to give the mono nosyl protected amino acid ethyl ester (0.093 g). The ester was dissolved in 4 M HCl in dioxan (5 mL) and water (1 mL) was added. The reaction was stirred overnight at 60° C. The solvents were evaporated in vacuo to afford 86 mg of the title compound. ¹H NMR conforms to structure.

### Preparation 2

### 3-(1-(2-nitrophenylsulfonamideo)ethyl)benzoic acid

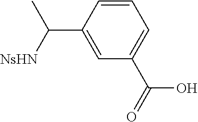

### Step 1. 3-(1-Amino-ethyl)-benzoic acid

Synthesised according to Method P, Step i:

A solution of 3-acetylbenzoic acid (0.164 g, 1.0 mmol) and ammonium formate (0.315 g, 5.0 mmol) in methanol (1 mL) was cooled to −78° C. in a schlenk tube and degassed by three freeze thaw cycles. The schlenk tube was heated to room temperature and dichloro(pentamethylcyclopentadienyl)rhodium(III) dimer (0.031 g, 5.0 μmol) was added. The schlenk tube was closed and the reaction stirred for 3 hours at 50° C. The mixture was cooled to room temperature. The product was filtered off; washed with methanol and dried in vacuo to afford 0.11 g of the title compound.

### Step 2. 3-(1-(2-nitrophenylsulfonamideo)ethyl)benzoic acid

Synthesised according to Method T, Step i:

3-(1-Amino-ethyl)-benzoic acid (0.10 g, 0.61 mmol) was dissolved in dioxan-water (1:1, 6 ml). Sodium carbonate (0.19 g, 1.83 mmol) and 2-nitro-benzenesulfonyl chloride (0.16 g, 0.73 mmol) were added and the temperature kept at 0° C. for 2 hours and at room temperature for 16 hours. The reaction mixture was acidified with 1M sodium hydrogensulfate and diluted with water (10 mL). The aqueous phase was extracted twice with DCM (15 mL). The combined organic extracts were dried over sodium sulfate, filtered and evaporated to give 0.162 g of the title compound. ¹H NMR conforms to structure.

### Preparation 3

### 3-(1-(N-methyl-2-nitrophenylsulfonamideo)ethyl)benzoic acid

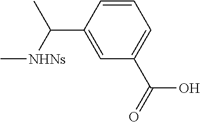

### Step 1. 3-(1-(N-methyl-2-nitrophenylsulfonamideo)ethyl)benzoic acid

Synthesised according to Method T, Step ii:

To a DCM preswollen 2-chloro trityl chloride resin (0.7 mmol) was added a DCM solution (4 mL) of 3-(1-(2-nitrophenylsulfonamideo)ethyl)benzoic acid (0.30 g, 0.85 mmol) followed by DIPEA (0.37 mL, 2.1 mmol). The reaction mixture was agitated overnight at room temperature. The resin was drained and washed with DCM (3×5 mL) and remaining chloride was quenched for 30 minutes with a MeOH-DCM solution (3.5 mL, 1:6). The resin was drained and washed with DCM (3×5 mL), DMF (3×5 mL) and DCM (3×5 mL). The resin was dried overnight at high vacuum. The loading of the resin was quantitative based on the mass increase of the resin.

The resin was suspended in anhydrous DCM (5 mL) followed by addition of triphenyl phosphine (0.92 g, 3.5 mmol), dry MeOH (0.14 mL, 3.5 mmol) and DEAD (0.55 mL, 3.5 mmol). The mixture was agitated for 1 hour at room temperature. The resin was drained and washed with DCM (3×5 mL), DMF (3×5 mL), MeOH (3×5 mL) and DCM (3×5 mL). The resin was cleaved with 5% TFA in DCM for 1 hour. The cleavage solution and the DCM washing solution was collected and co-evaporated with toluene. The title compound was used without further purification (229 mg, 90%).

### Preparation 4a

### 5-[1-(tert-Butoxycarbonyl-methyl-amino)-ethyl]-furan-2-carboxylic acid

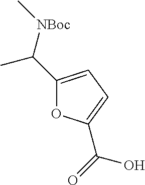

### The Title Compound was Synthesized According to Method R:

### Step 1. 5-Acetyl-furan-2-carboxylic acid ethyl ester

To stirred ice-cold solution of furan-2-carboxylic acid ethyl ester (5 g, 35.7 mmol) in acetic anhydride (12.5 mL, 142.8 mmol) was added BF₃×THF (8.96, 71.4 mmol). After the addition the temperature of the reaction was allowed to reach room temperature and the reaction was stirred for two days. The excess acetic anhydride was first evaporated under reduced pressure. The remaining acetic anhydride was quenched with aqueous sodium hydroxide (30 mL, 1M) and the crude was extracted with ethyl acetate (3×30 mL). The collected organic fractions were washed with brine, dried with sodium sulphate and concentrated in vacuo. The crude was distilled in a kughlrohr at 120-140° C./0.8 mbar to give 0.73 g of the title compound.

### Step 2. 5-(1-Methylamino-ethyl)-furan-2-carboxylic acid isopropyl/ethyl ester

To 5-acetyl-furan-2-carboxylic acid ethyl ester (0.73 g, 4.0 mmol) was added a THF solution of methyl amine (4 mL, 2M) and titanium(IV) isopropoxide (2.37 mL, 8.0 mmol). After the reaction was stirred for 16 hours at room temperature, methanol and sodium borohydride were added and the reaction was stirred at room temperature for another 16 hours. The titanium was quenched by adding a diol silicalgel (10 g, 10.9 mmol) to the reaction mixture followed by DCM (40 mL). The slurry was stirred for 3 hours at 50° C. The slurry was filtered and the organic phase concentrated in vacou. The crude was dissolved in ethyl acetate (20 mL) and washed with aqueous ammonia (20 mL, 10%) followed by extraction with aqueous potassium hydrogensulphate (20 mL, 10%). The acidic aqueous solution was washed with DCM (20 mL) then neutralised with aqueous sodium hydroxide (1M) followed by extraction with DCM (2×20 mL). The combined organic fractions were concentrated in vacou to give a mixture of 5-(1-Methylamino-ethyl)-furan-2-carboxylic acid isopropyl ester and 5-(1-Methylamino-ethyl)-furan-2-carboxylic acid ethyl ester (0.32 g).

### Step 3. 5-[1-(tert-Butoxycarbonyl-methyl-amino)-ethyl]-furan-2-carboxylic acid isopropyl/ethyl ester

To a dioxan solution of 5-(1-Methylamino-ethyl)-furan-2-carboxylic acid isopropyl and ethyl ester (0.3M, 1.5 mmol) was added DIPEA (0.4 mL, 2.3 mmol) and di-tert-butyl-dicarbonate (0.4 g, 1.84 mmol). After stirring overnight at room temperature the reaction was concentrated in vacou and dissolved in ethyl acetate (15 mL). The organic solvent was washed sequentially with aqueous potassium hydrogensulphate (15 mL, 10%), an aqueous saturated solution of sodium hydrogencarbonate (15 mL) and saturated brine (15 mL) followed by drying over sodium sulphate and concentration in vacou. The title compound (0.48 g) was obtained in a quantitative yield and used without further purification.

### Step 4. 5-[1-(tert-Butoxycarbonyl-methyl-amino)-ethyl]-furan-2-carboxylic acid

To the 5-[1-(tert-Butoxycarbonyl-methyl-amino)-ethyl]-furan-2-carboxylic acid isopropyl and ethyl ester (0.48 g, 1.5 mmol) was added a solution of lithium hydroxide (0.11 g, 4.5 mmol) in a mixture of methanol, THF and water (3.6 mL, 1/1/1). After stirring overnight at room temperature the reaction was acidified with aqueous potassium hydrogensulphate (5 mL, 10%) and extracted with DCM (2×10 mL). The collected organic fractions were dried over sodium sulphate and concentrated in vacuo to afford 0.4 g of the title compound.

### Preparation 4b

### 5-{(1R)-1-[(tert-butoxycarbonyl)(methyl)amino]ethyl}furan-2-carboxylic acid

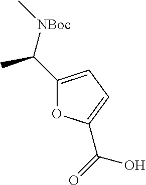

5-{(1R)-1-[(tert-butoxycarbonyl)(methyl)amino]ethyl}furan-2-carboxylic acid is commercially available from Netchem, Inc.

### Preparation 4c

### 5-{(1S)-1-[(tert-butoxycarbonyl)(methyl)amino]ethyl}furan-2-carboxylic acid

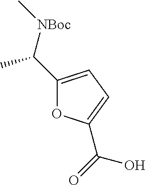

5-{(1S)-1-[(tert-butoxycarbonyl)(methyl)amino]ethyl}furan-2-carboxylic acid is commercially available from Netchem, Inc.

### Preparation 5a

### (1S)-5-[1-(2-Nitro-benzenesulfonylamino)-ethyl]-furan-2-carboxylic acid

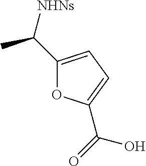

The chiral title compound is synthesised according to Chakraborty et al. Synlett, 2004, 2484-2488 and Ns protected according to the procedure described in Step 2 of Preparation 2.

### Preparation 5b

### 5-{(1R)-1-[(tert-butoxycarbonyl)amino]ethyl}furan-2-carboxylic acid

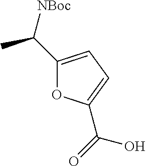

5-{(1R)-1-[(tert-butoxycarbonyl)amino]ethyl}furan-2-carboxylic acid is commercially available from Netchem, Inc.

### Preparation 5c

### 5-{(1S)-1-[(tert-butoxycarbonyl)amino]ethyl}furan-2-carboxylic acid

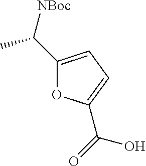

5-{(1S)-1-[(tert-butoxycarbonyl)amino]ethyl}furan-2-carboxylic acid is commercially available from Netchem, Inc.

### Preparation 6

### (1S)-5-{1-[Methyl-(2-nitro-benzenesulfonyl)-amino]-ethyl}-furan-2-carboxylic acid

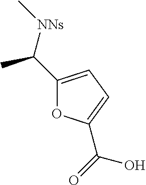

The chiral title compound is synthesised according to Preparation 5 and methylated according to the procedure described in Step 1 of Preparation 3.

### Preparation 7

### 6-[1-(tert-Butoxycarbonyl-methyl-amino)-ethyl]-pyridine-2-carboxylic acid

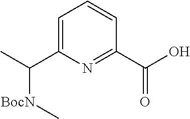

The title compound was prepared according to the procedure described in Step 2 to 4 of Preparation 4a from the commercial available methyl 6-acetylpicolinate.

### Preparation 8a

### 3-[1-(tert-Butoxycarbonyl-methyl-amino)-ethyl]-benzoic acid

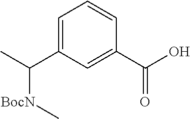

The title compound was prepared from methyl 3-acetylbenzoate according to the procedure described in Step 2 to 4 of Preparation 4a from the commercial available methyl 3-acetylbenzoate.

### Preparation 8b

### 3-{(1R)-1-[(tert-butoxycarbonyl)(methyl)amino]ethyl}benzoic acid

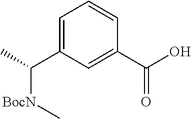

3-{(1R)-1-[(tert-butoxycarbonyl)(methyl)amino]ethyl}benzoic acid is commercially available from Netchem, Inc.

### Preparation 8c

### 3-{(1S)-1-[(tert-butoxycarbonyl)(methyl)amino]ethyl}benzoic acid

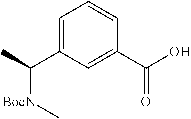

3-{(1S)-1-[(tert-butoxycarbonyl)(methyl)amino]ethyl}benzoic acid is commercially available from Netchem, Inc.

### Preparation of Intermediates 2b

### Preparation 9

### 2,8-Diaza-spiro[4.5]decane-2,3,8-tricarboxylic acid 8-tert-butyl ester 2-(9H-fluoren-9-ylmethyl) ester

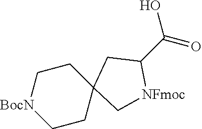

2,8-Diaza-spiro[4.5]decane-2,3,8-tricarboxylic acid 8-tert-butyl ester 2-(9H-fluoren-9-ylmethyl)ester is commercially available from Syntech, Inc.

### Preparation of Intermediates 2c

### Preparation 10

### 2-[[2-(9H-Fluoren-9-ylmethoxycarbonylamino)-butyl]-(2-nitro-benzenesulfonyl)-amino]-3-furan-2-yl-propionic acid

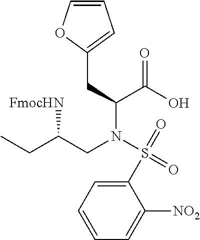

### Step 1-5. 2-[[2-(9H-Fluoren-9-ylmethoxycarbonylamino)-butyl]-(2-nitro-benzenesulfonyl)-amino]-3-furan-2-yl-propionic acid

Synthesised according to Method Z:

To a preswollen 2-chloro trityl chloride resin in DCM (1.4 mmol) was added a DCM solution (7 mL/g resin) of 2-(9H-Fluoren-9-ylmethoxycarbonylamino)-3-furan-2-yl-propionic acid (0.40 g, 1.05 mmol) followed by DIPEA (0.55 mL, 3.15 mmol). The reaction mixture was agitated overnight at room temperature. The resin was drained and washed with DCM (3×10 mL) and remaining chloride was quenched for 30 minutes with a MeOH-DCM solution (7 mL, 1:6). The resin was drained and washed with DCM (3×10 mL), DMF (3×10 mL) and DCM (3×10 mL) successively. The resin was dried overnight at high vacuum. The loading of the resin was quantitative based on the mass increase of the resin.

The Fmoc-amino acid resin (0.7 mmol) was Fmoc deprotected and preswollen in DCM followed by addition of a DCM solution (5 mL) of 2-nitro-benzenesulfonyl chloride (0.78 g, 3.5 mmol) and NMM (0.77 mL, 7.0 mmol). The reaction mixture was agitated for 3 hours at room temperature followed by washing with DCM (3×10 mL), DMF (3×10 mL) and DCM (3×10 mL). The resin (0.35 mmol) was suspended in anhydrous THF (3.6 mL) followed by addition of triphenyl phosphine (0.28 g, 1.05 mmol), (1-hydroxymethyl-propyl)-carbamic acid 9H-fluoren-9-ylmethyl ester (0.34, 1.05 mmol) and DEAD (0.17 mL, 1.05 mmol). The mixture was agitated for 2 hour at room temperature. The resin was drained and washed with DCM (3×5 mL), DMF (3×5 mL), MeOH (3×5 mL) and DCM (3×5 mL) successively. The Mitsunobu reaction was repeated two times. The product was cleaved from the resin with 5% TFA in DCM for 1 hour, filtered off and the resin washed with DCM. The combined DCM solution was co-evaporated with toluene. The crude product was purified using flash chromatography with silicagel as absorbent and gradient elution (DCM to 5% MeOH in DCM) to afford the product (89 mg, 40%).

### Preparation 11

### (S)-3-(3-cyanophenyl)-2-((S)-2-(N-methyl-2-nitrophenylsulfonamideo)-butanamideo)propanoic acid

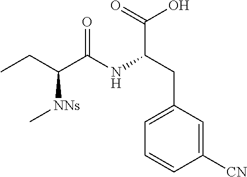

### Step 1-7. (S)-3-(3-cyanophenyl)-2-((S)-2-(N-methyl-2-nitrophenylsulfonamideo)-butanamideo)-propanoic acid

Synthesised according to Method X and Z:

To a DCM preswollen 2-chloro trityl chloride resin (1.4 mmol) was added a DCM solution (7 mL) of (S)-2-(((9H-fluoren-9-yl)methoxy)carbonyl)-3-(3-cyanophenyl)-propanoic acid (0.70 g, 1.70 mmol) followed by DIPEA (0.73 mL, 4.2 mmol). The reaction mixture was agitated overnight at room temperature. The resin was drained and washed with DCM (3×10 mL) and the chloride on the resin was quenched for 30 minutes with a MeOH-DCM solution (7 mL, 1:6). The resin was drained and washed with DCM (3×10 mL), DMF (3×10 mL) and DCM (3×10 mL). The resin was dried overnight at high vacuum. The loading of the resin was quantitative based on the mass increase of the resin.

The resin (0.7 mmol) was washed with DMF and Fmoc deprotected with 20% piperidine. A solution of (S)-2-(((9H-fluoren-9-yl)methoxy)carbonyl)butanoic acid (0.46 g, 1.40 mmol), PyBOP (0.73 g, 1.40 mmol), and DIPEA (0.73 mL, 4.2 mmol) in DMF/DCM (5 mL, 1:1) was added to a pre-swollen resin and the resulting mixture was agitated overnight at room temperature. After draining and washing with DCM (3×5 mL), DMF (3×5 mL) and DCM (3×5 mL) successively the ninhydrin test was negative. The resin was Fmoc deprotected and preswollen in DCM followed by addition of a DCM solution (5 mL) of 2-nitro-benzenesulfonyl chloride (0.78 g, 3.5 mmol) and NMM (0.77 mL, 7 mmol). The reaction mixture was agitated for 3 hours at room temperature followed by wash with DCM (3×5 mL), DMF (3×5 mL) and DCM (3×5 mL) successively. A small sample was cleaved and analysed by LC-MS. No starting material was observed and the major peak was the expected product. The resin was suspended in anhydrous DCM (5 mL) followed by addition of triphenyl phosphine (0.92 g, 3.5 mmol), dry MeOH (0.14 mL, 3.5 mmol) and DEAD (0.55 mL, 3.5 mmol). The mixture was agitated for 1 hour at room temperature. The resin was drained and washed with DCM (3×10 mL), DMF (3×5 mL), MeOH (3×5 mL) and DCM (3×5 mL) successively. The resin was cleaved with 5% TFA in DCM for 1 hour. The cleavage solution and the DCM washing solution was collected and co-evaporated with toluene. The crude product was purified using flash chromatography with silicagel as absorbent and gradient elution (DCM to 5% MeOH in DCM) to afford 142 mg, (43%) of the title compound as a transparent oil.

### Preparation of R⁸ Intermediates

### Preparation 12

### 1-phenyl-1H-pyrazol-5-amine

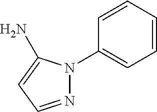

Title compound is commercially available from TCI America (catalog #A0174).

### Preparation 13

### 3-methyl-1-phenyl-1H-pyrazol-5-amine

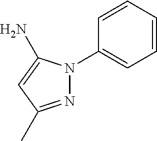

Title compound is commercially available from TCI America (catalog #A1311).

### Preparation 14

### 5-phenylthiazole-2,4-diamine

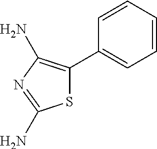

Title compound is commercially available from Acros Organics (catalog #11234-0010).

### Preparation 15

### 5-(trifluoromethyl)-4-phenylthiophen-3-amine

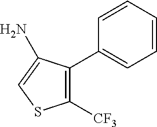

Title compound is commercially available from Acros Organics (catalog* SEWO3133DA).

### Preparation 16

### 4-phenyl-1H-pyrazol-3-amine

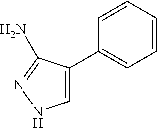

The title compound is synthesised according to E. L. Anderson et al; J. Med. Chem., 1964, 7, 259-268.

### Preparation 17

### 5-methyl-4-phenyl-1H-pyrazol-3-amine

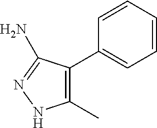

The title compound is synthesised according to E. L. Anderson et al; J. Med. Chem., 1964, 7, 259-268

### Preparation 18

### 3-phenyl-3H-1,2,3-triazol-4-amine

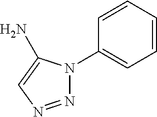

The title compound is synthesised according to K. M. Baines, T. W. Rourke, K. Vaughan; J. Org. Chem., 1981, 46, 856-859.

### Preparation 19

### 4-phenyl isoxazol-5-amine

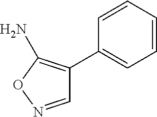

The title compound is synthesised according to H. Peeters, W. Vogt; EP 43024.

### Preparation 20

### 3-phenyl-1H-pyrazol-4-amine 3-phenyl-1H-pyrazol-4-amine

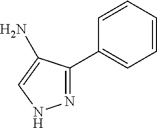

The title compound is synthesised according to C. Chen, K. Wilcoxen, J. R. McCarthy; Tetrahedron Lett., 1988, 39, 8229-8232.

### Preparation 21

### 1-methyl-3-phenyl-1H-pyrazol-4-amine

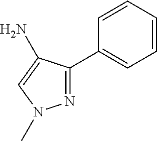

The title compound is synthesised according to C. Chen, K. Wilcoxen, J. R. McCarthy; Tetrahedron Lett., 1988, 39, 8229-8232.

### Preparation 22

### 1-methyl-5-phenyl-1H-pyrazol-4-amine

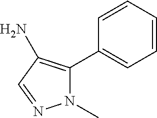

The title compound is synthesised according to C. Chen, K. Wilcoxen, J. R. McCarthy; Tetrahedron Lett, 1988, 39, 8229-8232.

### Preparation 23

### 3-methyl-4-phenylisoxazol-5-amine

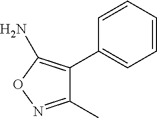

The title compound is synthesised according to H. Peeters, W. Vogt; EP 43024.

### Preparation 24

### 1-phenyl-1H-tetrazol-5-amine

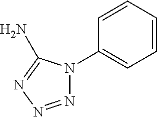

The title compound is synthesised according to R. A. Batey, D. A. Powell; Org. Lett., 2000, 2, 3237-3240.

### Preparation 25

### 4-phenyl-1,2,5-oxadiazol-3-amine

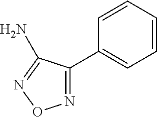

The title compound is synthesised according to R. Lakhan, O. P. Singh; Ind. J. Chem., 1987, 26B, 690-692.

### Preparation 26

### 1-amino-5-phenyl-1H-tetrazole

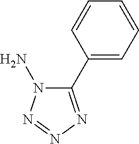

The title compound is synthesised according to T. L. Gilchrist, G. E. Gymer, C. W. Rees;/. Chem. Soc, Perkin Trans. 1, 1975, 1747-1750.

### Preparation 27

### 4-amino-3-phenyl-4H-1,2,4-triazole

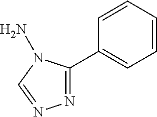

The title compound is synthesised according to A. A. B\acute{α}zler, N. Yildirim; J. Heterocyclic Chem., 1998, 35, 377-380.

### Preparation 28

### 3-phenylthiophen-2-amine

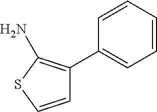

The title compound is synthesised according to Y. Yoshikawa et al; EP 737682 (U.S. Pat. No. 5,747,518).

### Preparation 29

### 2-phenylthiophen-3-amine

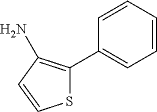

The title compound is synthesised according to Y. Yoshikawa et al; EP 737682 (U.S. Pat. No. 5,747,518).

### Preparation 30

### 4-phenylthiophen-3-amine

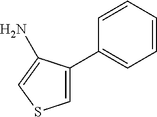

The title compound is synthesised according to G. Kirsch, D. Cagniant, P. Cagniant; J. Heterocyclic Chem., 1982, 19, 443-445.

### Preparation 31

### 5-amino-4-phenylthiazole-2-thiol

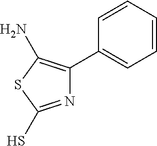

The title compound is synthesised according to A. H. Cook, I. Heilbron, A. L. Levy; J. Chem. Soc, 1947, 1598-1609.

### Preparation 32

### 2-(methylthio)-4-phenylthiazol-5-amine

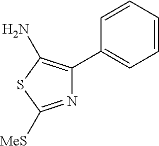

The title compound is synthesised according to A. H. Cook, I. Heilbron, A. L. Levy; J. Chem. Soc, 1947, 1598-1609.

### Preparation 33

### 5-amino-2-(methylsulfinyl)-4-phenylthiazole

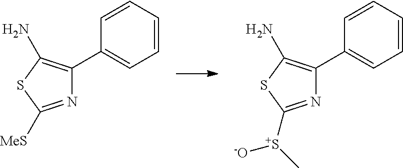

The title compound is synthesised according to WO2006/014361

### Preparation 34

### 5-amino-2-(methylsulfonyl)-4-phenylthiazole

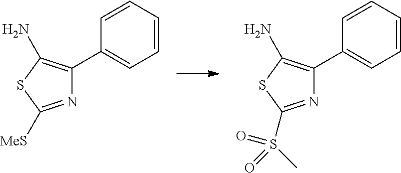

The title compound is synthesised according to WO2006/014361

### Preparation 35

### 5-amino-2-(tert-butylsulfanyl)-4-phenylthiazole

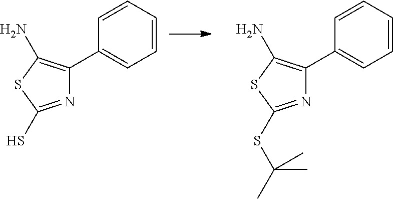

To a suspension of 5-amino-2-mercapto-4-phenylthiazole (210 mg, 1.01 mmol) in water (1.0 ml) and tert-butanol (82 mg, 1.1 mmol) is added concentrated sulfuric acid (3.0 ml) with cooling to ca. 20° C. After 1.5 hr at ambient temperature a further portion of tert-butanol in water (300 μl, 1.0 M, 300 μmol) is added. After 1.5 hr the mixture is poured into excess aqueous sodium bicarbonate and extracted three times into dichloromethane (total 120 ml). The combined organic phases are washed with brine, dried over sodium sulfate, filtered and concentrated in vacuo. Flash chromatography on silica gel (ethyl acetate/hexanes) yields 5-amino-2-(tert-butylsulfanyl)-4-phenylthiazole (220 mg, 82%).

### Preparation 36

### 5-amino-2-(tert-butylsulfinyl)-4-phenylthiazole

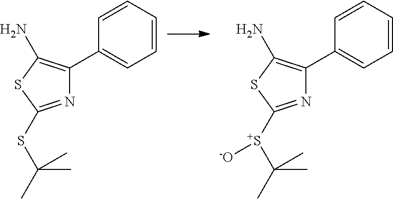

To 5-amino-2-(tert-butylsulfanyl)-4-phenylthiazole (102 mg, 385 μmol) in acetic acid (5.0 ml) is added aqueous hydrogen peroxide (218 μl, 30% wt, 1.9 mmol) dropwise at ambient temperature. After 5 hr the mixture is partitioned between dichloromethane (50 ml) and water (50 ml). The aqueous phase is separated and extracted with dichloromethane (20 ml). The combined organic phases are washed with saturated aqueous sodium bicarbonate, dried over sodium sulfate, filtered and concentrated in vacuo to yield essentially pure 5-amino-2-(tert-butylsulfinyl)-4-phenylthiazole (110 mg, quant.).

### Preparation 37

### 3-amino-4-phenyl-1,2,5-thiadiazole

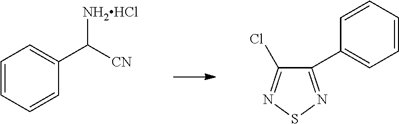

To a solution of sulfur monochloride (24.0 g, 178 mmol) in DMF (30 ml) at 0° C. is added α-aminophenylacetonitrile hydrochloride (10.0 g, 59.3 mmol) portionwise over 20 min. After 40 min the mixture is allowed to warm to ambient temperature for 20 min, diluted with DMF (20 ml) and stirred for a further 20 hr before pouring into ice-water. The mixture is extracted with ether (200 ml), filtered, and the extracted twice more with ether (2×50 ml). The combined organic phases are washed with brine, dried over magnesium sulfate and concentrated in vacuo to give 3-chloro-4-phenyl-1,2,5-thiadiazole as a mobile orange oil (10.1 g, 87%). Short-path distillation of this oil (9.35 g) at reduced pressure yields a clear, colorless oil (7.75 g, 83%) which crystallized on standing.

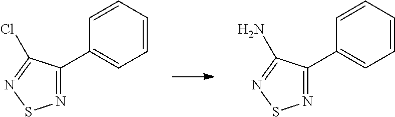

3-Chloro-4-phenyl-1,2,5-thiadiazole (3.19 g, 16.2 mmol) in THF (32 ml) at 0° C. is treated dropwise with a solution of lithium bis(trimethylsilyl)amide in THF (17.0 ml, 1.0 M, 17.0 mmol). After 10 min the mixture is allowed to warm to ambient temperature for 1.5 hr, treated with 1N hydrochloric acid, and extracted three times into ether (total 300 ml). The combined organic phases are washed with saturated aqueous sodium bicarbonate and brine, and dried over magnesium sulfate and concentrated in vacuo. The residue is dissolved in methanol (50 ml) and triethylamine (0.5 ml) is heated to reflux for 15 hr and again concentrated in vacuo. Flash chromatography on silica gel (ethyl acetate/hexanes) yields 3-amino-4-phenyl-1,2,5-thiadiazole (1.96 g, 68%) as a colorless solid.

### Preparation 38

### 5-amino-2-methyl-4-phenylthiazole

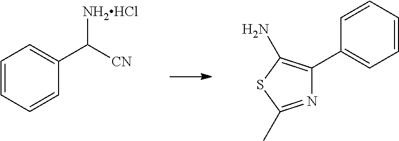

To a suspension of α-aminophenylacetonitrile hydrochloride (3.37 g, 20.0 mmol) and powdered sulfur (641 mg, 20.0 mmol) in ethanol (20 ml) at 0° C. is added triethylamine (4.18 ml, 30.0 mmol) and then acetaldehyde (2.3 ml, 41 mmol). The vessel is sealed and heated to 60-700 C for 1 hr. The cooled mixture is filtered and concentrated in vacuo, and the residue treated with ethanol (20 ml) and hydrochloric acid (20 ml, 1N) for 15 hr. The mixture is treated with aqueous sodium carbonate and extracted three times into ethyl acetate (total 300 ml). The combined organic phases are washed with brine, dried over sodium sulfate and concentrated in vacuo to give a dark brown oil. Flash chromatography on silica gel (ethyl acetate/hexanes) yields 5-amino-2-methyl-4-phenylthiazole (1.31 g, 34%), which crystallized from toluene.

### Preparation 39

### 5-amino-2-methyl-4-phenylthiazole

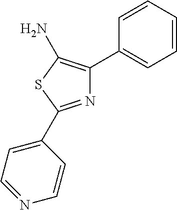

A suspension of α-aminophenylacetonitrile hydrochloride (1.69 g, 10.0 mmol), powdered sulfur (321 mg, 10.0 mmol) and 4-pyridinecarboxaldehyde (1.91 ml, 20.0 mmol) in ethanol (10 ml) is treated with triethylamine (2.09 ml, 15.0 mmol), and the mixture stirred at 500 C for 80 min. The cooled mixture is diluted with ethanol (5 ml) and treated with aqueous hydroxylamine (700 μl, 50% wt, 11 mmol) at ambient temperature for 15 hr, and diluted with dichloromethane (50 ml). Saturated aqueous sodium bicarbonate is added and the separated aqueous phase is extracted twice more with dichloromethane (total 100 ml). The combined organic phases are dried over sodium sulfate and concentrated in vacuo to give a dark brown oily foam (3.23 g). Flash chromatography on silica gel (ethyl acetate/hexanes) yields 5-amino-2-(4-pyridyl)-4-phenylthiazole (1.41 g, 56%).

### Preparation 40

### 2,4-diphenylthiazol-5-amine

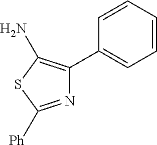

The title compound is synthesised according to K. Gewald, H. Schonfelder, U. Hain; J. Prakt. Chem., 1974, 361, 299-303.

### Preparation 41

### 4-phenyl-2-(pyridin-2-yl)thiazol-5-amine

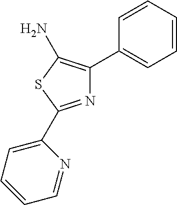

The title compound is synthesised according to K. Gewald, H. Schonfelder, U. Hain; J. Prakt. Chem., 1974, 361, 299-303.

### Preparation 42

### 4-phenyl-2-(pyridin-3-yl)thiazol-5-amine

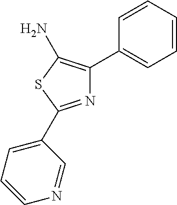

The title compound is synthesised according to K. Gewald, H. Schonfelder, U. Hain; J. Prakt. Chem., 1974, 361, 299-303.

### Preparation 43

### 5-amino-2-(Fmoc-amino)-4-phenylthiazole

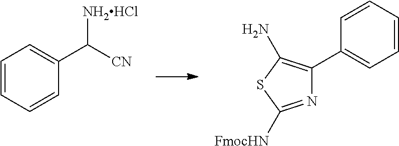

A suspension of α-aminophenylacetonitrile hydrochloride (3.19 g, 18.9 mmol) and Fmoc-isothiocyanate (5.31 g, 18.9 mmol) in DCM is treated with ethyldiisopropylamine (3.62 ml, 20.8 mmol) at 0° C. for 1 hr and then at ambient temperature for 3 hr. The mixture is poured into saturated aqueous sodium bicarbonate and extracted three times into ethyl acetate. The combined organic phases are washed with water and brine, and dried over sodium sulfate and concentrated in vacuo. Flash chromatography on silica gel (ethyl acetate/hexanes) yields 5-amino-2-(Fmoc-amino)-4-phenylthiazole (3.75 g, 48%).

### Preparation 44

### 5-amino-2-(1-imidazolyl)-4-phenylthiazole

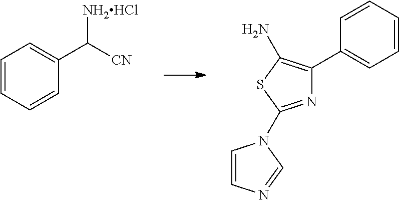

A suspension of α-aminophenylacetonitrile hydrochloride (5.01 g, 29.7 mmol) and thiocarbonyl diimidazole (5.30 g, 29.7 mmol) in DCM (100 ml) is treated with ethyldiisopropylamine (5.69 ml, 32.7 mmol) at 0° C. for 15 min and then at ambient temperature for 3 hr. The mixture is poured into saturated aqueous sodium bicarbonate (50 ml) and water (150 ml), and extracted three times into dichloromethane (total 300 ml). The combined organic phases are washed with brine, dried over magnesium sulfate and concentrated in vacuo to give dark brown oil (8.18 g). Flash chromatography on silica gel (ethyl acetate/hexanes) yields 5-amino-2-(1-imidazolyl)-4-phenylthiazole (2.47 g, 34%).

### Preparation 45

### 2-(acetylamino)-4-amino-5-phenylthiazole

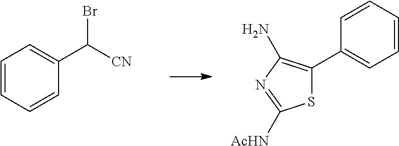

α-Bromophenylacetonitrile (1.08 g, 5.48 mmol) in ethanol (10 ml) is treated with N-acetylthiourea (649 mg, 5.49 mmol) at ambient temperature for 4 hr, and then heated to reflux for 3.5 hr. The cooled mixture is concentrated in vacuo and then partitioned between dichloromethane and saturated aqueous sodium bicarbonate. The organic phase is washed with brine, dried over sodium sulfate, filtered and concentrated in vacuo. Flash chromatography on silica gel (ethyl acetate/hexanes) yields 2-(acetylamino)-4-amino-5-phenylthiazole (295 mg, 23%).

### Preparation 46

### 2,5-diphenylthiazol-4-amine

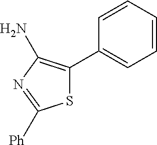

The title compound is prepared using the same procedures described in Preparation 45.

### Preparation 47

### 5-phenyl-2-(pyrazin-2-yl)thiazol-4-amine

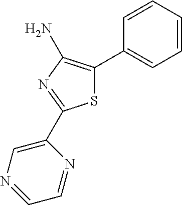

The title compound is prepared using the same procedures described in Preparation 45.

### Preparation 48

### 5-amino-1-(3′-nitrophenyl)pyrazole

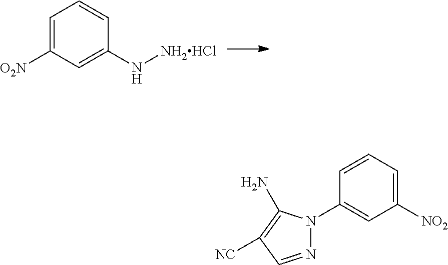

3-Nitrophenylhydrazine hydrochloride (7.03 g, 36.3 mmol), diisopropylethylamine (9.5 ml, 54.5 mmol), and ethanol (60 ml) are stirred under nitrogen at room temperature for 2 h. Ethoxymethylenemalononitrile (4.52 g, 36.3 mmol) is added, after which the reaction is refluxed for 1 h. Reaction is cooled to room temperature. The solvent is removed under reduced pressure until precipitate crashed out. The solid is filtered to yield 6.54 g of the cyclized product (78% yield).

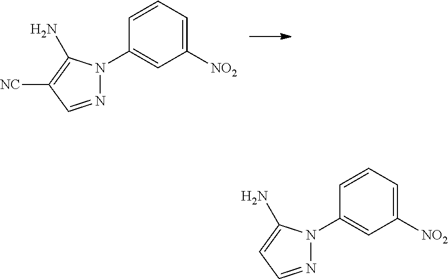

5-amino-1-(3′-nitrophenyl)-4-cyanopyrazole (559 mg, 2.44 mmol) and phosphoric acid (86%, 6 ml) are refluxed at 170° C. for 15 h. The reaction is cooled to room temperature and neutralized with ammonium hydroxide. The organics are extracted three times with diethyl ether (total 40 ml), washed with brine, and dried over magnesium sulfate. Removal of solvent gives 5-amino-1-(3′-nitrophenyl)-pyrazole as a yellow powder (398 mg, 80% yield).

### Preparation 49

### 1-(2-fluorophenyl)-1,1-pyrazol-5-amine

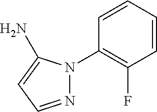

The title compound is prepared using the same procedures described in Preparation 48.

### Preparation 50

### 1-(3-chlorophenyl)-1,1-pyrazol-5-amine

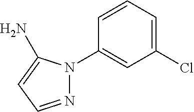

The title compound is prepared using the same procedures described in Preparation 48.

### Preparation 51

### 1-(3-fluorophenyl)-1,1-pyrazol-5-amine

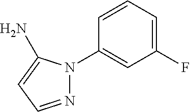

The title compound is prepared using the same procedures described in Preparation 48.

### Preparation 52

### 1-(3-bromophenyl)-1,1-pyrazol-5-amine

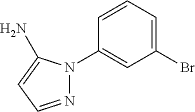

The title compound is prepared using the same procedures described in Preparation 48.

### Preparation 53

### 1-(3-trichloromethyl phenyl)-1,1-pyrazol-5-amine

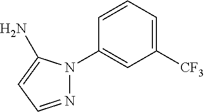

The title compound is prepared using the same procedures described in Preparation 48.

### Preparation 54

### 1-(pyridin-2-yl)-1,1-pyrazol-5-amine

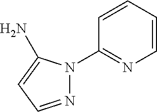

The title compound is prepared using the same procedures described in Preparation 48.

### Preparation 55

### 1-(3-methoxyphenyl)-1,1-pyrazol-5-amine

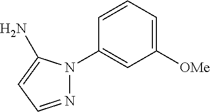

The title compound is isolated following decyanation of 5-amino-4-cyano-1-(3′-methoxyphenyl)pyrazole in Preparation 48.

### Preparation 56

### 1-(3-hydroxyphenyl)-1,1-pyrazol-5-amine

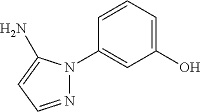

The title compound is prepared using the same procedures described in Preparation 48.

### Preparation 57

### 4-amino-5-phenyl-1,2,3-thiadiazole

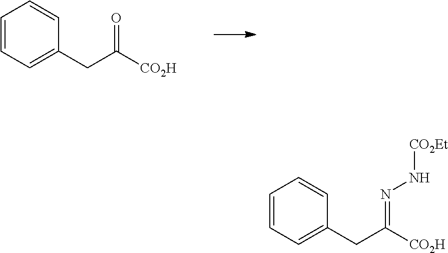

Phenylpyruvic acid (25 g, 149 mmol) and ethyl carbazate (16 g, 149 mmol) are refluxed in benzene (225 ml) for 2 hr, and the mixture concentrated in vacuo. The crude is dissolved in minimum warm dichloromethane to yield the hydrazone as a yellow precipitate upon cooling to ambient temperature, isolated by filtration (30.4 g, 81%) and used without further purification.

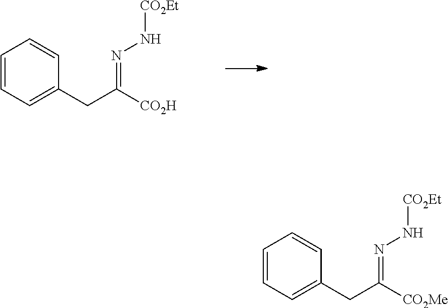

Diazomethane is generated by adding a solution of Diazald (N-methyl-N-nitroso-p-toluenesulfonaraide; 18.6 g, 86.9 mmol) in diethyl ether (180 ml) to a solution of potassium hydroxide (18.2 g, 325 mmol) in water (37 ml) and 2-(2-ethoxyethoxy)-ethanol (37 ml) at 65° C., dropwise over 45 min. Distillation thus produced an ethereal solution of diazomethane which is added directly to a stirred solution of the hydrazone (10.9 g, 43.5 mmol) in methanol (150 ml) at 0° C. The system is rinsed with excess diethyl ether until distillate became clear, the mixture treated with acetic acid (1 ml), and concentrated in vacuo. The resulting oil is partitioned between ethyl acetate (200 ml) and sodium bicarbonate (200 ml), and the organic phase dried over sodium sulfate. Filtration and concentration in vacuo yields the methyl ester as a yellow solid (10.2 g, 89%).

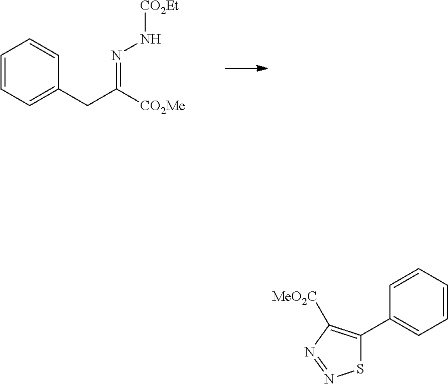

The hydrazone-methyl ester (10.2 g, 38.6 mmol) is treated with thionyl chloride (25 ml, 343 mmol) at ambient temperature for 24 hr, and the mixture concentrated in vacuo. Crystallization from hexanes yields the thiadiazole-methyl ester (4.81 g, 56%).

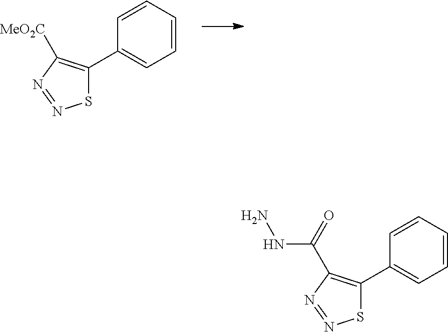

The thiadiazole-methyl ester (2.79 g, 12.7 mmol) is treated with hydrazine hydrate (1.09 ml, 93.9 mmol) in methanol (50 ml) at ambient temperature for 24 hr, and the resulting white precipitate recovered by filtration. Recrystallization from isopropanol yields the thiadiazole-hydrazide (3.99 g, 83%).

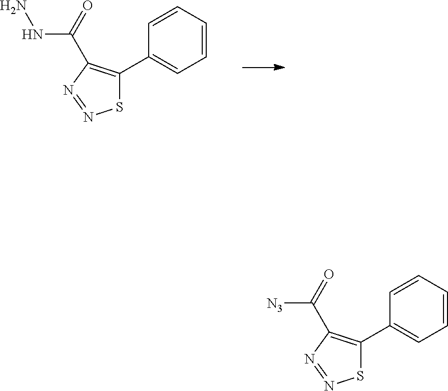

The thiadiazole-hydrazide (3.99 g, 18.1 mmol) in water (40 ml) and concentrated hydrochloric acid (1.8 ml, 21.9 mmol) is treated dropwise with a solution of sodium nitrite (1.52 g, 21.3 mmol) in water (15 ml) at 0° C. for 2 hr. The resulting precipitate is recovered by filtration to yield the thiadiazole-acid azide as an off-white solid (3.95 g, 94%).

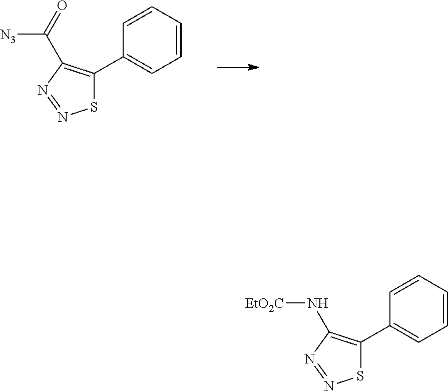

According to the procedures described in K. Masuda et at; Chem. Pharm. Bull., 1981, 29, 1743-1747, the thiadiazole-acid azide (3.95 g, 17.1 mmol) is at reflux in ethanol (40 ml) for 45 min, and the mixture concentrated in vacuo. Crystallization from benzene yields the ethyl carbamate (3.37 g, 74%).

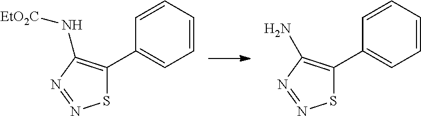

The ethyl carbamate (399 mg, 1.60 mmol) and hydrogen bromide in acetic acid (3 ml, 30% wt) are heated in a sealed vessel at 80° C. for 18 hr. The cooled mixture is partitioned between ethyl acetate (15 ml) and water (15 ml), and the organic phase concentrated in vacuo. Flash chromatography on silica gel (ethyl acetate/hexanes) yields 4-amino-5-phenyl-1,2,3-thiadiazole (136 mg, 49%).

### Preparation 58

### 4-amino-5-phenylisoxazole

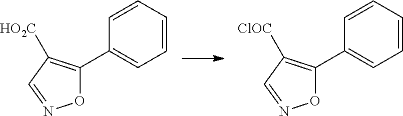

5-Phenyl-4-isoxazolecarboxylic acid (460 mg, 2.36 mmol) and thionyl chloride (1.71 ml, 23.6 mmol) are heated at reflux for 3 hr, and the mixture concentrated in vacuo to yield the acid chloride which is used without purification.

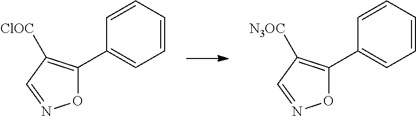

The crude acid chloride in acetone (7 ml) is treated with a solution of sodium azide (165 mg, 2.62 mmol) in water (2 ml) at 0° C. for 1.5 hr, and allowed to warm to ambient temperature and concentrated in vacuo. The resulting white solid is washed with water and dried in vacuo, and used without purification.

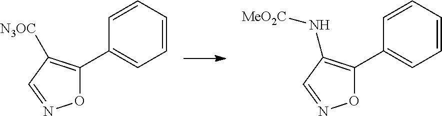

The acid azide (409 mg, 1.91 mmol) is heated at reflux in methanol for 6 hr, and the mixture concentrated in vacuo to yield the methyl carbamate as a white solid, used without purification.

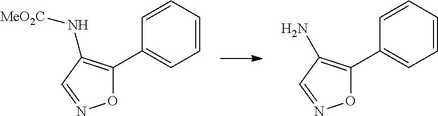

The methyl carbamate (378 mg, 1.73 mmol) is treated with hydrobromic acid (13 ml, 48% wt, 115 mmol), made homogeneous with acetic acid (2 ml), and heated at 65° C. for 48 hr, and allowed to cool. The mixture is neutralized with aqueous sodium hydroxide and extracted with ethyl acetate (2×125 ml). The combined organic phases are dried over sodium sulfate and concentrated in vacuo to yield 4-amino-5-phenylisoxazole as a white solid (193 mg, 70%).

### Preparation 59

### 5-amino-2-t-butyl-4-phenylthiazole

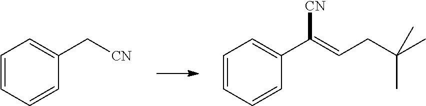

Benzyl cyanide (2.33 ml, 20 mmol) is treated with Verkade's base (2,8,9-trimethyl-2,5,8,9-tetraaza-1-phosphabicyclo[3.3.3]undecane (441 mg, 2.0 mmol) and 3,3-dimethylbutyraldehyde (2.64 ml, 200 mmol) in methanol (4 ml) and the mixture heated in a sealed vessel at 45° C. for 16 hr. The cooled mixture is concentrated in vacuo to yield the unsaturated nitrile as a colorless oil, used without purification.

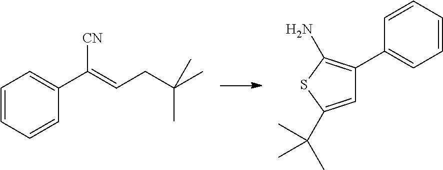

The nitrile (10.0 mmol), potassium carbonate (2.34 g, 23.4 mmol) and powdered sulfur (330 mg, 10.3 mmol) in ethanol (2 ml) are heated in a sealed vessel at 160° C. for 24 hr. The cooled mixture is diluted with water, extracted twice into diethyl ether and the combined organics concentrated in vacuo. Flash chromatography on silica gel (ethyl acetate/hexanes) yields 5-amino-2-t-butyl-4-phenylthiazole (75%).

### Preparation 60

### 5-methyl-3-phenylthiophen-2-amine

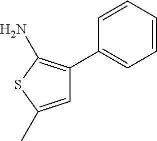

The title compound is prepared using the same procedures described in Preparation 59.

### Preparation 61

### 5-isopropyl-3-phenylthiophen-2-amine

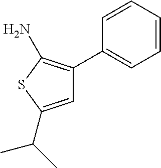

The title compound is prepared using the same procedures described in Preparation 59.

### Preparation 62

### 2-amino-5-chloro-3-phenylthiophene

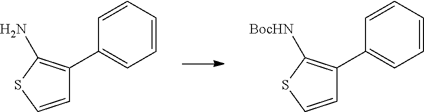

2-Amino-3-phenyl-thiophene (12.0 mmol) in THF (7 ml) is treated with di-tert-butyl dicarbonate (2.97 g, 13.3 mmol) and diisopropylethylamine (3.15 ml, 18.1 mmol) at ambient temperature for 60 hr, and the mixture concentrated in vacuo. Flash chromatography on silica gel (ethyl acetate/hexanes) yields 2-(N-Boc-amino)-3-phenyl-thiophene (1.98 g, 59%).

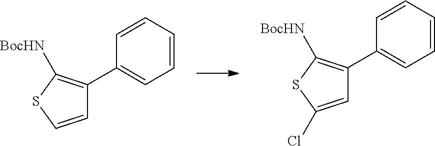

To 2-(N-Boc-amino)-3-phenyl-thiophene (89 mg, 0.32 mmol) in dichloromethane (4 ml) at 0° C. is slowly added N-chlorosuccinimide (48 mg, 0.36 mmol), and the mixture allowed to warm to ambient temperature for 16 hr. The mixture is diluted with dichloromethane, washed with water, and the organic phase concentrated in vacuo. Flash chromatography on silica gel (ethyl acetate/hexanes) yields 2-(N-Boc-amino)-5-chloro-3-phenyl-thiophene (66 mg, 66%).

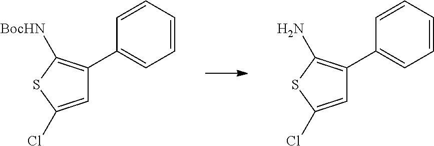

2-(N-Boc-amino)-5-chloro-3-phenyl-thiophene (66 mg, 0.21 mmol) is treated with trifluoroacetic acid (1 ml) in dichloromethane (3 ml) at ambient temperature for 1 hr. The mixture is diluted with DMF (1 ml) and the more volatile materials removed under reduced pressure. The resulting DMF solution of 2-amino-5-chloro-3-phenylthiophene is used in the subsequent coupling step without purification.

### Preparation 63

### 1-Methyl-4-(methylamino)-3-phenylpyrazole

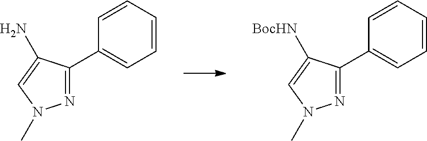

To 1-methyl-4-amino-3-phenylpyrazole (572 mg, 3.30 mmol) and di-tert-butyl dicarbonate (799 mg, 3.66 mmol) in THF (10 ml) and water (3 ml) is added dropwise saturated aqueous sodium bicarbonate (3 ml, 1.2 M, 3.6 mmol). The mixture is stirred at ambient temperature for 7 hr and then poured into aqueous citric acid (0.5 M) and extracted three times into ether (total 100 ml). The combined organic phases are washed with saturated aqueous sodium bicarbonate and brine, dried over magnesium sulfate and concentrated in vacuo to yield the crude carbamate as a brown oil (920 mg), used subsequently without purification.

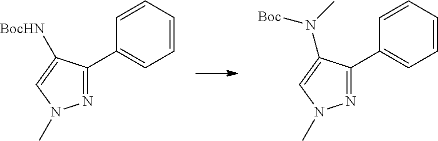

A suspension of sodium hydride in mineral oil (327 mg, 60% wt, 8.18 mmol) is washed with THF (2×5 ml) and suspended in THF (3.0 ml) at 0° C. To this is added dropwise the pyrazole (744 mg, 2.72 mmol) in THF (5.0 ml), and after 15 min, methyl iodide (187 μl, 3.00 mmol). After a further 30 min at 0° C. the mixture is allowed to warm to ambient temperature for 18 hr and then treated with saturated aqueous ammonium chloride and sufficient water to dissolve solids. The mixture is extracted three times into ether (total 120 ml), and the combined organic phases washed with brine, dried over magnesium sulfate and concentrated in vacuo to yield the crude N-methyl carbamate as a amber oil (750 mg, 96%), used without purification.

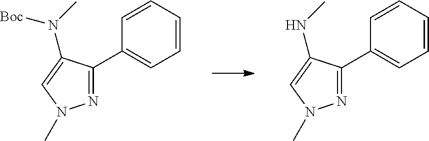

The crude N-methyl carbamate in DCM (1.0 ml) is treated with trifluoroacetic acid (1.0 ml) at ambient temperature for 40 min. The mixture is concentrated in vacuo, made homogeneous with dichloromethane and again concentrated to yield essentially pure I-methyl-4-(methylamino)-3-phenylpyrazole (150 mg, quant.) as a brown oil.

### Preparation 64

### N-methyl-4-phenyl-1,2,3-thiadiazol-5-amine

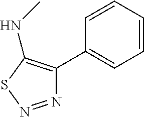

The title compound is prepared using the same procedures described in Preparation 63.

### Preparation 65

### tert Butyl-4-amino-3-phenyl pyrazole and 1-tert-butyl-4-amino-5-phenylpyrazole

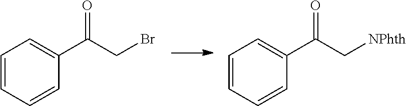

A solution of 2-bromoacetophenone (30.0 g, 151 mmol) in DMF (120 ml) is treated with potassium phthalimide (30.8 g, 166 mmol) portionwise at ambient temperature, and then heated to 40° C. for 3.5 hr. The cooled mixture is poured into water (600 ml) and extracted with chloroform (300 ml then 100 ml). The combined organic phases are washed with sodium hydroxide (200 ml, 0.2 N), water (2×100 ml) and brine (100 ml), dried over magnesium sulfate and concentrated in vacuo. The resulting cream solid is suspended in ether (100 ml), recovered by filtration, washed with ether (100 ml) and dried in vacuo to yield pure 2-phthalimido-acetophenone as a white solid (34.3 g, 86%).

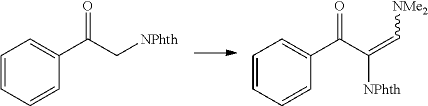

According to the procedures described in C. Chen, K. Wilcoxen, J. R. McCarthy; Tetrahedron Lett, 1988, 39, 8229-8232a suspension of 2-phthalimidoacetophenone (13.3 g, 50.0 mmol) in dimethylformamide dimethyl acetal (26.7 ml, 200 mmol) is heated at reflux for 28 hr and concentrated in vacuo. The resulting amber oil is crystallized form isopropanol (100 ml) and washed with isopropanol (2×5 ml) to yield of 3-(dimethylamino)-1-phenyl-2-phthalimido-2-propen-1-one as yellow needles (13.7 g, 85%).

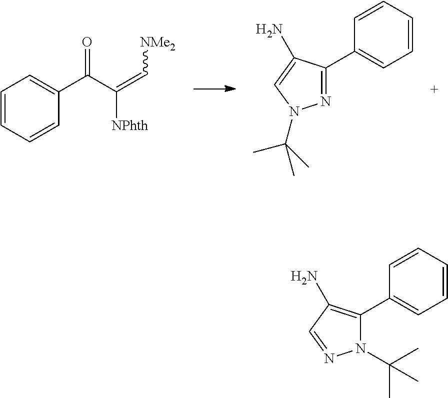

A mixture of 3-(dimethylamino)-1-phenyl-2-phthalimido-2-propen-1-one (3.00 g, 9.38 mmol) and tert-butyl hydrazine hydrochloride (1.29 g, 10.3 mmol) in ethanol (94 ml) and water (9.4 ml) is stirred at ambient temperature for 64 hr and then heated at reflux for 24 hr. The cooled mixture is treated with hydrazine (590 μl, 18.8 mmol) and returned to reflux for 75 min. On cooling and standing at ambient temperature a precipitate is formed. The mixture is filtered, the solid washed with a mixture of ethanol (5 ml) and water (0.5 ml), and the filtrate concentrated in vacuo. The residue is partitioned between ether (250 ml) and saturated aqueous sodium bicarbonate (50 ml) diluted with water (100 ml), and the aqueous phase extracted twice more with ether (2×50 ml). The combined organic phases are washed with brine, dried over sodium sulfate and concentrated in vacuo to give a pale solid (1.92 g). Flash chromatography on silica gel (ethyl acetate/hexanes) yields 1-tert butyl-4-amino-3-phenylpyrazole (1.52 g, 75% for 2 steps) and 1-tert-butyl-4-amino-5-phenylpyrazole (114 mg, 6% for 2 steps).

### Preparation 66

### 1-(2,2,2-trifluoroethyl)-3-phenyl-1H-pyrazol-4-amine and 1-(2,2,2-trifluoroethyl)-5-phenyl-IH-pyrazol-4-amine

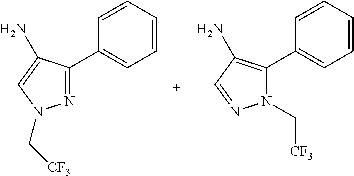

1-(2,2,2-trifluoroethyl)-3-phenyl-1H-pyrazol-4-amine and 1-(2,2,2-trifluoroethyl)-5-phenyl-1H-pyrazol-4-amine are prepared similarly from 2,2,2-trifluoroethylhydrazine according to the procedures described in Preparation 65.

### Preparation 67

### (R)-3-Phenylpyrrolidine

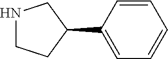

(R)-3-Phenylpyrrolidine is commercially available from Astatech, Inc.

### Preparation 68

### (R)-3-(4-Fluorophenyl)-pyrrolidine

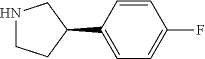

### Step 1. 3-(4-Fluoro-phenyl)-pyrrolidine-1-carboxylic acid tert-butyl ester

A solution of racemic 3-(4-fluorophenyl)-pyrrolidine (1.8 g, 10.9 mmol) in THF (30 ml) was treated with Boc₂O (2.9 ml, 13 mmol) and was 2M aq. NaHCO₃ solution (3 ml) at 0° C., slowly warmed to rt and stirred overnight until complete consumption of racemic 3-(4-fluorophenyl)-pyrrolidine as evidenced by TLC analysis. The reaction mixture was diluted with EtOAc, washed with water, dried (Na₂SO₄), filtered and evaporated followed by column chromatography purification (100-200 mesh silica gel, 5% EtOAc/Pet ether) to obtain pure racemic 3-(4-fluoro-phenyl)-pyrrolidine-1-carboxylic acid tert-butyl ester (2.3 g, 82%). The racemic sample of 3-(4-fluoro-phenyl)-pyrrolidine-1-carboxylic acid tert-butyl ester was separated by chiral HPLC(Chiralpak IC (250×4.6 mm), mobile phase, hexane, ethanol and diethyl amine 98/2/0.1, flow rate: 1 ml/min, diluents mobile phase) to obtain (S)-3-(4-fluoro-phenyl)-pyrrolidine-1-carboxylic acid tert-butyl ester ([α]_{D}=2.4° (c=2, MeOH)) and (R)-3-(4-fluoro-phenyl)-pyrrolidine-1-carboxylic acid tert-butyl ester ([α]_{D}=−5.6° (c=2, MeOH).

### Step 2. (R)-3-(4-Fluorophenyl)-pyrrolidine

A solution of (R)-3-(4-fluoro-phenyl)-pyrrolidine-1-carboxylic acid tert-butyl ester (700 mg, 2.65 mmol) was treated with solution of 4M HCl in 1,4-dioxane at 0-5° C. for 1 h until complete consumption of compound (R)-3-(4-fluoro-phenyl)-pyrrolidine-1-carboxylic acid tert-butyl ester as evidenced by TLC analysis. The reaction mixture was concentrated and neutralized by methanolic ammonia treatment. After evaporation, the residue was dried for several hours to obtain compound (R)-3-(4-fluorophenyl)-pyrrolidine (300 mg, 69%) which was used as such without further purification. The specific rotation of (R)-3-(4-fluoro-phenyl)-pyrrolidine ([α]_{D}=−1.06° (c=2, MeOH) was correlated with specific rotation of compound (R)-3-phenyl pyrrolidine ([α]_{D}=−12.4° (c=0.9, MeOH), J. Org. Chem. 1990, 55, 270-275)).

### Preparation of Intermediates 1a

### Preparation 69

### (3aS,7aS)-6-phenethyl-octahydro-1H-pyrrolo[2,3-c]pyridine

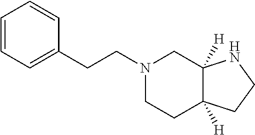

The chiral title compound is synthesised according to WO 2005/097791 and WO2006/107964 and the disclosures of which are incorporated herein by references.

### Preparation 70

### 2-(4-benzylphenyl)pyrrolidine

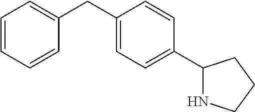

The title compound is synthesised according to WO 2005/097791 and the disclosures of which are incorporated herein by references.

### Preparation 71

### 2-(4-phenoxyphenyl)pyrrolidine

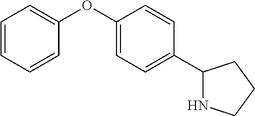

The title compound is synthesised according to WO 2005/097791 and the disclosures of which are incorporated herein by references.

### Preparation 72

### 3-(4-fluorophenoxy)-5-(pyrrolidin-2-yl)pyridine

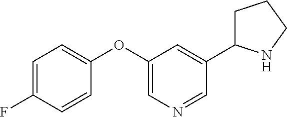

The title compound is synthesised according to WO 2005/097791 and the disclosures of which are incorporated herein by references.

### Preparation 73

### 5-fluoro-N-(4-fluorophenyl)-N-methyl-4-(pyrrolidin-2-yl)pyridin-2-amine

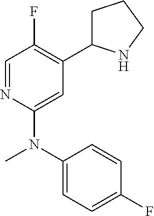

The title compound is synthesised according to WO 2005/097791 and the disclosures of which are incorporated herein by references.

### Preparation 74

### (S)-4-phenyl-2-(pyrrolidin-2-yl)thiazolo[4,5-c]pyridine

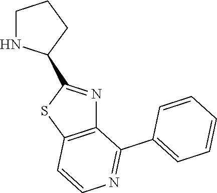

The title compound is synthesised according to WO 2007/106192 and the disclosures of which are incorporated herein by references.

### Preparation 75

### (S)-7-phenyl-2-(pyrrolidin-2-yl)thiazolo[5,4-c]pyridine

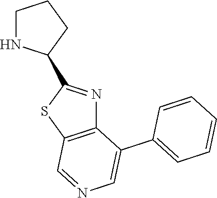

The title compound is synthesised according to WO 2007/106192 and the disclosures of which are incorporated herein by references.

### Preparation 76

### (S)-7-phenyl-2-(pyrrolidin-2-yl)thiazolo[5,4-b]pyridine

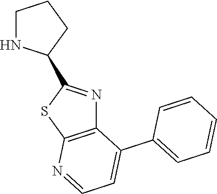

The title compound is synthesised according to WO 2007/106192 and the disclosures of which are incorporated herein by references.

### Preparation 77

### (S)-7-phenyl-2-(pyrrolidin-2-yl)thiazolo[5,4-d]pyrimidine

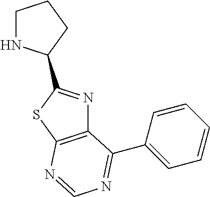

The title compound is synthesised according to WO 2007/106192 and the disclosures of which are incorporated herein by references.

### Preparation 78

### Pyrrolidine-2-carboxylic acid (4′-fluoro-biphenyl-2-yl)-amide

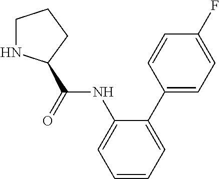

The title compound as well as the chloro substituted is synthesised according to WO 2007/106192 and the disclosures of which are incorporated herein by references.

### Preparation 79

### Pyrrolidine-2-carboxylic acid (3′-fluoro-biphenyl-2-yl)-amide

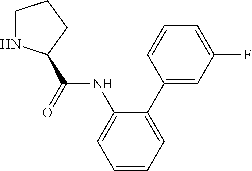

The title compound well as the chloro substituted is synthesised according to WO 2007/106192 and the disclosures

### Preparation 81

### Pyrrolidine-2-carboxylic acid (2′-fluoro-biphenyl-2-yl)-amide

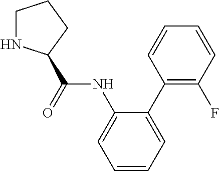

The title compound well as the chloro substituted is synthesised according to WO 2007/106192 and the disclosures of which are incorporated herein by references.

### Preparation 82

((R)-3-Phenylpyrrolidin-1-yl)((2S,4R)-4-phenylpyrrolidin-2-yl)methanone hydrochloride

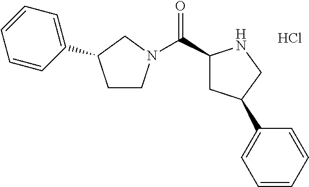

### Step 1. ((R)-3-Phenylpyrrolidin-1-yl)((2S,4R)-4-phenylpyrrolidin-2-yl)methanone hydrochloride

The title compound was synthesized according to Method AC.

(2S,4R)-1-(tert-butoxycarbonyl)-4-phenylpyrrolidine-2-carboxylic acid (0.50 g, 1.72 mmol) and DhbtOH (0.281 g, 1.72 mmol) were dissolved in tetrahydrofurane (10 ml). DIC (0.27 mL, 1.72 mmol) was slowly added at 0° C. Stirring was continued for 2 hours at 0° C. and at room temperature for 16 hours. Precipitated diisopropyl urea was filtered off and the filtrate was evaporated. The residue was redissolved in ethylacetate (2 ml) and more diisopropyl urea was precipitated by addition of heptane (8 ml), and removed by centrifugation. Evaporation afforded the Dhbt-ester (0.54 g, 72%). ¹H NMR conforms to structure.

(2S,4R)-4-Phenyl-pyrrolidine-1,2-dicarboxylic acid 1-tert-butyl ester 2-(4-oxo-4H-benzo[d][1,2,3]triazin-3-yl)ester (0.54 g, 1.24 mmol) was dissolved in acetonitril. (R)-3-phenylpyrrolidine (0.273 g, 1.49 mmol) and N,N-diisopropylethylamine (0.32 mL, 1.86 mmol) were added in the given order and stirred for 16 hours at rt. The reaction mixture was concentrated and the residue dissolved in dichloromethane (25 ml), which was washed successively with 10% citric acid in water (10 mL), water (10 mL), saturated aqueous bicarbonate (10 mL) and dried over sodium sulfate. Evaporation afforded the Boc protected title compound as a solid (0.458 g, 88%). ¹H NMR conforms to structure. To Boc protected title compound was added 4 M HCl in dioxan. The mixture was stirred for 1 hour at room temperature and then evaporated under reduced pressure. The title compound was used without further purification.

### Preparation 83

### ((R)-3-(4-Fluorophenyl)pyrrolidin-1-yl)((2S,4R)-4-(fluorophenyl)pyrrolidin-2-yl)methanone hydrochloride

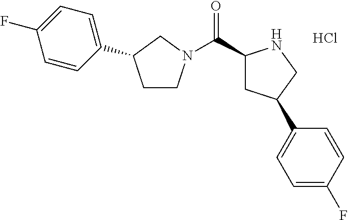

### Step 1. (2S,4R)-Di-tert-butyl-4-(4-fluorophenyl)-4-hydroxypyrrolidine-1,2-dicarboxylate

To a solution of (S)-di-tert-butyl-4-oxopyrrolidine-1,2-dicarboxylate 6 (1.0 g, 0.35 mmol) in anhydrous THF (5 ml) was slowly added (4-fluorophenyl)magnesium bromide (14 ml, 14.0 mmol) at −78° C. and stirred for 2 h at −78° C. and for another 2 h at room temperature. The reaction mixture was quenched with saturated NH₄Cl (10 mL), extracted with EtOAc (2×25 ml), washed with brine solution (15 ml). The organic phase was dried (Na₂SO₄), evaporated and purified by column chromatography (100-200 mesh silica gel, 5% EtOAc/Petrolum ether) to afford (2S,4R)-di-tert-butyl-4-(4-fluorophenyl)-4-hydroxypyrrolidine-1,2-dicarboxylate (300 mg, 23%) as a thick liquid. (TLC system: 15% EtOAc/Petrolum ether, R_{f} 0.37).

### Step 2. (S)-4-(4-Fluorophenyl)-2,5-dihydro-1H-pyrrole-2-carboxylic acid

To a solution of (2S,4R)-di-tert-butyl-4-(4-fluorophenyl)-4-hydroxypyrrolidine-1,2-dicarboxylate (1.4 g, 3.8 mmol) in anhydrous dichloromethane was added TFA (0.9 mL) slowly at 0° C. and stirred for 24 h at room temperature. The resulting mixture was evaporated, co evaporated with Et₂O (20 ml) affording (S)-4-(4-fluorophenyl)-2,5-dihydro-1H-pyrrole-2-carboxylic acid (450 mg, 59%) as a thick liquid. The crude was used without any further purification. (TLC system: 60% methanol/chloroform, R_{f} 0.25).

### Step 3. (2S,4R)-4-(4-Fluorophenyl)pyrrolidine-2-carboxylic acid

To a solution of (S)-4-(4-fluorophenyl)-2,5-dihydro-1H-pyrrole-2-carboxylic acid (150 mg, 0.7246 mmol) in methanol (4 ml) was added Pd/C (20 mg) and kept under H₂ atmosphere overnight at room temperature. The resulting mixture was filtered through celite, washed with methanol (50 mL) and evaporated to afford (2S,4R)-4-(4-fluorophenyl)pyrrolidine-2-carboxylic acid (120 mg, 70%) (TLC system: 20% methanol/chloroform, R_{f} 0.3)

### Step 4. (2S,4R)-1-(tert-Butoxycarbonyl)-4-(4-fluorophenyl)pyrrolidine-2-carboxylic acid

To a solution of (2S,4R)-4-(4-fluorophenyl)pyrrolidine-2-carboxylic acid (900 mg, 4.3062 mmol) in water/1,4-dioxane (10 ml, 1:1 v/v) were added NaHCO₃ (1.08 g, 12.91 mmol) and Boc₂O (0.951 mL, 4.3062 mmol) at 0° C. The reaction mixture was brought to room temperature and stirred for 3 h. The resulting mixture was acidified (pH˜6) with 1N aq. HCl and extracted with EtOAc (2×25 ml) and washed with brine solution (2×10 ml). The organic phase was dried (Na₂SO₄), evaporated and the residue was purified by column chromatography (100-200 mesh silica gel, 2%, 3%, 4% methanol/chloroform) affording (2S,4R)-1-(tert-Butoxycarbonyl)-4-(4-fluorophenyl)pyrrolidine-2-carboxylic acid (350 mg, 27%) as a yellow thick liquid. (TLC system: 10% methanol/chloroform, R_{f} 0.3).

### Step 5. ((R)-3-(4-Fluorophenyl)pyrrolidin-1-yl)((2S,4R)-4-(fluorophenyl)pyrrolidin-2-yl)methanone hydrochloride

The title compound was synthesized according to Method AC.

To a solution of compound (2S,4R)-1-(tert-butoxycarbonyl)-4-(4-fluorophenyl)pyrrolidine-2-carboxylic acid (60 mg, 0.894 mmol) and compound (R)-3-(4-Fluoro-phenyl)-pyrrolidine (38 mg, 0.2330 mmol) in anhydrous DMF/CH₂Cl₂ (3 ml, 1:1 v/v) were added HATU (88 mg, 0.2330 mmol) and DIPEA (0.11 ml, 0.6793 mmol) at 0° C. The reaction mixture was brought to room temperature and stirred for 2 h. The solvent was evaporated; residue was dissolved in EtOAc, washed with sat. aq. KHCO₃, water and, brine solution. The organic phase was dried (Na₂SO₄), evaporated and purified by column chromatography (100-200 mesh Silica gel, 1% methanol/chloroform) to afford compound (2S,4R)-tert-butyl 4-(4-fluorophenyl)-2-((R)-3-(4-fluorophenyl)pyrrolidine-1-carbonyl)pyrrolidine-1-carboxylate as a thick liquid (80 mg (90%) (TLC system: 10% methanol/chloroform, R_{f} 0.6).

To a solution of compound (2S,4R)-tert-butyl 4-(4-fluorophenyl)-2-((R)-3-(4-fluorophenyl)pyrrolidine-1-carbonyl)pyrrolidine-1-carboxylate (280 mg) in 1,4-dioxane (3 ml) was added 4M HCl in 1,4-dioxane (0.5 ml) at 0° C. and the reaction mixture was brought to room temperature. It was stirred for 2 h and the solvent was evaporated under vacuum. The resulting crude oil was precipitated with Et₂O, filtered and the precipitate was washed with Et₂O to afford ((R)-3-(4-fluorophenyl)pyrrolidin-1-yl)((2S,4R)-4-(fluorophenyl)pyrrolidin-2-yl)methanone hydrochloride as a brown solid (200 mg, 91%) (TLC system: 10% methanol/chloroform, R_{f} 0.2)

### Preparation 84

### (R)-3-Phenyl-1-(((2S,4R)-4-phenylpyrrolidin-2-yl)methyl)pyrrolidine, dihydrochloride

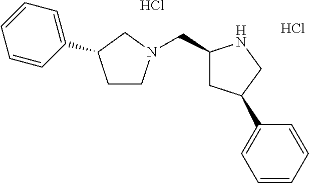

### Step 1. (R)-3-Phenyl-1-(((2S,4R)-4-phenylpyrrolidin-2-yl)methyl)pyrrolidine, dihydrochloride

The title compound was synthesized according to Method AD.

(2S,4R)-tert-Butyl-4-phenyl-2-((R)-3-phenylpyrrolidine-1-carbonyl)pyrrolidine-1-carboxylate (0.54 g, 1.28 mmol) was dissolved in dioxane (10 ml) under argon. Borane dimethylsulfide complex (0.64 ml, 6.40 mmol) was added and the reaction mixture was stirred at 80° C. for 16 hours. Excess reagent was carefully quenched with water followed by evaporation. 1% HCl in methanol (20 ml) was added to the residue, and the mixture was evaporated. The oil was triturated several times with 1% HCl in methanol, until the title compound as a solid residue was obtained (0.474 g, 98%).

### Preparation 85

### 1-Benzyl-5-((2S,4R)-4-phenylpyrrolidin-2-yl)-1H-tetrazole, trifluoro acetic acid

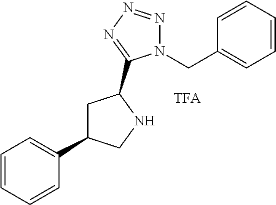

The title compound is synthesised according to Method AK.

### Step 1. (S)-2-(1H-Tetrazol-5-yl)-4-phenyl-pyrrolidine-1-carboxylic acid tert-butyl ester

To a solution of (S)-2-cyano-4-phenyl-pyrrolidine-1-carboxylic acid tert-butyl ester (500 mg, 2.55 mmol) in N,N-dimethyl-formamide (20 mL) is added sodium azide (174 mg, 2.68 mmol) and ammonium chloride (150 mg, 2.81 mmol). The solution is stirred at 93° C. over night. The solution is poured into 5% citric acid solution with ice, and the mixture was extracted with EtOAc. The organic extract was washed with brine, dried and concentrated under vacuum. The crude title compound is used directly in the next step without further purification.

### Step 2. (S)-2-(2-Benzyl-2H-tetrazol-5-yl)-4-phenyl-pyrrolidine-1-carboxylic acid tert-butyl ester

To a solution of crude (S)-2-(1H-tetrazol-5-yl)-4-phenyl-pyrrolidine-1-carboxylic acid tert-butyl ester in N,N-dimethyl-formamide (5 mL) is added K₂CO₃ (1.16 g, 8.4 mmol) and benzyl bromide (665 μL, 5.6 mmol). The solution is stirred at room temperature for 1 hour. The mixture is diluted with EtOAc and washed with brine. The organic layer is dried and concentrated under vacuum. The residue is purified by flash column chromatography to provide the title compound and the other regio isomer (S)-2-(1-Benzyl-1H-tetrazol-5-yl)-4-phenyl-pyrrolidine-1-carboxylic acid tert-butyl ester.

### Step 3. 1-Benzyl-5-((2S,4R)-4-phenylpyrrolidin-2-yl)-1H-tetrazole TFA salt

To a solution of (S)-2-(2-Benzyl-2H-tetrazol-5-yl)-4-phenyl-pyrrolidine-1-carboxylic acid tert-butyl ester in DCM (5 ml) is added triethylsilane (479 μL, 3.0 mmol) and then TFA (5 ml). The solution is stirred at room temperature for 1 hour and dried under vacuum. The crude oil is used directly in the next step without further purification.

### Preparation 86

### (2S,4R)-2-((naphthalen-1-yloxy)methyl)-4-phenylpyrrolidine hydrochloride

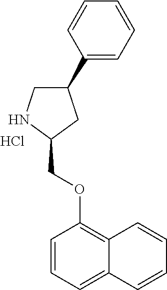

The title compound is synthesised according to Method AM.

### Step 1. (2S,4R)-tert-butyl 2-(hydroxymethyl)-4-phenylpyrrolidine-1-carboxylate

To a cold solution (0° C.) of (2S,4R)-1-(tert-butoxycarbonyl)-4-phenylpyrrolidine-2-carboxylic acid (0.291 g, 1.0 mmol) in anhydrous THF (5 mL) under nitrogen is added isobutyl carbonochloridate (0.136 g, 1.0 mmol) and DIPEA (0.129 g, 1.0 mmol). The reaction mixture is stirred 1 hour at 0 PC followed by 30 min at room temperature. The solution is cooled to 0 PC again and sodium borohydride (0.113 g, 3.0 mmol) is added in one portion. MeOH (10 mL) is added dropwise to the reaction mixture over a period of 10 min. The solution is stirred for additional 10 min, and neutralized with sodium acetate buffer (0.5 M, 4 mL). The organic solvents are evaporated under reduced pressure and the product is extracted with ethyl acetate (3×7 mL). The organic phase is washed with sodium acetate buffer (0.5 M, 10 mL), water (10 mL), 5% aqueous NaHCO₃ (10 mL), water (10 mL), dried over sodium sulphate and evaporated under reduced pressure. The residue is purified by silica gel column chromatography to afford the title compound.

### Step 2. (2S,4R)-tert-butyl 2-((naphth-1-yloxy)methyl)-4-phenylpyrrolidine-1-carboxylate

To a DCM solution of (2S,4R)-tert-butyl 2-(hydroxymethyl)-4-phenylpyrrolidine-1-carboxylate (0.138 g, 0.5 mmol) is added pyridine (0.237 g, 1.5 mmol) and tosyl chloride (0.143 g, 0.75 mmol). The reaction is stirred over night at room temperature. The solution is evaporated and the crude purified by silica gel column chromatography to afford the toslated product. The tosylated product is dissolved in THF and sodium 1-naphthalen-olate is added (83 mg, 0.5 mmol). The reaction is stirred over night at room temperature. The reaction is evaporated under reduced pressure and the residue purified by silica gel column chromatography to afford the title compound.

### Step 3. (2S,4R)-2-((naphthalen-1-yloxy)methyl)-4-phenylpyrrolidine hydrochloride

To (2S,4R)-tert-butyl 2-((naphth-1-yloxy)methyl)-4-phenylpyrrolidine-1-carboxylate (129 mg, 0.32 mmol) is added 4 M HCl in dioxan. The mixture is stirred for 1 hour at room temperature and then evaporated under reduced pressure. The title compound is used without further purification.

### Preparation 87

### 5-benzyl-3-((2S,4R)-4-phenylpyrrolidin-2-yl)-1,2,4-oxadiazole hydrochloride

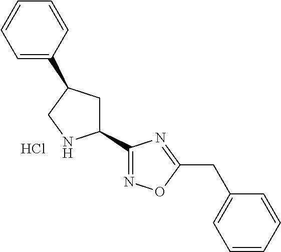

The title compound is synthesised according to Method AL.

### Step 1. ((2S,4R)-tert-butyl 2-(N′-hydroxycarbamimidoyl)-4-phenylpyrrolidine-1-carboxylate

To a solution of (S)-2-cyano-4-phenyl-pyrrolidine-1-carboxylic acid tert-butyl ester (500 mg, 2.55 mmol) in ethanol (10 mL) is added aqueous hydroxylamine (0.34 mL, 15.2 M, 5.1 mmol). The solution is stirred at room temperature over night. The solution is evaporated under reduced pressure and redissolved in EtOAc (10 mL) and washed with water (10 mL), brine (10 mL), dried and concentrated under vacuum. The crude oil is used directly in the next step without further purification.

### Step 2. (2S,4R)-tert-butyl 2-(5-benzyl-1,2,4-oxadiazol-3-yl)-4-phenylpyrrolidine-1-carboxylate

To ((2S,4R,Z)-tert-butyl 2-(N′-hydroxycarbamimidoyl)-4-phenylpyrrolidine-1-carboxylate (610 mg, 2.0 mmol) dissolved in dry dioxan (10 mL) is added DIPEA (516 mg, 4.0 mmol) and 2-phenylacetyl chloride (309 mg, 2.0 mmol). The stirred reaction is heated to 90° C. for 4 hours. The reaction mixture is cooled down to room temperature and evaporated under reduced pressure and the residue purified by silica gel column chromatography to afford the title compound.

### Step 3. 5-benzyl-34(2S,4R)-4-phenylpyrrolidin-2-yl)-1,2,4-oxadiazole hydrochloride

To (2S,4R)-tert-butyl 2-(5-benzyl-1,2,4-oxadiazol-3-yl)-4-phenylpyrrolidine-1-carboxylate is added 4 M HCl in dioxan. The mixture is stirred for 1 hour at room temperature and then evaporated under reduced pressure. The title compound is used without further purification.

### Preparation 88

### 4-benzyl-2-((2S,4R)-4-phenylpyrrolidin-2-yl)oxazole hydrochloride

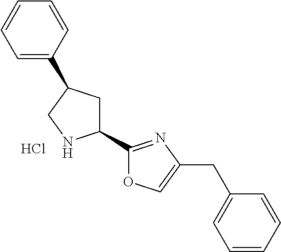

The title compound is synthesised according to Method AN.

### Step 1. (2S,4R)-tert-Butyl 24(1-hydroxy-3-phenylpropan-2-yl)carbamoyl)-4-phenylpyrrolidine-1-carboxylate

(2S,4R)-1-(tert-butoxycarbonyl)-4-phenylpyrrolidine-2-carboxylic acid (0.50 g, 1.72 mmol) and 3-hydroxybenzo[d][1,2,3]triazin-4(3H)-one (0.281 g, 1.72 mmol) are dissolved in THF (10 ml). Diisopropylcarbodiimide (0.27 mL, 1.72 mmol) was slowly added at 0° C. Stirring is continued for 2 hours at 0° C. and at room temperature for 16 hours. Precipitated diisopropyl urea is filtered off and the filtrate is evaporated. The residue is redissolved in ethylacetate (2 ml) and more diisopropyl urea is precipitated by addition of heptane (8 ml), which is removed by centrifugation. Evaporation afforded the Dhbt-ester. The Dhbt-ester (0.54 g, 1.24 mmol) is dissolved in dry acetonitril. 2-amino-3-phenylpropan-1-ol (225 mg, 1.49 mmol) and N,N-diisopropylethylamine (0.32 mL, 1.86 mmol) are added in the given order and stirred for 16 hours at room temperature. The reaction mixture is concentrated and the residue dissolved in DCM (25 ml), which is washed successively with 10% citric acid in water (10 mL), water (10 mL), saturated aqueous bicarbonate (10 mL) and dried over sodium sulfate. Evaporation affords the title compound as a solid.

### Step 2. (2S,4R)-tert-butyl 2-(4-benzyl-4,5-dihydrooxazol-2-yl)-4-phenylpyrrolidine-1-carbon/late

Burgess' reagent (1.05 mmol) is added in one portion to a stirred solution of (2S,4R)-tert-Butyl 2-((1-hydroxy-3-phenylpropan-2-yl)carbamoyl)-4-phenylpyrrolidine-1-carboxylate (425 mg, 1.0 mmol) in dry THF and the resulting solution is then heated at 70° C. for 12 hours under an argon atmosphere (Mink et al. (1998) Tetrahedron Lett. 39, 5709-5712). The reaction mixture is concentrated and the residue dissolved in DCM (25 ml), which is washed successively with 10% citric acid in water (10 mL), water (10 mL), saturated aqueous bicarbonate (10 mL) and dried over sodium sulfate. Evaporation and purification by silica gel column chromatography affords the title compound.

### Step 3. (2S,4R)-tert-butyl 2-(4-benzyloxazol-2-yl)-4-phenylpyrrolidine-1-carboxylate

A solution of (2S,4R)-tert-butyl 2-(4-benzyl-4,5-dihydrooxazol-2-yl)-4-phenylpyrrolidine-1-carboxylate (101 mg, 0.25 mmol) in DCM is cooled to 0° C. and 1.1 eq. of DBU is added. 1.1 eq. of bromotrichloromethane is introduced dropwise via a syringe over 10 min. The reaction is stirred under argon until completion after 8 hrs. The crude is purified by silica gel column chromatography to afford the title compound.

### Step 4. 4-benzyl-2-((2S,4R)-4-phenylpyrrolidin-2-yl)oxazole hydrochloride

To (2S,4R)-tert-butyl 2-(4-benzyloxazol-2-yl)-4-phenylpyrrolidine-1-carboxylate is added 4 M HCl in dioxan. The mixture is stirred for 1 hour at room temperature and then evaporated under reduced pressure. The title compound is used without further purification.

### Synthesis of Compounds of Formula (I) with Substructure (IIa)

### Example 1

### [5-(1-Methylamino-ethyl)-furan-2-yl]-[(2S,4R)-4-phenyl-2-((R)-3-phenyl-pyrrolidine-1-carbonyl)-pyrrolidin-1-yl]-methanone

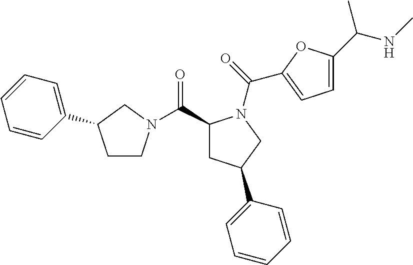

The title compound was synthesised according to Method A.

### Step 1. Methyl-O-{5-[(2S,4R)-4-phenyl-2-((R)-3-phenyl-pyrrolidine-1-carbonyl)-pyrrolidine-1-carbonyl]-furan-2-yl}-ethyl)-carbamic acid tert-butyl ester

((R)-3-Phenylpyrrolidin-1-yl)((2S,4R)-4-phenylpyrrolidin-2-yl)methanone hydrochloride (153.5 mg, 0.43 mmol) and 5-[1-(tert-Butoxycarbonyl-methyl-amino)-ethyl]-furan-2-carboxylic acid (137 mg, 0.51 mmol) were dissolved in DCM-dimethylformamide (1:1, 1 ml). HATU (194 mg, 0.51 mmol) and DIPEA (267 μl, 1.53 mmol) were added in the given order. The reaction was stirred at room temperature for 16 hours then evaporated to give a residue. The crude was dissolved in ethyl acetate (10 mL) and washed successively with aqueous potassium hydrogensulphate (10 mL, 10%), saturated aqueous potassium bicarbonate (10 mL), brine (10 mL) and dried over sodium sulfate. After filtration the organic solvent was evaporated in vacou and the crude was purified using flash chromatography with silicagel as absorbent and gradient elution (DCM to 70% ethyl acetate in DCM) to afford the product (182 mg, 74%). LC-MS: M+H⁺=573

### Step 2. (5-(1-(methylamino)ethyl)furan-2-yl)((2S,4R)-4-phenyl-2-(((R)-3-phenylpyrrolidin-1-yl)carbonyl)pyrrolidin-1-yl)methanone

To Methyl-(1-{5-[(2S,4R)-4-phenyl-2-((R)-3-phenyl-pyrrolidine-1-carbonyl)-pyrrolidine-1-carbonyl]-furan-2-yl}-ethyl)-carbamic acid tert-butyl ester was added 4 M HCl in dioxan (2 mL). The mixture was stirred for 2 hour at room temperature and then evaporated in vacou. The crude oil precipitated out when triturated with diethyl ether. The diethyl ether was removed and the precipitate was washed with ether (3×5 mL) and dried. To the hydrochlorid salt of (5-(1-(methylamino)ethyl)furan-2-yl)((2S,4R)-4-phenyl-2-(((R)-3-phenylpyrrolidin-1-yl)carbonyl)pyrrolidin-1-yl)methanone (30 mg, 0.06 mmol) dissolved in THF (2 mL) was added Si-carbonate (300 mg, 0.2 mmol, Silicycle, Inc). The reaction was agitated for 2 h at room temperature. The Si-carbonate was filtrate from the solvent and was washed with THF (2×2 mL). The combined solvent was evaporated in vacou to give 15.7 mg of the title compound in 55% yield. LC-MS: M+H⁺=472.6

### Example 2

### (5-(1-Aminoethyl)furan-2-yl)((2S,4R)-4-phenyl-2-(((R)-3-phenylpyrrolidin-1-yl)methyl)pyrrolidin-1-yl)methanone

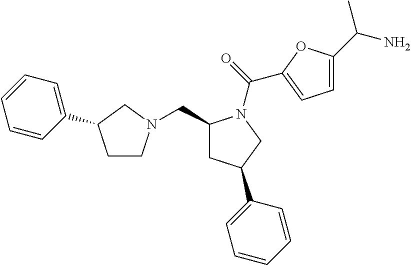

The title compound was synthesised according to Method A.

### Step 1. 2-nitro-N-(1-(54(2S,4R)-4-phenyl-2-(((R)-3-phenylpyrrolidin-1-yl)methyl)pyrrolidine-1-carbonyl)furan-2-yl)ethyl)benzenesulfonamide

(R)-3-phenyl-1-(((2S,4R)-4-phenylpyrrolidin-2-yl)methyl)pyrrolidine, dihydrochloride (43.7 mg, 0.113 mmol) and 5-[1-(2-nitro-benzenesulfonylamino)-ethyl]-furan-2-carboxylic acid (38.3 mg, 0.113 mmol) were dissolved in dimethylformamide (2 ml). PyBOP (33.6 mg, 0.113 mmol) and DIPEA were added in the given order. The reaction was stirred at room temperature for 16 hours then evaporated to give a residue, which was purified by LC-MS (C18 silicagel, linear gradient from 40% to 80% aqueous acetonitrile with 0.1% formic acid) to give 15.6 mg of the title compound (22%). LC-MS: M+H⁺=629.2

### Step 2. (5-(1-Aminoethyl)furan-2-yl)((2S,4R)-4-phenyl-2-(((R)-3-phenylpyrrolidin-1-yl)methyl)pyrrolidin-1-yl)methanone

Deprotection of 2-nitro-N-(1-(5-((2S,4R)-4-phenyl-2-(((R)-3-phenylpyrrolidin-1-yl)methyl)-pyrrolidine-1-carbonyl)furan-2-yl)ethyl)benzenesulfonamide (16 mg, 26 μmol) was performed by shaking a mixture of 4-mercaptoanisol (10 μL, 79 μmol), cesiumcarbonate (51 mg, 157 μmol) in tetrahydrofurane (2 ml) at room temperature for 16 hours. Purification on LC-MS (C18 silicagel, linear gradient from 30% to 70% aqueous acetonitrile with 0.1% formic acid) furnished a 9 mg of the title compound. LC-MS: M+H⁺=444.6

Examples 3 to 12 were synthesized according to procedures similar to those described in Examples 1 and 2.

### Example 3a

### [3-(1-Methylamino-ethyl)-phenyl]-[(2S,4R)-4-phenyl-2-((R)-3-phenyl-pyrrolidine-1-carbonyl)-pyrrolidin-1-yl]-methanone

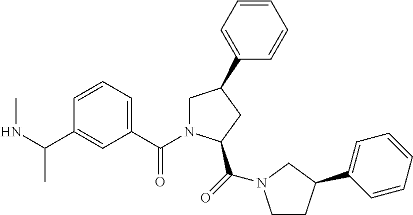

The title compound was synthesized from Preparation 8a and 82 according to the procedure described in Example 1. LC-MS: M+H⁺=482.6

### Example 3b

(3-(1(R)-(methylamino)ethyl)phenyl)((2S,4R)-4-phenyl-2-(((R)-3-phenylpyrrolidin-1-yl)methyl)pyrrolidin-1-yl)methanone

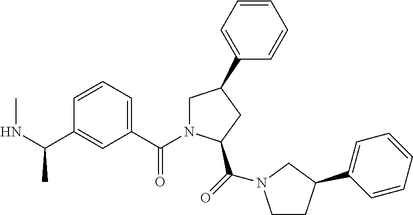

The title compound was synthesized from Preparation 8b and 82 according to the procedure described in Example 1. The residue was purified by column chromatography (100-200 mesh silica gel, methanol/chloroform, 2:98) to afford the title compound as an off white solid. (TLC system: chloroform/methanol 9:1, R_{f} 0.25). LC-MS: M+H⁺=482.6

### Example 3c

### (3-(1(S)-(methylamino)ethyl)phenyl)((2S,4R)-4-phenyl-2-(((R)-3-phenylpyrrolidin-1-yl)methyl)pyrrolidin-1-yl)methanone

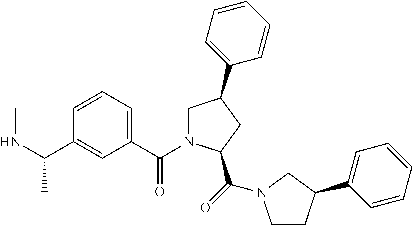

The title compound was synthesized from Preparation 8c and 82 according to the procedure described in Example 1. The residue was purified by column chromatography (100-200 mesh silica gel, methanol/chloroform, 2:98) to afford the title compound as an off white solid. (TLC system: chloroform/methanol 9:1, R_{f} 0.20). LC-MS: M+H⁺=482.6

### Example 4

### [6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone

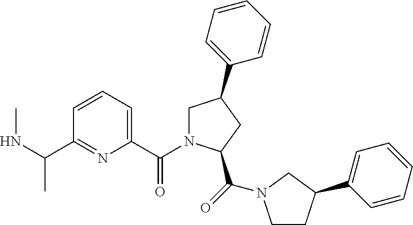

The title compound was synthesized from Preparation 7 and 82 according to the procedure described in Example 1. LC-MS: M+H⁺=483.6

### Example 5a

### [5-(1-Amino-ethyl)-furan-2-yl]-[(2S,4R)-4-phenyl-2-((R)-3-phenyl-pyrrolidine-1-carbonyl)-pyrrolidin-1-yl]-methanone

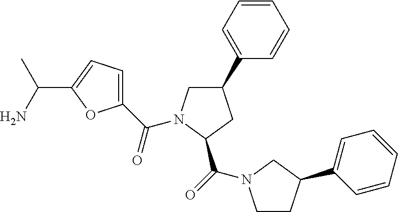

The title compound was synthesized from Preparation 5a and 82 according to the procedure described in Example 1. LC-MS: M+H⁺=458.6

### Example 5b

### ((2S,4R)-1-(54(R)-1-Aminoethyl)furan-2-carbonyl)-4-phenylpyrrolidin-2-yl)((R)-3-phenylpyrrolidin-1-yl)methanone

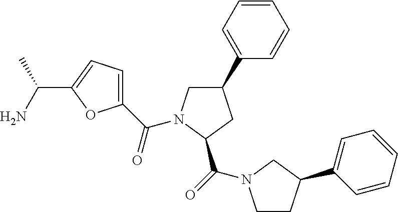

The title compound was synthesized from Preparation 5b and 82 according to the procedure described in Example 1. The residue was purified by column chromatography (100-200 mesh silica gel, methanol/chloroform, 2:98) to afford the title compound as off white solid. (TLC system: chloroform/methanol, 9:1, R_{f} 0.25). LC-MS: M+H⁺=458.6

### Example 5c

### ((2S,4R)-1-(5-((S)-1-Aminoethyl)furan-2-carbonyl)-4-phenyl pyrrolidin-2-yl)((R)-3-phenyl pyrrolidin-1-yl)methanone

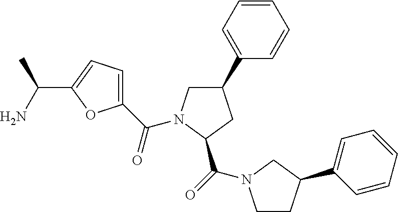

The title compound was synthesized from Preparation 5c and 82 according to the procedure described in Example 1. The residue was purified by column chromatography (100-200 mesh silica gel, methanol/chloroform, 2:98) to afford the title compound as off white solid. (TLC system: chloroform/methanol, 9:1, R_{f} 0.25). LC-MS: M+H⁺=458.6

### Example 6

### [3-(1-Amino-ethyl)-phenyl]-[(2S,4R)-4-phenyl-2-((R)-3-phenyl-pyrrolidine-1-carbonyl)-pyrrolidin-1-yl]-methanone

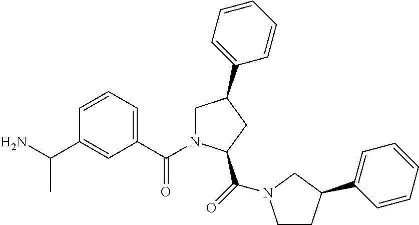

The title compound was synthesized from Preparation 2 and 82 according to the procedure described in Example 2. LC-MS: M+H⁺=468.6

### Example 7

### [6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone

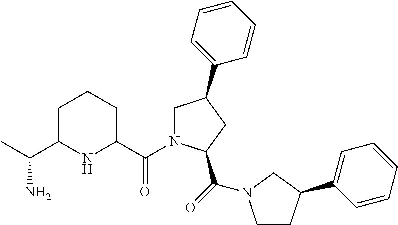

The title compound was synthesized from Preparation 1 and 82 according to the procedure described in Example 2. LC-MS: M+H⁺=475.6

### Example 8

### [6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone

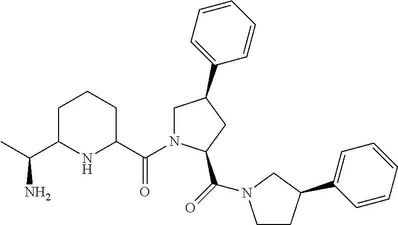

The title compound was synthesized from Preparation 1 and 82 according to the procedure described in Example 2. LC-MS: M+H⁺=475.6

### Example 9a

### {(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone

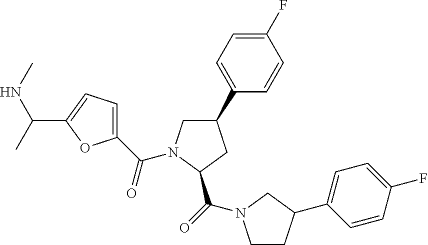

The title compound was synthesized from Preparation 4a and 83 with racemic 3-(4-fluoro-phenyl)-pyrrolidine according to the procedure described in Example 1. LC-MS: M+H⁺=508.6

### Example 9b

### {(2S,4R)-4-(4-Fluoro-phenyl)-2-[3(R)-(4-fluoro-phenyl)-pyrrolidine-1-carbonyl]-pyrrolidin-1-yl}-[5-(1(R)-methylamino-ethyl)-furan-2-yl]-methanone

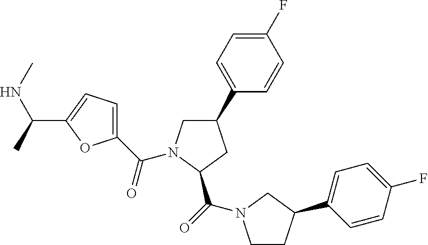

The title compound was synthesized from Preparation 4b and 83 according to the procedure described in Example 1. LC-MS: M+H⁺=508.6

### Example 9c

### {(2S,4R)-4-(4-Fluoro-phenyl)-2-[3(R)-(4-fluoro-phenyl)-pyrrolidine-1-carbonyl]-pyrrolidin-1-yl}-[5-(1(S)-methylamino-ethyl)-furan-2-yl]-methanone

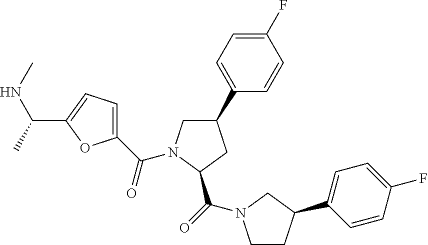

The title compound was synthesized from Preparation 4c and 83 according to the procedure described in Example 1. LC-MS: M+H⁺=508.6

### Example 10

### (5-(1-(methylamino)ethyl)furan-2-yl)((2S,4R)-4-phenyl-2-(((R)-3-phenyl pyrrolidin-1-yl)methyl)pyrrolidin-1-yl)methanone

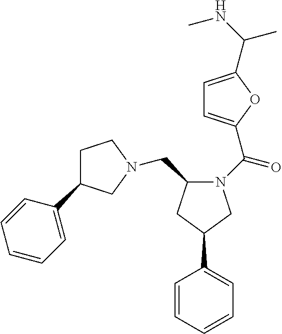

The title compound was synthesized from Preparation 4a and 84 according to the procedure described in Example 1. LC-MS: M+H⁺=458.6

### Example 11

### (3-(1-(methylamino)ethyl)phenyl)((2S,4R)-4-phenyl-2-(((R)-3-phenylpyrrolidin-1-yl)methyl)pyrrolidin-1-yl)methanone

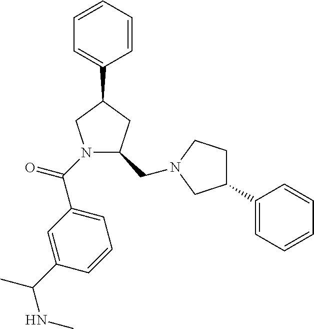

The title compound was synthesized from Preparation 8a and 84 according to the procedure described in Example 1. LC-MS: M+H⁺=468.7

### Example 12

### (6-(1-(methylamino)ethyl)pyridin-2-yl)((2S,4R)-4-phenyl-2-(((R)-3-phenylpyrrolidin-1-yl)methyl)pyrrolidin-1-yl)methanone

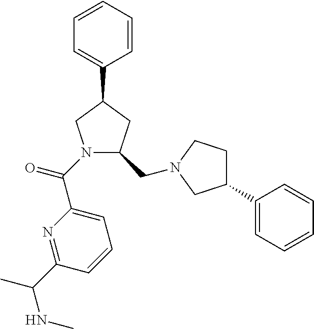

The title compound was synthesized from Preparation 7 and 84 according to the procedure described in Example 1. LC-MS: M+H⁺=469.6

### Synthesis of Compounds of Formula (I) with Substructure (IIIa)

### Example 13a

### (2S,4S)-4-Cyclohexyl-1-(2,8-diaza-spiro[4.5]decane-3-carbonyl)-pyrrolidine-2-carboxylic acid (R)-indan-1-ylamide

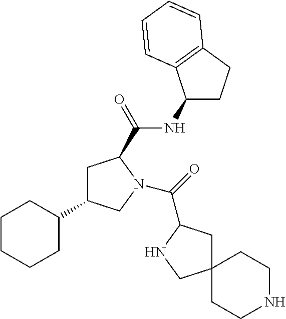

### Step 1. Reductive Amination on PL-IND Resin

To a DCM preswollen IND resin (0.5 mmol) (varian, Inc) was added a THF solution (5 mL) of (R)-2,3-dihydro-1H-inden-1-amine (0.15 g, 1.00 mmol) and trimethyl orthoformate (1.0 mL). The reaction mixture was agitated for 4 hours at room temperature. To the reaction mixture was added sodium cyanoborohydride (0.06 g, 1.00 mmol) and acetic acid (0.13 mL). The reactor was agitated and ventilated until the internal pressure lowered. The reaction mixture was agitated overnight at room temperature. The resin was drained and washed with THF (3×5 mL), Methanol (3×5 mL), DMF (3×5 mL) and DCM (3×5 mL) successively. The solid supported amine derivative was dried overnight at high vacuum. The loading of the resin was quantitative based from the mass increase of the resin. Remaining aldehyde on the resin was detected using the aldehyde test. See J. Comb. Chem. 2004, 6, 165-170.

### Step 2. Amide Coupling Reaction

To the solid supported (R)-2,3-dihydro-1H-inden-1-amine was added a DCM-DMF solution (3.0 mL, 1:1) of (2S,4R)-1-(((9H-fluoren-9-yl)methoxy)carbonyl)-4-phenylpyrrolidine-2-carboxylic acid (419 mg, 1.0 mmol), HATU, (722 mg, 1.0 mmol) and DIPEA (1.05 mL, 6.0 mmol). The reaction mixture was agitated overnight at room temperature. The resin was drained and washed with DCM (3×5 mL), DMF (3×5 mL) and DCM (3×5 mL) successively. The resin was dried overnight at high vacuum. The loading of the resin was calculated from the mass increase of the resin. A second load was performed if the loading was not good enough.

### Step 3. Fmoc Deprotection

To the DCM preswollen resin was added a solution of 20% v/v piperidine in DMF (20 mL per g resin) and agitated for 15 to 30 min. The resin was drained and washed with DCM (3×), DMF (3×) and DCM (3×) (10 mL solvent per g resin). The procedure was repeated once more.

### Step 4. Amide Coupling Reaction

To the solid supported (2S,4S)-4-cyclohexyl-N—((R)-2,3-dihydro-1H-inden-1-yl)pyrrolidine-2-carboxamide (0.25 mmol) was added a DCM-DMF solution (3.0 mL, 1:1) of 8-N-Boc-2-Fmoc-2,8-diazaspiro[4.5]decane-3-carboxylic acid (202.5 mg, 0.4 mmol), HATU, (145 mg, 3.8 mmol) and DIPEA (0.42 mL, 2.4 mmol). The reaction mixture was agitated overnight at room temperature. The resin was drained and washed with DCM (3×5 mL), DMF (3×5 mL) and DCM (3×5 mL) successively. A small sample was cleaved and analysed by LC-MS to see if any starting material was present.

### Step 5. Cleavage from PL-IND Resin

The Fmoc protected product was cleaved from the resin with 20% TFA in DCM after standing for 1 hour. The resin was filtered off and washed with DCM. The combined DCM solution was co-evaporated with 4 M HCl in dioxane (1 mL) and toluene. The crude oil precipitated out when triturated with diethyl ether. The diethyl ether was removed and the precipitate was washed with ether (3×5 mL) and dried.

### Step 6. (2S,4S)-4-Cyclohexyl-1-(2,8-diaza-spiro[4.5]decane-3-carbonyl)-pyrrolidine-2-carboxylic acid (R)-indan-1-ylamide

To 3-[(2S,4S)-4-cyclohexyl-2-((R)-indan-1-ylcarbamoyl)-pyrrolidine-1-carbonyl]-2,8-diaza-spiro[4.5]decane-2-carboxylic acid 9H-fluoren-9-ylmethyl ester, hydrochloride (50 mg) dissolved in methanol (1 mL) was added aqueous ammonia (1 mL, 33%). After stirring overnight the reaction mixture were concentrated in vacou and the crude purified on a MP-TsOH column. The crude was eluted with 2 M ammonium in methanol. The solvent was removed under reduced pressure and the crude was purified by aluminium oxide gel column chromatography to afford 12.8 mg of the title compound. LC-MS: M+H⁺=479.7

### Example 13b

### Diastereomeric pure (2S,4S)-4-Cyclohexyl-1-(2,8-diaza-spiro[4.5]decane-3-carbonyl)-pyrrolidine-2-carboxylic acid (R)-indan-1-ylamide

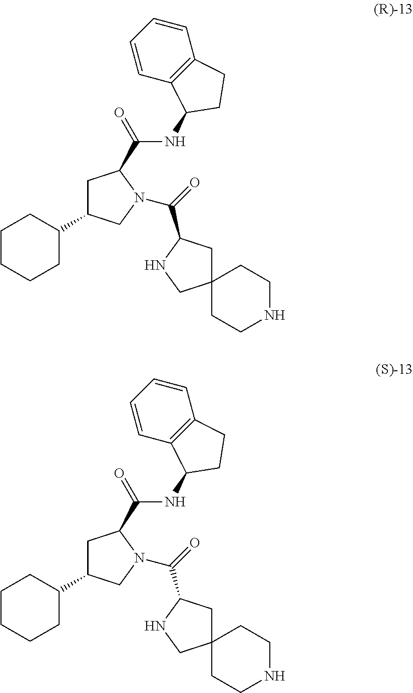

Diastereomeric pure compounds (R)-13 and (S)-13 from Example 13a was obtained from a six time up scaling of the step 1 to 5 in Example 13a followed by chiral HPLC separation of the diastereomeric mixture (Chiralpak ADH (250×4.6 mm), 5 micron, mobile phase, hexane, ethanol and TFA 80/20/0.1, flow rate: 0.8 ml/min). The absolute configuration of the two diastereomers is not known. To each diastereomer (240 mg) was added ethanolic NH₃ (6 mL) at 0° C. and stirred for 2 h at room temperature. The resulting mixture was evaporated, washed with petroleum ether and purified by column chromatography (100-200 mesh silica gel, 5% methanol in chloroform added 1% aq. NH₃) to afford two products as a hygroscopic solids. (60 mg, 36.8%) and (90 mg, 60%) (TLC system: 30% methanol in chloroform added 2% aq. NH₃, R_{f} 0.1 for both diastereomer). LC-MS: M+H⁺=479.7

### Example 14

### 2,8-Diaza-spiro[4.5]decane-3-carboxylic acid [(S)-cyclohexyl-((R)-indan-1-ylcarbamoyl)-methyl]-amide

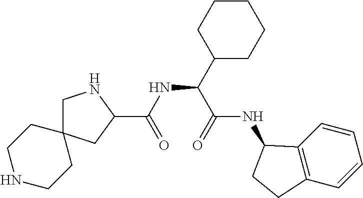

The title compound was synthesized from (R)-2,3-dihydro-1H-inden-1-amine, (S)-2-cyclohexyl-[(9H-fluoren-9-ylmethoxycarbonylamino)]-acetic acid and 8-N-Boc-2-Fmoc-2,8-diazaspiro[4.5]decane-3-carboxylic acid according to the procedure described in Example 13a. LC-MS: M+H⁺=439.6

### Synthesis of Compounds of Formula (I) with Substructure (IV)

### Example 15

### (2S,4R)-14(3R,5S)-1-((S)-2-((S)-2-aminopropanamideo)-3-(furan-2-yl)propanoyl)-3-phenylpyrrolidine-5-carbonyl)-N-methyl-4-phenylpyrrolidine-2-carboxamide

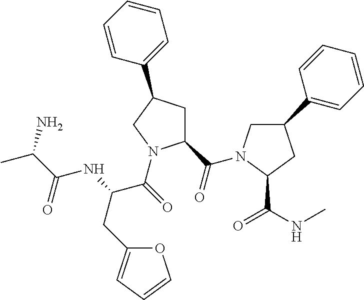

### Step 1-10. (2S,4R)-1-((3R,5S)-1-((S)-2-((S)-2-aminopropanamideo)-3-(furan-2-yl)propanoyl)-3-phenylpyrrolidine-5-carbonyl)-N-methyl-4-phenylpyrrolidine-2-carboxamide

To a DCM preswollen 3-({Methyl-Fmoc-amino}-methyl)-indol-1-yl]-acetyl AM resin (0.5 mmol) (novobiochem, Inc) was added a solution of 20% v/v piperidine in DMF (20 mL per g resin) and agitated for 30 to 45 min. The resin was drained and washed with DCM (3×), DMF (3×) and DCM (3×) (10 mL solvent per g resin). The procedure was repeated once more and the progress of the deprotection was monitored by the ninhydrin test.

### Step 2

To the Fmoc deprotected resin was added a DCM-DMF solution (3.0 mL, 1:1) of (2S,4R)-1-(((9H-fluoren-9-yl)methoxy)carbonyl)-4-phenylpyrrolidine-2-carboxylic acid (1.0 mmol), HATU, (1.0 mmol) and DIPEA (1.05 mL, 6.0 mmol). The reaction mixture was agitated overnight at room temperature. The resin was drained and washed with DCM (3×5 mL), DMF (3×5 mL) and DCM (3×5 mL) successively. The resin was dried overnight at high vacuum. The loading of the resin was calculated from the mass increase of the resin. A second load was performed if the loading was not good enough.

### Step 3-10

The resin was Fmoc deprotected and the following Fmoc amino acids ((2S,4R)-1-(((9H-fluoren-9-yl)methoxy)carbonyl)-4-phenylpyrrolidine-2-carboxylic acid, (S)-2-(((9H-fluoren-9-yl)methoxy)carbonyl)-3-(furan-2-yl)propanoic acid and (S)-2-(9H-fluoren-9-ylmethoxycarbonylamino)-propionic acid) were loaded in the same manner and the resin washed as mentioned. After each loading step a small sample was cleaved and analysed by LC-MS to see if any starting material was present. After the last Fmoc deprotection the product was cleaved from the resin with 20% TFA in DCM for 1 hour, filtered off and the resin washed with DCM. The combined DCM solution was co-evaporated with toluene. The crude was purified by LC-MS (C18 silicagel, linear gradient from 30% to 70% aqueous acetonitrile with 0.1% formic acid) to give the title compound (52 mg, 18%). LC-MS: M+H⁺=586.7

Examples 16 to 24 were synthesized according to procedures similar to those described in Example 15 or 2 or Step 1 in Example 13 combined with three Fmoc amino acid coupling reactions under Example 15.

### Example 16

### (2S,4R)-1-((3R,5S)-1-((S)-2-((S)-2-aminopropanamido)butanoyl)-3-phenylpyrrolidine-5-carbonyl)-N-methyl-4-phenylpyrrolidine-2-carboxamide

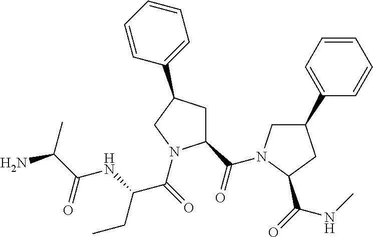

The title compound was synthesized from (2S,4R)-1-(((9H-fluoren-9-yl)methoxy)carbonyl)-4-phenylpyrrolidine-2-carboxylic acid, (2S,4R)-1-(((9H-fluoren-9-yl)methoxy)carbonyl)-4-phenylpyrrolidine-2-carboxylic acid, (S)-2-(9H-fluoren-9-ylmethoxycarbonylamino)-butyric acid and (S)-2-(9H-fluoren-9-ylmethoxycarbonylamino)-propionic acid according to the procedure described in Example 15. LC-MS: M+H⁺=534.7

### Example 17

### (2S,4R)-1-((S)-2-((R)-2-aminopropanamido)-3-(4-carbamoyl phenyl)propanoyl)-N—((R)-2,3-dihydro-1H-inden-1-yl)-4-phenylpyrrolidine-2-carboxamide

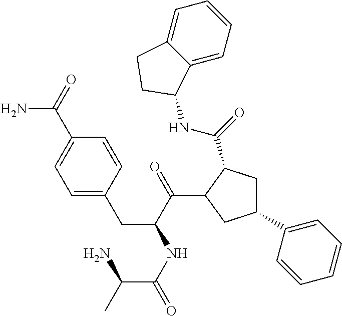

The title compound was synthesised according to procedures described in Step 1 for Example 13 using (R)-2,3-dihydro-1H-inden-1-amine followed by three Fmoc amino acid coupling reactions ((2S,4R)-1-(((9H-fluoren-9-yl)methoxy)carbonyl)-4-phenylpyrrolidine-2-carboxylic acid, Fmoc-L-4-carbamoylphenylalanin and (S)-2-(9H-fluoren-9-ylmethoxycarbonylamino)-propionic acid) according to the procedure described in Example 15. LC-MS: M+H⁺=568.7

### Example 18

### 1-((S)-2-((S)-2-aminopropanamido)-3-(3-carbamoyl phenyl)propanoyl)-N—((R)-2,3-dihydro-1H-inden-1-yl)-3-phenylazetidine-2-carboxamide

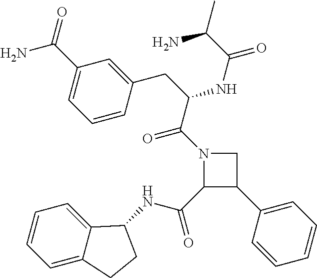

The title compound was synthesised according to procedures described in Step 1 for Example 13 using (R)-2,3-dihydro-1H-inden-1-amine followed by three Fmoc amino acid coupling reactions (trans 3-phenyl-azetidine-1,2-dicarboxylic acid 1-(9H-fluoren-9-ylmethyl)ester, Fmoc-L-4-carbamoylphenylalanin and (S)-2-(9H-fluoren-9-ylmethoxycarbonylamino)-propionic acid) according to the procedure described in Example 15. LC-MS: M+H⁺=554.7

### Example 19

### (2S,4R)-1-((S)-2-((S)-2-aminopropanamido)-3-(3-cyanophenyl)propanoyl)-N—((R)-2,3-dihydro-1H-inden-1-yl)-4-phenylpyrrolidine-2-carboxamide

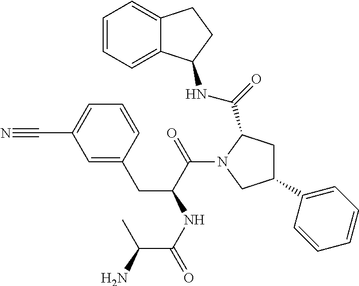

The title compound was synthesised according to procedures described in Step 1 for Example 13 using (R)-2,3-dihydro-1H-inden-1-amine followed by three Fmoc amino acid coupling reactions ((2S,4R)-1-(((9H-fluoren-9-yl)methoxy)carbonyl)-4-phenylpyrrolidine-2-carboxylic acid, Fmoc-L-3-cyanophenylalanin and (S)-2-(9H-fluoren-9-ylmethoxycarbonylamino)-propionic acid) according to the procedure described in Example 15. LC-MS: M+H⁺=550.7

### Example 20

### (2S,4R)-1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide

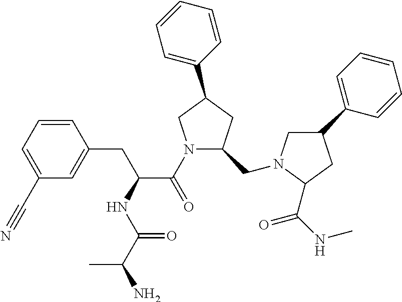

The title compound was synthesized from (2S,4R)-1-(((9H-fluoren-9-yl)methoxy)carbonyl)-4-phenylpyrrolidine-2-carboxylic acid, (2S,4R)-1-(((9H-fluoren-9-yl)methoxy)carbonyl)-4-phenylpyrrolidine-2-carboxylic acid, Fmoc-L-3-cyanophenylalanin and (S)-2-(9H-fluoren-9-ylmethoxycarbonylamino)-propionic acid according to the procedure described in Example 15. LC-MS: M+H⁺=621.7

### Example 21

### (2S,4R)-1-((3R,5S)-1-(2-((S)-2-aminopropanamido)-3-(1H-1,2,4-triazol-1-yl)propanoyl)-3-phenylpyrrolidine-5-carbonyl)-N-methyl-4-phenylpyrrolidine-2-carboxamide

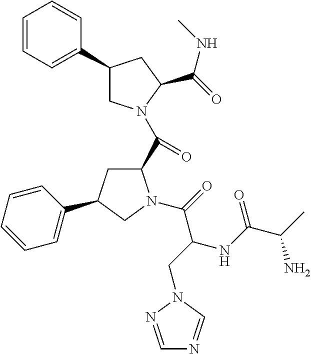

The title compound was synthesized from (2S,4R)-1-(((9H-fluoren-9-yl)methoxy)carbonyl)-4-phenylpyrrolidine-2-carboxylic acid, (2S,4R)-1-(((9H-fluoren-9-yl)methoxy)carbonyl)-4-phenylpyrrolidine-2-carboxylic acid, 2-(9H-Fluoren-9-ylmethoxycarbonylamino)-3-[1,2,4]triazol-1-yl-propionic acid and (S)-2-(9H-fluoren-9-ylmethoxycarbonylamino)-propionic acid according to the procedure described in Example 15. LC-MS: M+H⁺=587.7

### Example 22

### (S)—N—((S)-3-(3-cyanophenyl)-1-oxo-1-((2S,4R)-4-phenyl-2-(((R)-3-phenylpyrrolidin-1-yl)methyl)pyrrolidin-1-yl)propan-2-yl)-2-(methylamino)butanamide

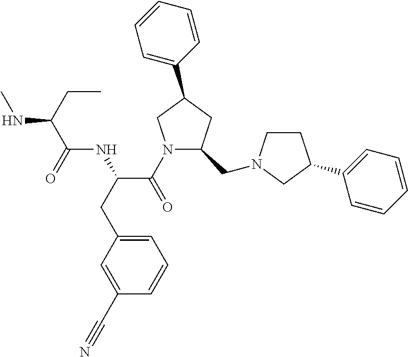

The title compound was synthesized from Preparation 11 and 84 according to the procedure described in Example 2. LC-MS: M+H⁺=578.8

### Example 23

### (2S,4R)-1-((S)-2-((R)-2-aminopropanamido)-3-(3-carbamoylphenyl)propanoyl)-N—((R)-2,3-dihydro-1H-inden-1-yl)-4-phenylpyrrolidine-2-carboxamide

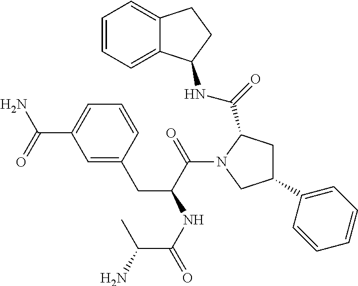

The title compound was synthesised according to procedures described in Step 1 for Example 13 using (R)-2,3-dihydro-1H-inden-1-amine followed by three Fmoc amino acid coupling reactions ((2S,4R)-1-(((9H-fluoren-9-yl)methoxy)carbonyl)-4-phenylpyrrolidine-2-carboxylic acid, Fmoc-L-3-carbamoylphenylalanin and (S)-2-(9H-fluoren-9-ylmethoxycarbonylamino)-propionic acid) according to the procedure described in Example 15. LC-MS: M+H⁺=568.7

### Example 24

### 1-((S)-2-((S)-2-aminopropanamido)-3-(3-carbamoylphenyl)propanoyl)-N—((R)-2,3-dihydro-1H-inden-1-yl)-2-phenylazetidine-3-carboxamide

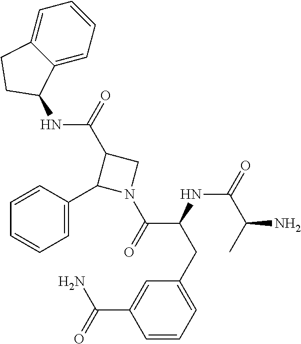

The title compound was synthesised according to procedures described in Step 1 for Example 13 using (R)-2,3-dihydro-1H-inden-1-amine followed by three Fmoc amino acid coupling reactions (trans 3-phenyl-azetidine-1,2-dicarboxylic acid 1-(9H-fluoren-9-ylmethyl)ester, Fmoc-L-3-carbamoylphenylalanin and (S)-2-(9H-fluoren-9-ylmethoxycarbonylamino)-propionic acid) according to the procedure described in Example 15. LC-MS: M+H⁺=554.7

### Assays

### Biotinylated BIR3 XIAP Domain Alphascreen Assay

Compounds were incubated with Streptavidin Donor Beads, Anti-FITC Acceptor Beads, biotinylated XIAP BIR3 and a FITC-labeled IAP-binding molecule. Following incubation for 2 hours the wells were read in an Envision reader. IC50s correspond to the concentration of compound which reduces the alphascreen signal to half of the maximum.

TABLE 6

| Example | IC50^{(a)} |
|---|---|
| Example 2 | XX |
| Example 5 | XXX |
| Example 6 | XX |
| Example 7 | X |
| Example 8 | XX |
| Example 15 | XXX |
| Example 16 | XXX |
| Example 17 | XXX |
| Example 18 | XXX |
| Example 19 | XXX |
| Example 20 | XXX |
| Example 21 | XXX |
| Example 22 | XXX |
| Example 23 | XXX |
| Example 24 | XXX |

^{(a)}“XXX” = IC50 between 0.1 nM and 100 nM; “XX” = IC50 between 100 nM and 1 μM; and “X” = IC50 above 1 μM.

### HIS-Tagged BIR3 XIAP Domain Alphascreen Assay

Compounds were incubated with Streptavidin Donor Beads, Ni-NTA Acceptor Beads, HIS-tagged XIAP BIR3 (RnD systems) and a biotin-labeled IAP-binding molecule. Following incubation for 2 hours the wells were read in an Envision reader. IC50s correspond to the concentration of compound which reduces the alphascreen signal to half of the maximum.

TABLE 7

| Example | IC50^{(a)} |
|---|---|
| Example 1 | XXX |
| Example 3 | XX |
| Example 9 | XXX |
| Example 10 | XX |
| Example 11 | XX |
| Example 13 | XX |
| Example 14 | XX |

^{(a)}“XXX” = IC50 between 0.1 nM and 100 nM; “XX” = IC50 between 100 nM and 1 μM; “X” = IC50 above 1 μM

cIAP Alphascreen Assay

Compounds are incubated with Streptavidin Donor Beads, Ni-NTA Acceptor Beads, HIS-tagged cIAP (RnD systems) and a biotin-labeled IAP-binding molecule. Following incubation for 2 hours the wells are read in an Envision reader. IC50s correspond to the concentration of compound which reduces the alphascreen signal to half of the maximum.

### Cell Proliferation Assay

The ability of compounds to inhibit tumor cell growth in vitro is monitored using the CellTiter 96 Aqueous Non-Radioactive Cell Proliferation Assay (Promega). Cell types used are SKOV3 (human ovarian cancer) and MDA-MB-231 (human mammary cancer). This assay is composed of solutions of a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine methosulfate) PMS. MTS is bioreduced by cells into a formazan product, the absorbance of which is measured at 490 nm. The conversion of MTS into the aqueous soluble formazan product is accomplished by dehydrogenase enzymes found in metabolically active cells. The quantity of formazan product as measured by the amount of 490 nm absorbance is directly proportional to the number of living cells in culture.

Xenograft Assay with SKOV3 Human Ovarian Cancer Cell Line or MDA-MB-231 Mammary Cancer Cell Line

Female CD-1 nude mice (approx. 25 g) are injected with 5 million SKOV3 human ovarian cancer cells or 1 million MDA-MB-231 human mammary cancer cells in 50% matrigel subcutaneously in the right flank. When tumors are approximately 100 mm³, treatment is initiated with compounds of formula (I). The ability of compounds of formula (I) to induce tumor stasis or regression is evaluated by measuring tumor volume. Tumor volume is measured with digital callipers and calculated as volume=(x*y²)/2, where x is the longest dimension and y is the width.

### Pharmacokinetics

Compounds of formula (I) are dissolved into saline and administered at various doses using different routes of administration including intravenous bolus, intravenous infusion, oral, and subcutaneous injection.

### Items

1. A compound of formula (I)

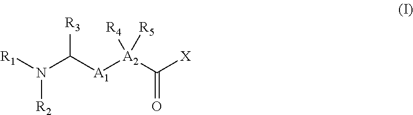

or a pharmaceutically acceptable salt, solvate or prodrug thereof,
wherein

### X is

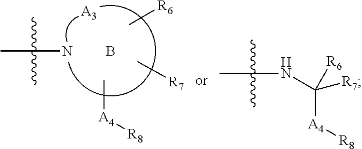

A₁ is selected from the group consisting of a single bond, —C(O)—, —NHC(O)—, —C(O)NH—, —SO₂—, —S(O)—, —C(S)—, and —CHZ₁—;
Z₁ is selected from the group consisting of H, C₁-C₆ alkyl, C₂-C₆ alkenyl, C₂-C₆ alkynyl, —(CH₂)ₘ—C₃-C₁₀ cycloalkyl, —(CH₂)ₘ-aryl, —(CH₂)ₘ-heterocyclyl, and —(CH₂)ₘ-heteroaryl, —CH₂—F, —(CH₂)ₘ—O—C₁-C₆ alkyl, —(CH₂)ₘ—O—C₃-C₆ cycloalkyl, —(CH₂)ₘ—O-aryl, —(CH₂)ₘ—O-heterocyclyl, —(CH₂)ₘ—O-heteroaryl, —(CH₂)ₘ—NHC₁-C₆ alkyl, —(CH₂)ₘ—NHC₃-C₆ cycloalkyl, —(CH₂)ₘ—NH-aryl, —(CH₂)ₘ—NH-heterocyclyl, and —(CH₂)ₘ—NH-heteroaryl.
A₂ is selected from the group consisting of cycloalkyl, aryl, heterocyclyl, heteroaryl, and —NHC(R⁴R⁵)—, wherein R⁴ and R⁵ independently are attached to cycloalkyl, aryl, heterocyclyl, or heteroaryl via any chemically feasible positions of the ring systems;
A₃ is a ring atom or moiety selected from the group consisting of C, S, O, N, —C(O)—, —NHC(O)—, and —C(O)NH—; when A3 is C it may optionally form a heterocyclic ring together with R⁴;
A₄ is a linker which is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂C(O)—, —CH₂CH₂NH—, —CH₂CH₂O—, —CH₂CH₂S—, —CH₂CH₂SO₂—, —CH₂C(O)CH₂—, —CH₂NHCH₂—, —CH₂OCH₂—, —CH₂SCH₂—, —CH₂SO₂CH₂—, —C(O)CH₂CH₂—, —NHCH₂CH₂—, —OCH₂CH₂—, —SCH₂CH₂—, —SO₂CH₂CH₂—, —CH₂C(O)NH—, —CH₂SO₂NH—, —CH₂NHC(O)—, —CH₂NHSO₂—, —C(O)NHCH₂—, —SO₂NHCH₂—, —NHC(O)CH₂—, —NHSO₂CH₂—, and —NHC(O)NH—;
B is selected from the group consisting of heterocyclic and heteroaromatic ring systems;
R¹ is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted;
R² is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-cycloalkyl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, wherein the heterocyclic ring optionally is substituted;
R³ is selected from the group consisting of H, hydroxy, halogen, C₁-C₆ alkyl, C₁-C₆ alkoxy, C₂-C₆ alkenyl, C₂-C₆ alkynyl, and C₃-C₁₀ cycloalkyl, wherein alkyl, alkenyl and alkynyl optionally are substituted;
R⁴ and R⁵ are each independently selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein any alkyl, alkenyl, alkynyl, cycloalkyl, aryl, heterocyclyl, and heteroaryl optionally are substituted;
Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}—heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, —N(R⁹)—SO₂—C₁-C₆ alkyl, —N(R⁹)—SO₂—(CH₂)_{q}—C₃-C₇ cycloalkyl, —N(R⁹)—SO₂—(CH₂)_{q}-aryl, —N(R⁹)—SO₂—(CH₂)_{q}-heterocyclyl, —N(R⁹)—SO₂—(CH₂)_{q}-heteroaryl, —SO₂—N(R¹⁰)(R¹¹), —N(R⁹)—C(O)—C₁-C₆ alkyl, —N(R⁹)—C(O)—(CH₂)_{q}—C₃-C₇ cycloalkyl, —N(R⁹)—C(O)—(CH₂)_{q}-aryl, —N(R⁹)—C(O)—(CH₂)_{q}-heterocyclyl, —N(R⁹)—C(O)—(CH₂)_{q}-heteroaryl, —C(O)—N(R¹⁰)(R¹¹), —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —CO(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)_{q}-heterocyclyl, and —O—C(O)—(CH₂)_{q}-heteroaryl, wherein any alkyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, wherein any heterocyclic ring optionally is substituted;
R⁶ and R⁷ are each independently selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein any alkyl, cycloalkyl, aryl, heterocyclyl, and heteroaryl optionally are substituted;
Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂), —C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —NH(R⁹), —N(R⁹)—SO₂—C₁-C₆ alkyl, —N(R⁹)—SO₂—(CH₂), —C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂), —C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—) N(R¹⁰(R¹¹), —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)ᵣ-heteroaryl, —CO(O)—C₁-C₁₀ alkyl, —O—C(O)—(CH₂), —C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl, wherein any alkyl, cycloalkyl, aryl, heterocyclyl, and heteroaryl optionally are substituted;
R⁸ is selected from the group consisting of C₃-C₁₀ cycloalkyl, aryl, heterocyclyl, heteroaryl, aryl-C₁-C₆ alkyl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted;
R⁹ is selected from the group consisting of H, C₁-C₆ alkyl, trifluoromethyl, trifluoroethyl, C₁-C₆ alkoxy, halogen-C₁-C₆ alkyl, —(CH₂)₀₋₂-aryl, —(CH₂)₀₋₂-heterocyclyl, and —(CH₂)₀₋₂-heteroaryl;
R¹⁰ and R¹¹ are each independently selected from the group consisting of H, C₁-C₄ alkyl, C₃-C₇ cycloalkyl, aryl, —(C₂)₁₋₆—C₃-C₇ cycloalkyl, —(C₂)₁₋₆-aryl, wherein alkyl, cycloalkyl, and aryl optionally are substituted, or R¹⁰ together with R¹¹ may form a heterocyclyl ring together with the nitrogen to which they are attached;
m is 0 or an integer from 1 to 5;
n is 0 or an integer from 1 to 6;
p is 0 or an integer from 1 to 6;
q is 0 or an integer from 1 to 6;
r is 0 or an integer from 1 to 6;
with the proviso that when A2 is —NHC(R⁴R⁵)—, then X is not

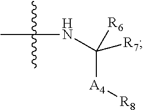

with the proviso that when A₁ is a single bond, A₂ is an oxazol ring, B is a pyrrolidinyl, R¹ and R² is H, R³ is selected from H or methyl, R⁴ and R⁵ is selected from H or methyl, and R⁸ is phenyl, 4-hydroxy-1-phenyl, or 3-indolyl, then at least one of R⁶ and R⁷ is different from H;
with the proviso that when A₁ is a —C(O)—, A₂ is —NHC(R⁴R⁵)—, B is pyrrolidinyl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, A₄ is a single bond, B is pyrrolidinyl, R¹ is H, R² is methyl, R³ is methyl, one of R⁴ and R⁵ is cyclohexyl, and one of R⁶ and R⁷ is H, then the other of R⁶ and R⁷ is not benzyloxy;
with the proviso that when A₁ is a —C(O)—, A₂ is —NHC(R⁴R⁵)—, B is octahydro-1H-pyrrolo[2,3-c]pyridin-1-yl, 7-oxooctahydro-1H-pyrrolo[2,3-c]pyridin-1-yl, octahydropyrrolo[2,3-c]azepin-1(2H)-yl, 8-oxooctahydropyrrolo[2,3-c]azepin-1(2H)-yl hexahydropyrrolo[3,4-b]pyrrol-1(2H)-yl, or 6-oxohexahydropyrrolo[3,4-b]pyrrol-1(2H)-yl, R¹ is H, R² is methyl, R³ is methyl or ethyl, and one of R⁴ and R⁵ is isopropyl, tert-butyl or cyclohexyl, then at least one of R⁶ and R⁷ is not H;
with the proviso that when A₁ is a —C(O)—, A₂ is —NHC(R⁴R⁵)—, B is 7-oxooctahydro-1H-pyrrolo[2,3-c]pyridinyl, A₄ is —CH₂CH₂—, R¹ is H, R² is methyl, R³ is methyl, one of R⁴ and R⁵ is isopropyl, R⁸ is phenyl, and one of R⁶ and R⁷ is H, then the other of R⁶ and R⁷ is not benzyloxy;
with the proviso that when A₁ is a —C(O)—, A₂ is —NHC(R⁴R⁵)—, A4 contains a —NHC(O)-fragment or is —CH₂—O—, B is pyrrolidinyl, R¹ and R² is H, R³ is methyl, ethyl, propyl or isopropyl, and R⁴ forms a heterocyclic ring with A₃, then at least one of R⁶ and R⁷ is not H; and
with the proviso that when A₁ is a —C(O)—, A₂ is —NHC(R⁴R⁵)—, A₄ contains a —NHC(O)-fragment, B is pyrrolidinyl, R³ is methyl, ethyl, propyl or isopropyl, and R⁴ forms a heterocyclic ring with A₃, then at least one of R⁶ and R⁷ is not H.

2. The compound according to item 1, wherein A1 is selected from the group consisting of a single bond, —C(O)—, SO₂, —S(O)—, and —CHZ₁—.

3. The compound according to any of the preceding items, wherein A1 is selected from the group consisting of a single bond, —C(O)— and —CHZ₁—.

4. The compound according to any of the preceding items, wherein Z₁ is selected from the group consisting of H, C₁-C₄ alkyl, —CH₂—F, —CH₂—C₃-C₆ cycloalkyl, —CH₂-aryl, —CH₂-heterocyclyl, —CH₂-heteroaryl, —CH₂—OC₁-C₆ alkyl, —CH₂—OC₃-C₆ cycloalkyl, —CH₂—O-aryl, —CH₂—O-heterocyclyl, —CH₂—O-heteroaryl, —CH₂—NHC₁-C₆ alkyl, NHC₃-C₆ cycloalkyl, —CH₂—NH-aryl, —CH₂—NH-heterocyclyl, and —CH₂—NH-heteroaryl.

5. The compound according to any of the preceding items, wherein A1 is a single bond.

6. The compound according to any of items 1-4, wherein A1 is —C(O)—.

7. The compound according to any of the preceding items, wherein A2 is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems.

8. The compound according to item 7, wherein A2 is selected from the group consisting of cyclopropyl, cyclobutyl, cyclopentyl, cyclohexyl, azetidinyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azetidinonyl, azepanylyl, oxazetidinyl, diazetidinyl, oxazepanylyl, diazepanylyl, pyrrolidinonyl, piperidinonyl, azepanylonyl, thioxoazetidinyl, phenyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, 5,6-dihydro-4H-cyclopenta[c]furanyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

9. The compound according to any of the preceding items, wherein A2 is selected from 5- or 6-membered cycloalkyl, aryl, heterocyclyl, and heteroaryl, and wherein R⁴ and R⁵ independently are attached to cycloalkyl, aryl, heterocyclyl, or heteroaryl via any chemically feasible positions of the ring systems.

10. The compound according to item 9, wherein A2 is selected from the group consisting of cyclopentyl, cyclohexyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azepanylyl, oxazepanyl, diazepanyl, pyrrolidinonyl, piperidinonyl, azepanylonyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, 5,6-dihydro-4H-cyclopenta[c]furanyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

11. The compound according to any of the preceding items, wherein A2 is selected from 5-membered cycloalkyl, heterocyclyl, and heteroaryl, wherein R⁴ and R⁵ independently are attached to cycloalkyl, aryl, heterocyclyl, or heteroaryl via any chemically feasible positions of the ring systems.

12. The compound according to item 11, wherein A2 is selected from the group consisting of cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrazolyl, triazolyl, and tetrazolyl.

13. The compound according to item 11, wherein A2 is selected from the group consisting of cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, pyrrolyl, furanyl, isoxazolyl, thienyl, thiazolyl, isothiazolyl, imidazolyl, oxadiazolyl, thiadiazolyl, oxathiazolyl, pyrazolyl, triazolyl, and tetrazolyl.

14. The compound according to item 11, wherein A2 is selected from the group consisting of cyclopentyl, pyrrolidinyl, tetrahydrofuranyl, isoxazolidinyl, oxazolidinyl, tetrahydrothiophene, isothiazolidinyl, thiazolidinyl, pyrazolidinyl, imidazolidinyl, dihydropyrrole, pyrrolidinonyl, cyclopentadienyl, isoxazolyl, isothiazolyl, oxadiazolyl, thiadiazolyl, oxathiazolyl, pyrazolyl, triazolyl, and tetrazolyl.

15. The compound according to item 11, wherein A2 is selected from 5-membered heterocyclyl, wherein R⁴ and R⁵ independently are attached to heterocyclyl via any chemically feasible positions of the ring system.

16. The compound according to item 11, wherein A2 is selected from 5-membered heteroaryl, wherein R⁴ and R⁵ independently are attached to heteroaryl via any chemically feasible positions of the ring system.

17. The compound according to item 11, wherein A2 is selected from the group consisting of pyrrolidinyl, tetrahydrofuranyl, dihydropyrrole, pyrrolidinonyl, cyclopentadienyl, isoxazolyl, isothiazolyl, oxadiazolyl, thiadiazolyl, oxathiazolyl, pyrazolyl, triazolyl, and tetrazolyl.

18. The compound according to any of items 1-6, wherein A2 is —NHC(R⁴R⁵)—.

19. The compound according to any of the preceding items, wherein A3 is C, and optionally forms a heterocyclic ring together with R⁴.

20. The compound according to any of the preceding items, wherein A3 forms a heterocyclic ring together with R⁴.

21. The compound according to any of the preceding items, wherein A3 is C.

22. The compound according to any of the preceding items, wherein A4 is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂O—, —CH₂OCH₂—, and —OCH₂CH₂—.

23. The compound according to any of the preceding items, wherein A4 is selected from the group consisting of —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂O—, —CH₂OCH₂—, and —OCH₂CH₂—.

24. The compound according to any of the preceding items, wherein A4 is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHSO₂—, —SO₂NH—, —CH₂CH₂CH₂—, —CH₂CH₂O—, —CH₂OCH₂—, and —OCH₂CH₂—.

25. The compound according to any of the preceding items, wherein A4 is selected from the group consisting of —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, and —SO₂NH—.

26. The compound according to any of the preceding items, wherein A4 is selected from the group consisting of single bond, —NH—, —O—, —S—, —SO₂—, —NHCH₂—, —CH₂NH—, —OCH₂—, —CH₂O—, —SCH₂—, —CH₂S—, —SO₂CH₂—, —CH₂SO₂—, —NHSO₂—, —SO₂NH—, —CH₂CH₂NH—, —CH₂CH₂S—, —CH₂CH₂SO₂—, —CH₂NHCH₂—, —CH₂OCH₂—, —CH₂SCH₂—, —CH₂SO₂CH₂—, —NHCH₂CH₂—, —OCH₂CH₂—, —SCH₂CH₂—, —SO₂CH₂CH₂—, —CH₂SO₂NH—, —CH₂NHSO₂—, —SO₂NHCH₂—, and —NHSO₂CH₂—.

27. The compound according to any of the preceding items, wherein A4 is a single bond.

28. The compound according to any of the preceding items, wherein A4 is selected from the group consisting of —CH₂—, —C(O)—, —NH—, —O—, —S—, and —SO₂—.

29. The compound according to any of the preceding items, wherein A4 is attached to B, via a ring atom next to the Nitrogen atom of B.

30. The compound according to any of the preceding items, wherein B is selected from the group consisting of 4 membered, 5 membered, 6 membered, and 7 membered heterocyclic and heteroaromatic ring systems.

31. The compound according to any of the preceding items, wherein B is selected from the group consisting of azetidine, 1,2-diazetidine, 1,3-diazetidine, 1,2-oxazetidine, 1,3-oxazetidine, 1,2-thiazetidine, 1,3-thiazetidine, 1,2-dihydroazete, pyrrolidine, pyrazolidine, imidazolidine, isoxazolidine, 1,3-oxazolidine, isothiazolidine, 1,3-thiazolidine, 2,3-dihydro-1H-pyrrole, 2,5-dihydro-1H-pyrrole, 2,5-dihydroisoxazole, 2,3-dihydro-1,3-oxazole, 2,5-dihydroisothiazole, 2,3-dihydro-1,3-thiazole, 2,3-dihydroisoxazole, 2,3-dihydroisothiazole, piperidine, hexahydropyridazine, hexahydropyrimidine, piperazine, 1,2-oxazinane, 1,3-oxazinane, morpholine, 1,2-thiazinane, 1,3-thiazinane, thiomorpholine, 1,2,3,4-tetrahydropyridine, 1,2,3,6-tetrahydropyridine, 1,2,3,6-tetrahydropyridine, 1,2-dihydropyridine, 1,4-dihydropyridine, 1,2,3,4-tetrahydropyridazine, 1,2,3,4-tetrahydropyrimidine, 1,2,3,4-tetrahydropyrazine, 5,6-dihydro-2H-1,2-oxazine, 3,6-dihydro-2H-1,3-oxazine, 3,4-dihydro-2H-1,4-oxazine, 5,6-dihydro-2H-1,2-thiazine, 3,6-dihydro-2H-1,3-thiazine, 3,4-dihydro-2H-1,4-thiazine, 3,6-dihydro-2H-1,2-oxazine, 3,4-dihydro-2H-1,3-oxazine, 3,4-dihydro-2H-1,2-oxazine, 1,2-dihydropyridine, 1,4-dihydropyridine, tetrahydropyrimidin-4(1H)-one, piperazin-2-one, 1,3,5-triazinan-2-one, piperidin-4-one, piperidin-3-one, azepane, 1,2-diazepane, 1,3-diazepane, 1,4-diazepane, 1,2-oxazepane, 1,3-oxazepane, 1,4-oxazepane, 1,2-thiazepane, 1,3-thiazepane, 1,4-thiazepane, 2,3,4,5-tetrahydro-1H-azepine, 2,3,4,7-tetrahydro-1H-azepine, 2,3,6,7-tetrahydro-1H-azepine, 2,3-dihydro-1H-azepine, 1H-azepine, 4,5-dihydro-1H-azepine, 2,3,4,5-tetrahydro-1H-1,2-diazepine, 2,3,4,5-tetrahydro-1H-1,3-diazepine, 2,3,4,5-tetrahydro-1H-1,4-diazepine, 4,5,6,7-tetrahydro-1H-1,4-diazepine, 2,5,6,7-tetrahydro-1,2-oxazepine, 2,3,6,7-tetrahydro-1,3-oxazepine, 2,3,4,7-tetrahydro-1,4-oxazepine, 4,5,6,7-tetrahydro-1,4-oxazepine, 2,5,6,7-tetrahydro-1,2-thiazepine, 2,3,6,7-tetrahydro-1,3-thiazepine, 2,3,4,7-tetrahydro-1,4-thiazepine, 4,5,6,7-tetrahydro-1,4-thiazepine, 2,3,4,5-tetrahydro-1,2-oxazepine, 2,3,6,7-tetrahydro-1,2-oxazepine, 2,3,4,7-tetrahydro-1,3-oxazepine, and 2,3,4,5-tetrahydro-1,4-oxazepine.

32. The compound according to any of the preceding items, wherein B is selected from the group consisting of 5 membered and 6 membered heterocyclic and heteroaromatic rings.

33. The compound according to any of the preceding items, wherein B is selected from the group consisting of pyrrolidine, pyrazolidine, imidazolidine, isoxazolidine, 1,3-oxazolidine, isothiazolidine, 1,3-thiazolidine, 2,3-dihydro-1H-pyrrole, 2,5-dihydro-1H-pyrrole, 2,5-dihydroisoxazole, 2,3-dihydro-1,3-oxazole, 2,5-dihydroisothiazole, 2,3-dihydro-1,3-thiazole, 2,3-dihydroisoxazole, 2,3-dihydroisothiazole, piperidine, hexahydropyridazine, hexahydropyrimidine, piperazine, 1,2-oxazinane, 1,3-oxazinane, morpholine, 1,2-thiazinane, 1,3-thiazinane, thiomorpholine, 1,2,3,4-tetrahydropyridine, 1,2,3,6-tetrahydropyridine, 1,2,3,6-tetrahydropyridine, 1,2-dihydropyridine, 1,4-dihydropyridine, 1,2,3,4-tetrahydropyridazine, 1,2,3,4-tetrahydropyrimidine, 1,2,3,4-tetrahydropyrazine, 5,6-dihydro-2H-1,2-oxazine, 3,6-dihydro-2H-1,3-oxazine, 3,4-dihydro-2H-1,4-oxazine, 5,6-dihydro-2H-1,2-thiazine, 3,6-dihydro-2H-1,3-thiazine, 3,4-dihydro-2H-1,4-thiazine, 3,6-dihydro-2H-1,2-oxazine, 3,4-dihydro-2H-1,3-oxazine, 3,4-dihydro-2H-1,2-oxazine, 1,2-dihydropyridine, 1,4-dihydropyridine, tetrahydropyrimidin-4(1H)—O-one, piperazin-2-one, 1,3,5-triazinan-2-one, piperidin-4-one, and piperidin-3-one.

34. The compound according to any of the preceding items, wherein B is selected from the group consisting of azetidin-1-yl, 1,2-diazetidin-1-yl, 1,3-diazetidin-1-yl, 1,2-oxazetidin-2-yl, 1,2-thiazetidin-2-yl, pyrrolidin-1-yl, imidazolidin-1-yl, 1,3-oxazolidin-3-yl, 1,3-thiazolidin-3-yl, piperidin-1-yl, 1,3-oxazinan-3-yl, morpholin-4-yl, and 3-oxopiperazin-1-yl, and 4-oxopiperidin-1-yl.

35. The compound according to any of the preceding items, wherein B is selected from the group consisting of azetidin-1-yl, pyrrolidin-1-yl, piperidin-1-yl, 2-oxo-piperazinyl, morpholin-4-yl, and piperazin-1-yl.

36. The compound according to any of the preceding items, wherein B is pyrrolidinyl.

37. The compound according to any of items 1-28, wherein B is selected from the group consisting of bicyclic, fused or spiro-cyclic heterocyclyl, and bicyclic, fused or spiro-cyclic heteroaryl rings.

38. The compound according to any of items 1-28, wherein B is selected from the group consisting of 2,3-dihydro-1H-indol-1-yl, 1,3-dihydro-2H-isoindol-2-yl, hexahydropyrrolo[2,3-e][1,3]oxazin-5(2H)-yl, hexahydro[1,3]oxazolo[4,5-c]pyridin-3(2H)-yl, tetrahydro-3aH-[1,3]oxazolo[4,5-e][1,3]oxazin-1(2H)-yl, hexahydro[1,3]thiazolo[4,5-c]pyridin-3(2H)-yl, hexahydropyrrolo[2,3-e][1,3]thiazin-5(2H)-yl, tetrahydro-3aH-[1,3]thiazolo[4,5-e][1,3]thiazin-1(2H)-yl, tetrahydro-3aH-[1,3]thiazolo[4,5-e][1,3]oxazin-1(2H)-yl, tetrahydro-3aH-[1,3]oxazolo[4,5-e][1,3]thiazin-1(2H)-yl, 3,4-dihydroisoquinolin-2(1H)-yl, 3,4-dihydroquinolin-1(2H)-yl, hexahydropyrrolo[3,4-b]pyrrol-5(1H)-yl, octahydropyrrolo[2,3-c]azepin-1(2H)-yl, 7-oxooctahydro-1H-pyrrolo[2,3-c]pyridin-1-yl, 8-oxooctahydropyrrolo[2,3-c]azepin-1(2H)-yl, 6-oxohexahydropyrrolo[3,4-b]pyrrol-1(2H)-yl, octahydro-1H-pyrrolo[2,3-c]pyridin-1-yl, octahydro-1H-pyrrolo[3,2-c]pyridin-1-yl, and 2,7-diazaspiro[4.5]dec-2-yl.

39. The compound according to any of items 1-28, wherein B is selected from the group consisting of octahydro-1H-pyrrolo[2,3-c]pyridin-1-yl, octahydro-1H-pyrrolo[3,2-c]pyridin-1-yl, octahydropyrrolo[2,3-c]azepin-1(2H)-yl, octahydro-2,7-naphthyridin-2(1H)-yl, 3,4-dihydroisoquinolin-2(1H)-yl, 3,4-dihydroquinolin-1(2H)-yl, hexahydropyrrolo[3,4-b]pyrrol-5(1H)-yl, octahydropyrrolo[2,3-c]azepin-1(2H)-yl, 7-oxooctahydro-1H-pyrrolo[2,3-c]pyridin-1-yl, 8-oxooctahydropyrrolo[2,3-c]azepin-1(2H)-yl, 6-oxohexahydropyrrolo[3,4-b]pyrrol-1(2H)-yl, and 2,7-diazaspiro[4.5]dec-2-yl.

40. The compound according to any of the preceding items, wherein X is

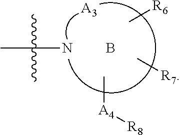

41. The compound according to any of items 1-24, wherein X is

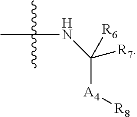

42. The compound according to any of the preceding items, wherein R¹ is selected from the group consisting of H, C₁-C₆ alkyl, C₃-C₁₀ cycloalkyl, aryl, heterocyclyl, and heteroaryl, wherein any alkyl, cycloalkyl, aryl, heterocyclyl, and heteroaryl optionally are substituted.

43. The compound according to any of the preceding items, wherein R¹ is selected from the group consisting of H and C₁-C₄ alkyl.

44. The compound according to any of the preceding items, wherein R¹ is H.

45. The compound according to any of the preceding items, wherein R² is selected from the group consisting of H, C₁-C₄ alkyl, C₁-C₄ alkoxy, C₂-C₄ alkenyl, C₂-C₄ alkynyl, C₃-C₆ cycloalkyl, aryl, heterocyclyl, heteroaryl, —(CH₂)₁₋₄-cycloalkyl, —(CH₂)₁₋₄-aryl, —(CH₂)₁₋₄-heterocyclyl, and —(C₂)₁₋₄-heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, wherein the heterocyclic ring optionally is substituted.

46. The compound according to any of the preceding items, wherein R² is selected from the group consisting of H, C₁-C₄ alkyl, C₁-C₄ alkoxy, C₂-C₄ alkenyl, C₂-C₄ alkynyl, wherein any alkyl, alkenyl and alkynyl optionally are substituted; or wherein R² together with R⁵ optionally may form a heterocyclic ring together with the nitrogen to which R² is attached, wherein the heterocyclic ring optionally is substituted.

47. The compound according to any of the preceding items, wherein R² is selected from the group consisting of H, C₁-C₄ alkyl, C₁-C₄ alkoxy, C₃-C₆ cycloalkyl, —(CH₂)₁₋₄-cycloalkyl, wherein any alkyl, cycloalkyl, optionally are substituted; or wherein R² together with R⁵ optionally may form a heterocyclic ring together with the nitrogen to which R² is attached, wherein the heterocyclic ring optionally is substituted.

48. The compound according to any of the preceding items, wherein R² is methyl.

49. The compound according to any of items 1-41, wherein R² is selected from the group consisting of C₃-C₁₀ cycloalkyl, aryl, heterocyclyl, heteroaryl, —(CH₂)₁₋₆-aryl, —(CH₂)₁₋₆-heterocyclyl, and —(CH₂)₁₋₆-heteroaryl, and wherein any cycloalkyl, aryl, heterocyclyl, and heteroaryl optionally are substituted.

50. The compound according to any of items 1-41, wherein R² is H.

51. The compound according to any of the preceding items, wherein R² together with R⁵ forms a heterocyclic ring together with the nitrogen to which R² is attached, wherein the heterocyclic ring optionally is substituted.

52. The compound according to any of the preceding items, wherein R² together with R⁵ forms a heterocyclic ring together with the nitrogen to which R² is attached, wherein the heterocyclic ring optionally is substituted, and wherein R² is a single bond.

53. The compound according to any of items 51-52, wherein the heterocyclic ring is substituted with one or more substituents selected from the group consisting of —F, —OH, —CF₃, C₁-C₄ alkyl, —CN, and —NO₂

54. The compound according to any of items 51-52, wherein R² together with R⁵ forms a heterocyclic ring selected from the group consisting of pyrrolidinyl, piperidinyl, azetidinyl, 1,2-diazetidinyl, 1,2-oxazetidinyl, 1,2-thiazetidinyl, pyrazolidinyl, isoxazolidinyl, imidazolidinyl, 1,3-oxazolidinyl, 1,3-thiazolidinyl, hexahydropyridazinyl, hexahydropyrimidinyl, piperazinyl, 1,2-oxazinanyl, 1,3-oxazinanyl, morpholinyl, 1,2-thiazinanyl, 1,3-thiazinanyl, and thiomorpholinyl, and wherein the ring optionally is substituted.

55. The compound according to any of item 51-52, wherein R² together with R⁵ forms a heterocyclic ring selected from the group consisting of azetidinyl, pyrrolidinyl, and piperidinyl, and wherein the ring optionally is substituted.

56. The compound according to any of the preceding items, wherein R³ is selected from the group consisting of H, hydroxy, halogen, C₁-C₄ alkyl, C₁-C₄ alkoxy, C₂-C₄ alkenyl, C₂-C₄ alkynyl, and C₃-C₆ cycloalkyl, wherein any alkyl, alkenyl and alkynyl optionally are substituted.

57. The compound according to any of the preceding items, wherein R³ is selected from the group consisting of H, hydroxy, and C₁-C₄ alkyl.

58. The compound according to any of the preceding items, wherein R³ is H.

59. The compound according to any of the preceding items, wherein R³ is selected from the group consisting of H, OH, methyl, ethyl, and —CH₂OH

60. The compound according to any of the preceding items, wherein R³ is selected from the group consisting of OH and —CH₂OH.

61. The compound according to any of the preceding items, wherein R³ is selected from the group consisting of fluoro and —CH₂F.

62. The compound according to any of the preceding items, wherein R⁴ and R⁵ each independently are selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, and —(CH₂)ₙ—Z₂, wherein Z₂ is as defined in item 1, and wherein any alkyl, alkenyl, alkynyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, and wherein any heterocyclic ring optionally is substituted.

63. The compound according to any of the preceding items, wherein R⁴ and R⁵ each independently are selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, and —(CH₂)ₙ—Z₂, wherein Z₂ is as defined in item 1, and wherein any alkyl, alkenyl, alkynyl, cycloalkyl, aryl, heterocyclyl, and heteroaryl optionally are substituted

64. The compound according to any of the preceding items, wherein R⁴ together with A3 forms a heterocyclic ring together with the nitrogen to which A3 is attached, and wherein the heterocyclic ring optionally is substituted.

65. The compound according to any of the preceding items, wherein R⁵ together with R² forms a heterocyclic ring together with the nitrogen to which R² is attached, and wherein the heterocyclic ring optionally is substituted.

66. The compound according to any of the preceding items, wherein R⁴ and R⁵ each independently are selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein n is 0 or an integer from 1 to 3; wherein Z₂ is as defined in item 1, and wherein any alkyl, cycloalkyl, heterocyclyl, and heteroaryl optionally are substituted.

67. The compound according to any of the preceding items, wherein R⁴ and R⁵ each independently are selected from the group consisting of H, hydroxyl, —NH₂, —CN, —SO₂, —NO₂, halogen, C₁-C₃ alkyl, C₁-C₃ alkyl substituted with fluoro, C₁-C₃ alkoxy, C₃-C₆ cycloalkyl, C₃-C₆ heterocyclyl, C₃-C₆ heteroaryl and —(CH₂)ₙ—Z₂, wherein n is 0 or 1, Z₂ is as defined in item 1, and wherein any alkyl, cycloalkyl, aryl, heterocyclyl, and heteroaryl optionally are substituted.

68. The compound according to any of the preceding items, wherein R⁴ and R⁵ each independently are selected from the group consisting of C₂-C₆ alkyl, C₂-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein n is 0 or 1, Z₂ is as defined in item 1, and wherein any alkyl, cycloalkyl, aryl, heterocyclyl, and heteroaryl optionally are substituted.

69. The compound according to any of items 1-67, wherein R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

70. The compound according to any of items 1-67, wherein R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

71. The compound according to any of items 1-67, wherein R⁴ and R⁵ each independently are selected from the group consisting of H, methyl, ethyl, propyl, isopropyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

72. The compound according to any of items 1-67, wherein R⁴ and R⁵ each independently are selected from the group consisting of H, methyl, ethyl, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

73. The compound according to any of items 1-67, wherein R⁴ and R⁵ each independently are selected from the group consisting of H, methyl, ethyl, propyl, isopropyl, methoxy, and ethoxy.

74. The compound according to any of items 1-67, wherein R⁴ and R⁵ each independently are selected from the group consisting of H, methyl, ethyl, methoxy, and ethoxy.

75. The compound according to any of the preceding items, wherein R⁴ and R⁵ each independently are selected from the group consisting of H, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, —CH₂CF₃, and —CF₂CF₃,

76. The compound according to any of the preceding items, wherein R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —SO₂, and —NO₂.

77. The compound according to any of items 1-66, wherein R⁴ and R⁵ each independently are selected from the group consisting of cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl.

78. The compound according to any of items 1-66, wherein R⁴ and R⁵ each independently are selected from the group consisting of bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, azetidinylmethyl, aziridinylmethyl, pyrrolidinylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, azetidinylethyl, aziridinylethyl, pyrrolidinylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl.

79. The compound according to any of the preceding items, wherein R⁴ and R⁵ each independently are selected from the group consisting of cyclohexyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl.

80. The compound according to any of the preceding items, wherein R⁴ and R⁵ each independently are selected from the group consisting of tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl.

81. The compound according to any of the preceding items, wherein R⁴ and R⁵ each independently are selected from the group consisting of H, methyl, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, and tert-butyl butyl.

82. The compound according to any of the preceding items, wherein R⁴ and R⁵ each independently are selected from the group consisting of H, hydroxyl, —NH₂, —CN, —F, —Cl, —Br, —CH₂OH, —O—CH₃, —CH₂F, —CHF₂, —CF₃, —CH₂Cl, —CH₂CH₂OH, —O—CH₂CH₃, —SO₂, —NO₂, ethyl, —CH₂CF₃, —CF₂CF₃, propyl, isopropyl, 2-methylpropyl, tert-butyl.

83. The compound according to any of the preceding items Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}—heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}heteroaryl, —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —OC(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)_{q}-heterocyclyl, and —O—C(O)—(CH₂)_{q}-heteroaryl, and wherein any alkyl, cycloalkyl, aryl, heterocyclyl, and heteroaryl optionally are substituted.

84. The compound according to any of the preceding items Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}—heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}—heteroaryl, and wherein any alkyl, cycloalkyl, aryl, heterocyclyl, and heteroaryl optionally are substituted.

85. The compound according to any of the preceding items, wherein Z₂ is selected from the group consisting of H, —OH, —NH₂, —CN, —SO₂, —NO₂, halogen, C₁-C₆ alkoxy, C₃-C₁₀ cycloalkyl, C₃-C₁₀ heterocyclyl, and C₃-C₁₀ heteroaryl, and wherein any alkyl, cycloalkyl, heterocyclyl, and heteroaryl optionally are substituted.

86. The compound according to any of the preceding items, wherein Z₂ is selected from the group consisting of H, —OH, —NH₂, —CN, —SO₂, —NO₂, halogen, C₁-C₃ alkoxy, C₃-C₆ cycloalkyl, C₃-C₆ heterocyclyl, and C₅-C₁₀ heteroaryl, and wherein any alkyl, cycloalkyl, heterocyclyl, and heteroaryl optionally are substituted.

87. The compound according to any of the preceding items, wherein Z₂ is selected from the group consisting of —H, methyl, —OH, —NH₂, —CN, —F, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, wherein any alkyl, cycloalkyl, aryl, heterocyclyl, and heteroaryl optionally are substituted.

88. The compound according to any of the preceding items, wherein substituents for any alkyl, cycloalkyl, aryl, heterocyclyl, and heteroaryl of R⁴, R⁵, and Z₂ is one or more substituents each independently selected from the group consisting of chloro, fluoro, hydroxyl, —C(O)NH₂, C₁-C₆ alkyl, C₁-C₆ alkoxy, and —CN.

89. The compound according to any of the preceding items, wherein R⁶ and R⁷ each independently are selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, and wherein any alkyl, cycloalkyl, aryl, heterocyclyl, and heteroaryl optionally are substituted; wherein Z₃ is selected from the group consisting of H, F, —OH, —NH₂, —NO₂, —CN, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —NH(R⁹), —N(R⁹)—SO₂—C₁-C₆ alkyl, —N(R⁹)—SO₂—(CH₂)ᵣ—C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂)ᵣ—C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—N(R¹⁰)(R¹¹), wherein any alkyl, cycloalkyl, aryl, heterocyclyl, and heteroaryl optionally are substituted; wherein p is 0, or an integer from 1 to 2; and wherein r is 0, or an integer from 1 to 2.

90. The compound according to any of the preceding items, wherein R⁶ and R⁷ each independently are selected from the group consisting of —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein Z₃ is as defined in item 1, and wherein any alkyl, cycloalkyl, aryl, heterocyclyl, and heteroaryl optionally are substituted.

91. The compound according to any of the preceding items, wherein at least one of R⁶ and R⁷ are different from H.

92. The compound according to any of the preceding items, wherein R⁶ and R⁷ both are H.

93. The compound according to any of the preceding items, wherein at least one of R⁶ and R⁷ each independently are C₁-C₆ alkyl, wherein the alkyl optionally is substituted.

94. The compound according to any of the preceding items, wherein at least one of R⁶ and R⁷ each independently are C₃-C₁₀ cycloalkyl, wherein the cycloalkyl optionally is substituted.

95. The compound according to any of the preceding items, wherein at least one of R⁶ and R⁷ each independently are aryl, wherein the aryl optionally is substituted.

96. The compound according to any of the preceding items, wherein at least one of R⁶ and R⁷ each independently are heterocyclyl, wherein the heterocyclyl optionally is substituted.

97. The compound according to any of the preceding items, wherein at least one of R⁶ and R⁷ each independently are heteroaryl, wherein the heteroaryl optionally is substituted.

98. The compound according to any of the preceding items, wherein at least one of R⁶ and R⁷ each independently are selected from the group consisting of cyclopropyl, cyclobutyl, cyclopentyl, cyclohexyl, bicyclo[2.2.2]octanyl, azetidinyl, tetrahydro-2H-pyranyl, piperidinyl, tetrahydro-2H-thiopyranyl, morpholinyl, piperazinyl, thiomorpholinylaziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, trichlorophenyl, cyclohexylmethyl, bicyclo[2.2.2]octanylmethyl, tetrahydro-2H-pyranylmethyl, piperidinylmethyl, tetrahydro-2H-thiopyranylmethyl, morpholinylmethyl, piperazinylmethyl, thiomorpholinylmethyl, cyclobutylmethyl, cyclopropylmethyl, cyclopentylmethyl, tetrahydrofuranylmethyl, pyrrolidinylmethyl, tetrahydrothienylmethyl, oxazolidinylmethyl, imidazolidinylmethyl, thiazolidinylmethyl, carbamoylbenzyl, cyanobenzyl, pyridinylmethyl, pyrimidinylmethyl, triazinylmethyl, pyrazinylmethyl, pyrrolylmethyl, triazolylmethyl, tetrazolylmethyl, pyrazolylmethyl, furanylmethyl, thienylmethyl, fluorobenzyl, hydroxybenzyl, chlorobenzyl, difluorobenzyl, dichlorobenzyl, trifluorobenzyl, trichlorobenzyl, cyclohexylethyl, bicyclo[2.2.2]octanylethyl, tetrahydro-2H-pyranylethyl, piperidinylethyl, tetrahydro-2H-thiopyranylethyl, morpholinylethyl, piperazinylethyl, thiomorpholinylethyl, cyclobutylethyl, cyclopropylethyl, cyclopentylethyl, tetrahydrofuranylethyl, pyrrolidinylethyl, tetrahydrothienylethyl, oxazolidinylethyl, imidazolidinylethyl, thiazolidinylethyl, carbamoylphenylethyl, cyanophenylethyl, pyridinylethyl, pyrimidinylethyl, triazinylethyl, pyrazinylethyl, pyrrolylethyl, triazolylethyl, tetrazolylethyl, pyrazolylethyl, furanylethyl, thienylethyl, fluorophenylethyl, hydroxyphenylethyl, chlorophenylethyl, difluorophenylethyl, dichlorophenylethyl, trifluorophenylethyl, and trichlorophenylethyl, and wherein any of the ring system optionally are substituted.

99. The compound according to any of the preceding items, wherein at least one of R⁶ and R⁷ each independently are a ring system selected from the group consisting of cyclopropyl, cyclobutyl, cyclopentyl, cyclohexyl, bicyclo[2.2.2]octanyl, aziridinyl, azetidinyl, tetrahydro-2H-pyranyl, piperidinyl, tetrahydro-2H-thiopyranyl, morpholinyl, piperazinyl, thiomorpholinyl, pyrrolidinyl, and tetrahydrofuranyl, and wherein the ring system optionally is substituted.

100. The compound according to any of the preceding items, wherein R⁶ and R⁷ each independently is phenyl optionally substituted with one to three substitutents selected from the group consisting of hydroxy, fluoro, chloro, bromo, iodo, methoxy, and ethoxy.

101. The compound according to any of the preceding items, wherein R⁶ and R⁷ each independently is phenyl, fluorophenyl, hydroxyphenyl, chlorophenyl, difluorophenyl, dichlorophenyl, trifluorophenyl, and trichlorophenyl.

102. The compound according to any of the preceding items, wherein at least one of R⁶ and R⁷ each independently are selected from the group consisting of methyl, —OH, —NH₂, —CN, —F, —Cl, —Br, —CH₂OH, methoxy, —CH₂F, —CHF₂, —CF₃, —CH₂Cl, —CH₂CH₂OH, ethoxy, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, and 3-methylpentyl.

103. The compound according to any of the preceding items, wherein R⁶ and R⁷ each independently are selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —O—(CH₂)ₚ—Z₃, and —(CH₂)ₚ—Z₃, wherein p is 0 or an integer from 1 to 3; wherein Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ—heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)ᵣ-heteroaryl, —CO(O)—C₁-C₁₀ alkyl, —O—C(O)—(CH₂)ᵣ—C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl; and wherein any alkyl, cycloalkyl, aryl, heterocyclyl, and heteroaryl optionally are substituted.

104. The compound according to any of the preceding items, wherein Z₃ is selected from the group consisting of —H, methyl, —OH, —NH₂, —CN, —F, —Cl, —Br, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, triazinyl, pyrazinyl, pyrrolyl, triazolyl, tetrazolyl, pyrazolyl, furanyl, thienyl, fluorophenyl, hydroxyphenyl, chlorophenyl, difluorophenyl, dichlorophenyl, trifluorophenyl, and trichlorophenyl.

105. The compound according to any of the preceding items, wherein Z₃ is selected from the group consisting of —H, methyl, —OH, —NH₂, —CN, —F, —Cl, —Br, —CH₂OH, —CH₂F, —CHF₂, —CF₃, —CH₂Cl, —CH₂CH₂OH, SO₂, NO₂, ethyl, —CH₂CF₃, —CF₂CF₃, propyl, 2-methylpropyl, tert-butyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, 3-ethylpentyl, 3-ethylpentan-2-yl, 3-ethylpentan-3-yl, cyclohexyl, bicyclo[2.2.2]octanyl, tetrahydro-2H-pyranyl, piperidinyl, tetrahydro-2H-thiopyranyl, morpholinyl, piperazinyl, thiomorpholinyl, cyclobutyl, cyclopropyl, cyclopentyl, azetidinyl, aziridinyl, pyrrolidinyl, tetrahydrofuranyl, pyrrolidinyl, tetrahydrothienyl, oxazolidinyl, imidazolidinyl, thiazolidinyl, carbamoylphenyl, cyanophenyl, pyridinyl, pyrimidinyl, pyrazinyl, pyrrolyl, pyrazolyl, furanyl, thienyl, fluorophenyl, hydroxyphenyl, chlorophenyl, difluorophenyl, dichlorophenyl, trifluorophenyl, and trichlorophenyl.

106. The compound according to any of the preceding items, wherein R⁸ is selected from the group consisting of C₃-C₆ cycloalkyl, aryl, heterocyclyl, heteroaryl, aryl-C₁-C₆ alkyl, C₃-C₆ cycloalkyl-aryl, aryl-C₃-C₆ cycloalkyl, C₃-C₆ cycloalkyl-heterocyclyl, heterocyclyl-C₃-C₆ cycloalkyl, C₃-C₆ cycloalkyl-heteroaryl, heteroaryl-C₃-C₆ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₆ cycloalkyl-O-aryl, aryl-O—C₃-C₆ cycloalkyl, C₃-C₆ cycloalkyl-O-heterocyclyl, heterocyclyl-O—C₃-C₆ cycloalkyl, C₃-C₆ cycloalkyl-O-heteroaryl, heteroaryl-O—C₃-C₆ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₆ cycloalkyl-C(O)-aryl, aryl-C(O)—C₃-C₆ cycloalkyl, C₃-C₆ cycloalkyl-C(O)-heterocyclyl, heterocyclyl-C(O)—C₃-C₆ cycloalkyl, C₃-C₆ cycloalkyl-C(O)-heteroaryl, heteroaryl-C(O)—C₃-C₆ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₆ cycloalkyl-CH₂-aryl, aryl-CH₂—C₃-C₆ cycloalkyl, C₃-C₆ cycloalkyl-CH₂-heterocyclyl, heterocyclyl-CH₂—C₃-C₆ cycloalkyl, C₃-C₆ cycloalkyl-CH₂-heteroaryl, heteroaryl-CH₂—C₃-C₆ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₆ cycloalkyl-CH₂CH₂-aryl, aryl-CH₂CH₂—C₃-C₆ cycloalkyl, C₃-C₆ cycloalkyl-CH₂CH₂-heterocyclyl, heterocyclyl-CH₂CH₂—C₃-C₆ cycloalkyl, C₃-C₆ cycloalkyl-CH₂CH₂-heteroaryl, heteroaryl-CH₂CH₂—C₃-C₆ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₆ cycloalkyl-NH-aryl, aryl-NH—C₃-C₆ cycloalkyl, C₃-C₆ cycloalkyl-NH-heterocyclyl, heterocyclyl-NH—C₃-C₆ cycloalkyl, C₃-C₆ cycloalkyl-NH-heteroaryl, heteroaryl-NH—C₃-C₆ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₆ cycloalkyl-N(Me)-aryl, aryl-N(Me)-C₃-C₆ cycloalkyl, C₃-C₆ cycloalkyl-N(Me)-heterocyclyl, heterocyclyl-N(Me)-C₃-C₆ cycloalkyl, C₃-C₆ cycloalkyl-N(Me)-heteroaryl, heteroaryl-N(Me)-C₃-C₆ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₆ cycloalkyl-NHC(O)-aryl, aryl-NHC(O)—C₃-C₆ cycloalkyl, C₃-C₆ cycloalkyl-NHC(O)-heterocyclyl, heterocyclyl-NHC(O)—C₃-C₆ cycloalkyl, C₃-C₆ cycloalkyl-NHC(O)-heteroaryl, heteroaryl-NHC(O)—C₃-C₆ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₆ cycloalkyl-C(O)NH-aryl, aryl-C(O)NH—C₃-C₆ cycloalkyl, C₃-C₆ cycloalkyl-C(O)NH-heterocyclyl, heterocyclyl-C(O)NH—C₃-C₆ cycloalkyl, C₃-C₆ cycloalkyl-C(O)NH-heteroaryl, heteroaryl-C(O)NH—C₃-C₆ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₆ cycloalkyl-NHC(O)NH-aryl, aryl-NHC(O)NH—C₃-C₆ cycloalkyl, C₃-C₆ cycloalkyl-NHC(O)NH-heterocyclyl, heterocyclyl-NHC(O)NH—C₃-C₆ cycloalkyl, C₃-C₆ cycloalkyl-NHC(O)NH-heteroaryl, heteroaryl-NHC(O)NH—C₃-C₆ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted.

107. The compound according to any of the preceding items, wherein R⁸ is selected from the group consisting of C₃-C₁₀ cycloalkyl, heterocyclyl, heteroaryl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; and wherein any cycloalkyl, aryl, heterocyclyl, and heteroaryl optionally may be substituted.

108. The compound according to any of the preceding items, wherein R⁸ is selected from the group consisting of C₃-C₁₀ cycloalkyl, heterocyclyl, heteroaryl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, heterocyclyl-heteroaryl, heteroaryl-heterocyclyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, heterocyclyl-O-heteroaryl, heteroaryl-O-heterocyclyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, heterocyclyl-C(O)-heteroaryl, heteroaryl-C(O)-heterocyclyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, heterocyclyl-CH₂-heteroaryl, heteroaryl-CH₂-heterocyclyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, heterocyclyl-CH₂CH₂-heteroaryl, heteroaryl-CH₂CH₂-heterocyclyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, heterocyclyl-NH-heteroaryl, heteroaryl-NH-heterocyclyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, heterocyclyl-N(Me)-heteroaryl, heteroaryl-N(Me)-heterocyclyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, heterocyclyl-NHC(O)-heteroaryl, heteroaryl-NHC(O)-heterocyclyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, heterocyclyl-C(O)NH-heteroaryl, heteroaryl-C(O)NH-heterocyclyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, heterocyclyl-NHC(O)NH-heteroaryl, and heteroaryl-NHC(O)NH-heterocyclyl; wherein cycloalkyl, heterocyclyl, and heteroaryl optionally may be substituted.

109. The compound according to any of the preceding items, wherein R⁸ is selected from the group consisting of C₃-C₁₀ cycloalkyl, aryl, heterocyclyl and heteroaryl; and wherein cycloalkyl, heterocyclyl, and heteroaryl optionally may be substituted.

110. The compound according to any of the preceding items, wherein R⁸ is selected from the group consisting of aryl-C(O)—C₃-C₁₀ cycloalkyl, aryl-C(O)-heteroaryl, aryl-C(O)-heterocyclyl, aryl-C(O)NH—C₃-C₁₀ cycloalkyl, aryl-C(O)NH-heteroaryl, aryl-C(O)NH-heterocyclyl, aryl-C₁-C₆ alkyl, aryl-C₃-C₁₀ cycloalkyl, aryl-CH₂—C₃-C₁₀ cycloalkyl, aryl-CH₂CH₂—C₃-C₁₀ cycloalkyl, aryl-CH₂CH₂-heteroaryl, aryl-CH₂CH₂-heterocyclyl, aryl-CH₂-heteroaryl, aryl-CH₂-heterocyclyl, aryl-heteroaryl, aryl-heterocyclyl, aryl-N(Me)-C₃-C₁₀ cycloalkyl, aryl-N(Me)-heteroaryl, aryl-N(Me)-heterocyclyl, aryl-NHC(O)—C₃-C₁₀ cycloalkyl, aryl-NHC(O)-heteroaryl, aryl-NHC(O)-heterocyclyl, aryl-NHC(O)NH—C₃-C₁₀ cycloalkyl, aryl-NHC(O)NH-heteroaryl, aryl-NHC(O)NH-heterocyclyl, aryl-NH—C₃-C₁₀ cycloalkyl, aryl-NH-heteroaryl, aryl-NH-heterocyclyl, aryl-O—C₃-C₁₀ cycloalkyl, aryl-O-heteroaryl, and aryl-O-heterocyclyl

111. The compound according to any of the preceding items, wherein R⁸ is selected from the group consisting of C₃-C₁₀ cycloalkyl-aryl, C₃-C₁₀ cycloalkyl-C(O)-aryl, C₃-C₁₀ cycloalkyl-C(O)-heteroaryl, C₃-C₁₀ cycloalkyl-C(O)-heterocyclyl, C₃-C₁₀ cycloalkyl-C(O)NH-aryl, C₃-C₁₀ cycloalkyl-C(O)NH-heteroaryl, C₃-C₁₀ cycloalkyl-C(O)NH-heterocyclyl, C₃-C₁₀ cycloalkyl-CH₂-aryl, C₃-C₁₀ cycloalkyl-CH₂CH₂-aryl, C₃-C₁₀ cycloalkyl-CH₂CH₂-heteroaryl, C₃-C₁₀ cycloalkyl-CH₂CH₂-heterocyclyl, C₃-C₁₀ cycloalkyl-CH₂-heteroaryl, C₃-C₁₀ cycloalkyl-CH₂-heterocyclyl, C₃-C₁₀ cycloalkyl-heteroaryl, C₃-C₁₀ cycloalkyl-heterocyclyl, C₃-C₁₀ cycloalkyl-N(Me)-aryl, C₃-C₁₀ cycloalkyl-N(Me)-heteroaryl, C₃-C₁₀ cycloalkyl-N(Me)-heterocyclyl, C₃-C₁₀ cycloalkyl-NH-aryl, C₃-C₁₀ cycloalkyl-NHC(O)-aryl, C₃-C₁₀ cycloalkyl-NHC(O)-heteroaryl, C₃-C₁₀ cycloalkyl-NHC(O)-heterocyclyl, C₃-C₁₀ cycloalkyl-NHC(O)NH-aryl, C₃-C₁₀ cycloalkyl-NHC(O)NH-heteroaryl, C₃-C₁₀ cycloalkyl-NHC(O)NH-heterocyclyl, C₃-C₁₀ cycloalkyl-NH-heteroaryl, C₃-C₁₀ cycloalkyl-NH-heterocyclyl, C₃-C₁₀ cycloalkyl-O-aryl, C₃-C₁₀ cycloalkyl-O-heteroaryl, and C₃-C₁₀ cycloalkyl-O-heterocyclyl.

112. The compound according to any of the preceding items, wherein R⁸ is selected from the group consisting of heteroaryl-C(O)NH-aryl, heteroaryl-aryl, heteroaryl-C(O)-aryl, heteroaryl-C(O)—C₃-C₁₀ cycloalkyl, heteroaryl-C(O)-heterocyclyl, heteroaryl-C(O)NH—C₃-C₁₀ cycloalkyl, heteroaryl-C(O)NH-heterocyclyl, heteroaryl-C₃-C₁₀ cycloalkyl, heteroaryl-CH₂-aryl, heteroaryl-CH₂—C₃-C₁₀ cycloalkyl, heteroaryl-CH₂CH₂-aryl, heteroaryl-CH₂CH₂—C₃-C₁₀ cycloalkyl, heteroaryl-CH₂CH₂-heterocyclyl, heteroaryl-CH₂-heterocyclyl, heteroaryl-heterocyclyl, heteroaryl-N(Me)-aryl, heteroaryl-N(Me)-C₃-C₁₀ cycloalkyl, heteroaryl-N(Me)-heterocyclyl, heteroaryl-NH-aryl, heteroaryl-NHC(O)-aryl, heteroaryl-NHC(O)—C₃-C₁₀ cycloalkyl, heteroaryl-NHC(O)-heterocyclyl, heteroaryl-NHC(O)NH-aryl, heteroaryl-NHC(O)NH—C₃-C₁₀ cycloalkyl, heteroaryl-NHC(O)NH-heterocyclyl, heteroaryl-NH—C₃-C₁₀ cycloalkyl, heteroaryl-NH-heterocyclyl, heteroaryl-O-aryl, heteroaryl-O—C₃-C₁₀ cycloalkyl, and heteroaryl-O-heterocyclyl.

113. The compound according to any of the preceding items, wherein R⁸ is selected from the group consisting of heterocyclyl-aryl, heterocyclyl-C(O)-aryl, heterocyclyl-C(O)—C₃-C₁₀ cycloalkyl, heterocyclyl-C(O)-heteroaryl, heterocyclyl-C(O)NH-aryl, heterocyclyl-C(O)NH—C₃-C₁₀ cycloalkyl, heterocyclyl-C(O)NH-heteroaryl, heterocyclyl-C₃-C₁₀ cycloalkyl, heterocyclyl-CH₂-aryl, heterocyclyl-CH₂—C₃-C₁₀ cycloalkyl, heterocyclyl-CH₂CH₂-aryl, heterocyclyl-CH₂CH₂—C₃-C₁₀ cycloalkyl, heterocyclyl-CH₂CH₂-heteroaryl, heterocyclyl-CH₂-heteroaryl, heterocyclyl-heteroaryl, heterocyclyl-N(Me)-aryl, heterocyclyl-N(Me)-C₃-C₁₀ cycloalkyl, heterocyclyl-N(Me)-heteroaryl, heterocyclyl-NH-aryl, heterocyclyl-NHC(O)-aryl, heterocyclyl-NHC(O)—C₃-C₁₀ cycloalkyl, heterocyclyl-NHC(O)-heteroaryl, heterocyclyl-NHC(O)NH-aryl, heterocyclyl-NHC(O)NH—C₃-C₁₀ cycloalkyl, heterocyclyl-NHC(O)NH-heteroaryl, heterocyclyl-NH—C₃-C₁₀ cycloalkyl, heterocyclyl-NH-heteroaryl, heterocyclyl-O-aryl, heterocyclyl-O—C₃-C₁₀ cycloalkyl, and heterocyclyl-O-heteroaryl.

114. The compound according to any of the preceding items, wherein R⁸ is selected from the group consisting of aryl-heterocyclyl and heteroaryl-heterocyclyl.

115. The compound according to any of the preceding items, wherein R⁸ is selected from the group consisting of azetidinyl, cyclopropanyl, cyclobutanyl, cyclopentanyl, cyclohexanyl, cyclohexanylcyclobutyl, cyclohexanylcyclopropyl, cyclohexylcyclohexyl, phenylcyclobutyl, phenylcyclobutyl, phenylcyclohexyl, phenoxycyclobutyl, phenoxycyclopentyl, phenoxycyclohexyl, benzylcyclobutyl, benzylcyclobutyl, benzylcyclohexyl, phenylaminocyclobutyl, phenylaminocyclobutyl, phenylaminocyclohexyl, 7-azabicyclo[4.2.0]octa-1,3,5-trienyl, 2,3-dihydro-1H-indolyl, 1,2,3,4-tetrahydroquinolinyl, 2,3-dihydro-1H-isoindolyl, 1,2,3,4-tetrahydroisoquinolinyl, phenylazetidinyl, phenylpyrrolidinyl, phenylpiperidinyl, phenylazetidinyl, phenylazetidinonyl, phenylpyrrolidinonyl, phenylpiperidinonyl, phenoxyazetidinyl, phenoxypyrrolidinyl, phenoxypiperidinyl, phenoxyazetidinyl, phenoxypyrrolidinyl, phenoxypiperidinyl, phenoxypiperidinyl, phenoxyazetidinonyl, phenoxypyrrolidinonyl, phenoxypiperidinonyl, benzylazetidinyl, benzylpyrrolidinyl, benzylpiperidinyl, benzylazetidinonyl, benzylpyrrolidinonyl, benzylpiperidinonyl, phenylaminoazetidinyl, phenylaminopyrrolidinyl, phenylaminopiperidinyl, phenylaminoazetidinyl, phenylaminoazetidinonyl, phenylaminopyrrolidinonyl, phenylaminopiperidinonyl, phenyl, phenylphenyl, benzylphenyl, phenoxyphenyl, phenylaminophenyl, phenylsulfanylphenyl, phenylcarbonylphenyl, naphtyl, phenalenyl, anthracenyl, phenylnaphtyl, 5-phenylnaphthalen-2-yl, phenylfuranyl, phenylpyrrolyl, phenylthiophenyl, phenylisoxazolyl, phenyloxazolyl, phenyloxadiazolyl, benzylisoxazolyl, benzyloxazolyl, benzyloxadiazolyl, thiazolyl, phenylthiazolyl, imidazolylthiazolyl, pyrazinylthiazolyl, phenylthiadiazolyl, [1,3]thiazolo[5,4-b]pyridinyl, [1,3]oxazolo[5,4-b]pyridinyl, 3H-imidazo[4,5-b]pyridinyl, [1,3]thiazolo[5,4-c]pyridinyl, [1,3]oxazolo[5,4-c]pyridinyl, 3H-imidazo[4,5-c]pyridinyl, [1,3]thiazolo[4,5-c]pyridinyl, [1,3]oxazolo[4,5-c]pyridinyl, 1H-imidazo[4,5-c]pyridinyl, [1,3]thiazolo[5,4-c]pyridazinyl, [1,3]oxazolo[5,4-c]pyridazinyl, 7H-imidazo[4,5-c]pyridazinyl, [1,3]thiazolo[5,4-d]pyrimidinyl, [1,3]oxazolo[5,4-d]pyrimidinyl, 9H-purinyl, [1,3]thiazolo[4,5-d]pyridazinyl, [1,3]oxazolo[4,5-d]pyridazinyl, 1H-imidazo[4,5-d]pyridazinyl, [1,3]thiazolo[5,4-d][1,2,3]triazinyl, [1,3]oxazolo[5,4-d][1,2,3]triazinyl, 7H-imidazo[4,5-d][1,2,3]triazinyl, phenylpyrazolyl, phenyltriazolyl, phenyltetrazolyl, benzylpyrazolyl, benzyltriazolyl, benzyltetrazolyl, naphatalenylcyclopropanyl, naphtalenylmethylcyclobutanyl, naphtalenylaminocyclopentanyl, napthalenyloxyazetidinyl, naphtalenylcarbonylpyrrolidinyl, naphatalenylpiperidinyl, naphtalenylmethylazetidinonyl, naphtalenylaminopyrrolidinonyl, napthalenyloxypiperidinonyl, naphtalenylcarbonylpyrazolyl, naphatalenyltriazolyl, naphtalenylmethyltetrazolyl, naphtalenylaminofuranyl, napthalenyloxypyrrolyl, naphtalenylcarbonylthienyl, and naphatalenyloxazolyl.

116. The compound according to any of the preceding items, wherein R⁸ is selected from the group consisting of phenyl, phenylcyclopentyl, phenylpyrrolidine, benzylpyrrolidine, phenoxypyrrolidine, and phenylaminopyrrolidine.

117. The compound according to any of the preceding items, wherein R⁸ is substituted with one or more substituents selected from the group consisting of halogen, hydroxyl, C₁-C₆ alkyl, C₁-C₆ alkoxy, —CN, —NO₂, —NH₂, —SO₂—C₁-C₆ alkyl, —S(O)—C₁-C₆ alkyl, C₂-C₆ alkenyl, C₂-C₆ alkynyl, C₃-C₁₀ cycloalkyl, aryl, heterocyclyl, and heteroaryl.

118. The compound according to any of the preceding items, wherein R⁸ is substituted with one or more substituents selected from the group consisting of halogen, hydroxyl, C₁-C₆ alkyl, C₁-C₆ alkoxy, —CN, —NO₂, —SO₂—C₁-C₆ alkyl, —NH₂, —SO₂—C₁-C₆ alkyl, —S(O)—C₁-C₆ alkyl, C₂-C₆ alkenyl, and C₂-C₆ alkynyl.

119. The compound according to any of the preceding items, wherein R⁸ is substituted with one or more substituents selected from the group consisting of fluoro, chloro, hydroxy, methoxy, ethoxy, methyl, ethyl, propyl, isopropyl, tert-butyl, sec-butyl, cyano, nitro, sulfanyl, methylsulfanyl, sulfonyl, and methylsulfonyl.

120. The compound according to any of the preceding items, wherein R⁹ is selected from the group consisting of H, C₁-C₄ alkyl, trifluoromethyl, trifluoroethyl, C₁-C₄ alkoxy, halogen-C₁-C₄ alkyl, —(CH₂)₀₋₂-aryl, —(CH₂)₀₋₂-heterocyclyl, and —(CH₂)₀₋₂-heteroaryl.

121. The compound according to any of the preceding items, wherein R⁹ is selected from the group consisting of H, methyl, ethyl, trifluoromethyl, —CH₂OH, —(CH₂)₀₋₁-aryl, and —(CH₂)₀₋₁-heteteroaryl.

122. The compound according to any of the preceding items, wherein R⁹ is selected from the group consisting of H, methyl, ethyl, trifluoromethyl, —CH₂OH, aryl, and heteroaryl.

123. The compound according to any of the preceding items, wherein R¹⁰ and R¹¹ each independently are selected from the group consisting of H, C₁-C₄ alkyl, C₃-C₇ cycloalkyl, aryl, —(CH₂)₁₋₂—C₃-C₇ cycloalkyl, —(CH₂)₁₋₂-aryl, wherein alkyl, cycloalkyl, and aryl optionally are substituted, or R¹⁰ together with R¹¹ may form a heterocyclyl ring together with the nitrogen to which they are attached.

124. The compound according to any of the preceding items, wherein R¹⁰ and R¹¹ each independently are selected from the group consisting of H, C₁-C₄ alkyl, C₃-C₇ cycloalkyl, aryl, —(CH₂)₁₋₂—C₃-C₇ cycloalkyl, —(CH₂)₁₋₂-aryl, wherein alkyl, cycloalkyl, and aryl optionally are substituted.

125. The compound according to any of the preceding items, wherein R¹⁰ together with R¹¹ forms a heterocyclyl ring together with the nitrogen to which they are attached.

126. The compound according to any of the preceding items, wherein R¹⁰ and R¹¹ each independently are selected from the group consisting of —H, methyl, ethyl, 2-methylpropyl, butyl, butan-2-yl, 2-methylbutyl, 2-methylbutan-2-yl, 3-methylbutan-2-yl, 3-methylbutyl, pentyl, pentan-2-yl, pentan-3-yl, 2-ethylbutyl, 3-methylpentan-3-yl, 3-methylpentan-2-yl, 3-methylpentyl, pyridinyl, pyridazinyl, imidazolyl, imidazolidinyl, pyrimidinyl, pyrazolyl, triazolyl, pyrazinyl, pyrazolinyl, pyrazolidinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl, aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, 1,4-dioxaspiro[4.2]heptyl, 2-oxopiperazinyl, 2-oxopiperidinyl, 2,8-diazaspiro[4.5]decanyl and 8-azaspiro[4.5]decanyl.

127. The compound according to any of the preceding items, wherein m is 0, or an integer from 1 to 3.

128. The compound according to any of the preceding items, wherein n is 0, or an integer from 1 to 3.

129. The compound according to any of the preceding items, wherein p is 0, or an integer from 1 to 3.

130. The compound according to any of the preceding items, wherein q is 0, or an integer from 1 to 3.

131. The compound according to any of the preceding items, wherein r is 0, or an integer from 1 to 3.

132. The compound according to any of the preceding items, having formula (II)

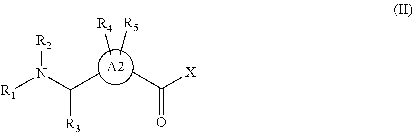

wherein R1, R2, R3, R4, R5, R6, R7, R8, A1, A2, A3, A4, and X is as defined in any of items 1-17, 19-131.

133. The compound according to item 132, having formula (IIa)

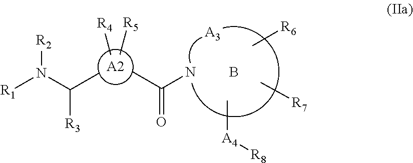

wherein R1, R2, R3, R4, R5, R6, R7, R8, A1, A2, A3, and A4 are as defined in any of items 1-17, 19-40, 42-132.

134. The compound according to item 132, having formula (IIb)

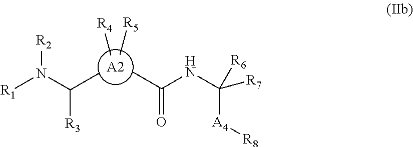

wherein R1, R2, R3, R4, R5, R6, R7, R8, A1, A2, A3, and A4 are as defined in any of items 1-17, 19-39, 41-132.

135. The compound according to any of items 1-131, having formula (III)

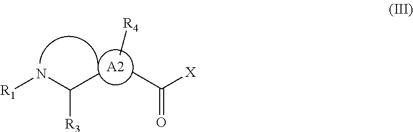

wherein R1, R2, R3, R4, R5, R6, R7, R8, A1, A2, A3, A4, and X are as defined in any of items 1-17, 19-47, 51-131.

136. The compound according to item 135, having formula (IIIa)

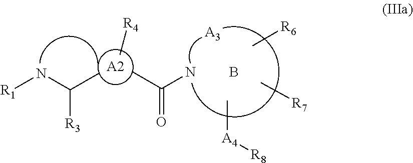

wherein R1, R2, R3, R4, R5, R6, R7, R8, A1, A2, A3, and A4 are as defined in any of items 1-17, 19-40, 42-47, 51-131.

137. The compound according to item 135, having formula (IIIb)

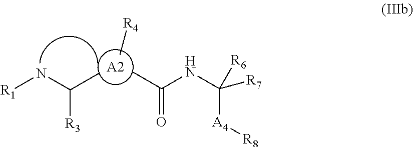

wherein R1, R2, R3, R4, R5, R6, R7, R8, A1, A2, A3, and A4 are as defined in any of items 1-17, 19-39, 41-47, 51-131.

138. The compound according to any of items 1-131, having formula (IV)

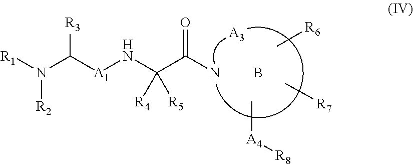

wherein R1, R2, R3, R4, R5, R6, R7, R8, A1, A2, A3, A4, and X is as defined in any of items 1-6, 18-40, 42-131.

139. The compound according to any of items 1-131, having formula (V)

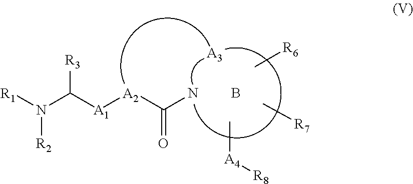

wherein R1, R2, R3, R4, R5, R6, R7, R8, A1, A2, A3, and A4 are as defined in any of items 1-6, 18-40, 42-131.

140. The compound according to item 1, wherein the compound is selected from the group consisting of
(5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone; and
(6-(1-(methylamino)ethyl)pyridin-2-yl)((2S,4R)-4-phenyl-2-(((R)-3-phenylpyrrolidin-1-yl)methyl)pyrrolidin-1-yl)methanone.

141. The compound according to item 1, wherein the compound is selected from the group consisting of
(5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone; and
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone.

142. The compound according to item 1, wherein the compound is selected from the group consisting of
(2S,4R)-1-((3R,5S)-1-(2-((S)-2-aminopropanamido)-3-(1H-1,2,4-triazol-1-yl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)butanoyl)-3-phenylpyrrolidine-5-carbonyl)-N-methyl-4-phenylpyrrolidine-2-carboxamide;
(2S,4R)-1-((S)-2-((R)-2-aminopropanamido)-3-(4-carbamoylphenyl)propanoyl)-N—((R)-2,3-dihydro-1H-inden-1-yl)-4-phenylpyrrolidine-2-carboxamide;
(2R,3R)-1-((S)-2-((S)-2-aminopropanamido)-3-(3-carbamoylphenyl)propanoyl)-N—((R)-2,3-dihydro-1H-inden-1-yl)-3-phenylazetidine-2-carboxamide;
(2S,4R)-1-((S)-2-((S)-2-aminopropanamido)-3-(3-cyanophenyl)propanoyl)-N—((R)-2,3-dihydro-1H-inden-1-yl)-4-phenylpyrrolidine-2-carboxamide;
(2S,4R)-1-((3R,5S)-1-((S)-2-((S)-2-aminopropanamido)-3-(3-cyanophenyl)propanoyl)-3-phenylpyrrolidine-5-carbonyl)-N-methyl-4-phenylpyrrolidine-2-carboxamide;
(2S,4R)-1-((3R,5S)-1-((S)-2-((S)-2-aminopropanamido)-3-(furan-2-yl)propanoyl)-3-phenylpyrrolidine-5-carbonyl)-N-methyl-4-phenylpyrrolidine-2-carboxamide;
(S)—N—((S)-3-(3-cyanophenyl)-1-oxo-1-((2S,4R)-4-phenyl-2-(((R)-3-phenylpyrrolidin-1-yl)methyl)pyrrolidin-1-yl)propan-2-yl)-2-(methylamino)butanamide;
(2S,4R)-1-((S)-2-((R)-2-aminopropanamido)-3-(3-carbamoylphenyl)propanoyl)-N—((R)-2,3-dihydro-1H-inden-1-yl)-4-phenylpyrrolidine-2-carboxamide; and
(2S,3S)-1-((S)-2-((S)-2-aminopropanamido)-3-(3-carbamoylphenyl)propanoyl)-N—((S)-2,3-dihydro-1H-inden-1-yl)-2-phenylazetidine-3-carboxamide.

143. The compound according to item 1, wherein the compound is selected from the group consisting of
[5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone; and
(6-(1-(methylamino)ethyl)pyridin-2-yl)((2S,4R)-4-phenyl-2-(((R)-3-phenylpyrrolidin-1-yl)methyl)pyrrolidin-1-yl)methanone.

144. The compound according to item 1, wherein the compound is selected from the group consisting of
(2S,4S)-4-Cyclohexyl-1-(2,8-diaza-spiro[4.5]decane-3-carbonyl)-pyrrolidine-2-carboxylic acid (R)-indan-1-ylamide; and
2,8-Diaza-spiro[4.5]decane-3-carboxylic acid [(S)-cyclohexyl-((R)-indan-1-ylcarbamoyl)-methyl]-amide.

145. A polymeric compound of formula (VI)

Y-(L)ₘ-[Y-(L)ₘ]ₙ—Y (VI)

or a pharmaceutically acceptable salt, solvate or prodrug thereof,
wherein
Y is a monomeric unit of formula (I), wherein the first and the second or further monomeric units are the same or different and independently are selected from the compounds as defined in any of items 1-144;
L is the same or different and is a covalent linker, linking any part of one monomeric unit of formula (I), to any part of a second or further monomeric unit of formula (I);
m is an integer of 1 to 4; and
n is an integer of 0 to 5.

146. The polymeric compound according to item 145, wherein linker L is selected from the group consisting of

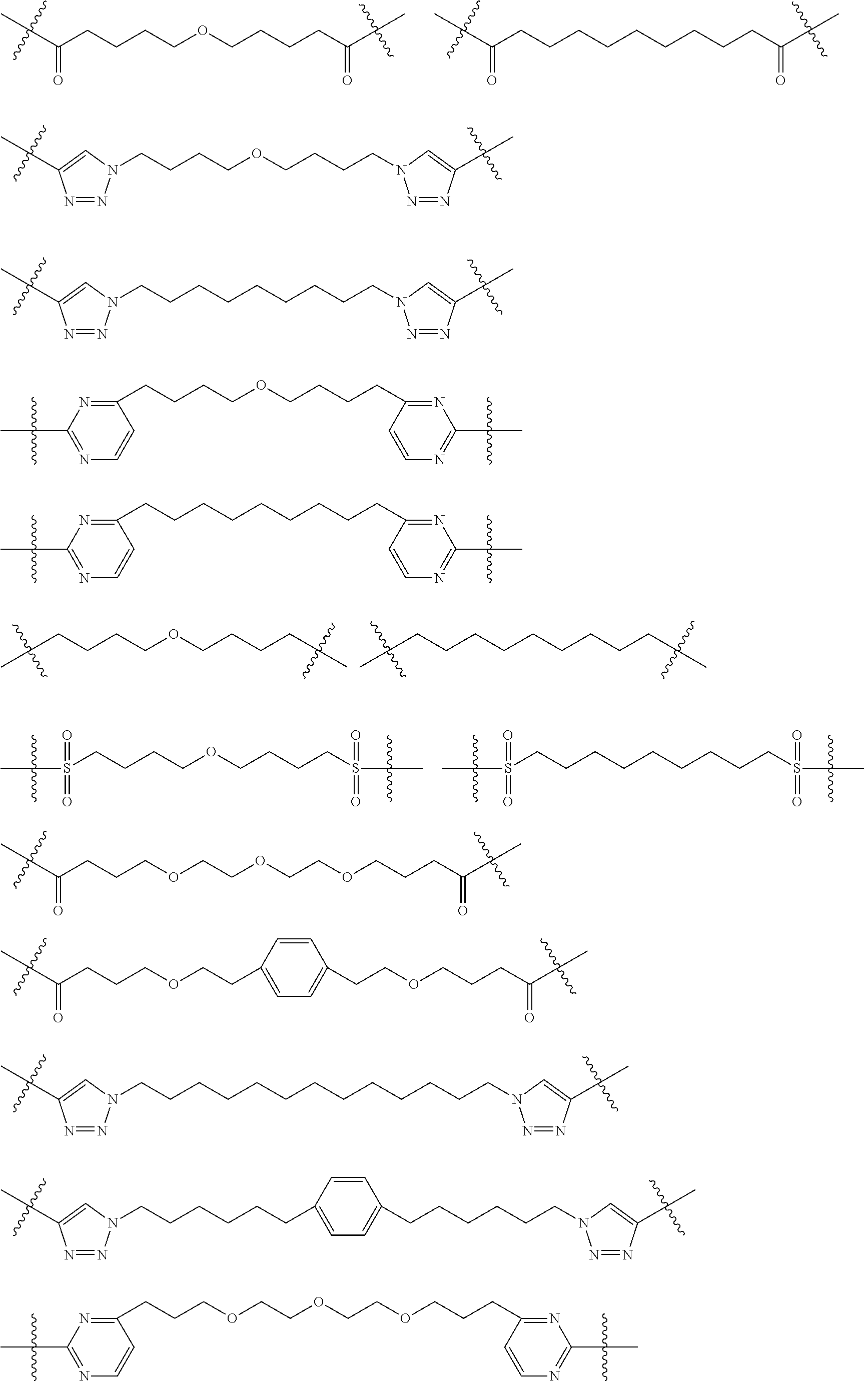

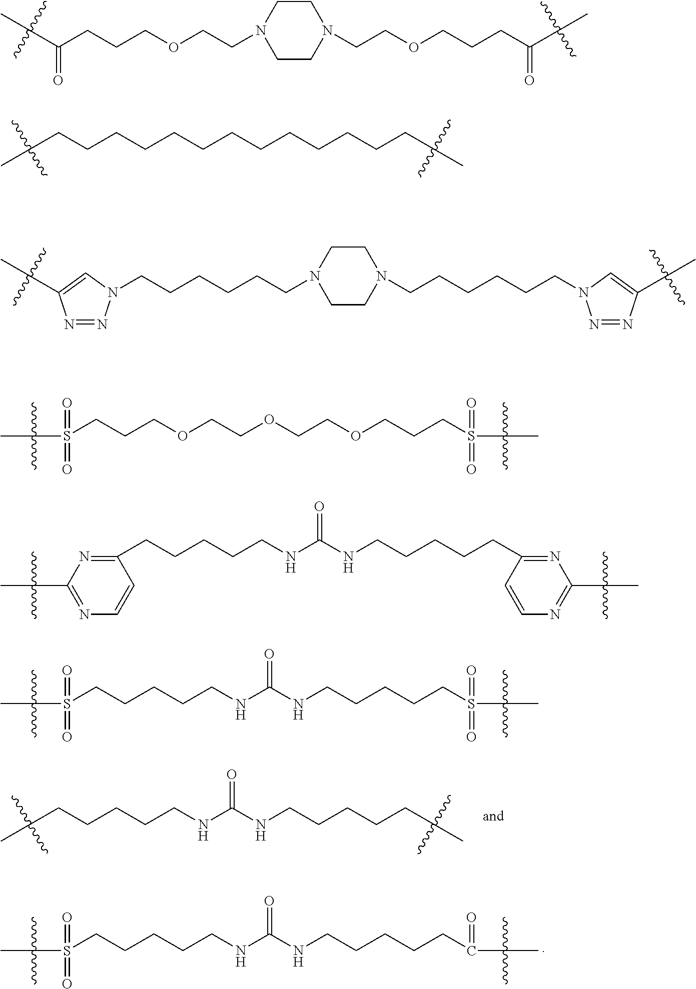

147. The polymeric compound according to any of items 145-146, wherein m is 1; and n is an integer of 0 to 2.

148. A compound of formula (VII)

Z-Lₘ-E (VII)

or a pharmaceutically acceptable salt, solvate or prodrug thereof,
wherein
Z is a compound of formula (I) as defined in any of items 1-144 or a polymeric compound of formula (VI) as defined in any of items 145-147;
L is a linker linking any part of Z to any part of E;
E is an entity selected from the group consisting of an affinity tag, such as e.g. a hexahistidine tag or biotin, a dye, such as e.g. fluorescein, an oligonucleotide, a protein, such as e.g. an antibody or biotin-binding protein, and a solid support; and
m is an integer of 1 to 4.

149. The polymeric compound according to item 148, wherein linker L is selected from the group consisting of

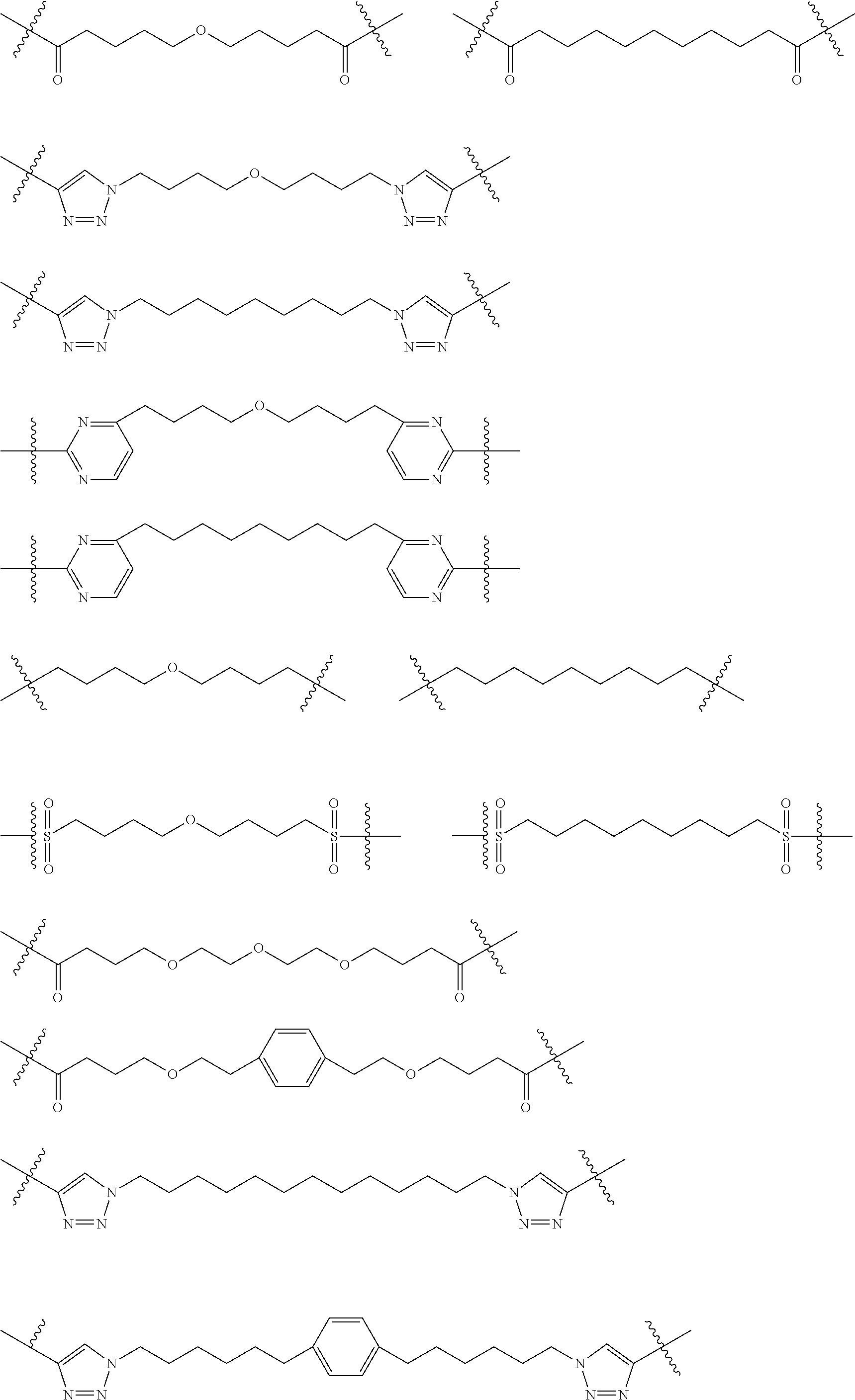

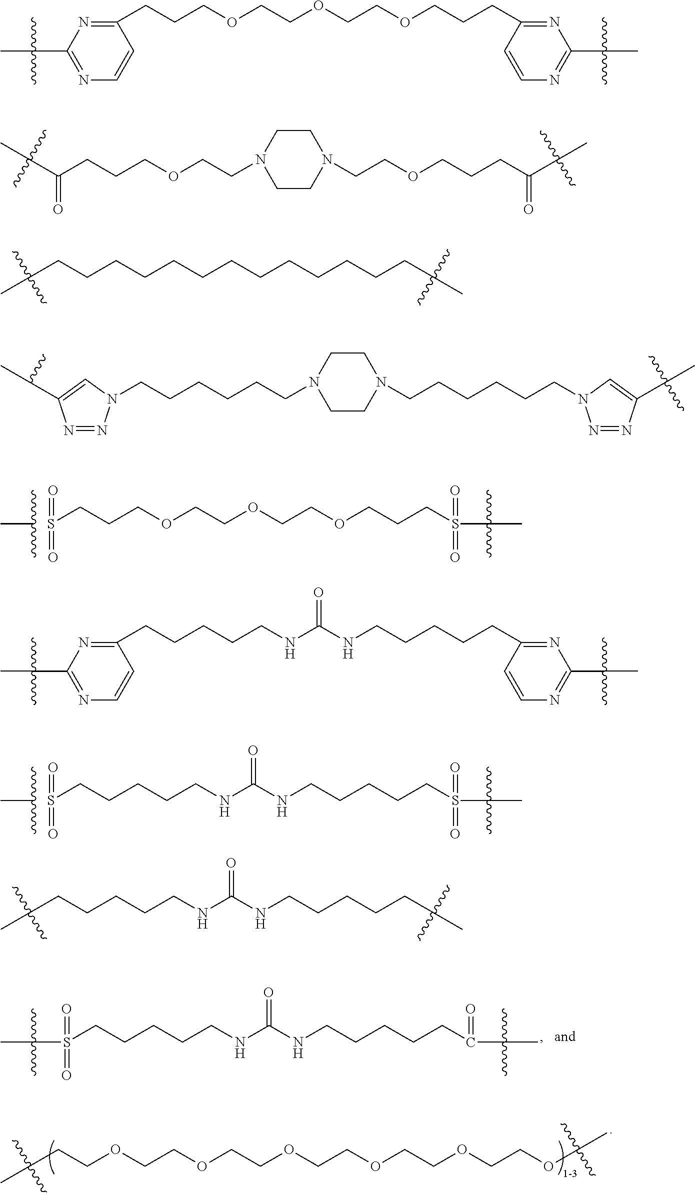

150. The polymeric compound according to any of items 148-149, wherein m is 1.

151. A compound as defined in any of items 1-144, 145-147, or 148-150 for use as a medicament.

152. A compound as defined in any of items 1-144, 145-147, or 148-150 for treating proliferative diseases.

153. A compound as defined in any of items 1-144, 145-147, or 148-150 for promoting apoptosis in proliferating cells.

154. A compound as defined in any of items 1-144, 145-147, or 148-150 for sensitizing cells to inducers of apoptosis.

155. Use of a compound as defined in any of items 1-144, 145-147, or 148-150 for the preparation of a medicament for the treatment of proliferative diseases.

156. The use according to item 155, wherein the disease is cancer.

157. Use of a compound as defined in any of items 1-144, 145-147, or 148-150 for the preparation of a medicament for promoting apoptosis in proliferating cells.

158. Use of a compound as defined in any of items 1-144, 145-147, or 148-150 for the preparation of a medicament for sensitizing cells to inducers of apoptosis.

159. The use according to any of items 155, 157 or 158, comprising a combination treatment with one or more additional active substances.

160. The use according to item 159, wherein the one or more additional active substances are selected from anticancer agents, antineoplastic agents, cytotoxic drugs, and anti-tumor antibiotics.

161. The use according to item 159, wherein the one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

162. A pharmaceutical composition comprising a compound as defined in any of items 1-144, 145-147, or 148-150, and optionally one or more pharmaceutically acceptable excipients, diluents or carriers.

163. The pharmaceutical composition according to item 162, further comprising one or more additional active substances.

164. The pharmaceutical composition according to item 163, wherein the one or more additional active substances are selected from anticancer agents, antineoplastic agents, cytotoxic drugs, and anti-tumor antibiotics.

165. The pharmaceutical composition according to any of items 163-164, wherein the one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

166. A method of treating a proliferative disease in a subject, said method comprises administering to said subject a therapeutically effective amount of a compound as defined in any of items 1-144, 145-147, or 148-150, or a pharmaceutical composition as defined in any of items 162-165, to a subject in need of such treatment.

167. The method according to item 166, wherein the compound is administered in combination with one or more additional active substances.

168. The method according to item 167, wherein the one or more additional active substances are selected from anticancer agents, antineoplastic agents, cytotoxic drugs, and anti-tumor antibiotics.

169. The method according to item 167, wherein the one or more additional active substances are selected from protease inhibitors, epidermal growth factor receptor kinase inhibitors, vascular endothelial growth factor receptor kinase inhibitors, antimetabolites, antimitotic agents, platinum coordination complexes, anti-tumor antibiotics, alkylating agents, and endocrine agents.

170. The method according to any of items 166-169, wherein the subject is a mammal, such as a human being.

1. A compound of formula (I) 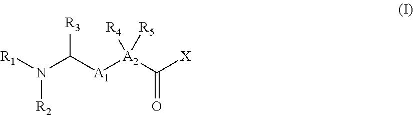
or a pharmaceutically acceptable salt, solvate or prodrug thereof,
wherein
X is 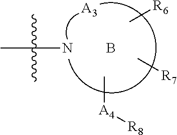
A₁ is a single bond,
A₂ is selected from the group consisting of cycloalkyl, aryl, heterocyclyl, and heteroaryl, wherein R⁴ and R⁵ independently are attached to cycloalkyl, aryl, heterocyclyl, or heteroaryl via any chemically feasible positions of the ring systems;
A₃ is a ring atom or moiety selected from the group consisting of C, S, O, N, —C(O)—, —NHC(O)—, and —C(O)NH—;
A₄ is a linker which is selected from the group consisting of single bond, —CH₂—, —C(O)—, —NH—, —O—, —S—, —SO₂—, —CH₂CH₂—, —C(O)CH₂—, —CH₂C(O)—, —NHCH₂—, —CH₂NH—, —OCH₂—, —CH₂O—, —SCH₂—, —CH₂S—, —SO₂CH₂—, —CH₂SO₂—, —NHC(O)—, —C(O)NH—, —NHSO₂—, —SO₂NH—, —CH₂CH₂CH₂—, —CH₂CH₂C(O)—, —CH₂CH₂NH—, —CH₂CH₂O—, —CH₂CH₂S—, —CH₂CH₂SO₂—, —CH₂C(O)CH₂—, —CH₂NHCH₂—, —CH₂OCH₂—, —CH₂SCH₂—, —CH₂SO₂CH₂—, —C(O)CH₂CH₂—, —NHCH₂CH₂—, —OCH₂CH₂—, —SCH₂CH₂—, —SO₂CH₂CH₂—, —CH₂C(O)NH—, —CH₂SO₂NH—, —CH₂NHC(O)—, —CH₂NHSO₂—, —C(O)NHCH₂—, —SO₂NHCH₂—, —NHC(O)CH₂—, —NHSO₂CH₂—, and —NHC(O)NH—;
B is selected from the group consisting of heterocyclic and heteroaromatic ring systems;
R¹ is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted;
R² is selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl, —(CH₂)₁₋₆-cycloalkyl, —(CH₂)₁₋₆-aryl, —(CH₂)₁₋₆-heterocyclyl, and —(CH₂)₁₋₆-heteroaryl, wherein any alkyl, alkenyl, alkynyl, cycloalkyl, aryl, heterocyclyl, and heteroaryl optionally are substituted; or wherein R² together with R⁵ optionally may form a heterocyclic ring together with the nitrogen to which R² is attached, or wherein R² together with R⁵ forms a heterocyclic ring together with the nitrogen to which R² is attached and where R² is a single bond, wherein the heterocyclic ring optionally is substituted;
R³ is selected from the group consisting of H, hydroxy, halogen, C₁-C₆ alkyl, C₁-C₆ alkoxy, C₂-C₆ alkenyl, C₂-C₆ alkynyl, and C₃-C₁₀ cycloalkyl, wherein alkyl, alkenyl and alkynyl optionally are substituted;
R⁴ and R⁵ are each independently selected from the group consisting of H, C₁-C₆ alkyl, C₁-C₆ alkoxy, C₂-C₆ alkenyl, C₂-C₆ alkynyl, C₃-C₁₀ cycloalkyl, aryl, heterocyclyl, heteroaryl-NH—(CH₂)ₙ—Z₂, —O—(CH₂)ₙ—Z₂, —CH₂—NH—(CH₂)ₙ—Z₂, —CH₂—O—(CH₂)ₙ—Z₂, —(CH₂)₂—NH—(CH₂)ₙ—Z₂, —(CH₂)₂—O—(CH₂)ₙ—Z₂, and —(CH₂)ₙ—Z₂, wherein any alkyl, alkenyl, alkynyl, cycloalkyl, aryl, heterocyclyl, and heteroaryl optionally are substituted;
Z₂ is selected from the group consisting of halogen, hydroxyl, —NH₂, —CN, —NO₂, C₁-C₆ alkoxy, C₂-C₆ alkenyl, C₂-C₆ alkynyl, C₃-C₁₀ cycloalkyl, aryl, heterocyclyl, heteroaryl, —O—C₁-C₆ alkyl, —C(O)—C₁-C₆ alkyl, —C(O)—(CH₂)_{q}—C₃-C₇ cycloalkyl, —C(O)—(CH₂)_{q}-aryl, —C(O)—(CH₂)_{q}-heterocyclyl, —C(O)—(CH₂)_{q}-heteroaryl, —O—(CH₂)_{q}—C₃-C₁₀ cycloalkyl, —O—(CH₂)_{q}-aryl, —O—(CH₂)_{q}-heterocyclyl, —O—(CH₂)_{q}-heteroaryl, —S(O)—C₁-C₆ alkyl, —S(O)—(CH₂)_{q}—C₃-C₇ cycloalkyl, —S(O)—(CH₂)_{q}-aryl, —S(O)—(CH₂)_{q}-heterocyclyl, —S(O)—(CH₂)_{q}-heteroaryl, —SO₂—C₁-C₆ alkyl, —SO₂—(CH₂)_{q}—C₃-C₇ cycloalkyl, —SO₂—(CH₂)_{q}-aryl, —SO₂—(CH₂)_{q}-heterocyclyl, —SO₂—(CH₂)_{q}-heteroaryl, —N(R⁹)—SO₂—C₁-C₆ alkyl, —N(R⁹)—SO₂—(CH₂)_{q}—C₃-C₇ cycloalkyl, —N(R⁹)—SO₂—(CH₂)_{q}-aryl, —N(R⁹)—SO₂—(CH₂)_{q}-heterocyclyl, —N(R⁹)—SO₂—(CH₂)_{q}-heteroaryl, —SO₂—N(R¹⁰ (R¹¹), —N(R⁹)—C(O)—C₁-C₆ alkyl, —N(R⁹)—C(O)—(CH₂)_{q}—C₃-C₇ cycloalkyl, —N(R⁹)—C(O)—(CH₂)_{q}-aryl, —N(R⁹)—C(O)—(CH₂)_{q}-heterocyclyl, —N(R⁹)—C(O)—(CH₂)_{q}-heteroaryl, —C(O)—N(R¹⁰ (R¹¹), —C(O)—O—C₁-C₆ alkyl, —C(O)—O—(CH₂)_{q}—C₃-C₇ cycloalkyl, —C(O)—O—(CH₂)_{q}-aryl, —C(O)—O—(CH₂)_{q}-heterocyclyl, —C(O)—O—(CH₂)_{q}-heteroaryl, —OC(O)—C₁-C₁₀ alkyl, —O—C(O)—(CH₂)_{q}—C₃-C₇ cycloalkyl, —O—C(O)—(CH₂)_{q}-aryl, —O—C(O)—(CH₂)ₚ-heterocyclyl, and —O—C(O)—(CH₂)_{q}-heteroaryl, wherein any alkyl, cycloalkyl, aryl, heterocyclyl, and heteroaryl optionally are substituted; and wherein R⁴ together with A3 optionally may form a heterocyclic ring together with the nitrogen to which A3 is attached, or R⁵ together with R² optionally may form a heterocyclic ring together with the nitrogen to which R² is attached, wherein any heterocyclic ring optionally is substituted;
R⁶ and R⁷ are each independently selected from the group consisting of H, —NH—C₁-C₆ alkyl, C₁-C₆ alkyl, C₃-C₁₀ cycloalkyl, aryl, heterocyclyl, heteroaryl, —NH—(CH₂)ₚ—Z₃, —N(—(CH₂)ₚ—Z₃)(—(CH₂)ₚ—Z₃), —O—(CH₂)ₚ—Z₃, —CH₂—NH—(CH₂)ₚ—Z₃, —CH₂—O—(CH₂)ₚ—Z₃, —(CH₂)₂—NH—(CH₂)ₚ—Z₃, —(CH₂)₂—O—(CH₂)ₚ—Z₃, and —(CH₂)ₚ—Z₃, wherein any alkyl, cycloalkyl, aryl, heterocyclyl, and heteroaryl optionally are substituted;
Z₃ is selected from the group consisting of H, halogen, hydroxyl, —NH₂, CN, NO₂, C₁-C₆ alkoxy, C₃-C₁₀ cycloalkyl, aryl, heterocyclyl, heteroaryl, —O—C₁-C₆ alkyl, —O—(CH₂)ᵣ—C₃-C₁₀ cycloalkyl, —O—(CH₂)ᵣ-aryl, —O—(CH₂)ᵣ-heterocyclyl, —O—(CH₂)ᵣ-heteroaryl, —C(O)—C₁-C₆ alkyl, —C(O)—(CH₂)ᵣ—C₃-C₇ cycloalkyl, —C(O)—(CH₂)ᵣ-aryl, —C(O)—(CH₂)ᵣ-heterocyclyl, —C(O)—(CH₂)ᵣ-heteroaryl, —S(O)—C₁-C₆ alkyl, —S(O)—(CH₂)ᵣ—C₃-C₇ cycloalkyl, —S(O)—(CH₂)ᵣ-aryl, —S(O)—(CH₂)ᵣ-heterocyclyl, —S(O)—(CH₂)ᵣ-heteroaryl, —SO₂—C₁-C₆ alkyl, —SO₂—(CH₂)ᵣ—C₃-C₇ cycloalkyl, —SO₂—(CH₂)ᵣ-aryl, —SO₂—(CH₂)ᵣ-heterocyclyl, —SO₂—(CH₂)ᵣ-heteroaryl, —NH(R⁹), —N(R⁹)—SO₂—C₁-C₆ alkyl, —N(R⁹)—SO₂—(CH₂), —C₃-C₇ cycloalkyl, —N(R⁹)—SO₂—(CH₂)ᵣ-aryl, —N(R⁹)—SO₂—(CH₂)ᵣ-heterocyclyl, —N(R⁹)—SO₂—(CH₂)ᵣ-heteroaryl, —SO₂—N(R¹⁰)(R¹¹), —N(R⁹)—C(O)—C₁-C₆ alkyl, —N(R⁹)—C(O)—(CH₂)ᵣ—C₃-C₇ cycloalkyl, —N(R⁹)—C(O)—(CH₂)ᵣ-aryl, —N(R⁹)—C(O)—(CH₂)ᵣ-heterocyclyl, —N(R⁹)—C(O)—(CH₂)ᵣ-heteroaryl, —N(R¹⁰)(R¹¹), —C(O)—N(R¹⁰)((R¹¹), —C(O)—O—C₁-C₆ alkyl, —C(O)—O—(CH₂)ᵣ—C₃-C₇ cycloalkyl, —C(O)—O—(CH₂)ᵣ-aryl, —C(O)—O—(CH₂)ᵣ-heterocyclyl, —C(O)—O—(CH₂)— heteroaryl, —OC(O)—C₁-C₁₀ alkyl, —O—C(O)—(CH₂)ᵣ—C₃-C₇ cycloalkyl, —O—C(O)—(CH₂)ᵣ-aryl, —O—C(O)—(CH₂)ᵣ-heterocyclyl, and —O—C(O)—(CH₂)ᵣ-heteroaryl, wherein any alkyl, cycloalkyl, aryl, heterocyclyl, and heteroaryl optionally are substituted;
R⁸ is selected from the group consisting of C₃-C₁₀ cycloalkyl, aryl, heterocyclyl, heteroaryl, aryl-C₁-C₆ alkyl, C₃-C₁₀ cycloalkyl-aryl, aryl-C₃-C₁₀ cycloalkyl, C₃-C₁₀ cycloalkyl-heterocyclyl, heterocyclyl-C₃-C₁₀ cycloalkyl, C₃-C₁₀ cycloalkyl-heteroaryl, heteroaryl-C₃-C₁₀ cycloalkyl, aryl-heterocyclyl, heterocyclyl-aryl, aryl-heteroaryl, heteroaryl-aryl, heterocyclyl-heteroaryl, heteroaryl-heterocyclyl, C₃-C₁₀ cycloalkyl-O-aryl, aryl-O—C₃-C₁₀ cycloalkyl, C₃-C₁₀ cycloalkyl-O-heterocyclyl, heterocyclyl-O—C₃-C₁₀ cycloalkyl, C₃-C₁₀ cycloalkyl-O-heteroaryl, heteroaryl-O—C₃-C₁₀ cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C₃-C₁₀ cycloalkyl-C(O)-aryl, aryl-C(O)—C₃-C₁₀ cycloalkyl, C₃-C₁₀ cycloalkyl-C(O)-heterocyclyl, heterocyclyl-C(O)—C₃-C₁₀ cycloalkyl, C₃-C₁₀ cycloalkyl-C(O)-heteroaryl, heteroaryl-C(O)—C₃-C₁₀ cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C₃-C₁₀ cycloalkyl-CH₂-aryl, aryl-CH₂—C₃-C₁₀ cycloalkyl, C₃-C₁₀ cycloalkyl-CH₂-heterocyclyl, heterocyclyl-CH₂—C₃-C₁₀ cycloalkyl, C₃-C₁₀ cycloalkyl-CH₂-heteroaryl, heteroaryl-CH₂—C₃-C₁₀ cycloalkyl, aryl-CH₂-heterocyclyl, heterocyclyl-CH₂-aryl, aryl-CH₂-heteroaryl, heteroaryl-CH₂-aryl, heterocyclyl-CH₂-heteroaryl, heteroaryl-CH₂-heterocyclyl, C₃-C₁₀ cycloalkyl-CH₂CH₂-aryl, aryl-CH₂CH₂—C₃-C₁₀ cycloalkyl, C₃-C₁₀ cycloalkyl-CH₂CH₂-heterocyclyl, heterocyclyl-CH₂CH₂—C₃-C₁₀ cycloalkyl, C₃-C₁₀ cycloalkyl-CH₂CH₂-heteroaryl, heteroaryl-CH₂CH₂—C₃-C₁₀ cycloalkyl, aryl-CH₂CH₂-heterocyclyl, heterocyclyl-CH₂CH₂-aryl, aryl-CH₂CH₂-heteroaryl, heteroaryl-CH₂CH₂-aryl, heterocyclyl-CH₂CH₂-heteroaryl, heteroaryl-CH₂CH₂-heterocyclyl, C₃-C₁₀ cycloalkyl-NH-aryl, aryl-NH—C₃-C₁₀ cycloalkyl, C₃-C₁₀ cycloalkyl-NH-heterocyclyl, heterocyclyl-NH—C₃-C₁₀ cycloalkyl, C₃-C₁₀ cycloalkyl-NH-heteroaryl, heteroaryl-NH—C₃-C₁₀ cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C₃-C₁₀ cycloalkyl-N(Me)-aryl, aryl-N(Me)-C₃-C₁₀ cycloalkyl, C₃-C₁₀ cycloalkyl-N(Me)-heterocyclyl, heterocyclyl-N(Me)-C₃-C₁₀ cycloalkyl, C₃-C₁₀ cycloalkyl-N(Me)-heteroaryl, heteroaryl-N(Me)-C₃-C₁₀ cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C₃-C₁₀ cycloalkyl-NHC(O)-aryl, aryl-NHC(O)—C₃-C₁₀ cycloalkyl, C₃-C₁₀ cycloalkyl-NHC(O)-heterocyclyl, heterocyclyl-NHC(O)—C₃-C₁₀ cycloalkyl, C₃-C₁₀ cycloalkyl-NHC(O)-heteroaryl, heteroaryl-NHC(O)—C₃-C₁₀ cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C₃-C₁₀ cycloalkyl-C(O)NH-aryl, aryl-C(O)NH—C₃-C₁₀ cycloalkyl, C₃-C₁₀ cycloalkyl-C(O)NH-heterocyclyl, heterocyclyl-C(O)NH—C₃-C₁₀ cycloalkyl, C₃-C₁₀ cycloalkyl-C(O)NH-heteroaryl, heteroaryl-C(O)NH—C₃-C₁₀ cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C₃-C₁₀ cycloalkyl-NHC(O)NH-aryl, aryl-NHC(O)NH—C₃-C₁₀ cycloalkyl, C₃-C₁₀ cycloalkyl-NHC(O)NH-heterocyclyl, heterocyclyl-NHC(O)NH—C₃-C₁₀ cycloalkyl, C₃-C₁₀ cycloalkyl-NHC(O)NH-heteroaryl, heteroaryl-NHC(O)NH—C₃-C₁₀ cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted;
R⁹ is selected from the group consisting of H, C₁-C₆ alkyl, trifluoromethyl, trifluoroethyl, C₁-C₆ alkoxy, halogen-C₁-C₆ alkyl, —(CH₂)₀₋₂-aryl, —(CH₂)₀₋₂-heterocyclyl, and —(CH₂)₀₋₂-heteroaryl;
R¹⁰ and R¹¹ are each independently selected from the group consisting of H, C₁-C₄ alkyl, C₃-C₇ cycloalkyl, aryl, —(CH₂)₁₋₆—C₃-C₇ cycloalkyl, —(CH₂)₁₋₆-aryl, wherein alkyl, cycloalkyl, and aryl optionally are substituted, or R¹⁰ together with R¹¹ may form a heterocyclyl ring together with the nitrogen to which they are attached;
m is 0 or an integer from 1 to 5;
n is 0 or an integer from 1 to 6;
p is 0 or an integer from 1 to 6;
q is 0 or an integer from 1 to 6;
r is 0 or an integer from 1 to 6; and
with the proviso that when A₁ is a single bond, A₂ is an oxazol ring, B is a pyrrolidinyl, R¹ and R² is H, R³ is selected from H or methyl, R⁴ and R⁵ is selected from H or methyl, and R⁸ is phenyl, 4-hydroxy-1-phenyl, or 3-indolyl, then at least one of R⁶ and R⁷ is different from H.

2. The compound according to claim 1, wherein A2 is selected from the group consisting of cyclopropyl, cyclobutyl, cyclopentyl, cyclohexyl, azetidinyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azetidinonyl, azepanylyl, oxazetidinyl, diazetidinyl, oxazepanylyl, diazepanylyl, pyrrolidinonyl, piperidinonyl, azepanylonyl, thioxoazetidinyl, phenyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, 5,6-dihydro-4H-cyclopenta[c]furanyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

3-19. (canceled)

20. The compound according to claim 1, wherein A4 is selected from the group consisting of —CH₂—, —C(O)—, —NH—, —O—, —S—, and —SO₂—.

21-23. (canceled)

24. The compound according to claim 1, wherein B is selected from the group consisting of 5 membered and 6 membered heterocyclic and heteroaromatic rings.

25. The compound according to claim 1, wherein B is selected from the group consisting of pyrrolidine, pyrazolidine, imidazolidine, isoxazolidine, 1,3-oxazolidine, isothiazolidine, 1,3-thiazolidine, 2,3-dihydro-1H-pyrrole, 2,5-dihydro-1H-pyrrole, 2,5-dihydroisoxazole, 2,3-dihydro-1,3-oxazole, 2,5-dihydroisothiazole, 2,3-dihydro-1,3-thiazole, 2,3-dihydroisoxazole, 2,3-dihydroisothiazole, piperidine, hexahydropyridazine, hexahydropyrimidine, piperazine, 1,2-oxazinane, 1,3-oxazinane, morpholine, 1,2-thiazinane, 1,3-thiazinane, thiomorpholine, 1,2,3,4-tetrahydropyridine, 1,2,3,6-tetrahydropyridine, 1,2,3,6-tetrahydropyridine, 1,2-dihydropyridine, 1,4-dihydropyridine, 1,2,3,4-tetrahydropyridazine, 1,2,3,4-tetrahydropyrimidine, 1,2,3,4-tetrahydropyrazine, 5,6-dihydro-2H-1,2-oxazine, 3,6-dihydro-2H-1,3-oxazine, 3,4-dihydro-2H-1,4-oxazine, 5,6-dihydro-2H-1,2-thiazine, 3,6-dihydro-2H-1,3-thiazine, 3,4-dihydro-2H-1,4-thiazine, 3,6-dihydro-2H-1,2-oxazine, 3,4-dihydro-2H-1,3-oxazine, 3,4-dihydro-2H-1,2-oxazine, 1,2-dihydropyridine, 1,4-dihydropyridine, tetrahydropyrimidin-4(1H)-one, piperazin-2-one, 1,3,5-triazinan-2-one, piperidin-4-one, and piperidin-3-one.

26. (canceled)

27. The compound according to claim 1, wherein B is selected from the group consisting of azetidin-1-yl, pyrrolidin-1-yl, piperidin-1-yl, 2-oxo-piperazinyl, morpholin-4-yl, and piperazin-1-yl.

28-32. (canceled)

33. The compound according to claim 1, wherein R¹ is selected from the group consisting of H, C₁-C₆ alkyl, C₃-C₁₀ cycloalkyl, aryl, heterocyclyl, and heteroaryl, wherein any alkyl, cycloalkyl, aryl, heterocyclyl, and heteroaryl optionally are substituted.

34-36. (canceled)

37. The compound according to claim 1, wherein R² is selected from the group consisting of H, C₁-C₄ alkyl, C₃-C₆ cycloalkyl, —(CH₂)₁₋₄-cycloalkyl, wherein any alkyl, cycloalkyl, optionally are substituted; or wherein R² together with R⁵ optionally may form a heterocyclic ring together with the nitrogen to which R² is attached, or wherein R² together with R⁵ forms a heterocyclic ring together with the nitrogen to which R² is a single bond, and wherein the heterocyclic ring optionally is substituted.

38-45. (canceled)

46. The compound according to claim 1, wherein R² together with R⁵ forms a heterocyclic ring selected from the group consisting of azetidinyl, pyrrolidinyl, and piperidinyl, and wherein the ring optionally is substituted.

47. (canceled)

48. The compound according to claim 1, wherein R³ is selected from the group consisting of H, hydroxy, and C₁-C₄ alkyl.

49-62. (canceled)

63. The compound according to claim 1, wherein R⁴ and R⁵ each independently are selected from the group consisting of H, methyl, ethyl, methoxy, and ethoxy.

64-88. (canceled)

89. The compound according to claim 1, wherein R⁶ and R⁷ each independently is phenyl optionally substituted with one to three substitutents selected from the group consisting of hydroxy, fluoro, chloro, bromo, iodo, methoxy, and ethoxy.

90-104. (canceled)

105. The compound according to claim 1, wherein R⁸ is selected from the group consisting of phenyl, phenylcyclopentyl, phenylpyrrolidine, benzylpyrrolidine, phenoxypyrrolidine, and phenylaminopyrrolidine.

106-107. (canceled)

108. The compound according to claim 1, wherein R⁸ is substituted with one or more substituents selected from the group consisting of fluoro, chloro, hydroxy, methoxy, ethoxy, methyl, ethyl, propyl, isopropyl, tert-butyl, sec-butyl, cyano, nitro, sulfanyl, methylsulfanyl, sulfonyl, and methylsulfonyl.

109-120. (canceled)

121. The compound according to claim 1, having formula (IIa) 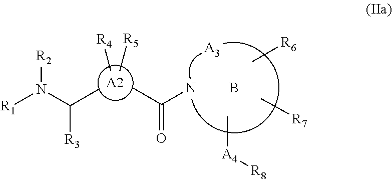
wherein R1, R2, R3, R4, R5, R6, R7, R8, A1, A2, A3, and A4 are as defined in claim 1 or a compound according to claim 1, having formula (IIIa) 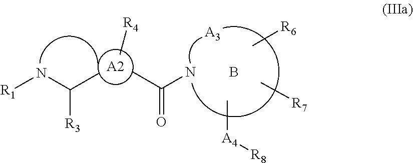
wherein R1, R2, R3, R4, R5, R6, R7, R8, A1, A2, A3, and A4 are as defined in claim 1.

122. (canceled)

123. The compound according to claim 1, wherein the compound is selected from the group consisting of
(5-(1-aminoethyl)furan-2-yl)((2S,4R)-4-phenyl-2-(((R)-3-phenylpyrrolidin-1-yl)methyl)pyrrolidin-1-yl)methanone;
[5-(1-Amino-ethyl)-furan-2-yl]-[(2S,4R)-4-phenyl-2-((R)-3-phenyl-pyrrolidine-1-carbonyl)-pyrrolidin-1-yl]-methanone;
[3-(1-Amino-ethyl)-phenyl]-[(2S,4R)-4-phenyl-2-((R)-3-phenyl-pyrrolidine-1-carbonyl)-pyrrolidin-1-yl]-methanone;
[6-((R)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[6-((S)-1-Amino-ethyl)-piperidin-2-yl]-[(2S,4R)-4-phenyl-2-((R)-3-phenyl-pyrrolidine-1-carbonyl)-pyrrolidin-1-yl]-methanone;
[5-(1-Methylamino-ethyl)-furan-2-yl]-[(2S,4R)-4-phenyl-2-((R)-3-phenyl-pyrrolidine-1-carbonyl)-pyrrolidin-1-yl]-methanone;
[3-(1-Methylamino-ethyl)-phenyl]-[(2S,4R)-4-phenyl-2-((R)-3-phenyl-pyrrolidine-1-carbonyl)-pyrrolidin-1-yl]-methanone;
[6-(1-Methylamino-ethyl)-pyridin-2-yl]-[(2S,4R)-4-phenyl-2-((R)-3-phenyl-pyrrolidine-1-carbonyl)-pyrrolidin-1-yl]-methanone;
{(2S,4R)-4-(4-Fluoro-phenyl)-2-[3-(4-fluoro-phenyl)-pyrrolidine-1-carbonyl]-pyrrolidin-1-yl}-[5-(1-methylamino-ethyl)-furan-2-yl]-methanone;
(5-(1-(methylamino)ethyl)furan-2-yl)((2S,4R)-4-phenyl-2-(((R)-3-phenylpyrrolidin-1-yl)methyl)pyrrolidin-1-yl)methanone;
(3-(1-(methylamino)ethyl)phenyl)((2S,4R)-4-phenyl-2-(((R)-3-phenylpyrrolidin-1-yl)methyl)pyrrolidin-1-yl)methanone;
(6-(1-(methylamino)ethyl)pyridin-2-yl)((2S,4R)-4-phenyl-2-(((R)-3-phenylpyrrolidin-1-yl)methyl)pyrrolidin-1-yl)methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(S)-methylamino-ethyl)-furan-2-yl]-methanone;
{(2S,4R)-4-(4-Fluoro-phenyl)-2-[3(R)-(4-fluoro-phenyl)-pyrrolidine-1-carbonyl]-pyrrolidin-1-yl}-[5-(1(R)-methylamino-ethyl)-furan-2-yl]-methanone;
(5-(1(S)-aminoethyl)furan-2-yl)((2S,4R)-4-phenyl-2-(((R)-3-phenylpyrrolidin-1-yl)methyl)pyrrolidin-1-yl)methanone;
(5-(1(R)-amino-ethyl)furan-2-yl)((2S,4R)-4-phenyl-2-(((R)-3-phenylpyrrolidin-1-yl)methyl)pyrrolidin-1-yl)methanone;
(3-(1(S)-(methylamino)ethyl)phenyl)((2S,4R)-4-phenyl-2-(((R)-3-phenylpyrrolidin-1-yl)methyl)pyrrolidin-1-yl)methanone;
(3-(1(R)-(methylamino)ethyl)phenyl)((2S,4R)-4-phenyl-2-(((R)-3-phenylpyrrolidin-1-yl)methyl)pyrrolidin-1-yl)methanone;
(2S,4S)-4-Cyclohexyl-1-(2,8-diaza-spiro[4.5]decane-3-carbonyl)-pyrrolidine-2-carboxylicacid(R)-indan-1-ylamide;
(2R,4R)-4-Cyclohexyl-1-(2,8-diaza-spiro[4.5]decane-3-carbonyl)-pyrrolidine-2-carboxylicacid(S)-indan(R)-1-ylamide; and
(2R,4R)-4-Cyclohexyl-1-(2,8-diaza-spiro[4.5]decane-3-carbonyl)-pyrrolidine-2-carboxylicacid(R)-indan(R)-1-ylamide.

124-127. (canceled)

128. A polymeric compound of formula (VI)
Y-(L)ₘ-[Y-(L)ₘ]ₙ-Y (VI)
or a pharmaceutically acceptable salt, solvate or prodrug thereof,
wherein
Y is a monomeric unit of formula (I), wherein the first and the second or further monomeric units are the same or different and independently are selected from the compounds as defined in claim 1;
L is the same or different and is a covalent linker, linking any part of one monomeric unit of formula (I), to any part of a second or further monomeric unit of formula (I);
m is an integer of 1 to 4; and
n is an integer of 0 to 5.

129-130. (canceled)

131. A compound of formula (VII)
Z-Lₘ-E (VII)
or a pharmaceutically acceptable salt, solvate or prodrug thereof,
wherein
Z is a compound of formula (I) as defined in claim 1 or a polymeric compound of formula (VI) as defined in claim 128;
L is a linker linking any part of Z to any part of E;
E is an entity selected from the group consisting of an affinity tag, such as e.g. a hexahistidine tag or biotin, a dye, such as e.g. fluorescein, an oligonucleotide, a protein, such as e.g. an antibody or biotin-binding protein, and a solid support; and
m is an integer of 1 to 4.

132-133. (canceled)

134. A compound as defined in claim 1 for use as a medicament.

135. A compound as defined in claim 1 for treating proliferative diseases, for promoting apoptosis in proliferating cells, or for sensitizing cells to inducers of apoptosis.

136-144. (canceled)

145. A pharmaceutical composition comprising a compound as defined in claim 1, and optionally one or more pharmaceutically acceptable excipients, diluents or carriers.

146. The pharmaceutical composition according to claim 145, further comprising one or more additional active substances wherein the one or more additional active substances are selected from anticancer agents, antineoplastic agents, cytotoxic drugs, and anti-tumor antibiotics or from protease inhibitors, epidermal growth factor receptor kinase inhibitors, vascular epidermal growth factor receptor kinase inhibitors, antimetabolites, antimitotic agents, platinum coordination complexes, anti-tumor antibiotics, alkylating agents, and endocrine agents.

147-148. (canceled)

149. A method of treating a proliferative disease in a subject, said method comprises administering to said subject a therapeutically effective amount of a compound as defined in claim 1, or a pharmaceutical composition as defined in claim 145, to a subject in need of such treatment.

150. The method according to claim 149, wherein the compound is administered in combination with one or more additional active substances, and where the one or more additional active substances are optionally selected from anticancer agents, antineoplastic agents, cytotoxic drugs, and anti-tumor antibiotics, or from protease inhibitors, epidermal growth factor receptor kinase inhibitors, vascular epidermal growth factor receptor kinase inhibitors, antimetabolites, antimitotic agents, platinum coordination complexes, anti-tumor antibiotics, alkylating agents, and endocrine agents.

151-152. (canceled)

153. The method according to claim 150, wherein the subject is a mammal.

154. The method of claim 153 wherein said mammal is a human being.
